(19) United States
(12) Patent Application Publication
(10) Pub. No.: US 2025/0137021 A1
(43) Pub. Date: May 1, 2025

(54) METHODS OF PRODUCING NOR-OPIOID AND NAL-OPIOID BENZYLISOQUINOLINE ALKALOIDS

(71) Applicant: Antheia, Inc., Menlo Park, CA (US)

(72) Inventors: Christina D. SMOLKE, Menlo Park, CA (US); Catherine THODEY, Mountain View, CA (US); Isis TRENCHARD, Menlo Park, CA (US)

(21) Appl. No.: 18/962,453

(22) Filed: Nov. 27, 2024

Related U.S. Application Data
(63) Continuation of application No. 17/846,935, filed on Jun. 22, 2022, now Pat. No. 12,180,525.
Application No. 17/846,935 is a continuation of application No. 16/918,529, filed on Jul. 1, 2020, now Pat. No. 11,396,668.
Application No. 16/918,529 is a continuation of application No. 16/127,084, filed on Sep. 10, 2018, now Pat. No. 10,738,335.
Application No. 16/127,084 is a continuation of application No. PCT/US2017/057237, filed on Oct. 18, 2017.
(60) Provisional application No. 62/409,837, filed on Oct. 18, 2016.
(60) Provisional application No. 62/473,215, filed on Mar. 17, 2017.

(51) Int. Cl. C12P 17/18; C12N 9/02; C12N 9/04; C12N 9/06; C12N 9/14; C12N 9/90; C12N 15/85; C12P 17/12

(52) U.S. Cl. CPC C12P 17/182; C12N 9/0006; C12N 9/0028; C12N 9/0071; C12N 9/14; C12N 9/90; C12N 15/85; C12P 17/12; C12P 17/18; C12Y 105/01027; C12Y 114/16002; C12Y 114/21004; C12N 2015/8518; C12Y 305/04016

(57) ABSTRACT

A method of demethylizing an opioid to a nor-opioid is provided. The method comprises contacting an opioid with at least one enzyme. Contacting the opioid with the at least one enzyme converts the opioid to a nor-opioid. A method of converting a nor-opioid to a nal-opioid is provided. The method comprises contacting a nor-opioid with at least one enzyme. Contacting the nor-opioid with the at least one enzyme converts the nor-opioid to a nal-opioid.

### CROSS-REFERENCE

This application is a continuation of U.S. application Ser. No. 17/846,935, filed Jun. 22, 2022, which is a continuation of U.S. application Ser. No. 16/918,529, filed on Jul. 1, 2020, which is a continuation of U.S. application Ser. No. 16/127,084, filed Sep. 10, 2018, now U.S. Pat. No. 10,738,335, which is a continuation application of International Patent Application No. PCT/US2017/057237, filed Oct. 18, 2017, which application claims the benefit of U.S. Provisional Application No. 62/409,837, filed Oct. 18, 2016, and U.S. Provisional Application No. 62/473,215, filed Mar. 17, 2017, which applications are incorporated herein by reference.

### SEQUENCE LISTING

The instant application contains a Sequence Listing which has been submitted electronically in ASCII format and is hereby incorporated by reference in its entirety. The contents of the electronic sequence listing 2025 Jan. 21 SL_ST26 062074-506C04US.xml; Size: 199,393 bytes; and Date of Creation: Jan. 21, 2025.

### BACKGROUND OF THE INVENTION

Medicinal opioids are used for treating moderate to severe pain, but may exhibit addictive properties. Due to the mechanism by which medicinal opioids relieve pain, these medications are among the most effective painkillers in modern medicine. Additionally, however, medicinal opioids are also widely abused. In addressing the use of medicinal opioids, policy makers are tasked with balancing the under-treatment of pain, while mitigating the risk for opioid abuse. Pharmacotherapies have proven effective in treating and preventing opioid addiction but the high cost of these therapeutics is a limiting factor in the scope and reach of treatment programs.

### SUMMARY OF THE INVENTION

The present disclosure provides methods for demethylating a first opioid to a second opioid. The present disclosure further provides methods for demethylating an opioid to a nor-opioid. Additionally, the present disclosure provides methods for altering an opioid to a nal-opioid. Further, the present disclosure provides engineered cells for producing a nor-opioid from an opioid present within the engineered cell. The present disclosure also provides engineered cells for producing a nal-opioid from a nor-opioid present within the engineered cell.

An aspect of the invention provides a method for demethylating a first opioid to a second opioid. The method comprises contacting the first opioid with at least one enzyme, wherein contacting the first opioid with the at least one enzyme converts the first opioid to a second opioid through loss of an O-linked methyl group, wherein the first opioid is not selected from the group consisting of codeine and thebaine.

Another aspect of the invention provides a method of demethylating an opioid to a nor-opioid. The method comprises contacting the first opioid with at least one enzyme, wherein contacting the first opioid with the at least one enzyme converts the first opioid to a second opioid through loss of an O-linked methyl group. The method also comprises contacting the second opioid with at least one enzyme, wherein contacting the opioid with the at least one enzyme converts the second opioid to a nor-opioid through loss of an N-linked methyl group.

An additional aspect of the invention provides another method of demethylating an opioid to a nor-opioid. The method comprises contacting the opioid with at least one enzyme, wherein contacting the opioid with the at least one enzyme converts the opioid to a nor-opioid through removal of an N-linked methyl group from the opioid, wherein the opioid is not thebaine when the opioid contacts the at least one enzyme in vitro.

A further aspect of the invention provides a method of altering an opioid to a nal-opioid. The method comprises contacting the opioid with at least a first enzyme, wherein contacting the opioid with the at least a first enzyme converts the opioid to a nor-opioid through removal of an N-linked methyl group from the opioid. The method also comprises contacting the nor-opioid with at least a second enzyme, wherein contacting the nor-opioid with the at least a second enzyme in the presence of a cofactor converts the nor-opioid to a nal-opioid through transfer of a sidechain from the cofactor.

Another aspect of the invention provides another method of altering an opioid to a nal-opioid. The method comprises contacting the first opioid with at least one enzyme, wherein contacting the first opioid with the at least one enzyme converts the first opioid to a second opioid through loss of an O-linked methyl group. The method also comprises contacting the second opioid with at least a second enzyme, wherein contacting the opioid with the at least a second enzyme converts the second opioid to a nor-opioid through loss of an N-linked methyl group. Additionally, the method comprises contacting the nor-opioid with at least a third enzyme, wherein contacting the nor-opioid with the at least a third enzyme in the presence of a cofactor converts the nor-opioid to a nal-opioid through transfer of a sidechain from the cofactor.

An additional aspect of the invention provides an engineered cell that produces a nor-opioid from an opioid present within the engineered cell, the engineered cell comprising a heterologous coding sequence encoding an N-demethylase produced by the engineered cell, wherein the N-demethylase converts the opioid within the engineered cell to the nor-opioid and wherein the nor-opioid is produced within the engineered cell.

A further aspect of the invention provides an engineered cell that produces a nal-opioid from a nor-opioid present within the engineered cell, the engineered cell comprising a heterologous coding sequence encoding an N-methyltransferase produced by the engineered cell, wherein the N-methyltransferase converts the nor-opioid within the engineered cell to the nal-opioid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the invention will be obtained by reference to the following detailed description that sets forth illustrative embodiments, in which the principles of the invention are utilized, and the accompanying drawings of which:

FIG. 1 illustrates examples of synthesis, recycling, and salvage pathways of tetrahydrobiopterin, in accordance with embodiments of the invention.

FIG. 2 illustrates a biosynthetic scheme for conversion of glucose to 4-HPA, dopamine, and 3,4-DHPA, in accordance with embodiments of the invention.

FIG. 3 illustrates a schematic example of (R)-1-benzylisoquinoline alkaloid formation, in accordance with embodiments of the invention.

FIG. 4 illustrates an amino acid sequence of a CYP—COR enzyme (SEQ ID NO: 1), in accordance with embodiments of the invention.

FIG. 5 illustrates a biosynthetic scheme for conversion of L-tyrosine to reticuline via norcoclaurine, in accordance with embodiments of the invention.

FIG. 6 illustrates a biosynthetic scheme for conversion of L-tyrosine to reticuline via norlaudanosoline, in accordance with embodiments of the invention.

FIG. 7 illustrates a biosynthetic scheme for conversion of L-tyrosine to morphinan alkaloids, in accordance with embodiments of the invention.

FIG. 8 illustrates a biosynthetic scheme for production of semi-synthetic opioids, in accordance with embodiments of the invention.

FIG. 9 illustrates tyrosine hydroxylase mutants that improve reticuline production from sugar in engineered yeast strains, in accordance with embodiments of the invention.

FIG. 10 illustrates coexpression of dihydrofolate reductase (DHFR) that improves L-DOPA production by tyrosine hydroxylase in engineered yeast strains, in accordance with embodiments of the invention.

FIG. 11A illustrates the addition of antioxidants to culture media that improves L-DOPA production by tyrosine hydroxylase in engineered yeast strains.

FIG. 11B illustrates the addition of antioxidants to culture media that increase BH₄ levels, in accordance with embodiments of the invention.

FIG. 12A illustrates a biosynthetic scheme for conversion of L-tyrosine to bisBIAs.

FIG. 12B illustrates yeast strains engineered to biosynthesize bisBIAs, in accordance with embodiments of the invention.

FIG. 13 illustrates a phylogenetic tree of cytochrome P450 oxidase-codeinone reductase-like (CYP—COR) fusions, in accordance with embodiments of the invention.

FIGS. 14A and 14B illustrate an LC/MS-MS analysis of yeast strains engineered to convert (S)-reticuline to salutaridine, in accordance with embodiments of the invention. FIG. 14A illustrates chromatogram traces showing reticuline and salutaridine for two epimerase variants (CYP-COR_89405, CYP-COR_4328) and a standard. FIG. 14B illustrates the same chromatogram traces for salutaridine in FIG. 14A as replotted to demonstrate co-elution with the standard.

FIG. 15 illustrates a chiral LC/MS-MS analysis of yeast strains engineered to convert racemic norlaudanosoline to (R)-reticuline, in accordance with embodiments of the invention.

FIG. 16A illustrates N-linked glycosylation status of heterologously expressed salutarideine synthase.

FIG. 16B illustrates engineered fusions of salutaridine synthase that eliminates N-linked glycosylation of the protein observed when heterologously expressed in yeast but not plants, in accordance with embodiments of the invention

FIG. 17A and FIG. 17B illustrate cheilanthifoline synthase-salutaridine synthase fusion designs (SEQ ID NOS 117, and 115-116, respectively, in order of appearance), in accordance with embodiments of the invention.

FIG. 18 illustrates salutaridine synthase codon-optimization and engineered fusions that improve activity in yeast, in accordance with embodiments of the invention.

FIG. 19A and FIG. 19B illustrate LC/MS-MS analyses of small scale batch fermentation in which engineered yeast catalyze the conversion of (R)-reticuline to thebaine and the conversion of rac-norlaudanosoline to thebaine, in accordance with embodiments of the invention.

FIG. 20 illustrates generation of a CODM enzyme variant exhibiting enhanced activity in yeast through random mutagenesis and screening, in accordance embodiments of the invention.

FIG. 21A, FIG. 21B, and FIG. 21C illustrate fermentation optimization for conversion of (R)-reticuline to thebaine by engineered yeast, in accordance with embodiments of the invention.

FIG. 22 illustrates yeast platform strains for the production of the key branch point intermediate reticuline from L-tyrosine, in accordance with embodiments of the invention.

FIG. 23 illustrates an enzyme having opioid 3-O-demethylase activity, in accordance with embodiments of the invention.

FIG. 24 illustrates an enzyme having opioid N-demethylase activity, in accordance with embodiments of the invention.

FIG. 25 illustrates an enzyme having N-methyltransferase activity, in accordance with embodiments of the invention.

FIG. 26 illustrates a biosynthesis scheme in a microbial cell, in accordance with embodiments of the invention.

FIG. 27A, FIG. 27B, and FIG. 27C illustrates the functional expression of BM3 variants, in accordance with embodiments of the invention.

FIG. 28 illustrates plasmid/YAC vectors for enzyme expression and engineering, in accordance with embodiments of the invention.

FIG. 29 illustrates the functional expression of CODM, in accordance with embodiments of the invention.

FIG. 30 illustrates a biosynthetic scheme for production of semi-synthetic opioids, in accordance with embodiments of the invention.

### DETAILED DESCRIPTION OF THE INVENTION

Nal-opioids are an important class of pharmacotherapies for combating opioid addiction and opioid-associated side effects. Opiates like codeine and morphine are plant molecules from opium poppy, which have a unique five-ring structure that allows them to bind μ-opioid receptors in the brain, spine, gut, and peripheral sensors and mimic the body's natural attenuation of pain. These same opiate molecules can be modified to act as antagonists, e.g., naltrexone and naloxone. In some examples opioid molecules can be modified by introducing chemical modifications that permit binding to opioid receptors but prevent activation of the downstream signaling response. Other molecules, such as buprenorphine, may bind to opioid receptors and act as mixed partial agonists.

The suite of antagonist and mixed partial-agonist opioids, collectively called nal-opioids, may form a toolkit of competitive modulators that can occupy receptor binding sites in patients that have ingested a strong opioid agonist. As examples, in the addicted patient population nal-opioids may be used for: (1) treating overdose, by administering a strong antagonist, such as naloxone; (2) detoxification, by managing symptoms with mixed partial agonists, such as buprenorphine; and/or (3) maintenance, by blocking the reward response, for example with a buprenorphine/naloxone combination drug. In the patient population with severe pain, nal-opioids may be used for: (4) prevention, e.g. through abuse deterrent combination agonist/antagonist formulations, such as morphine/naltrexone combinations, which may block euphoria and intoxication when the drug is misused; and/or (5) reducing side effects by administering peripherally acting antagonists that may displace opioid agonists from receptors in the gut where they may cause constipation, for example, a polymer conjugate of naloxone (Movantik™).

The raw starting materials for nal-opioid synthesis are natural opiates, such as thebaine, that are extracted from opium poppy drug crops. Traditionally, these molecules are then chemically modified to the semi-synthetic antagonists, weak agonists, and mixed partial agonists through a series of inefficient reaction steps that require the use of catalysts, solvents, reagents, and purification methods to isolate the nal-opioid product from the starting material and reaction intermediates. The current semi-synthetic production methods are inefficient and add substantial cost to the overall process.

The present disclosure provides methods for the production of diverse nal-opioids in engineered host cells. The present disclosure also provides methods for the production of diverse nor-opioids in engineered host cells. Additionally, the present disclosure provides methods for the production of an O-demethylase and an N-demethylase in engineered host cells. In particular cases, the disclosure provides methods for producing nor-opioid products through the demethylization of an opioid to a nor-opioid in an engineered host cell. In further particular cases, the present disclosure provides methods for producing diverse nal-opioids by modifying a nor-opioid with an enzyme that can add an N-linked side chain, such as an N-methyltransferase.

The present disclosure provides methods for the production of nal-opioids and nor-opioid compounds in engineered host cells. Throughout this disclosure the term “compound” may be used to refer to something comprising two or more elements, for example a nal-opioid molecule, or a nal-opioid composition. A nal-opioid compound may refer to a largely pure composition of a nal-opioid, or a composition of a nal-opioid which may or may not contain impurities.

### Nal-Opioids of Interest

Host cells which produce BIAs of interest are provided. In some examples, engineered strains of host cells such as the engineered strains of embodiments discussed herein may provide a platform for producing Nal-opioids of interest including, but not limited to: naltrexone, naloxone, nalmefene, nalorphine, nalorphine, nalodeine, naldemedine, naloxegol, 6β-naltrexol, naltrindole, methylnaltrexone, methylsamidorphan, alvimopan, axelopran, bevenpran, dinicotinate, levallorphan, samidorphan, buprenorphine, dezocine, eptazocine, butorphanol, levorphanol, nalbuphine, pentazocine, phenazocine, norbinaltorphimine, and diprenorphine.

### Nor-Opioids of Interest

Host cells which produce nor-opioids of interest are provided. In some examples, engineered strains of host cells such as the engineered strains of embodiments discussed herein may provide a platform for producing Nor-opioids of interest including, but not limited to: norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, nor-14-hydroxy-morphinone.

### Benzylisoquinoline Alkaloids (BIAs) of Interest

Host cells which produce BIAs of interest are provided. In some examples, engineered strains of host cells such as the engineered strains of embodiments discussed herein may provide a platform for producing benzylisoquinoline alkaloids of interest and modifications thereof across several structural classes including, but not limited to, precursor BIAs, benzylisoquinolines, promorphinans, morphinans and others. Each of these classes is meant to include biosynthetic precursors, intermediates, and metabolites thereof, of any convenient member of an engineered host cell biosynthetic pathway that may lead to a member of the class. Non-limiting examples of compounds are given below for each of these structural classes. In some cases, the structure of a given example may or may not be characterized itself as a benzylisoquinoline alkaloid. In some cases, the present chemical entities are meant to include all possible isomers, including single enantiomers, racemic mixtures, optically pure forms, mixtures of diastereomers, and intermediate mixtures.

BIA precursors may include, but are not limited to, norcoclaurine (NC) and norlaudanosoline (NL), as well as NC and NL precursors, such as tyrosine, tyramine, 4-hydroxyphenylacetaldehyde (4-HPA), 4-hydroxyphenylpyruvic acid (4-HPPA), L-3,4-dihydroxyphenylalanine (L-DOPA), 3,4-dihydroxyphenylacetaldehyde (3,4-DHPA), and dopamine. In some embodiments, the one or more BIA precursors are 3,4-dihydroxyphenylacetaldehyde (3,4-DHPA) and dopamine. In certain instances, the one or more BIA precursors are 4-hydroxyphenylacetaldehyde (4-HPA) and dopamine. In particular, NL and NC may be synthesized, respectively, from precursor molecules via a Pictet-Spengler condensation reaction, where the reaction may occur spontaneously or may by catalyzed by any convenient enzymes.

Benzylisoquinolines may include, but are not limited to, norcoclaurine, norlaudanosoline, coclaurine, 3′-hydroxycoclaurine, 4′-O-methylnorlaudanosoline, 4′-O-methyl-laudanosoline, N-methylnorcoclaurine, laudanosoline, N-methylcoclaurine, 3′-hydroxy-N-methylcoclaurine, reticuline, norreticuline, papaverine, laudanine, laudanosine, tetrahydropapaverine, 1,2-dihydropapaverine, and orientaline.

Promorphinans may include, but are not limited to, salutaridine, salutaridinol, and salutaridinol-7-O-acetate.

Morphinans may include, but are not limited to, thebaine, codeinone, codeine, morphine, morphinone, oripavine, neopinone, neopine, neomorphine, hydrocodone, dihydrocodeine, 14-hydroxycodeinone, oxycodone, 14-hydroxycodeine, morphinone, hydromorphone, dihydromorphine, dihydroetorphine, ethylmorphine, etorphine, metopon, buprenorphine, pholcodine, and heterocodeine.

### Host Cells

Any convenient cells may be utilized in the subject host cells and methods. In some cases, the host cells are non-plant cells. In some instances, the host cells may be characterized as microbial cells. In certain cases, the host cells are insect cells, vertebrate cells, mammalian cells, plant cells, fungal cells, bacterial cells, or yeast cells. Any convenient type of host cell may be utilized in producing the subject nor-opioid- or nal-opioid-producing cells, see, e.g., US2008/0176754, and US2014/0273109 the disclosures of which are incorporated by reference in their entirety. Host cells of interest include, but are not limited to, bacterial cells, such as Bacillus subtilis, Escherichia coli, Streptomyces, and Salmonella typhimuium cells, insect cells such as Drosophila melanogaster S2 and Spodoptera frugiperda Sf9 cells, mammalian cells such as HeLa and 293 cells, plant cells such as Tobacco BY-2 cells, and yeast cells such as Saccharomyces cerevisiae, Schizosaccharomyces pombe, and Pichia pastoris cells. In some examples, the host cells are yeast cells or E. coli cells. In some cases, the host cell is a yeast cell. In some instances, the host cell is from a strain of yeast engineered to produce a nor-opioid or nal-opioid BIA of interest, such as a northebaine or naloxone. In some instances, the host cell is from a strain of yeast engineered to produce an enzyme of interest. In some instances, the host cell is from a strain of yeast engineered to produce an O-demethylase. The O-demethylase may be able to convert a substrate, such as a first opioid, into a second opioid. In some instances, the host cell is from a strain of yeast engineered to produce an N-demethylase. The N-demethylase may be able to convert a substrate, such as a second opioid, into a nor-opioid. In some instances, the host cell is from a strain of yeast engineered to produce a methyltransferase. Additionally the methyltransferase may be able to convert a nor-opioid into a nal-opioid. In some instances, the host cell is from a strain of yeast engineered to produce an epimerase. The epimerase may have an oxidase and a reductase. Additionally, the epimerase may be able to convert an (S)-1-benzylisoquinoline alkaloid to an (R)-1-benzylisoquinoline alkaloid. Further, the epimerase may be separated into smaller enzymes that retain oxidase or reductase activity so as to be used to convert an (S)-1-benzylisoquinoline alkaloid to an (R)-1-benzylisoquinoline alkaloid.

Any of the host cells described in US2008/0176754 and US2014/0273109 by Smolke et al. may be adapted for use in the subject cells and methods. In certain embodiments, the yeast cells may be of the species Saccharomyces cerevisiae (S. cerevisiae). In certain embodiments, the yeast cells may be of the species Schizosaccharomyces pombe. In certain embodiments, the yeast cells may be of the species Pichia pastoris. Yeast is of interest as a host cell because cytochrome P450 proteins are able to fold properly into the endoplasmic reticulum membrane so that their activity is maintained. In examples, cytochrome P450 proteins are involved in some biosynthetic pathways of interest. In additional examples, cytochrome P450 proteins are involved in the production of BIAs of interest such as naloxone or naltrexone. In further examples, cytochrome P450 proteins are involved in the production of an enzyme of interest, such as an epimerase having an oxidase and a reductase.

Yeast strains of interest that may find use in the invention include, but are not limited to, CEN.PK (Genotype: MATα/α ura3-52/ura3-52 trp1-289/trp1-289 leu2-3_112/leu2-3_112 his3 Δ1/his3 Δ1 MAL2-8C/MAL2-8C SUC2/SUC2), S288C, W303, D273-10B, X2180, A364A, Σ1278B, AB972, SK1, and FL100. In certain cases, the yeast strain is any of S288C (MATα; SUC2 mal mel gal2 CUP1 flo1 flo8-1 hap1), BY4741 (MATα; his3Δ1; leu2Δ0; met15Δ0; ura3Δ0), BY4742 (MATα; his3Δ1; leu2Δ0; lys2Δ0; ura3Δ0), BY4743 (MATα/MATα; his3Δ1/his3Δ1; leu2Δ0/leu2Δ0; met15Δ0/MET15; LYS2/lys2Δ0; ura3Δ0/ura3Δ0), and WAT11 or W(R), derivatives of the W303-B strain (MATα; ade2-1; his3-11, -15; leu2-3, -112; ura3-1; canR; cyr+) which express the Arabidopsis thaliana NADPH-P450 reductase ATR1 and the yeast NADPH-P450 reductase CPR1, respectively. In another embodiment, the yeast cell is W303alpha (MATα; his3-11,15 trp1-1 leu2-3 ura3-1 ade2-1). The identity and genotype of additional yeast strains of interest may be found at EUROSCARF (web.uni-frankfurt.de/fb15/mikro/euroscarf/col_index.html).

In some instances the host cell is a fungal cell. In certain embodiments, the fungal cells may be of the Aspergillus species and strains include Aspergillus niger (ATCC 1015, ATCC 9029, CBS 513.88), Aspergillus oryzae (ATCC 56747, RIB40), Aspergillus terreus (NIH 2624, ATCC 20542) and Aspergillus nidulans (FGSC A4).

In certain embodiments, heterologous coding sequences may be codon optimized for expression in Aspergillus sp. and expressed from an appropriate promoter. In certain embodiments, the promoter may be selected from phosphoglycerate kinase promoter (PGK), MbfA promoter, cytochrome c oxidase subunit promoter (CoxA), SrpB promoter, TvdA promoter, malate dehydrogenase promoter (MdhA), beta-mannosidase promoter (ManB). In certain embodiments, a terminator may be selected from glucoamylase terminator (GlaA) or TrpC terminator. In certain embodiments, the expression cassette consisting of a promoter, heterologous coding sequence, and terminator may be expressed from a plasmid or integrated into the genome of the host. In certain embodiments, selection of cells maintaining the plasmid or integration cassette may be performed with antibiotic selection such as hygromycin or nitrogen source utilization, such as using acetamide as a sole nitrogen source. In certain embodiments, DNA constructs may be introduced into the host cells using established transformation methods such as protoplast transformation, lithium acetate, or electroporation. In certain embodiments, cells may be cultured in liquid ME or solid MEA (3% malt extract, 0.5% peptone, and ±1.5% agar) or in Vogel's minimal medium with or without selection.

In some instances the host cell is a bacterial cell. The bacterial cell may be selected from any bacterial genus. Examples of genuses from which the bacterial cell may come include Anabaena, Arthrobacter, Acetobacter, Acetobacterium, Bacillus, Bifidobacterium, Brachybacterium, Brevibacterium, Carnobacterium, Clostridium, Corynebacterium, Enterobacter, Escherichia, Gluconacetobacter, Gluconobacter, Hafnia, Halomonas, Klebsiella, Kocuria, Lactobacillus, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Serratia, Staphylococcus, Streptococcus, Streptomyces, Synechococcus, Synechocystis, Tetragenococcus, Weissella, and Zymomonas. Examples of bacterial species which may be used with the methods of this disclosure include Arthrobacter nicotianae, Acetobacter aceti, Arthrobacter arilaitensis, Bacillus cereus, Bacillus coagulans, Bacillus licheniformis, Bacillus pumilus, Bacillus sphaericus, Bacillus stearothermophilus, Bacillus subtilis, Bifidobacterium adolescentis, Brachybacterium tyrofermentans, Brevibacterium linens, Carnobacterium divergens, Corynebacterium flavescens, Enterococcus faecium, Gluconacetobacter europaeus, Gluconacetobacter johannae, Gluconobacter oxydans, Hafnia alvei, Halomonas elongata, Kocuria rhizophila, Lactobacillus acidifarinae, Lactobacillus jensenii, Lactococcus lactis, Lactobacillus yamanashiensis, Leuconostoc citreum, Macrococcus caseolyticus, Microbacterium foliorum, Micrococcus lylae, Oenococcus oeni, Pediococcus acidilactici, Propionibacterium acidipropionici, Proteus vulgaris, Pseudomonas fluorescens, Psychrobacter celer, Staphylococcus condimenti, Streptococcus thermophilus, Streptomyces griseus, Tetragenococcus halophilus, Weissella cibaria, Weissella koreensis, Zymomonas mobilis, Corynebacterium glutamicum, Bifidobacterium bifidum/breve/longum, Streptomyces lividans, Streptomyces coelicolor, Lactobacillus plantarum, Lactobacillus sakei, Lactobacillus casei, Pseudoalteromonas citrea, Pseudomonas putida, Clostridium ljungdahlii/aceticum/acetobutylicum/beijerinckii/butyricum, and Moorella themocellum/thermoacetica.

In certain embodiments, the bacterial cells may be of a strain of Escherichia coli. In certain embodiments, the strain of E. coli may be selected from BL21, DHSa, XLI-Blue, HB101, BL21, and K12. In certain embodiments, heterologous coding sequences may be codon optimized for expression in E. coli and expressed from an appropriate promoter. In certain embodiments, the promoter may be selected from T7 promoter, tac promoter, trc promoter, tetracycline-inducible promoter (tet), lac operon promoter (lac), lacO1 promoter. In certain embodiments, the expression cassette consisting of a promoter, heterologous coding sequence, and terminator may be expressed from a plasmid or integrated into the genome. In certain embodiments, the plasmid is selected from pUC19 or pBAD. In certain embodiments, selection of cells maintaining the plasmid or integration cassette may be performed with antibiotic selection such as kanamycin, chloramphenicol, streptomycin, spectinomycin, gentamycin, erythromycin or ampicillin. In certain embodiments, DNA constructs may be introduced into the host cells using established transformation methods such as conjugation, heat shock chemical transformation, or electroporation. In certain embodiments, cells may be cultured in liquid Luria-Bertani (LB) media at 37° C. with or without antibiotics.

In certain embodiments, the bacterial cells may be a strain of Bacillus subtilis. In certain embodiments, the strain of B. subtilis may be selected from 1779, GP25, RO—NN-1, 168, BSn5, BEST195, 1A382, and 62178. In certain embodiments, heterologous coding sequences may be codon optimized for expression in Bacillus sp. and expressed from an appropriate promoter. In certain embodiments, the promoter may be selected from grac promoter, p43 promoter, or trnQ promoter. In certain embodiments, the expression cassette consisting of the promoter, heterologous coding sequence, and terminator may be expressed from a plasmid or integrated into the genome. In certain embodiments, the plasmid is selected from pHP13 pE194, pC194, pHT01, or pHT43. In certain embodiments, integrating vectors such as pDG364 or pDG1730 may be used to integrate the expression cassette into the genome. In certain embodiments, selection of cells maintaining the plasmid or integration cassette may be performed with antibiotic selection such as erythromycin, kanamycin, tetracycline, and spectinomycin. In certain embodiments, DNA constructs may be introduced into the host cells using established transformation methods such as natural competence, heat shock, or chemical transformation. In certain embodiments, cells may be cultured in liquid Luria-Bertani (LB) media at 37° C. or M9 medium plus glucose and tryptophan.

### Genetic Modifications to Host Cells

The host cells may be engineered to include one or more modifications (such as two or more, three or more, four or more, five or more, or even more modifications) that provide for the production of BIAs of interest. Additionally or alternatively, the host cells may be engineered to include one or more modifications (such as two or more, three or more, four or more, five or more, or even more modifications) that provide for the production of enzymes of interest. In some cases, a modification is a genetic modification, such as a mutation, addition, or deletion of a gene or fragment thereof, or transcription regulation of a gene or fragment thereof. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2u or centromeric plasmid. In certain instances, the substrate inhibited copy of the enzyme is under the native cell transcriptional regulation. In some instances, the substrate inhibited copy of the enzyme is introduced with engineered constitutive or dynamic regulation of protein expression by placing it under the control of a synthetic promoter. In some examples, the object of one or more modifications may be a native gene. In some examples, the object of one or more modifications may be a non-native gene. In some examples, a non-native gene may be inserted into a host cell. In further examples, a non-native gene may be altered by one or more modifications prior to being inserted into a host cell.

An engineered host cell may overproduce one or more nor-opioid BIAs of interest. An engineered host cell may overproduce one or more nal-opioid BIAs of interest. By overproduce is meant that the cell has an improved or increased production of a nor-opioid and/or nal-opioid BIA molecule of interest relative to a control cell (e.g., an unmodified cell). By improved or increased production is meant both the production of some amount of the nor-opioid and/or nal-opioid BIA of interest where the control has no nor-opioid and/or nal-opioid BIA of interest production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some nor-opioid and/or nal-opioid BIA of interest production.

An engineered host cell may overproduce one or more nor-opioids. In some cases, the engineered host cell may produce some amount of the nor-opioid of interest where the control has no nor-opioid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some nor-opioid of interest production.

An engineered host cell may further overproduce one or more nal-opioids. In some cases, the engineered host cell may produce some amount of the nal-opioid of interest where the control has no nal-opioid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some nal-opioid of interest production.

An engineered host cell may overproduce one or more BIAs of interest. By overproduce is meant that the cell has an improved or increased production of a BIA molecule of interest relative to a control cell (e.g., an unmodified cell). By improved or increased production is meant both the production of some amount of the BIA of interest where the control has no BIA of interest production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some BIA of interest production.

An engineered host cell may overproduce one or more (S)-1-benzylisoquinoline alkaloids. In some cases, the engineered host cell may produce some amount of the (S)-1-benzylisoquinoline alkaloid of interest where the control has no (S)-1-benzylisoquinoline alkaloid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some (S)-1-benzylisoquinoline alkaloid of interest production.

An engineered host cell may further overproduce one or more (R)-1-benzylisoquinoline alkaloids. In some cases, the engineered host cell may produce some amount of the (R)-1-benzylisoquinoline alkaloid of interest where the control has no (R)-1-benzylisoquinoline alkaloid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some (R)-1-benzylisoquinoline alkaloid of interest production. An engineered host cell may further overproduce one or more of morphinan and pro-morphinan alkaloids.

In some cases, the engineered host cell is capable of producing an increased amount of (R)-reticuline relative to a control host cell that lacks the one or more modifications (e.g., as described herein). In certain instances, the increased amount of (R)-reticuline is about 10% or more relative to the control host cell, such as about 20% or more, about 30% or more, about 40% or more, about 50% or more, about 60% or more, about 80% or more, about 100% or more, about 2-fold or more, about 5-fold or more, or even about 10-fold or more relative to the control host cell. In some cases, (R)-reticuline is the product of an epimerization reaction within an engineered host cell. In these cases, (S)-reticuline may be the substrate of the epimerization reaction.

Additionally, an engineered host cell may overproduce one or more enzymes of interest. By overproduce is meant that the cell has an improved or increased production of an enzyme of interest relative to a control cell (e.g., an unmodified cell). By improved or increased production is meant both the production of some amount of the enzyme of interest where the control has no production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some enzyme of interest production.

An engineered host cell may overproduce one or more O-demethylase (ODM) enzymes. Examples of ODM enzymes that may be utilized in embodiments described herein are found in Table 3. In some cases, the engineered host cell may produce some amount of the ODM enzyme where the control has no ODM enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some ODM enzyme production.

An engineered host cell may overproduce one or more N-demethylase (NDM) enzymes. Examples of NDM enzymes that may be utilized in embodiments described herein are found in Table 4. In some cases, the engineered host cell may produce some amount of the ODM enzyme where the control has no NDM enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some NDM enzyme production.

An engineered host cell may overproduce one or more N-methyltransferase (NMT) enzymes. Examples of NMT enzymes that may be utilized in embodiments described herein are found in Table 5. In some cases, the engineered host cell may produce some amount of the NMT enzyme where the control has no NMT enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some NMT enzyme production.

An engineered host cell may overproduce one or more CYP—COR enzymes. In some cases, the engineered host cell may produce some amount of the CYP—COR enzyme where the control has no CYP—COR enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some CYP—COR enzyme production.

An engineered host cell may further overproduce one or more enzymes that are derived from the CYP—COR enzyme. In some cases, the engineered host cell may produce some amount of the enzymes that are derived from the CYP—COR enzyme, where the control has no production of enzymes that are derived from the CYP—COR enzyme, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some production of enzymes that are derived from the CYP—COR enzyme.

In some cases, the one or more (such as two or more, three or more, or four or more) modifications may be selected from: a substrate inhibition alleviating mutation in a biosynthetic enzyme gene; a product inhibition alleviating mutation in a biosynthetic enzyme gene; a cofactor recovery promoting mechanism; a feedback inhibition alleviating mutation in a biosynthetic enzyme gene; a transcriptional modulation modification of a biosynthetic enzyme gene; an inactivating mutation in an enzyme gene; an epimerization modification; a bisBIA generating modification; and a heterologous coding sequence that encodes an enzyme. A cell that includes one or more modifications may be referred to as an engineered cell.

### Substrate Inhibition Alleviating Mutations

In some instances, the engineered host cells are cells that include one or more substrate inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “substrate inhibition alleviating mutation” refers to a mutation that alleviates a substrate inhibition control mechanism of the cell.

A mutation that alleviates substrate inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of substrate inhibition control mechanisms and biosynthetic enzymes in the engineered host cell that are directed to regulation of levels of nal-opioid or nor-opioid BIAs of interest, or precursors thereof, may be targeted for substrate inhibition alleviation. The engineered host cell may include one or more substrate inhibition alleviating mutations in one or more biosynthetic enzyme genes. The one or more mutations may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more substrate inhibition alleviating mutations are present in a biosynthetic enzyme gene that is TyrH. In some embodiments, the engineered host cell may include one or more substrate inhibition alleviating mutations in one or more biosynthetic enzyme genes such as one of those genes described in Table 2.

In certain embodiments, the one or more substrate inhibition alleviating mutations are present in the TyrH gene. The TyrH gene encodes tyrosine hydroxylase, which is an enzyme that converts tyrosine to L-DOPA. However, TyrH is inhibited by its substrate, tyrosine. Mammalian tyrosine hydroxylase activity, such as that seen in humans or rats, can be improved through mutations to the TyrH gene that relieve substrate inhibition. In particular, substrate inhibition from tyrosine can be relieved by a point mutation W166Y in the TyrH gene. The point mutation W166Y in the TyrH gene may also improve the binding of the cosubstrate of tyrosine hydroxylase, BH₄, to catalyze the reaction of tyrosine to L-DOPA. The mutants of TyrH, when expressed in yeast strains to produce BIAs from sugar (such as those described in U.S. Provisional Patent Application Ser. No. 61/899,496) can significantly improve the production of BIAs.

Any convenient numbers and types of mutations may be utilized to alleviate a substrate inhibition control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more substrate inhibition alleviating mutations, such as 1, 2, 3, 4, 5, 6, 7, 8, 9, 10, 11, 12, 13, 14 or 15 substrate inhibition alleviating mutations in one or more biosynthetic enzyme genes within the engineered host cell.

### Cofactor Recovery Promoting Mechanisms

In some instances, the engineered host cells are cells that include one or more cofactor recovery promoting mechanisms (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “cofactor recovery promoting mechanism” refers to a mechanism that promotes a cofactor recovery control mechanism of the cell.

A variety of cofactor recovery control mechanisms and biosynthetic enzymes in the engineered host cell that are directed to regulation of levels of nor-opioid or nal-opioid BIAs of interest, or precursors thereof, may be targeted for cofactor recovery promotion. The engineered host cell may include one or more cofactor recovery promoting mechanism in one or more biosynthetic enzyme genes. In examples, the engineered host cell may include a heterologous coding sequence that encodes dihydrofolate reductase (DHFR). When DHFR is expressed, it may convert 7,8-dihydrobiopterin (BH₂) to the tetrahydrobiopterin (BH₄), thereby recovering BH₄ as a TyrH cosubstrate. In some examples, the engineered host cell may include one or more cofactor recovery promoting mechanisms in one or more biosynthetic enzyme genes such as one of those genes described in Table 2.

Any convenient numbers and types of mechanisms may be utilized to promote a cofactor recovery control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more cofactor recovery promoting mechanisms such as 1, 2, 3, 4, 5, 6, 7, 8, 9, 10, 11, 12, 13, 14 or 15 cofactor recovery promoting mechanisms in one or more biosynthetic enzyme genes within the engineered host cell.

### Product Inhibition Alleviating Mutations

In some instances, the engineered host cells are cells that include one or more product inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “product inhibition alleviating mutation” refers to a mutation that alleviates a short term and/or long term product inhibition control mechanism of an engineered host cell. Short term product inhibition is a control mechanism of the cell in which there is competitive binding at a cosubstrate binding site. Long term product inhibition is a control mechanism of the cell in which there is irreversible binding of a compound away from a desired pathway.

A mutation that alleviates product inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of product inhibition control mechanisms and biosynthetic enzymes in the engineered host cell that are directed to regulation of levels of nor-opioid or nal-opioid BIAs of interest may be targeted for product inhibition alleviation. The engineered host cell may include one or more product inhibition alleviating mutations in one or more biosynthetic enzyme genes. The mutation may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more product inhibition alleviating mutations are present in a biosynthetic enzyme gene that is TyrH. In some embodiments, the engineered host cell includes one or more product inhibition alleviating mutations in one or more biosynthetic enzyme genes such as one of those genes described in Table 2.

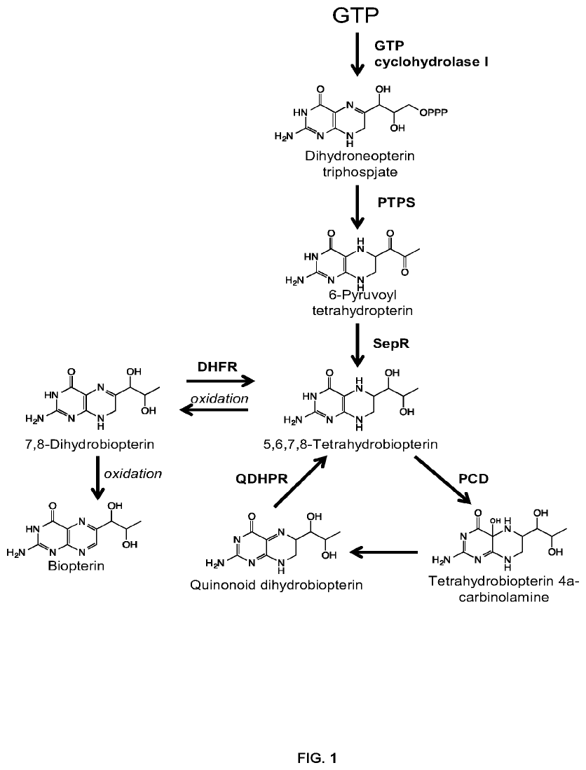

In certain embodiments, the one or more product inhibition alleviating mutations are present in the TyrH gene. The TyrH gene encodes tyrosine hydroxylase, which is an enzyme that converts tyrosine to L-DOPA. TyrH requires tetrahydrobiopterin (BH₄) as a cosubstrate to catalyze the hydroxylation reaction. Some microbial strains, such as Saccharomyces cerevisiae, do not naturally produce BH₄, but can be engineered to produce this substrate through a four-enzyme synthesis and recycling pathway, as illustrated in FIG. 1. FIG. 1 illustrates examples of synthesis, recycling, and salvage pathways of tetrahydrobiopterin, in accordance with embodiments of the invention. FIG. 1 provides the use of the enzymes PTPS, pyruvoyl tetrahydropterin synthase; SepR, sepiapterin reductase; PCD, pterin 4a-carbinolamine dehydratase; QDHPR, dihydropteridine reductase; and DHFR, dihydrofolate reductase. Of the enzymes that are illustrated in FIG. 1, yeast synthesizes an endogenous GTP cyclohydrolase I. GTP and dihydroneopterin triphosphate are naturally synthesized in yeast. Additionally, other metabolites in FIG. 1 are not naturally produced in yeast.

TyrH is inhibited by its product L-DOPA, as well as other catecholamines, particularly dopamine. Mammalian tyrosine hydroxylase activity, such as from humans or rats, can be improved through mutations that relieve product inhibition. For example, short term product inhibition, such as competitive binding at the cosubstrate binding site, can be relieved by a point mutation W166Y on the TyrH gene. In particular, the point mutation W166Y on the TyrH gene may improve binding of the cosubstrate. Additionally, short term product inhibition to relieve competitive binding at the cosubstrate binding site may be improved by a point mutation S40D on the TyrH gene. Short term product inhibition may also be improved by the joint mutations of R37E, R38E on the TyrH gene. In particular, R37E, R38E mutations may together specifically improve tyrosine hydroxylase activity in the presence of dopamine.

Additionally, long term product inhibition may be relieved by point mutations on the TyrH gene. Long term product inhibition relief may include the irreversible binding of catecholamine to iron in the active site such that there is less catecholamine present to act as a product inhibitor of tyrosine hydroxylase activity. Long term product inhibition can be relieved by the mutations E332D and Y371F, respectively, in the TyrH gene.

Combinations of the mutations can be made (such as two or three or more mutations at once) to relieve multiple types of substrate and product inhibition to further improve the activity of TyrH. The mutants of TyrH, when expressed in yeast strains to produce nor-opioid and/or nal-opioid BIAs of interest from sugar (such as those described in U.S. Provisional Patent Application Ser. No. 61/899,496) can significantly improve the production of nor-opioid and/or nal-opioid products.

Any convenient numbers and types of mutations may be utilized to alleviate a product inhibition control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more product inhibition alleviating mutations, such as 1, 2, 3, 4, 5, 6, 7, 8, 9, 10, 11, 12, 13, 14 or 15 product inhibition alleviating mutations in one or more biosynthetic enzyme genes within the engineered host cell.

### Feedback Inhibition Alleviating Mutations

In some instances, the engineered host cells are cells that include one or more feedback inhibition alleviating mutations (such as two or more, three or more, four or more, five or more, or even more) in one or more biosynthetic enzyme genes of the cell. In some cases, the one or more biosynthetic enzyme genes are native to the cell (e.g., is present in an unmodified cell). Additionally or alternatively, in some examples the one or more biosynthetic enzyme genes are non-native to the cell. As used herein, the term “feedback inhibition alleviating mutation” refers to a mutation that alleviates a feedback inhibition control mechanism of an engineered host cell. Feedback inhibition is a control mechanism of the cell in which an enzyme in the synthetic pathway of a regulated compound is inhibited when that compound has accumulated to a certain level, thereby balancing the amount of the compound in the cell. A mutation that alleviates feedback inhibition reduces the inhibition of a regulated enzyme in the engineered host cell relative to a control cell. In this way, engineered host cell provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the host cell or a downstream product thereof.

A variety of feedback inhibition control mechanisms and biosynthetic enzymes that are directed to regulation of levels of BIAs of interest may be targeted for alleviation in the host cell. The host cell may include one or more feedback inhibition alleviating mutations in one or more biosynthetic enzyme genes native to the cell. The one or more mutations may be located in any convenient biosynthetic enzyme genes where the biosynthetic enzyme is subject to regulatory control. In some embodiments, the one or more biosynthetic enzyme genes may encode one or more enzymes selected from a 3-deoxy-d-arabinose-heptulosonate-7-phosphate (DAHP) synthase and a chorismate mutase. In some embodiments, the one or more biosynthetic enzyme genes encode a 3-deoxy-d-arabinose-heptulosonate-7-phosphate (DAHP) synthase. In some instances, the one or more biosynthetic enzyme genes may encode a chorismate mutase. In certain instances, the one or more feedback inhibition alleviating mutations may be present in a biosynthetic enzyme gene selected from ARO4 and ARO7. In certain instances, the one or more feedback inhibition alleviating mutations may be present in a biosynthetic enzyme gene that is ARO4. In certain instances, the one or more feedback inhibition alleviating mutations are present in a biosynthetic enzyme gene that is ARO7. In some embodiments, the engineered host cell may include one or more feedback inhibition alleviating mutations in one or more biosynthetic enzyme genes such as one of those genes described in Table 2.

Any convenient numbers and types of mutations may be utilized to alleviate a feedback inhibition control mechanism. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2μ or centromeric plasmid. In certain instances, the feedback inhibited copy of the enzyme is under the native cell transcriptional regulation. In some instances, the feedback inhibited copy of the enzyme is introduced with engineered constitutive or dynamic regulation of protein expression by placing it under the control of a synthetic promoter.

In certain embodiments, the one or more feedback inhibition alleviating mutations may be present in the ARO4 gene. ARO4 mutations of interest may include, but are not limited to, substitution of the lysine residue at position 229 with a leucine, a substitution of the glutamine residue at position 166 with a lysine residue, or a mutation as described by Hartmann M, et al. ((2003) Proc Natl Acad Sci USA 100 (3): 862-867) or Fukuda, et al. ((1992) J Ferment Bioeng 74 (2): 117-119). In some instances, mutations for conferring feedback inhibition may be selected from a mutagenized library of enzyme mutants. Examples of such selections may include rescue of growth of o-fluoro-D,L-phenylalanine or growth of aro3 mutant yeast strains in media with excess tyrosine as described by Fukuda, et al. ((1990) Breeding of Brewing Yeast Producing a Large Amount of Beta-Phenylethyl Alcohol and Beta-Phenylethyl Acetate. Agr Biol Chem Tokyo 54 (1): 269-271).

In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more feedback inhibition alleviating mutations, such as 1, 2, 3, 4, 5, 6, 7, 8, 9, 10, 11, 12, 13, 14 or 15 feedback inhibition alleviating mutations in one or more biosynthetic enzyme genes within the engineered host cell.

### Transcriptional Modulation Modifications

The host cells may include one or more transcriptional modulation modifications (such as two or more, three or more, four or more, five or more, or even more modifications) of one or more biosynthetic enzyme genes of the cell. In some examples, the one or more biosynthetic enzyme genes are native to the cell. In some examples, the one or more biosynthetic enzyme genes are non-native to the cell. Any convenient biosynthetic enzyme genes of the cell may be targeted for transcription modulation. By transcription modulation is meant that the expression of a gene of interest in a modified cell is modulated, e.g., increased or decreased, enhanced or repressed, relative to a control cell (e.g., an unmodified cell). In some cases, transcriptional modulation of the gene of interest includes increasing or enhancing expression. By increasing or enhancing expression is meant that the expression level of the gene of interest is increased by 2-fold or more, such as by 5-fold or more and sometimes by 25-, 50-, or 100-fold or more and in certain embodiments 300-fold or more or higher, as compared to a control, i.e., expression in the same cell not modified (e.g., by using any convenient gene expression assay). Alternatively, in cases where expression of the gene of interest in a cell is so low that it is undetectable, the expression level of the gene of interest is considered to be increased if expression is increased to a level that is easily detectable. In certain instances, transcriptional modulation of the gene of interest includes decreasing or repressing expression. By decreasing or repressing expression is meant that the expression level of the gene of interest is decreased by 2-fold or more, such as by 5-fold or more and sometimes by 25-, 50-, or 100-fold or more and in certain embodiments 300-fold or more or higher, as compared to a control. In some cases, expression is decreased to a level that is undetectable. Modifications of host cell processes of interest that may be adapted for use in the subject host cells are described in U.S. Publication No. 20140273109 (Ser. No. 14/211,611) by Smolke et al., the disclosure of which is herein incorporated by reference in its entirety.

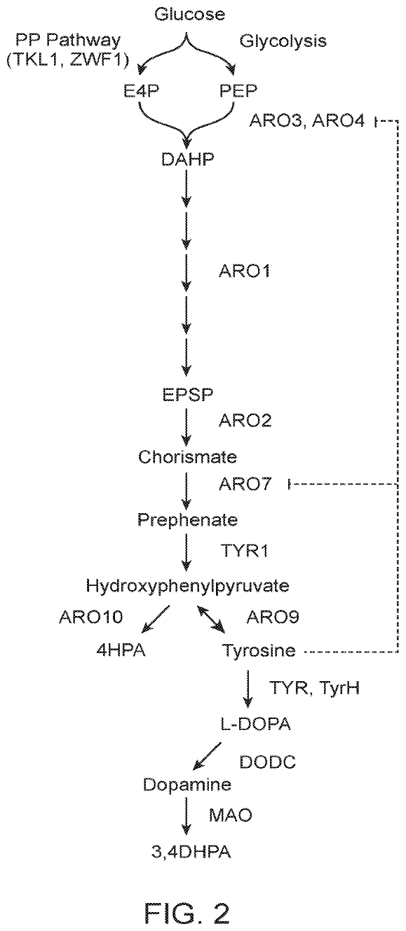

Any convenient biosynthetic enzyme genes may be transcriptionally modulated, and include but are not limited to, those biosynthetic enzymes described in FIG. 2. In particular, FIG. 2 illustrates a biosynthetic scheme for conversion of glucose to 4-HPA, dopamine, and 3,4-DHPA, in accordance with embodiments of the invention. Examples of enzymes described in FIG. 2 include ARO3, ARO4, ARO1, ARO7, TYR1, TYR, TyrH, DODC, MAO, ARO10, ARO9, and TKL. In some instances, the one or more biosynthetic enzyme genes may be selected from ARO10, ARO9, and TKL. In some cases, the one or more biosynthetic enzyme genes may be ARO10. In certain instances, the one or more biosynthetic enzyme genes may be ARO9. In some embodiments, the one or more biosynthetic enzyme genes may be TKL. In some embodiments, the host cell includes one or more transcriptional modulation modifications to one or more genes such as one of those genes described in Table 2.

In some embodiments, the transcriptional modulation modification may include a substitution of a strong promoter for a native promoter of the one or more biosynthetic enzyme genes or the expression of an additional copy(ies) of the gene or genes under the control of a strong promoter. The promoters driving expression of the genes of interest may be constitutive promoters or inducible promoters, provided that the promoters may be active in the host cells. The genes of interest may be expressed from their native promoters. Additionally or alternatively, the genes of interest may be expressed from non-native promoters. Although not a requirement, such promoters may be medium to high strength in the host in which they are used. Promoters may be regulated or constitutive. In some embodiments, promoters that are not glucose repressed, or repressed only mildly by the presence of glucose in the culture medium, may be used. There are numerous suitable promoters, examples of which include promoters of glycolytic genes such as the promoter of the B. subtilis tsr gene (encoding fructose biphosphate aldolase) or GAPDH promoter from yeast S. cerevisiae (coding for glyceraldehyde-phosphate dehydrogenase) (Bitter G. A., Meth. Enzymol. 152:673-684 (1987)). Other strong promoters of interest include, but are not limited to, the ADHI promoter of baker's yeast (Ruohonen L., et al, J. Biotechnol. 39:193-203 (1995)), the phosphate-starvation induced promoters such as the PHO5 promoter of yeast (Hinnen A., et al, in Yeast Genetic Engineering, Barr P. J., et al. eds, Butterworths (1989), the alkaline phosphatase promoter from B. licheniformis (Lee. J. W. K., et al, J. Gen. Microbiol. 137:1127-1133 (1991)), GPD1, and TEF1. Yeast promoters of interest include, but are not limited to, inducible promoters such as Gal1-10, Gal1, GalL, GalS, repressible promoter Met25, tetO, and constitutive promoters such as glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), MRP7 promoter, etc. In some instances, the strong promoter is GPD1. In certain instances, the strong promoter is TEF1. Autonomously replicating yeast expression vectors containing promoters inducible by hormones such as glucocorticoids, steroids, and thyroid hormones are also known and include, but are not limited to, the glucorticoid responsive element (GRE) and thyroid hormone responsive element (TRE), see e.g., those promoters described in U.S. Pat. No. 7,045,290. Vectors containing constitutive or inducible promoters such as alpha factor, alcohol oxidase, and PGH may be used. Additionally any promoter/enhancer combination (as per the Eukaryotic Promoter Data Base EPDB) could also be used to drive expression of genes of interest. It is understood that any convenient promoters specific to the host cell may be selected, e.g., E. coli T7 promoter, lac promoter or tetO promoter. In some cases, promoter selection may be used to optimize transcription, and hence, enzyme levels to maximize production while minimizing energy resources.

### Inactivating Mutations

The engineered host cells may include one or more inactivating mutations to an enzyme of the cell (such as two or more, three or more, four or more, five or more, or even more). The inclusion of one or more inactivating mutations may modify the flux of a synthetic pathway of an engineered host cell to increase the levels of a nor-opioid or nal-opioid BIAs of interest or a desirable enzyme or precursor leading to the same. In some examples, the one or more inactivating mutations are to an enzyme native to the cell. Additionally or alternatively, the one or more inactivating mutations are to an enzyme non-native to the cell. As used herein, by “inactivating mutation” is meant one or more mutations to a gene or regulatory DNA sequence of the cell, where the mutation(s) inactivates a biological activity of the protein expressed by that gene of interest. In some cases, the gene is native to the cell. In some instances, the gene encodes an enzyme that is inactivated and is part of or connected to the synthetic pathway of a nor-opioid and/or nal-opioid BIAs of interest produced by the host cell. In some instances, an inactivating mutation is located in a regulatory DNA sequence that controls a gene of interest. In certain cases, the inactivating mutation is to a promoter of a gene. Any convenient mutations (e.g., as described herein) may be utilized to inactivate a gene or regulatory DNA sequence of interest. By “inactivated” or “inactivates” is meant that a biological activity of the protein expressed by the mutated gene is reduced by 10% or more, such as by 20% or more, 30% or more, 40% or more, 50% or more, 60% or more, 70% or more, 80% or more, 90% or more, 95% or more, 97% or more, or 99% or more, relative to a control protein expressed by a non-mutated control gene. In some cases, the protein is an enzyme and the inactivating mutation reduces the activity of the enzyme.

In some examples, the engineered host cell includes an inactivating mutation in an enzyme native to the cell. Any convenient enzymes may be targeted for inactivation. Enzymes of interest may include, but are not limited to those enzymes, described in Table 2 whose action in the synthetic pathway of the engineered host cell tends to reduce the levels of a nor-opioid or nal-opioid of interest. In some cases, the enzyme has glucose-6-phosphate dehydrogenase activity. In certain embodiments, the enzyme that includes an inactivating mutation is ZWF1. In some cases, the enzyme has alcohol dehydrogenase activity. In some embodiments, the enzyme that includes an inactivating mutation is selected from ADH2, ADH3, ADH4, ADH5, ADH6, ADH7, and SFA1. In certain embodiments, the enzyme that includes an inactivating mutation(s) is ADH2. In certain embodiments, the enzyme that includes an inactivating mutation(s) is ADH3. In certain embodiments, the enzyme that includes an inactivating mutation(s) is ADH4. In certain embodiments, the enzyme that includes an inactivating mutation(s) is ADH5. In certain embodiments, the enzyme that includes an inactivating mutation(s) is ADH6. In certain embodiments, the enzyme that includes an inactivating mutation(s) is ADH7. In some cases, the enzyme has aldehyde oxidoreductase activity. In certain embodiments, the enzyme that includes an inactivating mutation is selected from ALD2, ALD3, ALD4, ALD5, and ALD6. In certain embodiments, the enzyme that includes an inactivating mutation(s) is ALD2. In certain embodiments, the enzyme that includes an inactivating mutation(s) is ALD3. In certain embodiments, the enzyme that includes an inactivating mutation(s) is ALD4. In certain embodiments, the enzyme that includes an inactivating mutation(s) is ALD5. In certain embodiments, the enzyme that includes an inactivating mutation(s) is ALD6. In some embodiments, the host cell includes one or more inactivating mutations to one or more genes described in Table 2.

### Epimerization Modifications

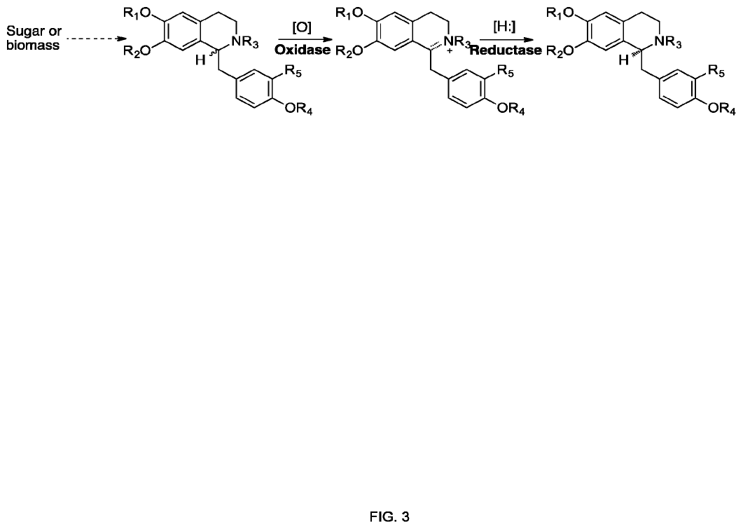

Some methods, processes, and systems provided herein describe the conversion of (S)-1-benzylisoquinoline alkaloids to (R)-1-benzylisoquinoline alkaloids. Some of these methods, processes, and systems may comprise an engineered host cell. In some examples, the conversion of (S)-1-benzylisoquinoline alkaloids to (R)-1-benzylisoquinoline alkaloids is a key step in the conversion of a substrate to a diverse range of alkaloids. In some examples, the conversion of (S)-1-benzylisoquinoline alkaloids to (R)-1-benzylisoquinoline alkaloids comprises an epimerization reaction. In some cases, epimerization of a substrate alkaloid may be performed by oxidizing an (S)-substrate to the corresponding Schiff base or imine intermediate, then stereospecifically reducing this intermediate to an (R)-product as provided in FIG. 3 and as represented generally in Scheme 1. As provided in Scheme 1, R₁, R₂, R₃, and R₄ may be H or CH₃. R₅ may be H, OH, or OCH₃.

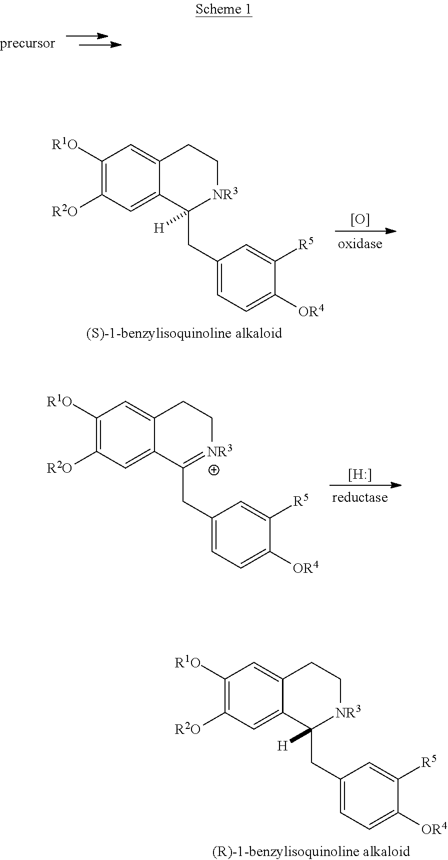

In some examples, the conversion of the (S)-substrate to the (R)-product may involve at least one oxidation reaction and at least one reduction reaction. In some cases, an oxidation reaction is optionally followed by a reduction reaction. In some cases, at least one of the oxidation and reduction reactions is carried out in the presence of an enzyme. In some cases, at least one of the oxidation and reduction reactions is catalyzed by an enzyme. In some cases, the oxidation and reduction reactions are both carried out in the presence of at least one enzyme. In some cases, at least one enzyme is useful to catalyze the oxidation and reduction reactions. The oxidation and reduction reactions may be catalyzed by the same enzyme.

In some methods, processes and systems described herein, an oxidation reaction may be performed in the presence of an enzyme. In some examples, the enzyme may be an oxidase. The oxidase may use an (S)-1-benzylisoquinoline as a substrate. The oxidase may convert the (S)-substrate to a corresponding imine or Schiff base derivative. The oxidase may be referred to as 1,2-dehydroreticuline synthase (DRS). Non-limiting examples of enzymes suitable for oxidation of (S)-1-benzylisoquinoline alkaloids in this disclosure include a cytochrome P450 oxidase, a 2-oxoglutarate-dependent oxidase, and a flavoprotein oxidase. For example, (S)-tetrahydroprotoberberine oxidase (STOX, E.C 1.3.3.8) may oxidize (S)-norreticuline and other (S)-1-benzylisoquinoline alkaloids to 1,2-dehydronorreticuline and other corresponding 1,2-dehydro products. In some examples, a protein that comprises an oxidase domain of any one of the preceding examples may perform the oxidation. In some examples, the oxidase may catalyze the oxidation reaction within a host cell, such as an engineered host cell, as described herein.

In some examples, a reduction reaction may follow the oxidation reaction. The reduction reaction may be performed by an enzyme. In some examples, the reductase may use an imine or Schiff base derived from a 1-benzylisoquinoline as a substrate. The reductase may convert the imine or Schiff base derivative to an (R)-1-benzylisoquinoline. The reductase may be referred to as 1,2-dehydroreticuline reductase (DRR). Non-limiting examples of enzymes suitable for reduction of an imine or Schiff base derived from an (S)-1-benzylisoquinoline alkaloid include an aldo-keto reductase (e.g., a codeinone reductase-like enzyme (EC 1.1.1.247)) and a short chain dehydrogenase (e.g., a salutaridine reductase-like enzyme (EC 1.1.1.248)). In some examples, a protein that comprises a reductase domain of any one of the preceding examples may perform the reduction. In a further embodiment, the reduction is stereospecific. In some examples, the reductase may catalyze the reduction reaction within a host cell, such as an engineered host cell, as described herein.

An example of an enzyme that can perform an epimerization reaction that converts (S)-1-benzylisoquinoline alkaloids to (R)-1-benzylisoquinoline alkaloids includes an epimerase having an oxidase domain and a reductase domain. In particular, the epimerase may have a cytochrome P450 oxidase 82Y2-like domain. Additionally, the epimerase may have a codeinone reductase-like domain. Further, an epimerase having a cytochrome P450 oxidase 82Y2-like domain and also having a codeinone reductase-like domain may be referred to as a CYP—COR enzyme. In particular, a CYP—COR enzyme may be a fusion enzyme. The CYP—COR enzyme may also be referred to as DRS-DRR (dehydroreticuline synthase-dehydroreticuline reductase).

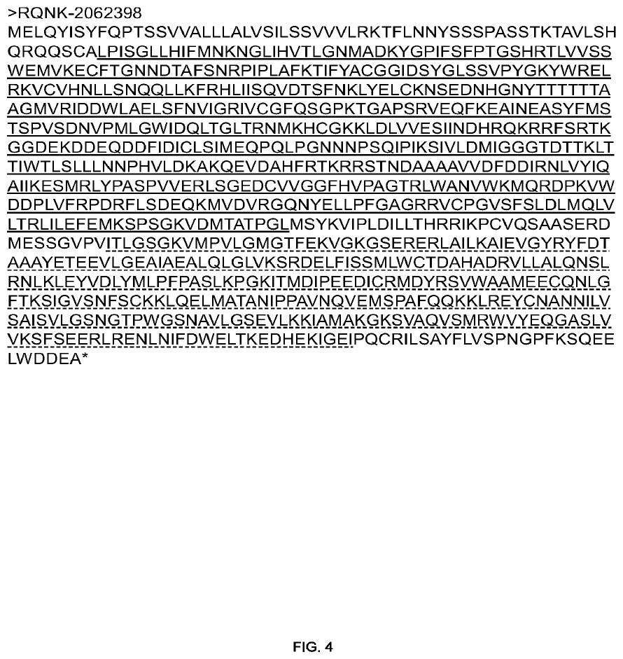

An example of an amino acid sequence of a CYP—COR enzyme that may be used to perform the conversion of (S)-1-benzylisoquinoline alkaloids to (R)-1-benzylisoquinoline alkaloids is provided in FIG. 4. In particular, FIG. 4 illustrates an amino acid sequence of a CYP—COR enzyme, in accordance with embodiments of the invention. As seen in FIG. 4, underlined text denotes the cytochrome P450 CYP82Y2-like domain (59% identity to AFB74617.1). The dotted underlined text denotes the aldo-keto reductase NADPH-dependent codeinone reductase-like domain (75% identity to ACM44066.1). Additional amino acid sequences of a CYP—COR enzyme are set forth in Table 1. An amino acid sequence for an epimerase that is utilized in converting an (S)-1-benzylisoquinoline alkaloid to an (R)-1-benzylisoquinoline alkaloid may be 75% or more identical to a given amino acid sequence as listed in Table 1. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an epimerase that converts (S)-1-benzylisoquinoline alkaloid to (R)-1-benzylisoquinoline alkaloid, wherein the epimerase comprises an amino acid sequence selected from the group consisting of: SEQ ID NOs: 1, 2, 3, 4, 5, 6, 7, 8, 9, 10, 11, 12, 13, 14, and 15. The epimerase that is produced within the engineered host cell may be recovered and purified so as to form a biocatalyst. In some cases, the epimerase may be split into one or more enzymes. Additionally, one or more enzymes that are produced by splitting the epimerase may be recovered from the engineered host cell. These one or more enzymes that result from splitting the epimerase may also be used to catalyze the conversion of (S)-1-benzylisoquinoline alkaloids to (R)-1-benzylisoquinoline alkaloids. In particular, the one or more enzymes that are recovered from the engineered host cell that produces the epimerase may be used in a process for converting an (S)-1-benzylisoquinoline alkaloid to an (R)-1-benzylisoquinoline alkaloid. The process may include contacting the (S)-1-benzylisoquinoline alkaloid with an epimerase in an amount sufficient to convert said (S)-1-benzylisoquinoline alkaloid to (R)-1-benzylisoquinoline alkaloid. In examples, the (S)-1-benzylisoquinoline alkaloid may be contacted with a sufficient amount of the one or more enzymes such that at least 5% of said (S)-1-benzylisoquinoline alkaloid is converted to (R)-1-benzylisoquinoline alkaloid. In further examples, the (S)-1-benzylisoquinoline alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said (S)-1-benzylisoquinoline alkaloid is converted to (R)-1-benzylisoquinoline alkaloid.

The one or more enzymes that may be used to convert an (S)-1-benzylisoquinoline alkaloid to an (R)-1-benzylisoquinoline alkaloid may contact the (S)-1-benzylisoquinoline alkaloid in vitro. Additionally, or alternatively, the one or more enzymes that may be used to convert an (S)-1-benzylisoquinoline alkaloid to an (R)-1-benzylisoquinoline alkaloid may contact the (S)-1-benzylisoquinoline alkaloid in vivo. Additionally, the one or more enzymes that may be used to convert an (S)-1-benzylisoquinoline alkaloid to an (R)-1-benzylisoquinoline alkaloid may be provided to a cell having the (S)-1-benzylisoquinoline alkaloid within, or may be produced within an engineered host cell.

In some examples, the methods provide for engineered host cells that produce an alkaloid product, wherein the epimerization of an (S)-substrate to an (R)-product may comprise a key step in the production of an alkaloid product. In some examples, the alkaloid produced is an (R)-1-benzylisoquinoline alkaloid. In still other embodiments, the alkaloid produced is derived from an (R)-1-benzylisoquinoline alkaloid, including, for example, 4-ring promorphinan and 5-ring morphinan alkaloids. In another embodiment, an (S)-1-benzylisoquinoline alkaloid is an intermediate toward the product of the engineered host cell. In still other embodiments, the alkaloid product is selected from the group consisting of morphinan and promorphinanalkaloids.

In some examples, the (S)-substrate is an (S)-1-benzylisoquinoline alkaloid selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)- orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, (S)-4′-O-methylnorlaudanosoline.

In some examples, the (S)-substrate is a compound of Formula I:

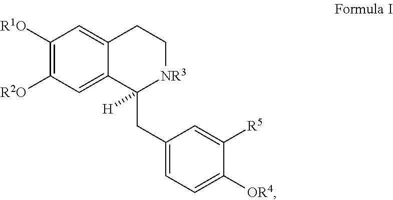

or a salt thereof, wherein:
R¹, R², R³, and R⁴ are independently selected from hydrogen and methyl; and
R⁵ is selected from hydrogen, hydroxy, and methoxy.

In some other examples, at least one of R¹, R², R³, R⁴, and R⁵ is hydrogen.

In still other examples, the (S)-substrate is a compound of Formula II:

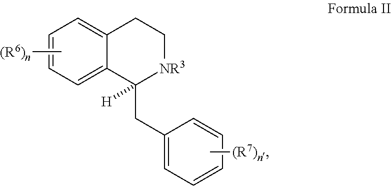

or a salt thereof, wherein:
R³ is selected from hydrogen and C₁-C₄ alkyl;
R⁶ and R⁷ are independently selected at each occurrence from hydroxy, fluoro, chloro, bromo, carboxaldehyde, C₁-C₄ acyl, C₁-C₄ alkyl, and C₁-C₄ alkoxy;
n is 0, 1, 2, 3, or 4; and
n′ is 0, 1, 2, 3, 4 or 5.

When a bond is drawn across a ring, it means substitution may occur at a non-specific ring atom or position. For example, in Formula II shown above, the hydrogen of any —CH— in the 6-membered ring may be replaced with R⁷ to form —CR⁷—.

In some examples, R⁶ and R⁷ are independently methyl or methoxy. In some other examples, n and n′ are independently 1 or 2. In still other embodiments, R³ is hydrogen or methyl.

In some examples, the methods provide for engineered host cells that produce alkaloid products from (S)-reticuline. The epimerization of (S)-reticuline to (R)-reticuline may comprise a key step in the production of diverse alkaloid products from a precursor. In some examples, the precursor is L-tyrosine or a sugar (e.g., glucose). The diverse alkaloid products can include, without limitation, morphinan and promorphinan alkaloids.

Any suitable carbon source may be used as a precursor toward an epimerized 1-benzylisoquinoline alkaloid. Suitable precursors can include, without limitation, monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., corn steep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine). In some examples, a 1-benzylisoquinoline alkaloid may be added directly to an engineered host cell, including, for example, norlaudanosoline, laudanosoline, norreticuline, and reticuline. In still further embodiments, a 1-benzylisoquinoline alkaloid may be added to the engineered host cell as a single enantiomer (e.g., an (S)-1-benzylisoquinoline alkaloid), or a mixture of enantiomers, including, for example, a racemic mixture.

In some examples, the methods provide for the epimerization of a stereocenter of a 1-benzylisoquinoline alkaloid, or a derivative thereof. In a further embodiment, the method comprises contacting the 1-benzylisoquinoline alkaloid with at least one enzyme. The at least one enzyme may invert the stereochemistry of a stereocenter of a 1-benzylisoquinoline alkaloid, or derivative thereof, to the opposite stereochemistry. In some examples, the at least one enzyme converts an (S)-1-benzylisoquinoline alkaloid to an (R)-1-benzylisoquinoline alkaloid. In some examples of this conversion of an (S)-1-benzylisoquinoline alkaloid to an (R)-1-benzylisoquinoline alkaloid utilizing the at least one enzyme, the (S)-1-benzylisoquinoline alkaloid is selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)- orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, and (S)-4′-O-methylnorlaudanosoline.

In still other embodiments, the 1-benzylisoquinoline alkaloid that is epimerized may comprise two or more stereocenters, wherein only one of the two or more stereocenters is inverted to produce a diastereomer of the substrate (e.g., (S, R)-1-benzylisoquinoline alkaloid converted to (R, R)-1-benzylisoquinoline alkaloid). In examples where only one stereocenter of a 1-benzylisoquinoline alkaloid is inverted when contacted with the at least one enzyme, the product is referred to as an epimer of the 1-benzylisoquinoline alkaloid.

In some examples, the 1-benzylisoquinoline alkaloid is presented to the enzyme as a single stereoisomer. In some other examples, the 1-benzylisoquinoline alkaloid is presented to the enzyme as a mixture of stereoisomers. In still further embodiments, the mixture of stereoisomers may be a racemic mixture. In some other examples, the mixture of stereoisomers may be enriched in one stereoisomer as compared to another stereoisomer.

In some examples, an 1-benzylisoquinoline alkaloid, or a derivative thereof, is recovered. In some examples, the 1-benzylisoquinoline alkaloid is recovered from a cell culture. In still further embodiments, the recovered 1-benzylisoquinoline alkaloid is enantiomerically enriched in one stereoisomer as compared to the original mixture of 1-benzylisoquinoline alkaloids presented to the enzyme. In still further embodiments, the recovered 1-benzylisoquinoline alkaloid has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. “Diastereoisomers” or “diastereomers” are stereoisomers that have at least two asymmetric atoms but are not mirror images of each other. The term “epimer” as used herein refers to a compound having the identical chemical formula but a different optical configuration at a particular position. For example, the (R,S) and (S,S) stereoisomers of a compound are epimers of one another. In some examples, a 1-benzylisoquinoline alkaloid is converted to its epimer (e.g., epi-1-benzylisoquinoline alkaloid). The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms that can be defined, in terms of absolute stereochemistry, as (R)- or (S)-.

TABLE 1
Example partial and full-length amino acid sequences of CYP-COR fusion
enzymes.

| | | SEQ. ID |
|---|---|---|
| Sequence | Description | NO. |
| MELQYISYFQPTSSVVALLLALVSILSSVVVLRKTFLNNYSS | P. somniferum | SEQ. ID |
| SPASSTKTAVLSHQRQQSCALPISGLLHIFMNKNGLIHVTLG | plant source; | NO. 1 |
| NMADKYGPIFSFPTGSHRTLVVSSWEMVKECFTGNNDTAF | full-length | |
| SNRPIPLAFKTIFYACGGIDSYGLSSVPYGKYWRELRKVCV | amino acid | |
| HNLLSNQQLLKFRHLIISQVDTSFNKLYELCKNSEDNHGNY | sequence | |
| TTTTTTAAGMVRIDDWLAELSFNVIGRIVCGFQSGPKTGAP | >RQNK- | |
| SRVEQFKEAINEASYFMSTSPVSDNVPMLGWIDQLTGLTRN | 2062398 | |
| MKHCGKKLDLVVESIINDHRQKRRFSRTKGGDEKDDEQDD | (also FPYZ- | |
| FIDICLSIMEQPQLPGNNNPSQIPIKSIVLDMIGGGTDTTKLTT | 2037562, | |
| IWTLSLLLNNPHVLDKAKQEVDAHFRTKRRSTNDAAAAVV | BMRX- | |
| DFDDIRNLVYIQAIIKESMRLYPASPVVERLSGEDCVVGGFH | 2007040, and | |
| VPAGTRLWANVWKMQRDPKVWDDPLVFRPDRFLSDEQK | MLPX- | |
| MVDVRGQNYELLPFGAGRRVCPGVSFSLDLMQLVLTRLIL | 2016197) | |
| EFEMKSPSGKVDMTATPGLMSYKVIPLDILLTHRRIKPCVQ | | |
| SAASERDMESSGVPVITLGSGKVMPVLGMGTFEKVGKGSE | | |
| RERLAILKAIEVGYRYFDTAAAYETEEVLGEAIAEALQLGL | | |
| VKSRDELFISSMLWCTDAHADRVLLALQNSLRNLKLEYVD | | |
| LYMLPFPASLKPGKITMDIPEEDICRMDYRSVWAAMEECQ | | |
| NLGFTKSIGVSNFSCKKLQELMATANIPPAVNQVEMSPAFQ | | |
| QKKLREYCNANNILVSAISVLGSNGTPWGSNAVLGSEVLK | | |
| KIAMAKGKSVAQVSMRWVYEQGASLVVKSFSEERLRENL | | |
| NIFDWELTKEDHEKIGEIPQCRILSAYFLVSPNGPFKSQEEL | | |
| WDDEA* | | |
| MELQYISYFQPTSSVVALLLALVSILSSVVVLRKTFLNNYSS | P. somniferum | SEQ. ID |
| SPASSTKTAVLSHQRQQSCALPISGLLHIFMNKNGLIHVTLG | plant source; | NO. 2 |
| NMADKYGPIFSFPTGSHRTLVVSSWEMVKECFTGNNDTAF | full-length | |
| SNRPIPLAFKTIFYACGGIDSYGLSSVPYGKYWRELRKVCV | amino acid | |
| HNLLSNQQLLKFRHLIISQVDTSFNKLYELCKNSEDNHGNY | sequence | |
| TTXLLLPQLAWRQPWKLYYXTTTTAAGMVRIDDWLAELS | >KKCW- | |
| FNVIGRIVCGFQSGPKTGAPSRVEQFKEAINEASYFMSTSPV | 2026866 | |
| SDNVPMLGWIDQLTGLTRNMKHCGKKLDLVVESIINDHRQ | (also FPYZ- | |
| KRRFSRTKGGDEKDDEQDDFIDICLSIMEQPQLPGNNNPSQI | 2037562, | |
| PIKSIVLDMIGGGTDTTKLTTIWTLSLLLNNPHVLDKAKQE | MLPX- | |
| VDAHFRTKRRSTNDAAAAVVDFDDIRNLVYIQAIIKESMRL | 2016197) | |
| YPASPVVERLSGEDCVVGGFHVPAGTRLWANVWKMQRDP | | |
| KVWDDPLVFRPDRFLSDEQKMVDVRGQNYELLPFGAGRR | | |
| VCPGVSFSLDLMQLVLTRLILEFEMKSPSGKVDMTATPGL | | |
| MSYKVIPLDILLTHRRIKPCVQSAASERDMESSGVPVITLGS | | |
| GKVMPVLGMGTFEKVGKGSERERLAILKAIEVGYRYFDTA | | |
| AAYETEEVLGEAIAEALQLGLVKSRDELEISSMLWCTDAHA | | |
| DRVLLALQNSLRNLKLEYVDLYMLPFPASLKPGKITMDIPE | | |
| EDICRMDYRSVWAAMEECQNLGFTKSIGVSNFSCKKLQEL | | |
| MATANIPPAVNQVEMSPAFQQKKLREYCNANNILVSAISVL | | |
| GSNGTPWGSNAVLGSEVLKKIAMAKGKSVAQVSMRWVY | | |
| EQGASLVVKSFSEERLRENLNIFDWELTKEDHEKIGEIPQCR | | |
| ILSAYFLVSPNGPFKSQEELWDDEA* | | |
| MELQYISYFQPTSSVVALLLALVSILSSVVVLRKTFLNNYSS | P. somniferum | SEQ. ID |
| SPASSTKTAVLSHQRQQSCALPISGLLHIFMNKNGLIHVTLG | plant source; | NO. 3 |
| NMADKYGPIFSFPTGSHRTLVVSSWEMVKECFTGNNDTAF | partial-length | |
| SNRPIPLAFKTIFYACGGIDSYGLSSVPYGKYWRELRKVCV | amino acid | |
| HNLLSNQQLLKFRHLIISQVDTSFNKLYELCKNSEDNHGNY | sequence | |
| TTTTTTAAGMVRIDDWLAELSFNVIGRIVCGFQSGPKTGAP | >SUFP- | |
| SRVEQFKEAINEASYFMSTSPVSDNVPMLGWIDQLTGLTRN | 2025636 | |
| MKHCGKKLDLVVESIINDHRQKRRFSRTKGGDEKDDEQDD | | |
| FIDICLSIMEQPQLPGNNNPSQIPIKSIVLDMIGGGTDTTKLTT | | |
| IWTLSLLLNNPHVLDKAKQEVDAHFRTKRRSTNDAAAAVV | | |
| DFDDIRNLVYIQAIIKESMRLYPASPVVERLSGEDCVVGGFH | | |
| VPAGTRLWANVWKMQRDPKVWDDPLVFRPDRFLSDEQK | | |
| MVDVRGQNYELLPFGAGRRVCPGVSFSLDLMQLVLTRLIL | | |
| EFEMKSPSGKVDMTATPGLMSYKVIPLDILLTHRRIKPCVQ | | |
| SAASERDMESSGVPVITLGSGKVMPVLGMGTFEKVGKGSE | | |
| RERLAILKAIEVGYRYFDTAAAYETEEVLGEAIAEALQLGL | | |
| VKSRDELFISSMLWCTDAHADRVLLALQNSLRNLKLEYVD | | |
| LYMLPFPASLKPGKITMDIPEEDICRMDYRXVSKPWLH* | | |
| MRWHRXIDSYGLSSVPYGKYWRELRKVCVHNLLSNQQLL | P. somniferum | SEQ. ID |
| KFRHLIISQVDTSFNKLYELCKNSEDNQGNYPTTTTAAGMV | plant source; | NO. 4 |
| RIDDWLAELSFNVIGRIVCGFQSGPKTGAPSRVEQFKEAINE | partial-length | |
| ASYFMSTSPVSDNVPMLGWIDQLTGLTRNMKHCGKKLDL | amino acid | |
| VVESIINDHRQKRRFSRTKGGDEKDDEQDDFIDICLSIMEQP | sequence | |
| QLPGNNNPSQIPIKSIVLDMIGGGTDTTKLTTIWTLSLLLNNP | >MIKW- | |
| HVLDKAKQEVDAHFRTKRRSTNDAAAAVVDFDDIRNLVYI | 2013651 | |
| QAIIKESMRLYPASPVVERLSGEDCVVGGFHVPAGTRLWA | | |
| NVWKMQRDPKVWDDPLVFRPDRFLSDEQKMVDVRGQNY | | |
| ELLPFGAGRRVCPGVSFSLDLMQLVLTRLILEFEMKSPSGK | | |
| VDMTATPGLMSYKVIPLDILLTHRRIKPCVQSAASERDMES | | |
| SGVPVITLGSGKVMPVLGMGTFEKVGKGSERERLAILKAIE | | |
| VGYRYFDTAAAYETEEVLGEAIAEALQLGLVKSRDELFISS | | |
| MLWCTDAHADRVLLALQNSLRNLKLEYVDLYMLPFPASL | | |
| KPGKITMDIPEEDICRMDYRSVWAAMEECQNLGFTKSIGVS | | |
| NFSCKKLQELMATANIPPAVNQVEMSPAFQQKKLREYCNA | | |
| NNILVSAISVLGSNGTPWGSNAVLGSEVLKKIAMAKGKSV | | |
| AQVSMRWVYEQGASLVVKSFSEERLRENLNIFDWELTKED | | |
| HEKIGEIPQCRILSAYFLVSPNGPFKSQEELWDDEA* | | |
| MELQYISYFQPTSSVVALLLALVSILSSVVVLRKTFLNNYSS | P. setigerum | SEQ. ID |
| SPASSTKTAVLSHQRQQSCALPISGLLHIFMNKNGLIHVTLG | plant source; | NO. 5 |
| NMADKYGPIFSFPTGSHRTLVVSSWEMVKECFTGNNDTAF | full-length | |
| SNRPIPLAFKTIFYACGGIDSYGLSSVPYGKYWRELRKVCV | amino acid | |
| HNLLSNQQLLKFRHLIISQVDTSFNKLYELCKNSEDNQGNY | sequence | |
| TTTTTAAGMVRIDDWLAELSFNVIGRIVCGFQSGPKTGAPS | >EPRK- | |
| RVEQFKEAINEASYFMSTSPVSDNVPMLGWIDQLTGLTRN | 2027940 | |
| MKHCGKKLDLVVESIINDHRQKRRFSRTKGGDEKDDEQDD | (also FPYZ- | |
| FIDICLSIMEQPQLPGNNNPSQIPIKSIVLDMIGGGTDTTKLTT | 2037562, | |
| IWTLSLLLNNPHVLDKAKQEVDAHFRTKRRSTNDAAAAVV | STDO- | |
| DFDDIRNLVYIQAIIKESMRLYPASPVVERLSGEDCVVGGFH | 2019715, | |
| VPAGTRLWANVWKMQRDPKVWDDPLVFRPDRFLSDEQK | FNXH- | |
| MVDVRGQNYELLPFGAGRRVCPGVSFSLDLMQLVLTRLIL | 2029312, | |
| EFEMKSPSGKVDMTATPGLMSYKVIPLDILLTHRRIKPCVQ | MLPX- | |
| SAASERDMESSGVPVITLGSGKVMPVLGMGTFEKVGKGSE | 2016196, | |
| RERLAILKAIEVGYRYFDTAAAYETEEVLGEAIAEALQLGL | MLPX- | |
| VKSRDELFISSMLWCTDAHADRVLLALQNSLRNLKLEYVD | 2016197) | |
| LYMLPFPASLKPGKITMDIPEEDICRMDYRSVWAAMEECQ | | |
| NLGFTKSIGVSNFSCKKLQELMATANIPPAVNQVEMSPAFQ | | |
| QKKLREYCNANNILVSAISVLGSNGTPWGSNAVLGSEVLK | | |
| KIAMAKGKSVAQVSMRWVYEQGASLVVKSFSEERLRENL | | |
| NIFDWELTKEDHEKIGEIPQCRILSAYFLVSPNGPFKSQEEL | | |
| WDDEA* | | |
| MELQYISYFQPTSSVVALLLALVSILSSVVVLRKTFLNNYSS | P. setigerum | SEQ. ID |
| SPASSTKTAVLSHQRQQSCALPISGLLHIFMNKNGLIHVTLG | plant source; | NO. 6 |
| NMADKYGPIFSFPTGSHRTLVVSSWEMVKECFTGNNDTAF | partial-length | |
| SNRPIPLAFKTIFYACGGIDSYGLSSVPYGKYWRELRKVCV | amino acid | |
| HNLLSNQQLLKFRHLIISQVDTSFNKLYELCKNSEDNQGNY | sequence | |
| TTTTTAAGMVRIDDWLAELSFNVIGRIVCGFQSGPKTGAPS | >QCOU- | |
| RVEQFKEAINEASYFMSTSPVSDNVPMLGWIDQLTGLTRN | 2000833 | |
| MKHCGKKLDLVVESIINDHRQKRRFSRTKGGDEKDDEQDD | | |
| FIDICLSIMEQPQLPGNNNPSQIPIKSIVLDMIGGGTDTTKLTT | | |
| IWTLSLLLNNPHVLDKAKQEVDAHFRTKRRSTNDAAAAVV | | |
| DFDDIRNLVYIQALYPASPVVERLSGEDCVVGGFHVPAGTR | | |
| LWANVWKMQRDPKVWDDPLVFRPDRFLSDEQKMVDVRG | | |
| QNYELLPFGAGRRVCPGVSFSLDLMQLVLTRLILEFEMKSP | | |
| SGKVDMTATPGLMSYKVIPLDILLTHRRIKPCVQSAASERD | | |
| MESSGVPVITLGSGKVMPVLGMGTFEKVGKGSERERLAIL | | |
| KAIEVGYRYFDTAAAYETEEVLGEAIAEALQLGLVKSRDEL | | |
| FISSMLWCTDAHADRVLLALQNSLRNLKLEYVDLYMLPFP | | |
| ASLKPGKITMDIPEEDICRMDYRSVWAAMEE | | |
| MELQYFSYFQPTSSVVALLLALVSILFSVVVLRKTFSNNYSS | P. bracteatum | SEQ. ID |
| PASSTETAVLCHQRQQSCALPISGLLHVFMNKNGLIHVTLG | plant source; | NO. 7 |
| NMADKYGPIFSFPTGSHRTLVVSSWEMVKECFTGNNDTAF | full-length | |
| SNRPIPLAFQTIFYACGGIDSYGLSSVPYGKYWRELRKVCV | amino acid | |
| HNLLSNQQLLKFRHLIISQVDTSFNKLYELCKNSEDNQGMV | sequence | |
| RMDDWLAQLSFNVIGRIVCGFQSDPKTGAPSRVEQFKEVIN | >SSDU- | |
| EASYFMSTSPVSDNVPMLGWIDQLTGLTRNMKHCGKKLD | 2015634 | |
| LVVESIIKDHRQKRRFSRTKGGDEKDDEQDDFIDICLSIMEQ | (also SSDU- | |
| PQLPGNNSPPQIPIKSIVLDMIGGGTDTTKLTTIWTLSLLLNN | 2015636, | |
| PHVLDKAKQEVDAHFRKKRRSTDDAAAAVVDFDDIRNLV | ZSNV- | |
| YIQAIIKESMRLYPASPVVERLSGEDCVVGGFHVPAGTRLW | 2027701, | |
| ANVWKMQRDPKVWDDPLVFRPERFLSDEQKMVDVRGQN | RRID- | |
| YELLPFGAGRRICPGVSFSLDLMQLVLTRLILEFEMKSPSGK | 2004435) | |
| VDMTATPGLMSYKVVPLDILLTHRRIKSCVQLASSERDMES | | |
| SGVPVITLSSGKVMPVLGMGTFEKVGKGSERERLAILKAIE | | |
| VGYRYFDTAAAYETEEVLGEAIAEALQLGLIESRDELFISSM | | |
| LWCTDAHPDRVLLALQNSLRNLKLEYLDLYMLPFPASLKP | | |
| GKITMDIPEEDICRMDYRSVWSAMEECQNLGFTKSIGVSNF | | |
| SSKKLQELMATANIPPAVNQVEMSPAFQQKKLREYCNANN | | |
| ILVSAVSILGSNGTPWGSNAVLGSEVLKQIAMAKGKSVAQ | | |
| VSMRWVYEQGASLVVKSFSEERLRENLNIFDWELTKEDNE | | |
| KIGEIPQCRILTAYFLVSPNGPFKSQEELWDDKA* | | |
| MELQYFSYFQPTSSVVALLLALVSILFSVVVLRKTFSNNYSS | P. bracteatum | SEQ. ID |
| PASSTETAVLCHQRQQSCALPISGLLHVFMNKNGLIHVTLG | plant source; | NO. 8 |
| NMADKYGPIFSFPTGSHRTLVVSSWEMVKECFTGNNDTAF | full-length | |
| SNRPIPLAFQTIFYACGGIDSYGLSSVPYGKYWRELRKVCV | amino acid | |
| HNLLSNQQLLKFRHLIISQVDTSFNKLYELCKNSEDNQGMV | sequence | |
| RMDDWLAQLSFNVIGRIVCGFQSDPKTGAPSRVEQFKEVIN | >TMWO- | |
| EASYFMSTSPVSDNVPMLGWIDQLTGLTRNMKHCGKKLD | 2027322 | |
| LVVESIIKDHRQKRRFSRTKGGDEKDDEQDDFIDICLSIMEQ | (also RRID- | |
| PQLPGNNSPPQIPIKSIVLDMIGGGTDTTKLTTIWTLSLLLNN | 2004435) | |
| PHVLDKAKQEVDAHFRKKRRSTDDAAAAVVDFDDIRNLV | | |
| YIQAIIKESMRLYPASPVVERLSGEDCVVGGFHVPAGTRLW | | |
| ANVWKMQRDPKVWDDPLVFRPERFLSDEQKMVDVRGQN | | |
| YELLPFGAGRRICPGVSFSLDLMQLVLTRLILEFEMKSPSGK | | |
| VDMTATPGLMSYKVVPLDILLTHRRIKSCVQLASSERDMES | | |
| SGVPVITLSSGKVMPVLGMGTFEKVGKGSERERLAILKAIE | | |
| VGYRYFDTAAAYETEEVLGEAIAEALQLGLIESRDELFISSM | | |
| LWCTDAHPDRVLLALQNSLRNLKLEYLDLYMLPFPASLKP | | |
| GKITMDIPEEDICRMDYRSVWSAMEECQNLGFTKSIGVSNF | | |
| SCKKLQELMATANIPPAVNQVEMSPAFQQKKLREYCNANN | | |
| ILVSAVSILGSNGTPWGSNAVLGSEVLKQIAMAKGKSVAQ | | |
| VSMRWVYEQGASLVVKSFSEERLRENLNIFDWELTKEDNE | | |
| KIGEIPQCRILTAYFLVSPNGPFKSQEELWDDKA* | | |
| SSPASSTETAVLCHQRQQSCALPISGLLHIFMNKNGLIHVTL | P. bracteatum | SEQ. ID |
| GNMADKYGPIFSFPTGSHRILVVSSWEMVKECFTGNNDTA | plant source; | NO. 9 |
| FSNRPIPLAFKTIFYACRGIDSYGLSSVPYGKYWRELRKVCV | partial-length | |
| HNLLSNQQLLKFRHLIISQVDTSFNKLYELCKNSEDNQGMV | amino acid | |
| RMDDWLAQLSFSVIGRIVCGFQSDPKTGAPSRVEQFKEAIN | sequence | |
| EASYFMSTSPVSDNVPMLGWIDQLTGLTRNMTHCGKKLDL | >pbr.PBRST1PF_ | |
| VVESIINDHRQKRRFSRTKGGDEKDDEQDDFIDICLSIMEQP | 89405 | |
| QLPGNNNPPKIPIKSIVLDMIGAGTDTTKLTIIWTLSLLLNNP | | |
| NVLAKAKQEVDAHFETKKRSTNEASVVVDFDDIGNLVYIQ | | |
| AIIKESMRLYPVSPVVERLSSEDCVVGGFHVPAGTRLWANV | | |
| WKMQRDPKVWDDPLVFRPERFLSDEQKMVDVRGQNYEL | | |
| LPFGAGRRICPGVSFSLDLMQLVLTRLILEFEMKSPSGKVD | | |
| MTATPGLMSYKVVPLDILLTHRRIKSCVQLASSERDMESSG | | |
| VPVITLRSGKVMPVLGMGTFEKAGKGSERERLAILKAIEVG | | |
| YRYFDTAAAYETEEVLGEAIAEALQLGLIKSRDELFISSML | | |
| WCTDAHPDRVLLALQNSLRNLKLEYVDLYMLPFPASLKPG | | |
| KITMDIPEEDICPMDYRSVWSAMEECQNLGLTKSIGVSNFS | | |
| CKKLEELMATANIPPAVNQVEMSPAFQQKKLREYCNANNI | | |
| LVSAVSILGSNGTPWGSNAVLGSEVLKKIAMAKGKSVAQV | | |
| SMRWVYEQGASLVVKSFSEERLRENLNIFDWQLTKEDNEK | | |
| IGEIPQCRILSAYFLVSPKGPFKSQEELWDDKA* | | |
| SSPASSTETAVLCHQRQQSCALPISGLLHIFMNKNGLIHVTL | P. bracteatum | SEQ. ID |
| GNMADKYGPIFSFPTGSHRILVVSSWEMVKECFTGNNDTFF | plant source; | NO. 10 |
| SNRPIPLAFKIIFYAGGVDSYGLALVPYGKYWRELRKICVH | partial-length | |
| NLLSNQQLLKFRHLIISQVDTSFNKLYELCKNSEDNQGMVR | amino acid | |
| MDDWLAQLSFSVIGRIVCGFQSDPKTGAPSRVEQFKEAINE | sequence | |
| ASYFMSTSPVSDNVPMLGWIDQLTGLTRNMTHCGKKLDL | >pbr.PBRST1PF_ | |
| VVESIINDHRQKRRFSRTKGGDEKDDEQDDFIDICLSIMEQP | 4328 | |
| QLPGNNNPPKIPIKSIVLDMIGGGTDTTKLTTIWTLSLLLNNP | | |
| HVLDKAKQEVDAHFLTKRRSTNDAAVVDFDDIRNLVYIQA | | |
| IIKESMRLYPASPVVERLSGEDCVVGGFHVPAGTRLWVNV | | |
| WKMQRDPNVWADPMVFRPERFLSHGQKKMVDVRGKNYE | | |
| LLPFGAGRRICPGISFSLDLMQLVLTRLILEFEMKSPSGKVD | | |
| MTATPGLMSYKVVPLDILLTHRRIKSCVQLASSERDMESSG | | |
| VPVITLRSGKVMPVLGMGTFEKAGKGSERERLAILKAIEVG | | |
| YRYFDTAAAYETEEVLGEAIAEALQLGLIKSRDELFISSML | | |
| WCTDAHPDRVLLALQNSLRNLKLEYVDLYMLPFPASLKPG | | |
| KITMDIPEEDICPMDYRSVWSAMEECQNLGLTKSIGVSNFS | | |
| CKKLEELMATANIPPAVNQVEMSPAFQQKKLREYCNANNI | | |
| LVSAVSILGSNGTPWGSNAVLGSEVLKKIAMAKGKSVAQV | | |
| SMRWVYEQGASLVVKSFSEERLRENLNIFDWQLTKEDNEK | | |
| IGEIPQCRILSAYFLVSPKGPFKSQEELWDDKA* | | |
| SSPASSTETAVLCHQRQQSCALPISGLLHIFMNKNGLIHVTL | P. bracteatum | SEQ. ID |
| GNMADKYGPIFSFPTGSHRILVVSSWEMVKECFTGNNDTFF | plant source; | NO. 11 |
| SNRPIPLAFKIIFYAGGVDSYGLALVPYGKYWRELRKICVH | partial-length | |
| NLLSNQQLLNFRHLIISQVDTSFNKLYDLSNKKKNTTTDSG | amino acid | |
| TVRMDDWLAQLSFNVIGRIVCGFQTHTETSATSSVERFTEA | sequence | |
| IDEASRFMSIATVSDTFPWLGWIDQLTGLTRKMKHYGKKL | >pbr.PBRST1PF_ | |
| DLVVESIIEDHRQNRRISGTKQGDDFIDICLSIMEQPQIIPGN | 12180 | |
| NDPPRQIPIKSIVLDMIGGGTDTTKLTTTWTLSLLLNNPHVL | | |
| EKAREEVDAHFGTKRRPTNDDAVMVEFDDIRNLVYIQAIIK | | |
| ESMRLYPASPVVERLSGEDCVVGGFHVPAGTRLWVNVWK | | |
| MQRDPNVWADPMVFRPERFLSDEQKMVDVRGQNYELLPF | | |
| GAGRRICPGVSFSLDLMQLVLTRLILEFEMKSPSGKVDMTA | | |
| TPGLMSYKVVPLDILLTHRRIKSCVQLASSERDMESSGVPVI | | |
| TLRSGKVMPVLGMGTFEKAGKGSERERLAILKAIEVGYRY | | |
| FDTAAAYETEEVLGEAIAEALQLGLIKSRDELFISSMLWCT | | |
| DAHPDRVLLALQNSLRNLKLEYVDLYMLPFPASLKPGKIT | | |
| MDIPEEDICPMDYRSVWSAMEECQNLGLTKSIGVSNFSCKK | | |
| LEELMATANIPPAVNQVEMSPAFQQKKLREYCNANNILVS | | |
| AVSILGSNGTPWGSNAVLGSEVLKKIAMAKGKSVAQVSMR | | |
| WVYEQGASLVVKSFSEERLRENLNIFDWQLTKEDNEKIGEI | | |
| PQCRILSAYFLVSPKGPFKSQEELWDDKA* | | |
| VALRKKILKNYYSSSSSTATAVSHQWPKASRALPLIDLLHV | P. bracteatum | SEQ. ID |
| FFNKTDLMHVTLGNMADKFGPIFSFPTGSHRTLVVSSWEK | plant source; | NO. 12 |
| AKECFTGNNDIVFSGRPLPLAFKLIFYAGGIDSYGISQVPYG | partial-length | |
| KKWRELRNICVHNILSNQQLLKFRHLMISQVDNSFNKLYEV | amino acid | |
| CNSNKDEGDSATSTTAAGIVRMDDWLGKLAFDVIARIVCG | sequence | |
| FQSQTETSTTSSMERFTEAMDEASRFMSVTAVSDTVPWLG | >pbr.PBRST1PF_ | |
| WIDQLTGLKRNMKHCGKKLNLVVKSIIEDHRQKRRLSSTK | 4329 | |
| KGDENIIDEDEQDDFIDICLSIMEQPQLPGNNNPPKIPIKSIVL | | |
| DMIGGGTDTTKLTTIWTLSLLLNNPHVLDKAKQEVDAHFL | | |
| TKRRSTNDAAVVDFDDIRNLVYIQAIIKESMRLYPASPVVE | | |
| RLSGEDCVVGGFHVPAGTRLWVNVWKMQRDPNVWADP | | |
| MVFRPERFLSDEQKMVDVRGQNYELLPFGAGRRICPGVSFS | | |
| LDLMQLVLTRLILEFEMKSPSGKVDMTATPGLMSYKVVPL | | |
| DILLTHRRIKSCVQLASSERDMESSGVPVITLRSGKVMPVLG | | |
| MGTFEKAGKGSERERLAILKAIEVGYRYFDTAAAYETEEVL | | |
| GEAIAEALQLGLIKSRDELFISSMLWCTDAHPDRVLLALQN | | |
| SLRNLKLEYVDLYMLPFPASLKPGKITMDIPEEDICPMDYRS | | |
| VWSAMEECQNLGLTKSIGVSNFSCKKLEELMATANIPPAV | | |
| NQVEMSPAFQQKKLREYCNANNILVSAVSILGSNGTPWGS | | |
| NAVLGSEVLKKIAMAKGKSVAQVSMRWVYEQGASLVVKS | | |
| FSEERLRENLNIFDWQLTKEDNEKIGEIPQCRILSAYFLVSPK | | |
| GPFKSQEELWDDKA* | | |
| MELQYFSYFQPTSSVVALLLALVSILFSVVVLRKTFSNNYSS | P. bracteatum | SEQ. ID |
| PASSTETAVLCHQRQQSCALPISGLLHVFMNKNGLIHVTLG | plant source; | NO. 13 |
| NMADKYGPIFSFPTGSHRTLVVSSWEMVKECFTGNNDTAF | partial-length | |
| SNRPIPLAFQTIFYACGGIDSYGLSSVPYGKYWRELRKVCV | amino acid | |
| HNLLSNQQLLKFRHLIISQVDTSFNKLYELCKNSEDNQGMV | sequence | |
| RMDDWLAQLSFNVIGRIVCGFQSDPKTGAPSRVEQFKEVIN | >SSDU- | |
| EASYFMSTSPVSDNVPMLGWIDQLTGLTRNMKHCGKKLD | 2015635 | |
| LVVESIIKDHRQKRRFSRTKGGDEKDDEQDDFIDICLSIMEQ | | |
| PQLPGNNSPPQIPIKSIVLDMIGGGTDTTKLTTIWTLSLLLNN | | |
| PHVLDKAKQEVDAHFRKKRRSTDDAAAAVVDFDDIRNLV | | |
| YIQAIIKESMRLYPASPVVERLSGEDCVVGGFHVPAGTRLW | | |
| ANVWKMQRDPKVWDDPLVFRPERFLSDEQKMVDVRGQN | | |
| YELLPFGAGRRICPGVSFSLDLMQLVLTRLILEFEMKSPSGK | | |
| VDMTATPGLMSYKVVPLDILLTHRRIKSCVQLASSERDMES | | |
| SGVPVITLSSGKVMPVLGMGTFEKVGKGSERERLAILKAIE | | |
| VGYRYFDTAAAYETEEVLGEAIAEALQLGLIESRDELFISSM | | |
| LWCTDAHPDRVLLALQNSLRNLKLEYLDLYMLPFPASLKP | | |
| GKITMDIPEEDICRMDYRSVWSAMEECQNLGFTKSIGVSNF | | |
| SSKKLQELMATANIPPAVNQVEMSPAFQQKKLREYCNANN | | |
| ILVSAVSILGSNGTPWGSNAVLGSEVLKQIAMAKGKSVAQ | | |
| VSMRWVXKFSAYAIVWSLFFGHRICITLYSFLIRNVAYICIT | | |
| Y* | | |
| MELQYFSYFQPTSSVVALLLALVSILFSVVVLRKTFSNNYSS | P. bracteatum | SEQ. ID |
| PASSTETAVLCHQRQQSCALPISGLLHVFMNKNGLIHVTLG | plant source; | NO. 14 |
| NMADKYGPIFSFPTGSHRTLVVSSWEMVKECFTGNNDTAF | partial-length | |
| SNRPIPLAFQTIFYACGGIDSYGLSSVPYGKYWRELRKVCV | amino acid | |
| HNLLSNQQLLKFRHLIISQVDTSFNKLYELCKNSEDNQGMV | sequence | |
| RMDDWLAQLSFNVIGRIVCGFQSDPKTGAPSRVEQFKEVIN | >SSDU- | |
| EASYFMSTSPVSDNVPMLGWIDQLTGLTRNMKHCGKKLD | 2015637 | |
| LVVESIIKDHRQKRRFSRTKGGDEKDDEQDDFIDICLSIMEQ | | |
| PQLPGNNSPPQIPIKSIVLDMIGGGTDTTKLTTIWTLSLLLNN | | |
| PHVLDKAKQEVDAHFRKKRRSTDDAAAAVVDFDDIRNLV | | |
| YIQAIIKESMRLYPASPVVERLSGEDCVVGGFHVPAGTRLW | | |
| ANVWKMQRDPKVWDDPLVFRPERFLSDEQKMVDVRGQN | | |
| YELLPFGAGRRICPGVSFSLDLMQLVLTRLILEFEMKSPSGK | | |
| VDMTATPGLMSYKVVPLDILLTHRRIKSCVQLASSERDMES | | |
| SGVPVITLSSGKVMPVLGMGTFEKVGKGSERERLAILKAIE | | |
| VGYRYFDTAAAYETEEVLGEAIAEALQLGLIESRDELFISSM | | |
| LWCTDAHPDRVLLALQNSLRQVFLMQIRLIYICTYQQVHL | | |
| NIYFQINEFVLCDMYRNLKLEY | | |
| LNNYSSSPASSTKTAVLSHQRQQSCALPISGLLHIFMNKNGL | C. majus | SEQ. ID |
| IHVTLGNMADKYGPIFSFPTGSHRTLVVSSWEMVKECFTGN | plant | NO. 15 |
| NDTAFSNRPIPLAFKTIFYACGGIDSYGLSSVPYGKYWRELR | source; | |
| KVCVHNLLSNQQLLKFRHLIISQVDTSFNKLYELCKNSEDN | partial- | |
| QGNYPTTTTAAGMVRIDDWLAELSFNVIGRIVCGFQSGPKT | length amino | |
| GAPSRVEQFKEAINEASYFMSTSPVSDNVPMLGWIDQLTGL | acid sequence | |
| TRNMKHCGKKLDLVVESIINDHRQKRRFSRTKGGDEKDDE | >chm.CMAST2PF_ | |
| QDDFIDICLSIMEQPQLPGNNNPSQIPIKSIVLDMIGGGTDTT | 14984 | |
| KLTTIWTLSLLLNNPHVLDKAKQEVDAHFRTKRRSTNDAA | | |
| AAVVDFDDIRNLVYIQAIIKESMRLYPASPVVERLSGEDCV | | |
| VGGFHVPAGTRLWANVWKMQRDPKVWDDPLVFRPDRFL | | |
| SDEQKMVDVRGQNYELLPFGAGRRVCPGVSFSLDLMQLV | | |
| LTRLILEFEMKSPSGKVDMTATPGLMSYKVIPLDILLTHRRI | | |
| KPCVQSAASERDMESSGVPVITLGSGKVMPVLGMGTFEKV | | |
| GKGSERERLAFLKAIEVGYRYFDTAAAYETEEFLGEAIAEA | | |
| LQLGLIKSRDELFITSKLWPCDAHPDLVVPALQNSLRNLKL | | |
| EYVDLYMLPFPASLKPGKITMDIPEEDICRMDYRSVWAAM | | |
| EECQNLGFTKSIGVSNFSCKKLQELMATANIPPAVNQVEMS | | |
| PAFQQKKLREYCNANNILVSAISVLGSNGTPWGSNAVLGSE | | |
| VLKKIAMAKGKSVAQVSMRWVYEQGASLVVKSFSEERLR | | |
| ENLNIFDWELTKEDHEKIGEIPQCRILSAYFLVSPNGPFKSQE | | |
| ELWDDEA* | | |

### BisBIA Generating Modifications

Some methods, processes, and systems provided herein describe the production of bisbenzylisoquinoline alkaloids (bisBIAs). BisBIAs are dimeric molecules that may be formed by coupling reactions between two BIA monomers. In examples, bisBIAs may be formed by carbon-oxygen coupling reactions. In other examples, bisBIAs may be formed by carbon-carbon coupling reactions. In some examples, the bisBIA dimeric molecule is a homodimer, comprising two identical BIA monomers. In examples, an engineered host cell may produce one BIA monomer. In these examples, the BIA monomers may form homodimers when contacted with one or more coupling enzymes. In other examples, the bisBIA dimeric molecule is a heterodimer, comprising two different BIA monomers. For example, a bisBIA may be a heterodimer that comprises BIA monomers that are enantiomers of each other. In some examples, an engineered host cell may produce two or more BIA monomers. In these examples, the BIA monomers may form homodimers and heterodimers when contacted with one or more coupling enzymes.

Some of these methods, processes, and systems that describe the production of bisBIAs may comprise an engineered host cell. In some examples, the engineered host cell may be engineered to produce BIA monomers which, in turn, may be used as building block molecules for forming bisBIAs. Examples of BIA monomers that may be used to form bisBIAs include coclaurine, N-methylcoclaurine, laudanine, norcoclaurine, norlaudanosoline, 6-O-methyl-norlaudanosoline, 3′-hydroxy-N-methylcoclaurine, 3′-hydroxycoclaurine, reticuline, norreticuline, norlaudanine, laudanosine, and papaverine. In particular, engineered host cells may synthesize BIA monomers from norcoclaurine or norlaudanosoline by expression of heterologous enzymes including O-methyltransferases, N-methyltransferases, and 3′-hydroxylases. Examples of O-methyltransferases may include norcoclaurine 6-O-methyltransferase (6OMT) from Thalicrum flavum, Nelumbo nucifera, Populus euphratica, or another species. Further examples of O-methyltransferases may include catechol O-methyltransferase (COMT) from Homo sapiens, Mus musculus, Rattus norvegicus, Gorilla gorilla, or another species. Further examples of N-methyltransferases may include coclaurine N-methyltransferase (CNMT) from T. flavum, N. nucifera, Aristolochia fimbriata, or another species. Examples of 3′hydroxylases may include N-methylcoclaurine 3′-hydroxylase (CYP80B1) from Eschscholzia californica, T. flavum, N. nucifera, or another species.

The engineered host cells may produce either(S) or (R) enantiomers of any given BIA monomer. Additionally or alternatively, the engineered host cells may produce a mixture of both enantiomers. The ratio of(S) and (R) enantiomers may be determined by the substrate and product specificities of the one or more enzymes that synthesize the BIA monomers. Alternatively, the amount of each enantiomer present may be modified by the expression of an additional enzyme or enzymes that perform the epimerization of one stereoisomer into another, as discussed above.

These BIA monomers may be fused into a dimeric bisBIA scaffold. In particular, the BIA monomers may be fused into a dimeric bisBIA scaffold utilizing one or more enzymes that are produced by the engineered host cell. Additionally or alternatively, the BIA monomers may be fused into a dimeric bisBIA scaffold utilizing one or more enzymes that are provided to the BIA monomers from a source that is external to the engineered host cell. The one or more enzymes may be used to form carbon-oxygen and/or carbon-carbon coupling reactions to fuse two BIA monomers at one, two, or three positions. In some examples, two BIA monomers may be linked by an ether bridge. In some examples, a direct carbon-carbon bond may be used to connect the two BIA monomers. In some examples, a bisBIA that is formed by fusing two BIA monomers may comprise one diphenyl ether linkage. In some examples, two BIA monomers may be fused to form a bisBIA that comprises two diphenyl ether linkages. In some examples, a bisBIA that is formed from two BIA monomers may comprise three diphenyl ether linkages. In some examples, the bisBIA may comprise one diphenyl ether linkage and one benzyl phenyl ether linkage. In some cases, the bisBIA may comprise one benzyl phenyl ether linkage and two diphenyl ether linkages.

In examples, the BIA monomers may be contacted with a sufficient amount of the one or more enzymes that may be used to form coupling reactions to fuse two BIA monomers such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said BIA monomers are converted to bisBIAs. The one or more enzymes that may be used to dimerize the BIA monomers into bisBIAs may contact the BIA monomers in vitro. Additionally, or alternatively, the one or more enzymes that may be used to dimerize the BIA monomers into bisBIAs may contact the BIA monomers in vivo. Additionally, the one or more bisBIA dimerizing enzyme may be expressed in a host cell that produces BIA monomers. Alternatively, the BIA monomers may be provided to the engineered host cell that expresses the bisBIA dimerizing enzyme. Alternatively, the one or more bisBIA dimerizing enzymes may be provided to a cell having BIA monomers within.

In some examples, the bisbenzylisoquinoline alkaloid is a compound of any one of Formulas Va-Vu:

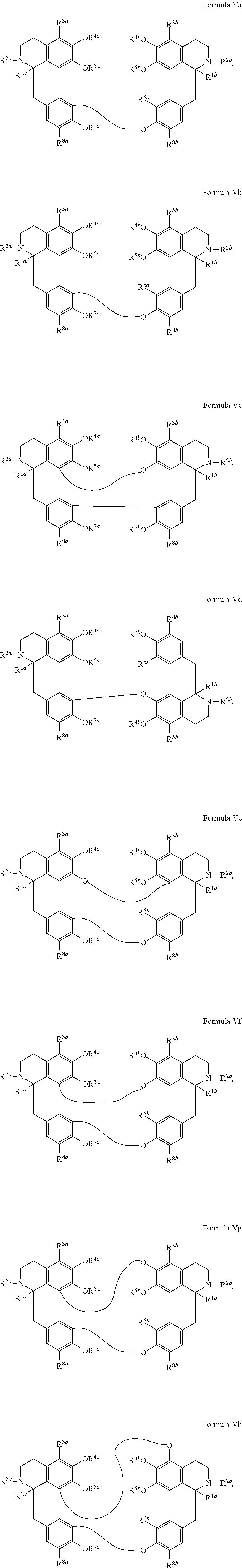

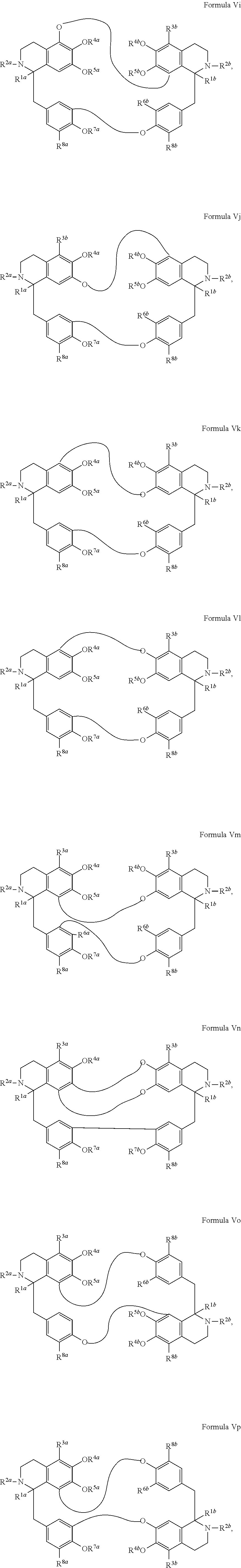

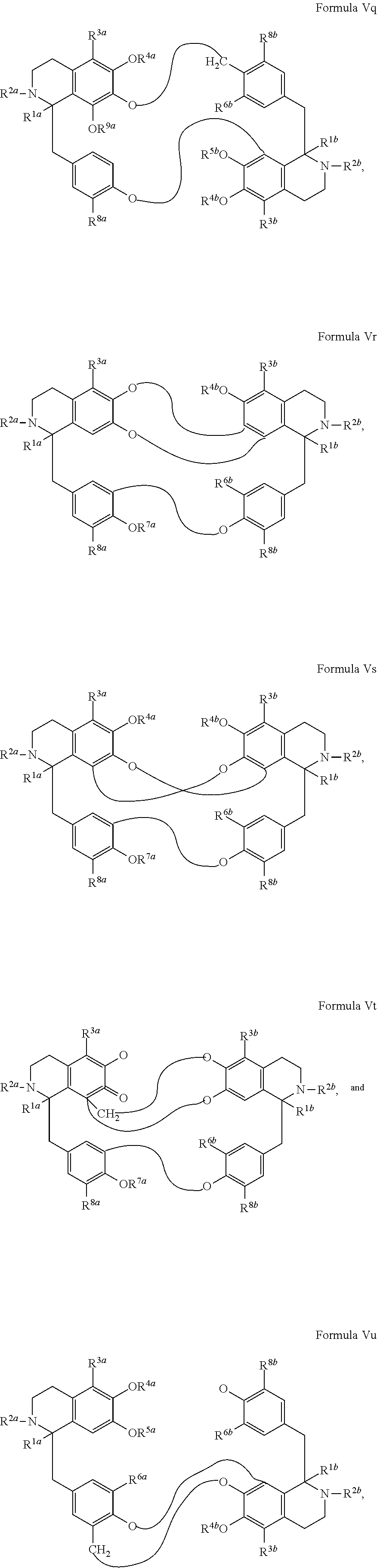

or a salt thereof, wherein:
R^{1a}, R^{1b}, R^{2a}, and R^{2b} are independently selected from hydrogen and C₁-C₄ alkyl; R^{3a}, R^{3b}, R^{6a}, R^{6b}, R^{8a}, and R^{8b} are independently selected from hydrogen, hydroxy, fluoro, chloro, bromo, carboxaldehyde, C₁-C₄ acyl, C₁-C₄ alkyl, and C₁-C₄ alkoxy;
R^{4a} and R^{5a} are independently selected from hydrogen and C₁-C₄ alkyl, or R^{4a} and R^{5a} together form a methylene bridge;
R^{4b} and R^{5b} are independently selected from hydrogen and C₁-C₄ alkyl, or R^{4b} and R^{5b} together form a methylene bridge; and
R^{7a}, R^{7b}, and R^{9a} are independently selected from hydrogen and C₁-C₄ alkyl.

In some examples, R^{1a} and R^{1b} are each hydrogen; R^{2a} and R^{2b} are each methyl; R^{3a} and R^{3b} are each hydrogen; R^{4a} and R^{5a} are independently hydrogen or methyl; R^{4b} and R^{5b} are independently hydrogen or methyl, or R^{4b} and R^{5b} together form a methylene bridge; R^{6a}, R^{6b}, R^{8a}, and R^{8b} are each hydrogen; and R^{7a}, R^{7b}, and R^{9a} are independently hydrogen or methyl.

As illustrated above, the bisBIA compounds of Formulas Va, Vb, and Vd are formed by fusing two BIA monomers using a carbon-oxygen coupling reaction. Additionally, the bisBIA compounds of Formulas Vc, Vf, and Vh are formed by fusing two BIA monomers using both a carbon-oxygen coupling reaction and a carbon-carbon coupling reaction. Further, the bisBIA compounds of Formulas Ve, Vg, Vi, Vj, Vk, Vl, Vm, Vo, Vp, and Vq are formed by fusing two BIA monomers using two carbon-oxygen coupling reactions. The bisBIA compound of Formula Vn is formed by fusing two BIA monomers using two carbon-oxygen coupling reactions and a carbon-carbon coupling reaction. Additionally, the bisBIA compound of Formula Vr is formed by fusing two BIA monomers using three carbon-oxygen coupling reactions.

The one or more enzymes that may be used to form the coupling reactions may include known cytochrome P450s such as Berberis stolonifera CYP80A1 or similar cytochrome P450 enzymes from other plants that naturally synthesize these compounds. Alternatively, the coupling reaction may be performed by an enzyme that is not a cytochrome P450. The one or more enzymes that may be used to form the coupling reactions may be engineered to accept non-native substrates. Accordingly, the one or more enzymes that may be used to form the coupling reactions may be used to generate non-natural bisBIA molecules. In examples, the one or more enzymes may fuse a natural BIA monomer with a non-natural BIA monomer to produce a non-natural bisBIA molecule. In other examples, the one or more enzymes may fuse two non-natural BIA monomers to produce a non-natural bisBIA molecule. Enzyme engineered strategies may be used to identify one or more enzymes that may be used to form the coupling reactions that fuse BIA monomers to produce bisBIAs. In examples, enzyme engineering strategies may include site directed mutagenesis, random mutagenesis and screening, DNA shuffling, and screening.

Once bisBIAs are formed, the bisBIAs may be further derivatized or modified. The bisBIAs may be derivatized or modified utilizing one or more enzymes that are produced by the engineered host cell. In particular, the bisBIAs may be derivatized or modified by contacting the bisBIAs with one or more enzymes that are produced by the engineered host cell. Additionally or alternatively, the bisBIAs may be derivatized or modified by contacting the bisBIAs with one or more enzymes that are provided to the bisBIAs from a source that is external to the engineered host cell. The one or more enzymes that may be used to derivatize or modify the bisBIAs may be used to perform tailoring reactions. Examples of tailoring reactions include oxidation, reduction, O-methylation, N-methylation, O-demethylation, acetylation, methylenedioxybridge formation, and O,O-demethylenation. A bisBIA may be derivatized or modified using one or more tailoring reactions.

Examples of tailoring reactions are provided in Table 8. In some examples, tailoring enzymes may be used to catalyze carbon-carbon coupling reactions performed on a bisBIA, or a derivative thereof. Examples of tailoring enzymes that may be used to catalyze carbon-carbon coupling reactions include a Berberine bridge enzyme (BBE) from Papaver somniferum, Eschscholzia californica, Coptis japonica, Berberis stolonifer, Thalictrum flavum, or another species; Salutaridine synthase (SalSyn) from Papaver somniferum or another species; and Corytuberine synthase (CorSyn) from Coptis japonica or another species. Non-limiting examples of reactions that can be catalyzed by tailoring enzymes are shown in Scheme 2, wherein R^{a}, R^{b}, R^{c}, and R^{d} are independently selected from hydrogen, hydroxy, fluoro, chloro, bromo, carboxaldehyde, C₁-C₄ acyl, C₁-C₄ alkyl, and C₁-C₄ alkoxy. In some examples, R^{a}, R^{b}, and the carbon atoms to which they are attached optionally form a carbocycle or heterocycle. In some examples, R^{c}, R^{d}, and the carbon atoms to which they are attached optionally form a carbocycle or heterocycle.

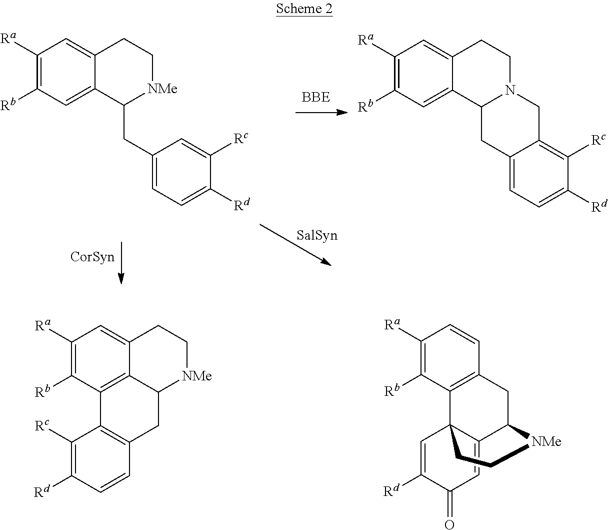

In some examples, tailoring enzymes may be used to catalyze oxidation reactions performed on a bisBIA, or a derivative thereof. Examples of tailoring enzymes that may be used to catalyze oxidation reactions include a Tetrahydroprotoberberine oxidase (STOX) from Coptis japonica, Argemone mexicana, Berberis wilsonae, or another species; Dihydrobenzophenanthridine oxidase (DBOX) from Papaver somniferum or another species; Methylstylopine hydroxylase (MSH) from Papaver somniferum or another species; and Protopine 6-hydroxylase (P6H) from Papaver somniferum, Eschscholzia californica, or another species.

Tailoring enzymes may also be used to catalyze methylenedioxy bridge formation reactions performed on a bisBIA, or a derivative thereof. Examples of tailoring enzymes that may be used to catalyze methylenedioxy bridge formation reactions include a Stylopine synthase (StySyn or STS) from Papaver somniferum, Eschscholzia californica, Argemone mexicana, or another species; Cheilanthifoline synthase (CheSyn or CFS) from Papaver somniferum, Eschscholzia californica, Argemone mexicana, or another species; and Canadine synthase (CAS) from Thalictrum flavum, Coptis chinensis, or another species.

In other examples, tailoring enzymes may be used to catalyze O-methylation reactions performed on a bisBIA, or a derivative thereof. Examples of tailoring enzymes that may be used to catalyze O-methylation reactions include a Norcoclaurine 6-O-methyltransferase (6OMT) from Papaver somniferum, Thalictrum flavum, Coptis japonica, Papaver bracteatum, or another species; 3′hydroxy-N-methylcoclaurine 4′-O-methyltransferase (4′OMT) from Papaver somniferum, Thalictrum flavum, Coptis japonica, Coptis chinensis, or another species; Reticuline 7-O-methyltransferase (7OMT) from Papaver somniferum, Eschscholzia californica, or another species; and Scoulerine 9-O-methyltransferase (90MT) from Papaver somniferum, Thalictrum flavum, Coptis japonica, Coptis chinensis, or another species.

Additionally, tailoring enzymes may be used to catalyze N-methylation reactions performed on a bisBIA, or a derivative thereof. Examples of tailoring enzymes that may be used to catalyze N-methylation reactions include Coclaurine N-methyltransferase (CNMT) from Papaver somniferum, Thalictrum flavum, Coptis japonica, or another species; Tetrahydroprotoberberine N-methyltransferase (TNMT) from Papaver somniferum, Eschscholzia californica, Papaver bracteatum, or another species.

Further, tailoring enzymes may be used to catalyze O-demethylation reactions performed on a bisBIA, or a derivative thereof. Examples of tailoring enzymes that may be used to catalyze O-demethylation reactions include Thebaine demethylase (T6ODM) from Papaver somniferum or another species; and Codeine demethylase (CODM) from Papaver somniferum, or another species.

Tailoring enzymes may also be used to catalyze reduction reactions performed on a bisBIA, or a derivative thereof. Examples of tailoring enzymes that may be used to catalyze reduction reactions include Salutaridine reductase (SalR) from Papaver somniferum, Papaver bracteatum, or another species; Codeinone reductase (COR) from Papaver somniferum or another species; and Sanguinarine reductase (SanR) from Eschscholzia californica or another species. In other examples, tailoring enzymes may be used to catalyze acetylation reactions performed on a bisBIA, or a derivative thereof. An example of a tailoring enzyme that may be used to catalyze acetylation reactions includes Salutaridine acetyltransferase (SalAT) from Papaver somniferum or another species.

### O-Demethylation Modifications

Some methods, processes, and systems provided herein describe the conversion of a first benzylisoquinoline alkaloid to a second benzylisoquinoline alkaloid by the removal of an O-linked methyl group. Some of these methods, processes, and systems may comprise an engineered host cell. In some examples, the conversion of a first benzylisoquinoline alkaloid to a second benzylisoquinoline alkaloid is a key step in the conversion of a substrate to a nor-opioids or nal-opioids. In some examples, the conversion of a first alkaloid to a second alkaloid comprises a demethylase reaction.

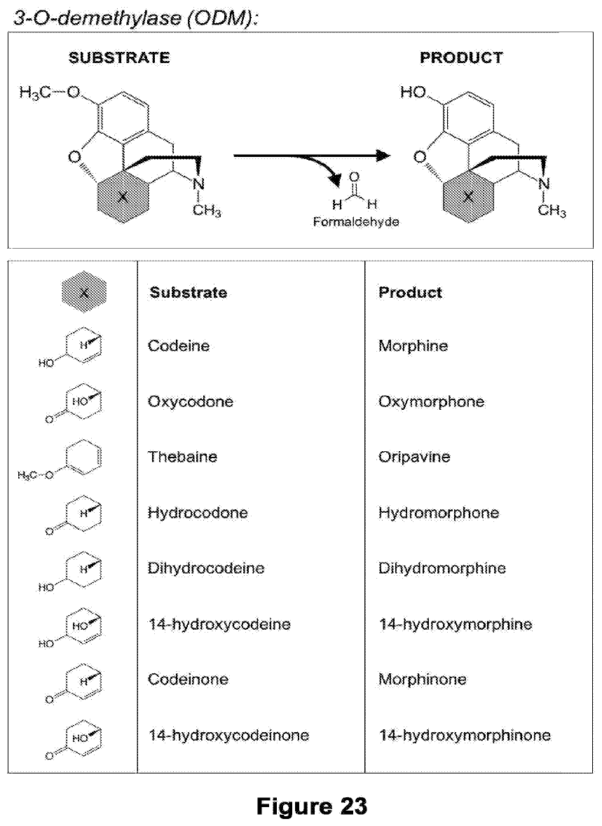

FIG. 23 illustrates an enzyme having opioid 3-O-demethylase activity, in accordance with embodiments of the invention. Specifically, the enzyme may act on any morphinan alkaloid structure to remove the methyl group from the oxygen bound to carbon 3.

Examples of amino acid sequences of ODM enzymes are set forth in Table 3. An amino acid sequence for an ODM that is utilized in converting a first alkaloid to a second alkaloid may be 75% or more identical to a given amino acid sequence as listed in Table 3. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an ODM that converts a first alkaloid to a second alkaloid, wherein the ODM comprises a given amino acid sequence as listed in Table 3. An engineered host cell may be provided that produces one or more ODM enzymes. The ODM that is produced within the engineered host cell may be recovered and purified so as to form a biocatalyst. The process may include contacting the first alkaloid with an ODM in an amount sufficient to convert said first alkaloid to a second alkaloid. In examples, the first alkaloid may be contacted with a sufficient amount of the one or more enzymes such that at least 5% of said first alkaloid is converted to a second alkaloid. In further examples, the first alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said first alkaloid is converted to a second alkaloid.

The one or more enzymes that may be used to convert a first alkaloid to a second alkaloid may contact the first alkaloid in vitro. Additionally, or alternatively, the one or more enzymes that may be used to convert a first alkaloid to a second alkaloid may contact the first alkaloid in vivo. In some examples, the one or more enzymes that may be used to convert a first alkaloid to a second alkaloid may be provided to a cell having the first alkaloid within. In some examples, the one or more enzymes that may be used to convert a first alkaloid to a second alkaloid may be produced within an engineered host cell.

In some examples, the methods provide for engineered host cells that produce an alkaloid product, wherein the O-demethylation of a substrate to a product may comprise a key step in the production of an alkaloid product. In some examples, the alkaloid produced is a nor-opioid or a nal-opioid. In still other embodiments, the alkaloid produced is derived from a nor-opioid or a nal-opioid. In another embodiment, a first alkaloid is an intermediate toward the product of the engineered host cell. In still other embodiments, the alkaloid product is selected from the group consisting of morphine, oxymorphine, oripavine, hydromorphone, dihydromorphine, 14-hydroxymorphine, morphinone, and 14-hydroxymorphinone.

In some examples, the substrate alkaloid is an opioid selected from the group consisting of codeine, oxycodone, thebaine, hydrocodone, dihydrocodeine, 14-hydroxycodeine, codeinone, and 14-hydroxycodeinone.

### N-Demethylation Modifications

Some methods, processes, and systems provided herein describe the conversion of a first alkaloid to a second alkaloid by the removal of an N-linked methyl group. Some of these methods, processes, and systems may comprise an engineered host cell. In some examples, the conversion of a first alkaloid to a second alkaloid is a key step in the conversion of a substrate to a nor-opioids or nal-opioids. In some examples, the conversion of a first alkaloid to a second alkaloid comprises a demethylase reaction.

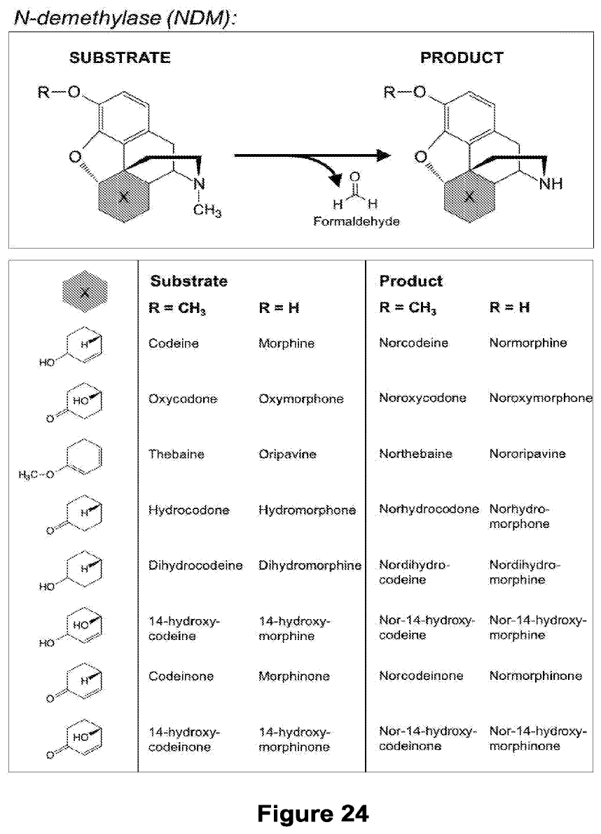

FIG. 24 illustrates an enzyme having opioid N-demethylase activity, in accordance with embodiments of the invention. Specifically, the enzyme may act on any morphinan alkaloid structure to remove the methyl group from the nitrogen.

Examples of an amino acid sequence of an N-demethylase enzyme that may be used to perform the conversion a first alkaloid to a second alkaloid are provided in Table 4. An amino acid sequence for an NDM that is utilized in converting a first alkaloid to a second alkaloid may be 75% or more identical to a given amino acid sequence as listed in Table 4. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an NDM that converts a first alkaloid to a second alkaloid, wherein the NDM comprises an amino acid sequence as listed in Table 4. An engineered host cell may be provided that produces one or more NDM enzymes. The NDM that is produced within the engineered host cell may be recovered and purified so as to form a biocatalyst. The process may include contacting the first alkaloid with an NDM in an amount sufficient to convert said first alkaloid to a second alkaloid. In examples, the first alkaloid may be contacted with a sufficient amount of the one or more enzymes such that at least 5% of said first alkaloid is converted to a second alkaloid. In further examples, the first alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said first alkaloid is converted to a second alkaloid.

The one or more enzymes that may be used to convert a first alkaloid to a second alkaloid may contact the first alkaloid in vitro. Additionally, or alternatively, the one or more enzymes that may be used to convert a first alkaloid to a second alkaloid may contact the first alkaloid in vivo. In some examples, the one or more enzymes that may be used to convert a first alkaloid to a second alkaloid may be provided to a cell having the first alkaloid within. In some examples, the one or more enzymes that may be used to convert a first alkaloid to a second alkaloid may be produced within an engineered host cell.

In some examples, the methods provide for engineered host cells that produce an alkaloid product, wherein the N-demethylation of a substrate to a product may comprise a key step in the production of an alkaloid product. In some examples, the alkaloid produced is a nor-opioid or a nal-opioid. In still other embodiments, the alkaloid produced is derived from a nor-opioid or a nal-opioid. In another embodiment, a first alkaloid is an intermediate toward the product of the engineered host cell. In still other embodiments, the alkaloid product is selected from the group consisting of norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone.

In some examples, the substrate alkaloid is an opioid selected from the group consisting of codeine, oxycodone, thebaine, hydrocodone, dihydrocodeine, 14-hydroxycodeine, codeinone, and 14-hydroxycodeinone, morphine, oxymorphone, oripavine, hydromorphone, dihydromorphine, 14-hydroxy-morphine, morphinone, or 14-hydroxy-morphinone.

### N-Methyltransferase Modifications

Some methods, processes, and systems provided herein describe the conversion of a first alkaloid to a second alkaloid by the addition of an N-linked sidechain group. Some methods, processes, and systems provided herein describe the conversion of a first alkaloid to a second alkaloid by the transfer of a sidechain group from a cosubstrate to the first alkaloid. Some of these methods, processes, and systems may comprise an engineered host cell. In some examples, the conversion of a first alkaloid to a second alkaloid is a key step in the conversion of a substrate to a nal-opioid. In some examples, the conversion of a first alkaloid to a second alkaloid comprises a methyltransferase reaction.

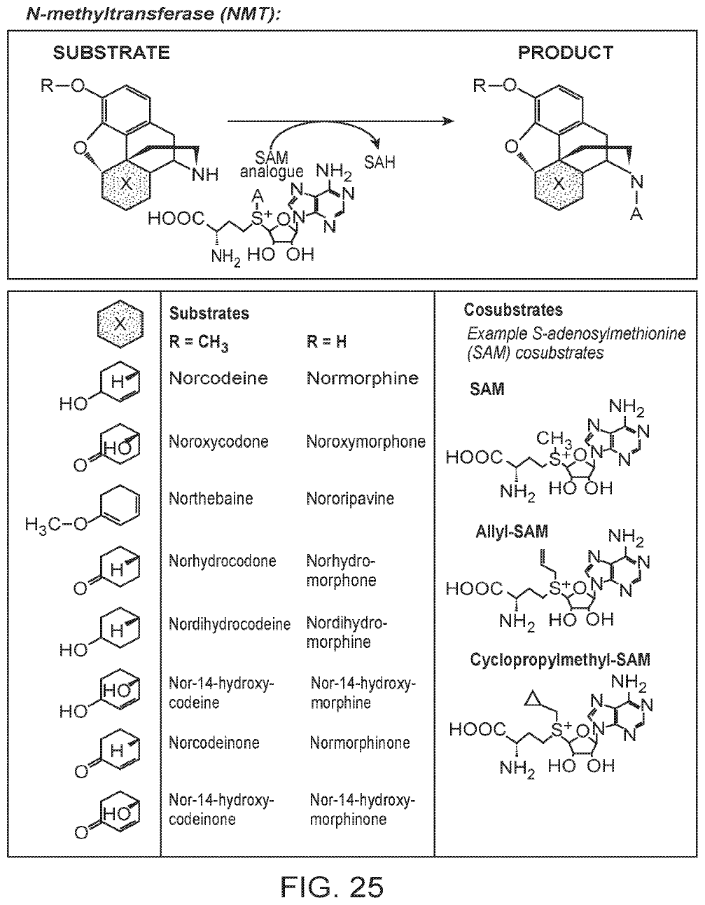

FIG. 25 illustrates an enzyme having N-methyltransferase activity, in accordance with embodiments of the invention. Specifically, the enzyme may act on any morphinan alkaloid structure to add a methyl group or other carbon moiety to the nitrogen. S-Adenosyl methionine (SAM) may act as the donor of the functional group (methyl, allyl, cyclopropylmethyl, or other).

Examples of amino acid sequences of NMT enzymes are set forth in Table 5. An amino acid sequence for an NMT that is utilized in converting a first alkaloid to a second alkaloid may be 75% or more identical to a given amino acid sequence as listed in Table 5. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an NMT that converts a first alkaloid to a second alkaloid, wherein the NMT comprises an amino acid sequence as provided in Table 5. An engineered host cell may be provided that produces one or more NMT enzymes. The NMT that is produced within the engineered host cell may be recovered and purified so as to form a biocatalyst. The process may include contacting the first alkaloid with an NMT in an amount sufficient to convert said first alkaloid to a second alkaloid. In examples, the first alkaloid may be contacted with a sufficient amount of the one or more enzymes such that at least 5% of said first alkaloid is converted to a second alkaloid. In further examples, the first alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said first alkaloid is converted to a second alkaloid.

The one or more enzymes that may be used to convert a first alkaloid to a second alkaloid may contact the first alkaloid in vitro. Additionally, or alternatively, the one or more enzymes that may be used to convert a first alkaloid to a second alkaloid may contact the first alkaloid in vivo. In some examples, the one or more enzymes that may be used to convert a first alkaloid to a second alkaloid may be provided to a cell having the first alkaloid within. In some examples, the one or more enzymes that may be used to convert a first alkaloid to a second alkaloid may be produced within an engineered host cell.

In some examples, the methods provide for engineered host cells that produce an alkaloid product, wherein the N-methyltransferase of a substrate to a product may comprise a key step in the production of an alkaloid product. In some examples, the alkaloid produced is a nal-opioid. In still other embodiments, the alkaloid produced is derived from a nor-opioid or a nal-opioid. In another embodiment, a first alkaloid is an intermediate toward the product of the engineered host cell. In still other embodiments, the alkaloid product is selected from the group including naloxone, naltrexone, and nalmefene.

In some examples, the substrate alkaloid is an opioid selected from the group consisting of norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone. In some examples, the cosubstrate is S-adenosylmethionine, Allyl-S-adenosylmethionine, or cyclopropylmethyl-S-adenosylmethionine.

### Heterologous Coding Sequences

In some instances, the engineered host cells harbor one or more heterologous coding sequences (such as two or more, three or more, four or more, five or more) which encode activity (ies) that enable the engineered host cells to produce desired enzymes of interest and/or BIAs of interest, e.g., as described herein. As used herein, the term “heterologous coding sequence” is used to indicate any polynucleotide that codes for, or ultimately codes for, a peptide or protein or its equivalent amino acid sequence, e.g., an enzyme, that is not normally present in the host organism and may be expressed in the host cell under proper conditions. As such, “heterologous coding sequences” includes multiple copies of coding sequences that are normally present in the host cell, such that the cell is expressing additional copies of a coding sequence that are not normally present in the cells. The heterologous coding sequences may be RNA or any type thereof, e.g., mRNA, DNA or any type thereof, e.g., cDNA, or a hybrid of RNA/DNA. Coding sequences of interest include, but are not limited to, full-length transcription units that include such features as the coding sequence, introns, promoter regions, 3′-UTRs, and enhancer regions.

The engineered host cells may also be modified to possess one or more genetic alterations to accommodate the heterologous coding sequences. Alterations of the native host genome include, but are not limited to, modifying the genome to reduce or ablate expression of a specific protein that may interfere with the desired pathway. The presence of such native proteins may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product.

Heterologous coding sequences include but are not limited to sequences that encode enzymes, either wild-type or equivalent sequences, that are normally responsible for the production of BIAs of interest in plants. In some cases, the enzymes for which the heterologous sequences code may be any of the enzymes in the 1-BIA pathway, and may be from any convenient source. The choice and number of enzymes encoded by the heterologous coding sequences for the particular synthetic pathway may be selected based upon the desired product. In certain embodiments, the host cells may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more heterologous coding sequences, such as 1, 2, 3, 4, 5, 6, 7, 8, 9, 10, 11, 12, 13, 14, or 15 heterologous coding sequences.

As used herein, the term “heterologous coding sequences” also includes the coding portion of the peptide or enzyme, i.e., the cDNA or mRNA sequence, of the peptide or enzyme, as well as the coding portion of the full-length transcriptional unit, i.e., the gene including introns and exons, as well as “codon optimized” sequences, truncated sequences or other forms of altered sequences that code for the enzyme or code for its equivalent amino acid sequence, provided that the equivalent amino acid sequence produces a functional protein. Such equivalent amino acid sequences may have a deletion of one or more amino acids, with the deletion being N-terminal, C-terminal, or internal. Truncated forms are envisioned as long as they have the catalytic capability indicated herein. Fusions of two or more enzymes are also envisioned to facilitate the transfer of metabolites in the pathway, provided that catalytic activities are maintained.

Operable fragments, mutants, or truncated forms may be identified by modeling and/or screening. In some cases, this is achieved by deletion of, for example, N-terminal, C-terminal, or internal regions of the protein in a step-wise fashion, followed by analysis of the resulting derivative with regard to its activity for the desired reaction compared to the original sequence. If the derivative in question operates in this capacity, it is considered to constitute an equivalent derivative of the enzyme proper.

In examples, some heterologous proteins may show occurrences where they are incorrectly processed when expressed in a recombinant host. For example, plant proteins such as cytochrome P450 enzymes expressed in microbial production hosts may have occurrences of incorrect processing. In particular, salutaridine synthase may undergo N-linked glycosylation when heterologously expressed in yeast. This N-linked glycosylation may not be observed in plants, which may be indicative of incorrect N-terminal sorting of the nascent SalSyn transcript so as to reduce the activity of the enzyme in the heterologous microbial host. In such examples, protein engineering directed at correcting N-terminal sorting of the nascent transcript so as to remove the N-linked glycosylation pattern may result in improved activity of the salutaridine synthase enzyme in the recombinant production host. This is explained further in Example 8 below.

Some aspects of the invention also relate to heterologous coding sequences that code for amino acid sequences that are equivalent to the native amino acid sequences for the various enzymes. An amino acid sequence that is “equivalent” is defined as an amino acid sequence that is not identical to the specific amino acid sequence, but rather contains at least some amino acid changes (deletions, substitutions, inversions, insertions, etc.) that do not essentially affect the biological activity of the protein as compared to a similar activity of the specific amino acid sequence, when used for a desired purpose. The biological activity refers to, in the example of an epimerase, its catalytic activity. Equivalent sequences are also meant to include those which have been engineered and/or evolved to have properties different from the original amino acid sequence. Mutable properties of interest include catalytic activity, substrate specificity, selectivity, stability, solubility, localization, etc.

In some instances, the expression of each type of enzyme is increased through additional gene copies (i.e., multiple copies), which increases intermediate accumulation and/or BIA of interest production. Some embodiments of the invention include increased BIA of interest production in a host cell through simultaneous expression of multiple species variants of a single or multiple enzymes. In some cases, additional gene copies of a single or multiple enzymes are included in the host cell. Any convenient methods may be utilized including multiple copies of a heterologous coding sequence for an enzyme in the host cell.

In some examples, the engineered host cell includes multiple copies of a heterologous coding sequence for an enzyme, such as 2 or more, 3 or more, 4 or more, 5 or more, or even 10 or more copies. In certain embodiments, the engineered host cell includes multiple copies of heterologous coding sequences for one or more enzymes, such as multiple copies of two or more, three or more, four or more, etc. In some cases, the multiple copies of the heterologous coding sequence for an enzyme are derived from two or more different source organisms as compared to the host cell. For example, the engineered host cell may include multiple copies of one heterologous coding sequence, where each of the copies is derived from a different source organism. As such, each copy may include some variations in explicit sequences based on inter-species differences of the enzyme of interest that is encoded by the heterologous coding sequence.

The engineered host cell medium may be sampled and monitored for the production of BIAs of interest. The BIAs of interest may be observed and measured using any convenient methods. Methods of interest include, but are not limited to, LC-MS methods (e.g., as described herein) where a sample of interest is analyzed by comparison with a known amount of a standard compound. Additionally, there are other ways that BIAs of interest may be observed and/or measured. Examples of alternative ways of observing and/or measuring BIAs include GC-MS, UV-vis spectroscopy, NMR, LC-NMR, LC-UV, TLC, capillary electrophoresis, among others. Identity may be confirmed, e.g., by m/z and MS/MS fragmentation patterns, and quantitation or measurement of the compound may be achieved via LC trace peaks of know retention time and/or EIC MS peak analysis by reference to corresponding LC-MS analysis of a known amount of a standard of the compound.

Additionally, a culture of the engineered host cell may be sampled and monitored for the production of enzymes of interest, such as a CYP—COR enzyme. The enzymes of interest may be observed and measured using any convenient methods. Methods of interest include enzyme activity assays, polyacrylamide gel electrophoresis, carbon monoxide spectroscopy, and western blot analysis.

### Methods

Methods for Culturing Host Cells for BIA production

As summarized above, some aspects of the invention include methods of preparing nor-opioid and nal-opioid BIAs of interest. Additionally, some aspects of the invention include methods of preparing enzymes of interest. As such, some aspects of the invention include culturing an engineered host cell under conditions in which the one or more host cell modifications (e.g., as described herein) are functionally expressed such that the cell converts starting compounds of interest into product nor-opioid and/or nal-opioid BIAs of interest. Also provided are methods that include culturing an engineered host cell under conditions suitable for protein production such that one or more heterologous coding sequences are functionally expressed and convert starting compounds of interest into product enzymes or nor-opioid and/or nal-opioid BIAs of interest. In examples, the method is a method of preparing a nor-opioid and/or nal-opioid BIA of interest that includes culturing an engineered host cell (e.g., as described herein); adding a starting compound to the cell culture; and recovering the nor-opioid and/or nal-opioid from the cell culture. In some examples, the method is a method of preparing an enzyme that includes culturing an engineered host cell (e.g., as described herein); adding a starting compound to the cell culture; and recovering the enzyme from the cell culture.

Fermentation media may contain suitable carbon substrates. The source of carbon suitable to perform the methods of this disclosure may encompass a wide variety of carbon containing substrates. Suitable substrates may include, without limitation, monosaccharides (e.g., glucose, fructose, galactose, xylose), oligosaccharides (e.g., lactose, sucrose, raffinose), polysaccharides (e.g., starch, cellulose), or a combination thereof. In some cases, unpurified mixtures from renewable feedstocks may be used (e.g., cornsteep liquor, sugar beet molasses, barley malt). In some cases, the carbon substrate may be a one-carbon substrate (e.g., methanol, carbon dioxide) or a two-carbon substrate (e.g., ethanol). In other cases, other carbon containing compounds may be utilized, for example, methylamine, glucosamine, and amino acids.

Any convenient methods of culturing engineered host cells may be employed for producing the nor-opioid and/or nal-opioid BIAs of interest. The particular protocol that is employed may vary, e.g., depending on the engineered host cell, the heterologous coding sequences, the enzymes of interest, the nor-opioid and/or nal-opioid BIAs of interest, etc. The cells may be present in any convenient environment, such as an environment in which the cells are capable of expressing one or more functional heterologous enzymes. In some embodiments, the cells are cultured under conditions that are conducive to enzyme expression and with appropriate substrates available to allow production of nor-opioid and/or nal-opioid BIAs of interest in vivo. In some embodiments, the functional enzymes are extracted from the engineered host for production of nor-opioid and/or nal-opioid BIAs of interest under in vitro conditions. In some instances, the engineered host cells are placed back into a multicellular host organism. The engineered host cells are in any phase of growth, including, but not limited to, stationary phase and log-growth phase, etc. In addition, the cultures themselves may be continuous cultures or they may be batch cultures.

Cells may be grown in an appropriate fermentation medium at a temperature between 14-40° C. Cells may be grown with shaking at any convenient speed (e.g., 200 rpm). Cells may be grown at a suitable pH. Suitable pH ranges for the fermentation may be between pH 5-9. Fermentations may be performed under aerobic, anaerobic, or microaerobic conditions. Any suitable growth medium may be used. Suitable growth media may include, without limitation, common commercially prepared media such as synthetic defined (SD) minimal media or yeast extract peptone dextrose (YEPD) rich media. Any other rich, defined, or synthetic growth media appropriate to the microorganism may be used.

Cells may be cultured in a vessel of essentially any size and shape. Examples of vessels suitable to perform the methods of this disclosure may include, without limitation, multi-well shake plates, test tubes, flasks (baffled and non-baffled), and bioreactors. The volume of the culture may range from 10 microliters to greater than 10,000 liters.

The addition of agents to the growth media that are known to modulate metabolism in a manner desirable for the production of alkaloids may be included. In a non-limiting example, cyclic adenosine 2′3′-monophosphate may be added to the growth media to modulate catabolite repression.

Any convenient cell culture conditions for a particular cell type may be utilized. In certain embodiments, the host cells that include one or more modifications are cultured under standard or readily optimized conditions, with standard cell culture media and supplements. As one example, standard growth media when selective pressure for plasmid maintenance is not required may contain 20 g/L yeast extract, 10 g/L peptone, and 20 g/L dextrose (YPD). Host cells containing plasmids are grown in synthetic complete (SC) media containing 1.7 g/L yeast nitrogen base, 5 g/L ammonium sulfate, and 20 g/L dextrose supplemented with the appropriate amino acids required for growth and selection. Alternative carbon sources which may be useful for inducible enzyme expression include, but are not limited to, sucrose, raffinose, and galactose. Cells are grown at any convenient temperature (e.g., 30° C.) with shaking at any convenient rate (e.g., 200 rpm) in a vessel, e.g., in test tubes or flasks in volumes ranging from 1-1000 mL, or larger, in the laboratory.

Culture volumes may be scaled up for growth in larger fermentation vessels, for example, as part of an industrial process. The industrial fermentation process may be carried out under closed-batch, fed-batch, or continuous chemostat conditions, or any suitable mode of fermentation. In some cases, the cells may be immobilized on a substrate as whole cell catalysts and subjected to fermentation conditions for alkaloid production.

Batch fermentation is a closed system, in which the composition of the medium is set at the beginning of the fermentation and not altered during the fermentation process. The desired organism(s) are inoculated into the medium at the beginning of the fermentation. In some instances, the batch fermentation is run with alterations made to the system to control factors such as pH and oxygen concentration (but not carbon). In this type of fermentation system, the biomass and metabolite compositions of the system change continuously over the course of the fermentation. Cells typically proceed through a lag phase, then to a log phase (high growth rate), then to a stationary phase (growth rate reduced or halted), and eventually to a death phase (if left untreated).

A continuous fermentation is an open system, in which a defined fermentation medium is added continuously to the bioreactor and an equal amount of fermentation media is continuously removed from the vessel for processing. Continuous fermentation systems are generally operated to maintain steady state growth conditions, such that cell loss due to medium being removed must be balanced by the growth rate in the fermentation. Continuous fermentations are generally operated at conditions where cells are at a constant high cell density. Continuous fermentations allow for the modulation of one or more factors that affect target product concentration and/or cell growth.

The liquid medium may include, but is not limited to, a rich or synthetic defined medium having an additive component described above. Media components may be dissolved in water and sterilized by heat, pressure, filtration, radiation, chemicals, or any combination thereof. Several media components may be prepared separately and sterilized, and then combined in the fermentation vessel. The culture medium may be buffered to aid in maintaining a constant pH throughout the fermentation.

Process parameters including temperature, dissolved oxygen, pH, stirring, aeration rate, and cell density may be monitored or controlled over the course of the fermentation. For example, temperature of a fermentation process may be monitored by a temperature probe immersed in the culture medium. The culture temperature may be controlled at the set point by regulating the jacket temperature. Water may be cooled in an external chiller and then flowed into the bioreactor control tower and circulated to the jacket at the temperature required to maintain the set point temperature in the vessel.

Additionally, a gas flow parameter may be monitored in a fermentation process. For example, gases may be flowed into the medium through a sparger. Gases suitable for the methods of this disclosure may include compressed air, oxygen, and nitrogen. Gas flow may be at a fixed rate or regulated to maintain a dissolved oxygen set point.

The pH of a culture medium may also be monitored. In examples, the pH may be monitored by a pH probe that is immersed in the culture medium inside the vessel. If pH control is in effect, the pH may be adjusted by acid and base pumps which add each solution to the medium at the required rate. The acid solutions used to control pH may be sulfuric acid or hydrochloric acid. The base solutions used to control pH may be sodium hydroxide, potassium hydroxide, or ammonium hydroxide.

Further, dissolved oxygen may be monitored in a culture medium by a dissolved oxygen probe immersed in the culture medium. If dissolved oxygen regulation is in effect, the oxygen level may be adjusted by increasing or decreasing the stirring speed. The dissolved oxygen level may also be adjusted by increasing or decreasing the gas flow rate. The gas may be compressed air, oxygen, or nitrogen.

Stir speed may also be monitored in a fermentation process. In examples, the stirrer motor may drive an agitator. The stirrer speed may be set at a consistent rpm throughout the fermentation or may be regulated dynamically to maintain a set dissolved oxygen level.

Additionally, turbidity may be monitored in a fermentation process. In examples, cell density may be measured using a turbidity probe. Alternatively, cell density may be measured by taking samples from the bioreactor and analyzing them in a spectrophotometer. Further, samples may be removed from the bioreactor at time intervals through a sterile sampling apparatus. The samples may be analyzed for alkaloids produced by the host cells. The samples may also be analyzed for other metabolites and sugars, the depletion of culture medium components, or the density of cells.

In another example, a feed stock parameter may be monitored during a fermentation process. In particular, feed stocks including sugars and other carbon sources, nutrients, and cofactors that may be added into the fermentation using an external pump. Other components may also be added during the fermentation including, without limitation, anti-foam, salts, chelating agents, surfactants, and organic liquids.

Any convenient codon optimization techniques for optimizing the expression of heterologous polynucleotides in host cells may be adapted for use in the subject host cells and methods, see e.g., Gustafsson C., et al. (2004) Trends Biotechnol, 22, 346-353, which is incorporated by reference in its entirety.

The subject method may also include adding a starting compound to the cell culture. Any convenient methods of addition may be adapted for use in the subject methods. The cell culture may be supplemented with a sufficient amount of the starting materials of interest (e.g., as described herein), e.g., an amount in the mM to UM range such as between about 1-5 mM of a starting compound. It is understood that the amount of starting material added, the timing and rate of addition, the form of material added, etc., may vary according to a variety of factors. The starting material may be added neat or pre-dissolved in a suitable solvent (e.g., cell culture media, water, or an organic solvent). The starting material may be added in concentrated form (e.g., 10× over desired concentration) to minimize dilution of the cell culture medium upon addition. The starting material may be added in one or more batches, or by continuous addition over an extended period of time (e.g., hours or days).

Methods for Isolating Products from the Fermentation Medium

The subject methods may also include recovering the nor-opioid and/or nal-opioid BIAs of interest from the cell culture. Any convenient methods of separation and isolation (e.g., chromatography methods or precipitation methods) may be adapted for use in the subject methods to recover the nor-opioid and/or nal-opioid BIAs of interest from the cell culture. Filtration methods may be used to separate soluble from insoluble fractions of the cell culture. In some cases, liquid chromatography methods (e.g., reverse phase HPLC, size exclusion, or normal phase chromatography) may be used to separate the BIA of interest from other soluble components of the cell culture. In some cases, extraction methods (e.g., liquid extraction, pH based purification, solid phase extraction, affinity chromatography, ion exchange, etc.) may be used to separate the nor-opioid and/or nal-opioid BIAs of interest from other components of the cell culture.

The produced alkaloids may be isolated from the fermentation medium using methods known in the art. A number of recovery steps may be performed immediately after (or in some instances, during) the fermentation for initial recovery of the desired product. Through these steps, the alkaloids (e.g., nor-opioids or nal-opioids) may be separated from the cells, cellular debris and waste, and other nutrients, sugars, and organic molecules may remain in the spent culture medium. This process may be used to yield a nor-opioid or nal-opioid-enriched product.

In an example, a product stream having a nor-opioid or nal-opioid product is formed by providing engineered yeast cells and a feedstock including nutrients and water to a batch reactor. In particular, the engineered yeast cells may be subjected to fermentation by incubating the engineered yeast cells for a time period of at least about 5 minutes to produce a solution comprising the nor-opioid or nal-opioid product and cellular material. Once the engineered yeast cells have been subjected to fermentation, at least one separation unit may be used to separate the nor-opioid or nal-opioid product from the cellular material to provide the product stream comprising the nor-opioid or nal-opioid product. In particular, the product stream may include the nor-opioid or nal-opioid product as well as additional components, such as a clarified yeast culture medium. Additionally, a nor-opioid or nal-opioid product may comprise one or more nor-opioids or nal-opioids of interest, such as one or more nor-opioid or nal-opioid compounds.

Different methods may be used to remove cells from a bioreactor medium that include an enzyme and/or nor-opioid or nal-opioid of interest. In examples, cells may be removed by sedimentation over time. This process of sedimentation may be accelerated by chilling or by the addition of fining agents such as silica. The spent culture medium may then be siphoned from the top of the reactor or the cells may be decanted from the base of the reactor. Alternatively, cells may be removed by filtration through a filter, a membrane, or other porous material. Cells may also be removed by centrifugation, for example, by continuous flow centrifugation or by using a continuous extractor.

Different methods may be used to remove cells from a bioreactor medium that include a BIA of interest such as naloxone or naltrexone. In examples, cells may be removed by sedimentation over time. This process of sedimentation may be accelerated by chilling or by the addition of fining agents such as silica. The spent culture medium may then be siphoned from the top of the reactor or the cells may be decanted from the base of the reactor. Alternatively, cells may be removed by filtration through a filter, a membrane, or other porous material. Cells may also be removed by centrifugation, for example, by continuous flow centrifugation or by using a continuous extractor.

If some valuable nor-opioid and/or nal-opioid BIAs of interest are present inside the cells, the cells may be permeabilized or lysed and the cell debris may be removed by any of the methods described above. Agents used to permeabilize the cells may include, without limitation, organic solvents (e.g., DMSO) or salts (e.g., lithium acetate). Methods to lyse the cells may include the addition of surfactants such as sodium dodecyl sulfate, or mechanical disruption by bead milling or sonication.

Nor-opioid and/or nal-opioid BIAs of interest may be extracted from the clarified spent culture medium through liquid-liquid extraction by the addition of an organic liquid that is immiscible with the aqueous culture medium. In examples, the use of liquid-liquid extraction may be used in addition to other processing steps. Examples of suitable organic liquids include, but are not limited to, isopropyl myristate, ethyl acetate, chloroform, butyl acetate, methylisobutyl ketone, methyl oleate, toluene, oleyl alcohol, ethyl butyrate. The organic liquid may be added to as little as 10% or as much as 100% of the volume of aqueous medium.

In some cases, the organic liquid may be added at the start of the fermentation or at any time during the fermentation. This process of extractive fermentation may increase the yield of nor-opioid and/or nal-opioid BIAs of interest from the host cells by continuously removing nor-opioids and/or nal-opioids to the organic phase.

Agitation may cause the organic phase to form an emulsion with the aqueous culture medium. Methods to encourage the separation of the two phases into distinct layers may include, without limitation, the addition of a demulsifier or a nucleating agent, or an adjustment of the pH. The emulsion may also be centrifuged to separate the two phases, for example, by continuous conical plate centrifugation.

Alternatively, the organic phase may be isolated from the aqueous culture medium so that it may be physically removed after extraction. For example, the solvent may be encapsulated in a membrane.

In examples, nor-opioid and/or nal-opioid BIAs of interest may be extracted from a fermentation medium using adsorption methods. In examples, nor-opioids or nal-opioids of interest may be extracted from clarified spent culture medium by the addition of a resin such as Amberlite® XAD4 or another agent that removes nor-opioids or nal-opioids by adsorption. The nor-opioids or nal-opioids of interest may then be released from the resin using an organic solvent. Examples of suitable organic solvents include, but are not limited to, methanol, ethanol, ethyl acetate, or acetone.

Nor-opioids or nal-opioids of interest may also be extracted from a fermentation medium using filtration. At high pH, the nor-opioids or nal-opioids of interest may form a crystalline-like precipitate in the bioreactor. This precipitate may be removed directly by filtration through a filter, membrane, or other porous material. The precipitate may also be collected by centrifugation and/or decantation.

The extraction methods described above may be carried out either in situ (in the bioreactor) or ex situ (e.g., in an external loop through which media flows out of the bioreactor and contacts the extraction agent, then is recirculated back into the vessel). Alternatively, the extraction methods may be performed after the fermentation is terminated using the clarified medium removed from the bioreactor vessel.

Methods for Purifying Products from Alkaloid-Enriched Solutions

Subsequent purification steps may involve treating the post-fermentation solution enriched with nor-opioid or nal-opioid product(s) of interest using methods known in the art to recover individual product species of interest to high purity.

In one example, nor-opioids or nal-opioids of interest extracted in an organic phase may be transferred to an aqueous solution. In some cases, the organic solvent may be evaporated by heat and/or vacuum, and the resulting powder may be dissolved in an aqueous solution of suitable pH. In a further example, the BIAs of interest may be extracted from the organic phase by addition of an aqueous solution at a suitable pH that promotes extraction of the nor-opioids or nal-opioids of interest into the aqueous phase. The aqueous phase may then be removed by decantation, centrifugation, or another method.

The nor-opioid or nal-opioid-containing solution may be further treated to remove metals, for example, by treating with a suitable chelating agent. The nor-opioid or nal-opioid of interest-containing solution may be further treated to remove other impurities, such as proteins and DNA, by precipitation. In one example, the nor-opioid or nal-opioid of interest-containing solution is treated with an appropriate precipitation agent such as ethanol, methanol, acetone, or isopropanol. In an alternative example, DNA and protein may be removed by dialysis or by other methods of size exclusion that separate the smaller alkaloids from contaminating biological macromolecules.

In further examples, the solution containing nor-opioids or nal-opioids of interest may be extracted to high purity by continuous cross-flow filtration using methods known in the art.

If the solution contains a mixture of nor-opioids or nal-opioids of interest, it may be subjected to acid-base treatment to yield individual nor-opioid or nal-opioid of interest species using methods known in the art. In this process, the pH of the aqueous solution is adjusted to precipitate individual nor-opioids or nal-opioids.

For high purity, small-scale preparations, the nor-opioids or nal-opioids may be purified in a single step by liquid chromatography.

### LCMS Method:

The BIA compounds of interest such as naloxone or naltrexone may be separated using liquid chromatography, and detected and quantified using mass spectrometry. Compound identity may be confirmed by characteristic elution time, mass-to-charge ratio (m/z) and fragmentation patterns (MS/MS). Quantitation may be performed by comparison of compound peak area to a standard curve of a known reference standard compound. Additionally, BIAs of interest may be detected by alternative methods such as GC-MS, UV-vis spectroscopy, NMR, LC-NMR, LC-UV, TLC, and capillary electrophoresis.

### Purpald Assay Method

For high throughput screening of demethylation reactions a purpald assay may be used. For example, demethylation catalyzed by 2-oxoglutarate dependent dioxygenases produces formaldehyde a as product as shown in the generalized chemical equation: [substrate]+2-oxoglutarate+O2↔[product]+formaldehyde+succinate+CO₂. Purpald reagent in alkaline conditions undergoes a color change in the presence of formaldehyde that can be quantified to concentrations as low as 1 nM with a spectrophotometer at 510 nm.

### Yeast-Derived Alkaloid APIs Versus Plant-Derived APIs

The clarified yeast culture medium (CYCM) may contain a plurality of impurities. The clarified yeast culture medium may be dehydrated by vacuum and/or heat to yield an alkaloid-rich powder. This product is analogous to the concentrate of poppy straw (CPS), which is exported from poppy-growing countries and purchased by Active Pharmaceutical Ingredients (API) manufacturers. For the purposes of this invention, CPS is a representative example of any type of purified plant extract from which the desired alkaloids product(s) may ultimately be further purified. Table 9 and Table 10 highlight the impurities in these two products that may be specific to either CYCM or CPS or may be present in both. Accordingly, these nor-opioids or nal-opioids may be assessed for impurities based on non-pigment impurities. By analyzing a product of unknown origin for a subset of these impurities, a person of skill in the art could determine whether the product originated from a yeast or plant production host.

API-grade pharmaceutical ingredients are highly purified molecules. As such, impurities that could indicate the plant- or yeast- origin of an API (such as those listed in Table 9 and Table 10) may not be present at the API stage of the product. Indeed, many of the API products derived from yeast strains of the present invention may be largely indistinguishable from the traditional plant-derived APIs. In some cases, however, conventional alkaloid compounds may be subjected to chemical modification using chemical synthesis approaches, which may show up as chemical impurities in plant-based products that require such chemical modifications. For example, chemical derivatization may often result in a set of impurities related to the chemical synthesis processes. In certain situations, these modifications may be performed biologically in the yeast production platform, thereby avoiding some of the impurities associated with chemical derivation from being present in the yeast-derived product. In particular, these impurities from the chemical derivation product may be present in an API product that is produced using chemical synthesis processes but may be absent from an API product that is produced using a yeast-derived product. Alternatively, if a yeast-derived product is mixed with a chemically-derived product, the resulting impurities may be present but in a lesser amount than would be expected in an API that only or primarily contains chemically-derived products. In this example, by analyzing the API product for a subset of these impurities, a person of skill in the art could determine whether the product originated from a yeast production host or the traditional chemical derivatization route.

Non-limiting examples of impurities that may be present in chemically-derivatized morphinan APIs but not in biosynthesized APIs include a codeine-O (6)-methyl ether impurity in API codeine; 8,14-dihydroxy-7,8-dihydrocodeinone in API oxycodone; and tetrahydrothebaine in API hydrocodone. The codeine-O (6)-methyl ether may be formed by chemical over-methylation of morphine. The 8,14-dihydroxy-7,8-dihydrocodeinone in API oxycodone may be formed by chemical over-oxidation of thebaine. Additionally, the tetrahydrothebaine in API hydrocodone may be formed by chemical over-reduction of thebaine.

However, in the case where the yeast-derived compound and the plant-derived compound are both subjected to chemical modification through chemical synthesis approaches, the same impurities associated with the chemical synthesis process may be expected in the products. In such a situation, the starting material (e.g., CYCM or CPS) may be analyzed as described above.

### Host Cell Derived Nal-Opioids Vs Chemically Derived Nal-Opioids

Nal-opioids produced by chemical synthesis may contain a plurality of impurities. These impurities may arise from many different causes, for example, unreacted starting materials, incomplete reactions, the formation of byproducts, persistence of intermediates, dimerization, or degradation. An example of an unreacted starting material could be oxymorphone remaining in a preparation of naltrexone. An example of an impurity arising from an incomplete reaction could be 3-O-Methylbuprenorphine resulting from the incomplete 3-O-demethylation of thebaine. Chemical modification can result in the addition or removal of functional groups at off-target sites. For example, the oxidation of C10 to create 10-hydroxynaltrexone and 10-ketonaltrexone during naltrexone synthesis, or the removal of the 6-O-methyl group to give 6-O-desmethylbuprenorphine during buprenorphine synthesis. Impurites may arise from the persistence of reaction intermediates, for example the persistence of N-oxides like oxymorphone N-oxide formed during the N-demethylation process. Another source of impurities is dimerization, the conjugation of two opioid molecules, for example two buprenorphine molecules (2,2′-bisbuprenorphine), two naltrexone molecules (2,2′-bisnaltrexone), or two naloxone molecules (2,2′-bisnaloxone). Impurities may arise from degradation of starting materials, reaction intermediates, or reaction products. The extreme physical conditions used in chemical syntheses may make the presence of degradation more likely. An example of an impurity that may arise from degradation is dehydrobuprenorphine produced by oxidizing conditions during buprenorphine synthesis.

Nal-opioids produced by enzyme catalysis in a host cell may contain different impurities than nal-opioids produced by chemical synthesis. Nal-opioids produced by enzyme catalysis in a host cell may contain fewer impurities than nal-opioids produced by chemical synthesis. Nal-opioids produced by enzyme catalysis in a host cell may lack certain impurities that are found in nal-opioids produced by chemical synthesis. In examples, key features of enzyme synthesis may include, (1) enzymes target a specific substrate and residue with high fidelity; (2) enzymes perform reactions in the mild physiological conditions within the cell which do not compromise the stability of the molecules; and (3) enzymes are engineered to be efficient catalysts that drive reactions to completion.

Table 11 highlights some of the impurities that may be specific to chemically produced nal-opioids. Accordingly, nal-opioids may be assessed for impurities to determine the presence or absence of any impurity from Table 11. By analyzing a product of unknown origin for a subset of these impurities, a person of skill in the art could determine whether the product originated from a chemical or enzymatic synthesis.

### Methods of Engineering Host Cells

Also included are methods of engineering host cells for the purpose of producing nor-opioid and/or nal-opioid BIAs of interest. Inserting DNA into host cells may be achieved using any convenient methods. The methods are used to insert the heterologous coding sequences into the engineered host cells such that the host cells functionally express the enzymes and convert starting compounds of interest into product nor-opioid and/or nal-opioid BIAs of interest.

Any convenient promoters may be utilized in the subject engineered host cells and methods. The promoters driving expression of the heterologous coding sequences may be constitutive promoters or inducible promoters, provided that the promoters are active in the engineered host cells. The heterologous coding sequences may be expressed from their native promoters, or non-native promoters may be used. Such promoters may be low to high strength in the host in which they are used. Promoters may be regulated or constitutive. In certain embodiments, promoters that are not glucose repressed, or repressed only mildly by the presence of glucose in the culture medium, are used. Promoters of interest include but are not limited to, promoters of glycolytic genes such as the promoter of the B. subtilis tsr gene (encoding the promoter region of the fructose bisphosphate aldolase gene) or the promoter from yeast S. cerevisiae gene coding for glyceraldehyde 3-phosphate dehydrogenase (GPD, GAPDH, or TDH3), the ADH1 promoter of baker's yeast, the phosphate-starvation induced promoters such as the PHO5 promoter of yeast, the alkaline phosphatase promoter from B. licheniformis, yeast inducible promoters such as Gal1-10, Gal1, GalL, GalS, repressible promoter Met25, tetO, and constitutive promoters such as glyceraldehyde 3-phosphate dehydrogenase promoter (GPD), alcohol dehydrogenase promoter (ADH), translation-elongation factor-1-α promoter (TEF), cytochrome c-oxidase promoter (CYC1), MRP7 promoter, etc. Autonomously replicating yeast expression vectors containing promoters inducible by hormones such as glucocorticoids, steroids, and thyroid hormones may also be used and include, but are not limited to, the glucorticoid responsive element (GRE) and thyroid hormone responsive element (TRE). These and other examples are described U.S. Pat. No. 7,045,290, which is incorporated by reference, including the references cited therein. Additional vectors containing constitutive or inducible promoters such as a factor, alcohol oxidase, and PGH may be used. Additionally any promoter/enhancer combination (as per the Eukaryotic Promoter Data Base EPDB) could also be used to drive expression of genes. Any convenient appropriate promoters may be selected for the host cell, examples of promoters that could be used in an E. coli cell include T7, lac and tetO promoters. One may also use promoter selection to optimize transcript, and hence, enzyme levels to maximize production while minimizing energy resources.

Any convenient vectors may be utilized in the subject engineered host cells and methods. Vectors of interest include vectors for use in yeast and other cells. The types of yeast vectors may be broken up into 4 general categories: integrative vectors (YIp), autonomously replicating high copy-number vectors (YEp or 2μ plasmids), autonomously replicating low copy-number vectors (YCp or centromeric plasmids) and vectors for cloning large fragments (YACs). Vector DNA is introduced into prokaryotic or eukaryotic cells via any convenient transformation or transfection techniques. DNA of another source (e.g. PCR-generated double stranded DNA product, or synthesized double stranded or single stranded oligonucleotides) may be used to engineer the yeast by integration into the genome. Any single transformation event may include one or several nucleic acids (vectors, double stranded or single stranded DNA fragments) to genetically modify the host cell.

### Utility

The engineered host cells and methods disclosed herein, e.g., as described above, find use in a variety of applications. Applications of interest include, but are not limited to: research applications and therapeutic applications. Methods disclosed herein find use in a variety of different applications including any convenient application where the production of nor-opioid and/or nal-opioid BIAs of interest.

The subject engineered host cells and methods find use in a variety of therapeutic applications. Therapeutic applications of interest include those applications in which the preparation of pharmaceutical products that include nor-opioids or nal-opioids is of interest. The engineered host cells described herein produce nor-opioids or nal-opioids of interest and enzymes of interest. Reticuline is a major branch point intermediate of interest in the synthesis of BIAs including engineering efforts to produce end products such as opioid products. The subject host cells may be utilized to produce nor-opioids or nal-opioids of interest from simple and inexpensive starting materials that may find use in the production of BIAs of interest, including reticuline, and BIA end products, such as nor-opioids or nal-opioids. As such, the subject host cells find use in the supply of therapeutically active nor-opioids or nal-opioids of interest.

In some instances, the engineered host cells and methods find use in the production of commercial scale amounts of nor-opioids or nal-opioids thereof where chemical synthesis of these compounds is low yielding and not a viable means for large-scale production. In certain cases, the host cells and methods are utilized in a fermentation facility that would include bioreactors (fermenters) of e.g., 5,000-200,000 liter capacity allowing for rapid production of nor-opioids or nal-opioids of interest thereof for therapeutic products. Such applications may include the industrial-scale production of nor-opioids or nal-opioids of interest from fermentable carbon sources such as cellulose, starch, and free sugars.

The subject engineered host cells and methods find use in a variety of research applications. The subject host cells and methods may be used to analyze the effects of a variety of enzymes on the biosynthetic pathways of a variety of nor-opioid and/or nal-opioid BIAs of interest. In addition, the engineered host cells may be engineered to produce nor-opioid and/or nal-opioid BIAs of interest that find use in testing for bioactivity of interest in as yet unproven therapeutic functions. In some cases, the engineering of host cells to include a variety of heterologous coding sequences that encode for a variety of enzymes elucidates the high yielding biosynthetic pathways towards nor-opioid and/or nal-opioid BIAs of interest. In certain cases, research applications include the production of nor-opioid and/or nal-opioid BIAs of interest for therapeutic molecules of interest that may then be further chemically modified or derivatized to desired products or for screening for increased therapeutic activities of interest. In some instances, host cell strains are used to screen for enzyme activities that are of interest in such pathways, which may lead to enzyme discovery via conversion of nor-opioid or nal-opioid metabolites produced in these strains.

The subject engineered host cells and methods may be used as a production platform for plant specialized metabolites. The subject host cells and methods may be used as a platform for drug library development as well as plant enzyme discovery. For example, the subject engineered host cells and methods may find use in the development of natural product based drug libraries by taking yeast strains producing interesting scaffold molecules, such as norcodeine, or northebaine, and further functionalizing the compound structure through combinatorial biosynthesis or by chemical means. By producing drug libraries in this way, any potential drug hits are already associated with a production host that is amenable to large-scale culture and production. As another example, these subject engineered host cells and methods may find use in plant enzyme discovery. The subject host cells provide a clean background of defined metabolites to express plant EST libraries to identify new enzyme activities. The subject host cells and methods provide expression methods and culture conditions for the functional expression and increased activity of plant enzymes in yeast.

### Kits and Systems

Some aspects of the invention further include kits and systems, where the kits and systems may include one or more components employed in methods disclosed herein, e.g., engineered host cells, starting compounds, heterologous coding sequences, vectors, culture medium, etc., as described herein. In some embodiments, the subject kit includes an engineered host cell (e.g., as described herein), and one or more components selected from the following: starting compounds, a heterologous coding sequence and/or a vector including the same, vectors, growth feedstock, components suitable for use in expression systems (e.g., cells, cloning vectors, multiple cloning sites (MCS), bi-directional promoters, an internal ribosome entry site (IRES), etc.), and a culture medium.

Any of the components described herein may be provided in the kits, e.g., host cells including one or more modifications, starting compounds, culture medium, etc. A variety of components suitable for use in making and using heterologous coding sequences, cloning vectors and expression systems may find use in the subject kits. Kits may also include tubes, buffers, etc., and instructions for use. The various reagent components of the kits may be present in separate containers, or some or all of them may be pre-combined into a reagent mixture in a single container, as desired.

Also provided are systems for producing nor-opioid and/or nal-opioid BIAs of interest, where the systems may include engineered host cells including one or more modifications (e.g., as described herein), starting compounds, culture medium, a fermenter and fermentation equipment, e.g., an apparatus suitable for maintaining growth conditions for the host cells, sampling and monitoring equipment and components, and the like. A variety of components suitable for use in large scale fermentation of yeast cells may find use in the subject systems.

In some cases, the system includes components for the large scale fermentation of engineered host cells, and the monitoring and purification of nor-opioid or nal-opioid compounds produced by the fermented host cells. In certain embodiments, one or more starting compounds (e.g., as described herein) are added to the system, under conditions by which the engineered host cells in the fermenter produce one or more desired nor-opioid or nal-opioid products of interest. In some instances, the host cells produce a nor-opioid or nal-opioid of interest (e.g., as described herein). In certain cases, the nor-opioid or nal-opioid products of interest are opioid antagonists, such as naloxone, naltrexone, nalmefene, or nalorphine. In certain cases, the nor-opioid or nal-opioid products of interest are opioid antagonists such as naltrindole or norbinaltorphimine. In some examples, the nor-opioid or nal-opioid products of interest are partial agonists such as buprenorphine.

In some cases, the system includes processes for monitoring and or analyzing one or more nor-opioid and/or nal-opioid BIAs of interest compounds produced by the subject host cells. For example, a LC-MS analysis system as described herein, a chromatography system, or any convenient system where the sample may be analyzed and compared to a standard, e.g., as described herein. The fermentation medium may be monitored at any convenient times before and during fermentation by sampling and analysis. When the conversion of starting compounds to nor-opioid or nal-opioid products of interest is complete, the fermentation may be halted and purification of the nor-opioid or nal-opioid products may be done. As such, in some cases, the subject system includes a purification component suitable for purifying the nor-opioid or nal-opioid products of interest from the host cell medium into which it is produced. The purification component may include any convenient means that may be used to purify the nor-opioid or nal-opioid products of interest produced by fermentation, including but not limited to, silica chromatography, reverse-phase chromatography, ion exchange chromatography, HIC chromatography, size exclusion chromatography, liquid extraction, and pH extraction methods. In some cases, the subject system provides for the production and isolation of enzyme and/or nor-opioid or nal-opioid fermentation products of interest following the input of one or more starting compounds to the system.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Discussion of Enzyme List

The host cells may be engineered to include one or more modifications (such as two or more, three or more, four or more, five or more, or even more modifications) that provide for the production of nor-opioid and/or nal-opioid BIAs of interest. Table 2 provides a list of exemplary genes that may be acted upon by one or more modifications so as to provide for the production of nor-opioid and/or nal-opioid BIAs of interest and/or enzymes of interest in an engineered host cell.

Modifications of genes as provided in Table 2 may be used to produce nor-opioid and/or nal-opioid BIAs of interest from engineered host cells that are supplied with a medium containing the minimal nutrients required for growth. This minimal medium may contain a carbon source, a nitrogen source, amino acids, vitamins, and salts. For example, modifications of genes as provided in Table 2 may be used to produce nor-opioid and/or nal-opioid BIAs of interest from engineered host cells that are fed sugar. Additionally, modifications of one or more genes as provided in Table 2 may be used to augment the biosynthetic processes of host cells that may be engineered for drug production.

Additionally, the use of these modifications to provide for the production of nor-opioid and/or nal-opioid BIAs of interest and/or enzymes of interest in engineered host cells is not readily apparent from the mere identification of enzymes that may be produced by the genes. In particular, synthetic pathways that have been reconstructed in host cells, such as yeast cells, as described herein comprise a variety of enzymes that do not act together in nature within a single organism. Additionally, some of the enzymes discussed herein do not act for nor-opioid and/or nal-opioid BIA biosynthesis in their natural context. Further, some of the enzymes described herein are not evolved to function in particular host cells, such as yeast cells, and are not evolved to function together. Further, some of the nor-opioids or nal-opioids produced do not occur naturally. In these cases, it would not be obvious that the enzymes would exhibit sufficient activity in the context of the synthetic nor-opioid and/or nal-opioid pathway in a host cell, such as yeast, to have sufficient flux through the pathway to produce downstream nor-opioid or nal-opioid end products.

For example, plant enzymes are often difficult to functionally express in heterologous microbial hosts, such as yeast. In many cases the enzymes may be misfolded, not correctly localized within the host cell, and/or incorrectly processed. The differences in protein translation and processing between yeast and plants can lead to these enzymes exhibiting substantially reduced to no detectable activities in the yeast host. These challenges arise commonly for endomembrane localized enzymes, such as cytochrome P450s, which are strongly represented in the BIA pathways which produce precursors for nor-opioids or nal-opioids. Even reduced enzyme activities may pose a substantial challenge to engineering yeast to produce complex BIAs, which requires sufficient activity at each step to ensure high-level accumulation of the desired BIA products.

Additionally, there are endogenous enzymes/pathways in some host cells, such as yeast, that may act on many of the early precursors in the BIA pathway (i.e., intermediates from tyrosine to norcoclaurine), and thus it may not be readily apparent that there would be sufficient flux through the heterologous pathway to achieve substantial BIA production given these competing endogenous pathways. For example, the Erlich pathway (Hazelwood, et al. 2008. Appl. Environ. Microbiol. 74:2259-66; Larroy, et al. 2003. Chem. Biol. Interact. 143-144:229-38; Larroy, et al. 2002. Eur. J. Biochem. 269:5738-45) in yeast is the main endogenous pathway that would act to convert many of the intermediates in the early BIA pathway to undesired products and divert flux from the synthetic pathway.

Further, many of the enzymes as discussed herein, and as provided in Table 2, may function under very specific regulation strategies, including spatial regulation, in the native plant hosts, which may be lost upon transfer to the heterologous yeast host. In addition, plants present very different biochemical environments than yeast cells under which the enzymes are evolved to function, including pH, redox state, and substrate, cosubstrate, coenzyme, and cofactor availabilities. Given the differences in biochemical environments and regulatory strategies between the native hosts and the heterologous yeast hosts, it is not obvious that the enzymes would exhibit substantial activities when in the context of the yeast environment and further not obvious that they would work together to direct simple precursors such as sugar to complex BIA compounds. Maintaining the activities of the enzymes in the yeast host is particularly important as many of the pathways have many reaction steps (>10), such that if these steps are not efficient then one would not expect accumulation of desired downstream products.

In addition, in the native plant hosts, the associated metabolites in these pathways may be localized across different cell and tissue types. In several examples, there are cell types that may be specialized for biosynthesis and cell types that may be synthesized for metabolite accumulation. This type of cell specialization may be lost when expressing the pathways within a heterologous yeast host, and may play an important role in controlling the toxicity of these metabolites on the cells. Thus, it is not obvious that yeast could be successfully engineered to biosynthesize and accumulate these metabolites without being harmed by the toxicity of these compounds.

As one example, in the native plant hosts, the enzyme BBE is reported to have dynamic subcellular localization. In particular, the enzyme BBE initially starts in the ER and then is sorted to the vacuole (Bird and Facchini. 2001. Planta. 213:888-97). It has been suggested that the ER-association of BBE in plants (Alcantara, et al. 2005. Plant Physiol. 138:173-83) provides the optimal basic pH (pH ˜8.8) for BBE activity (Ziegler and Facchini. 2008. Annu. Rev. Plant Biol. 59:735-69). As another example, there is evidence that sanguinarine biosynthesis occurs in specialized vesicles within plant cells (Amann, et al. 1986. Planta. 167:310-20), but only some of the intermediates accumulate in the vesicles. This may occur so as to sequester them from other enzyme activities and/or toxic effects.

As another example, the biosynthetic enzymes in the morphinan pathway branch are all localized to the phloem, which is part of the vascular tissue in plants. In the phloem, the pathway enzymes may be further divided between two cell types: the sieve elements common to all plants, and the laticifer which is a specialized cell type present only in certain plants which make specialized secondary metabolites. The upstream enzymes (i.e., from NCS through to SalAT) are predominantly in the sieve elements, and the downstream enzymes (i.e., T6ODM, COR, CODM) are mostly in the laticifer (Onoyovwe, et al. 2013. Plant Cell. 25:4110-22). Additionally, it was discovered that the final steps in the noscapine biosynthetic pathway take place in the laticifer (Chen and Facchini. 2014. Plant J. 77:173-84). This compartmentalization is thought to be highly important for regulating biosynthesis by isolating or trafficking intermediates, providing optimal pH, enhancing supply of cofactors, although the nature of the poppy laticifer microenvironment is still under investigation (Ziegler and Facchini. 2008. Annu. Rev. Plant Biol. 59:735-69). Further, it is predicted that several of the enzymes may function as multi-enzyme complexes or metabolic channels common to plant secondary metabolism (Kempe, et al. 2009. Phytochemistry. 70:579-89; Allen, et al. 2004. Nat. Biotechnol. 22:1559-66). When biosynthetic enzymes are combined from different hosts and/or expressed recombinantly in a heterologous yeast cell it is not clear that these complexes or channels will form as they would in the native host. In an additional example, in Coptis japonica, berberine is biosynthesized in root tissues and then accumulated within the rhizome via the action of specialized ATP-binding cassette transport proteins (shitan, et al. 2013. Phytochemistry. 91:109-16). In opium poppy, morphinan alkaloids are accumulated within the latex (cytoplasm of laticifer cells) (Martin, et al. 1967. Biochemistry. 6:2355-63).

Further, even without these considerations, it is also the case that the plant enzymes for several of the steps in the pathways described herein have not yet been characterized. For example, the conversion of tyrosine to the early benzylisoquinoline alkaloid scaffold norcoclaurine has not yet been characterized. Thus, for several of the steps in the pathways described herein, alternative biosynthetic scheme were produced by bringing together enzyme activities that do not normally occur together in nature for the biosynthesis of BIAs or identifying new enzyme activities from genome sequence information to use in the reconstructed pathways.

For example, the two-step conversion of tyrosine to dopamine may be achieved by combining at least 5 mammalian enzymes and 1 bacterial enzyme, which do not naturally occur together and were not evolved to function in the context of this pathway or with plant enzymes. In these instances, it may not be obvious to utilize these enzymes for the biosynthesis of compounds they were not evolved for in nature and that they would function effectively in the context of a heterologous microbial host and this pathway.

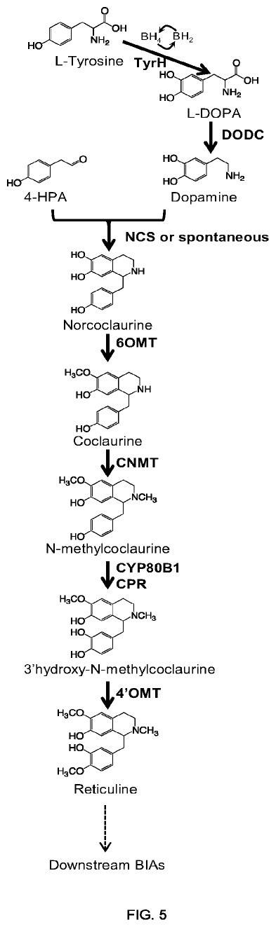

Examples of the genes that are the object of modifications so as to produce nor-opioid and/or nal-opioid BIAs of interest and/or enzymes of interest are discussed below. Additionally, the genes are discussed in the context of a series of Figures that illustrate pathways that are used in generating BIAs and nor-opioid and/or nal-opioid BIAs of interest and/or enzymes of interest.

[TLK1] In some examples, the engineered host cell may modify the expression of the enzyme transketolase. Transketolase is encoded by the TKL1 gene. In examples, transketolase catalyzes the reaction of fructose-6-phosphate+glyceraldehyde-3-phosphate↔xylulose-5-phosphate+erythrose-4-phosphate, as referenced in FIG. 2. An engineered host cell may be modified to include constitutive overexpression of the TKL1 gene in the engineered host cell. Additionally or alternatively, the engineered host cell may be modified to synthetically regulate the expression of the TKL1 gene in the engineered host cell. In examples, the engineered host cell may be modified to incorporate a copy, copies, or additional copies, of the TKL1 gene. Additionally or alternatively, the engineered host cell may be modified to incorporate the introduction of a strong promoter element for the overexpression of the TKL1 gene within the engineered host cell. The TKL1 gene may be derived from Saccharomyces cerevisiae or another species. In some examples, the TKL1 gene may be 100% similar to the naturally occurring gene.

[ZWF1] In some examples, the engineered host cell may modify the expression of the enzyme glucose-6-phosphate dehydrogenase. Glucose-6-phosphate dehydrogenase is encoded by the ZWF1 gene. In examples, glucose-6-phosphate dehydrogenase catalyzes the reaction of glucose-6-phosphate→6-phosphogluconolactone, as referenced in FIG. 2. An engineered host cell may be modified to delete the coding region of the ZWF1 gene in the engineered host cell. Alternatively, the engineered host cell may be modified to disable the functionality of the ZWF1 gene, such as by introducing an inactivating mutation.

[ARO4] In some examples, the engineered host cell may modify the expression of the enzyme 3-deoxy-D-arabino-heptulosonate-7-phosphate (DAHP) synthase. DAHP synthase is encoded by the ARO4 gene. In examples, DAHP synthase catalyzes the reaction of erythrose-4-phosphate+phosphoenolpyruvic acid→DAHP, as referenced in FIG. 2. An engineered host cell may modify the ARO4 gene to incorporate one or more feedback inhibition alleviating mutations. In particular, a feedback inhibition alleviating mutation (e.g., ARO4FBR) may be incorporated as a directed mutation to a native ARO4 gene at the original locus; as an additional copy introduced as a genetic integration at a separate locus; or as an additional copy on an episomal vector such as a 2-μm or centromeric plasmid. The identifier “FBR” in the mutation ARO4FBR refers to feedback resistant mutants and mutations. The feedback inhibited copy of the DAHP synthase enzyme may be under a native yeast transcriptional regulation, such as when the engineered host cell is a yeast cell. Alternatively, the feedback inhibited copy of the DAHP synthase enzyme may be introduced to the engineered host cell with engineered constitutive or dynamic regulation of protein expression by placing it under the control of a synthetic promoter. In some cases, the ARO4 gene may be derived from Saccharomyces cerevisiae. In some cases, the ARO4 gene may be 100% similar to the naturally occurring gene. Examples of modifications to the ARO4 gene include a feedback inhibition resistant mutation, K229L, or Q166K.

[ARO7] In some examples, the engineered host cell may modify the expression of the enzyme chorismate mutase. Chorismate mutase is encoded by the ARO7 gene. In examples, chorismate mutase catalyzes the reaction of chorismate→prephenate, as referenced in FIG. 2. An engineered host cell may modify the ARO7 gene to incorporate one or more feedback inhibition alleviating mutations. In particular, a feedback inhibition alleviating mutation (e.g., ARO7^{FBR}) may be incorporated as a directed mutation to a native ARO7 gene at the original locus; as an additional copy introduced as a genetic integration at a separate locus; or as an additional copy on an episomal vector such as a 2-μm or centromeric plasmid. The identifier “FBR” in the mutation ARO7^{FBR} refers to feedback resistant mutants and mutations. The feedback inhibited copy of the chorismate mutase enzyme may be under a native yeast transcriptional regulation, such as when the engineered host cell is a yeast cell. Alternatively, the feedback inhibited copy of the chorismate mutase enzyme may be introduced to the engineered host cell with engineered constitutive or dynamic regulation of protein expression by placing it under the control of a synthetic promoter. In some cases, the ARO7 gene may be derived from Saccharomyces cerevisiae. In some cases, the ARO7 gene may be 100% similar to the naturally occurring gene. Examples of modifications to the ARO7 gene include a feedback inhibition resistant mutation or T226I.

[ARO10] In some examples, the engineered host cell may modify the expression of the enzyme phenylpyruvate decarboxylase. Phenylpyruvate decarboxylase is encoded by the ARO10 gene. In examples, phenylpyruvate decarboxylase catalyzes the reaction of hydroxyphenylpyruvate→4-hydroxyphenylacetate (4HPA), as referenced in FIG. 2. An engineered host cell may be modified to include constitutive overexpression of the ARO10 gene in the engineered host cell. Additionally or alternatively, the engineered host cell may be modified to synthetically regulate the expression of the ARO10 gene in the engineered host cell. In examples, the engineered host cell may be modified to incorporate a copy, copies, or additional copies, of the ARO10 gene. Additionally or alternatively, the engineered host cell may be modified to incorporate the introduction of a strong promoter element for the overexpression of the ARO10 gene within the engineered host cell. The ARO10 gene may be derived from Saccharomyces cerevisiae or another species. In some examples, the ARO10 gene may be 100% similar to the naturally occurring gene.

[ADH2-7, SFA1] In some examples, the engineered host cell may modify the expression of alcohol dehydrogenase enzymes. Alcohol dehydrogenase enzymes may be encoded by one or more of the ADH2, ADH3, ADH4, ADH5, ADH6, ADH7, and SFA1 genes. In examples, alcohol dehydrogenase catalyzes the reaction of 4HPA→tyrosol. An engineered host cell may be modified to delete the coding region of one or more of the ADH2, ADH3, ADH4, ADH5, ADH6, ADH7, and SFA1 genes in the engineered host cell. Alternatively, the engineered host cell may be modified to disable the functionality of one or more of the ADH2, ADH3, ADH4, ADH5, ADH6, ADH7, and SFA1 genes, such as by introducing an inactivating mutation.

[ALD2-6] In some examples, the engineered host cell may modify the expression of aldehyde oxidase enzymes. Aldehyde oxidase enzymes may be encoded by one or more of the ALD2, ALD3, ALD4, ALD5, and ALD6 genes. In examples, aldehyde oxidase catalyzes the reaction of 4HPA→hydroxyphenylacetic acid. An engineered host cell may be modified to delete the coding region of one or more of the ALD2, ALD3, ALD4, ALD5, and ALD6 genes in the engineered host cell. Alternatively, the engineered host cell may be modified to disable the functionality of one or more of the ALD2, ALD3, ALD4, ALD5, and ALD6 genes, such as by introducing an inactivating mutation.

[ARO9] In some examples, the engineered host cell may modify the expression of the enzyme aromatic aminotransferase. Aromatic aminotransferase is encoded by the ARO9 gene. In examples, aromatic aminotransferase catalyzes the reaction of hydroxyphenylpyruvate +glutamate→tyrosine+alpha-ketogluterate, as referenced in FIG. 2. An engineered host cell may be modified to include constitutive overexpression of the ARO9 gene in the engineered host cell. Additionally or alternatively, the engineered host cell may be modified to synthetically regulate the expression of the ARO9 gene in the engineered host cell. In examples, the engineered host cell may be modified to incorporate a copy, copies, or additional copies, of the ARO9 gene. Additionally or alternatively, the engineered host cell may be modified to incorporate the introduction of a strong promoter element for the overexpression of the ARO9 gene within the engineered host cell. The ARO9 gene may be derived from Saccharomyces cerevisiae or another species. In some examples, the ARO9 gene may be 100% similar to the naturally occurring gene.

[TYR] In some examples, the engineered host cell may modify the expression of the enzyme tyrosinase. Tyrosinase is encoded by the TYR gene. In examples, tyrosinase catalyzes the reaction of tyrosine→L-DOPA, as referenced in FIG. 2. In other examples, tyrosinase catalyzes the reaction of L-DOPA→dopaquinone. An engineered host cell may be modified to include constitutive expression of the TYR gene in the engineered host cell. Additionally or alternatively, the engineered host cell may be modified to synthetically regulate the expression of the TYR gene in the engineered host cell. In examples, the engineered host cell may be modified to incorporate a copy, copies, or additional copies, of the TYR gene. Additionally or alternatively, the engineered host cell may be modified to incorporate the introduction of a strong promoter element for the overexpression of the TYR gene within the engineered host cell. The TYR gene may be derived from Ralstonia solanacearum, Agaricus bisporus, or another species. In some examples, the TYR gene may be 100% similar to the naturally occurring gene.

[TyrH] In some examples, the engineered host cell may modify the expression of the enzyme tyrosine hydroxylase. Tyrosine hydroxylase is encoded by the TyrH gene. In examples, tyrosine hydroxylase catalyzes the reaction of tyrosine→L-DOPA, as referenced in FIGS. 2 and 5. An engineered host cell may be modified to include constitutive expression of the TyrH gene in the engineered host cell. Additionally or alternatively, the engineered host cell may be modified to synthetically regulate the expression of the TyrH gene in the engineered host cell. In examples, the engineered host cell may be modified to incorporate a copy, copies, or additional copies, of the TyrH gene. Additionally or alternatively, the engineered host cell may be modified to incorporate the introduction of a strong promoter element for the overexpression of the TyrH gene within the engineered host cell. The TyrH gene may be derived from Homo sapiens, Rattus norvegicus, Mus musculus, or another species. In some examples, the TyrH gene may be 100% similar to the naturally occurring gene.

[DODC] In some examples, the engineered host cell may modify the expression of the enzyme L-DOPA decarboxylase. L-DOPA decarboxylase is encoded by the DODC gene. In examples, L-DOPA decarboxylase catalyzes the reaction of L-DOPA→dopamine, as referenced in FIGS. 2 and 5. An engineered host cell may be modified to include constitutive expression of the DODC gene in the engineered host cell. Additionally or alternatively, the engineered host cell may be modified to synthetically regulate the expression of the DODC gene in the engineered host cell. In examples, the engineered host cell may be modified to incorporate a copy, copies, or additional copies, of the DODC gene. Additionally or alternatively, the engineered host cell may be modified to incorporate the introduction of a strong promoter element for the overexpression of the DODC gene within the engineered host cell. The DODC gene may be derived from Pseudomonas putida, Rattus norvegicus, or another species. In some examples, the DODC gene may be 100% similar to the naturally occurring gene.

[TYDC] In some examples, the engineered host cell may modify the expression of the enzyme tyrosine/DOPA decarboxylase. Tyrosine/DOPA decarboxylase is encoded by the TYDC gene. In examples, tyrosine/DOPA decarboxylase catalyzes the reaction of L-DOPA→dopamine, as referenced in FIG. 2. An engineered host cell may be modified to include constitutive expression of the TYDC gene in the engineered host cell. Additionally or alternatively, the engineered host cell may be modified to synthetically regulate the expression of the TYDC gene in the engineered host cell. In examples, the engineered host cell may be modified to incorporate a copy, copies, or additional copies, of the TYDC gene. Additionally or alternatively, the engineered host cell may be modified to incorporate the introduction of a strong promoter element for the overexpression of the TYDC gene within the engineered host cell. The TYDC gene may be derived from Papaver somniferum or another species. In some examples, the TYDC gene may be 100% similar to the naturally occurring gene.

[MAO] In some examples, the engineered host cell may modify the expression of the enzyme monoamine oxidase. Monoamine oxidase is encoded by the MAO gene. In examples, monoamine oxidase catalyzes the reaction of dopamine→3,4-DHPA, as referenced in FIG. 2. An engineered host cell may be modified to include constitutive expression of the MAO gene in the engineered host cell. Additionally or alternatively, the engineered host cell may be modified to synthetically regulate the expression of the MAO gene in the engineered host cell. In examples, the engineered host cell may be modified to incorporate a copy, copies, or additional copies, of the MAO gene. Additionally or alternatively, the engineered host cell may be modified to incorporate the introduction of a strong promoter element for the overexpression of the MAO gene within the engineered host cell. In some cases, the MAO gene may be codon optimized for expression in Saccharomyces cerevisiae. The MAO gene may be derived from Escherichia coli, Homo sapiens, Micrococcus luteus, or another species. In some examples, the MAO gene may be 77% similar to the naturally occurring gene.

[NCS] In some examples, the engineered host cell may modify the expression of the enzyme norcoclaurine synthase. Norcoclaurine synthase is encoded by the NCS gene. In examples, norcoclaurine synthase catalyzes the reaction of 4HPA+dopamine→(S)-norcoclaurine, as referenced in FIG. 5. In particular, FIG. 5 illustrates a biosynthetic scheme for conversion of L-tyrosine to reticuline via norcoclaurine, in accordance with embodiments of the invention. FIG. 5 provides the use of the enzymes TyrH, tyrosine hydroxylase; DODC, DOPA decarboxylase; NCS, norcoclaurine synthase, as discussed herein; 6OMT, 6-O-methyltransferase; CNMT, coclaurine N-methyltransferase; CYP80B1, cytochrome P450 80B1; CPR, cytochrome P450 NADPH reductase; 4′OMT, 3′hydroxy-N-methylcoclaurine 4′-O-methyltransferase. L-DOPA, L-3,4-dihydroxyphenylalanine; and 4-HPA, 4-hydroxyphenylacetylaldehyde. Of the enzymes that are illustrated in FIG. 5, 4-HPA and L-tyrosine are naturally synthesized in yeast. All other metabolites shown are not naturally produced in yeast. Additionally, although TyrH is depicted as catalyzing the conversion of L-tyrosine to L-DOPA, other enzymes may also be used to perform this step as described in the specification. For example, tyrosinases may also be used to perform the conversion of L-tyrosine to L-DOPA. In addition, other enzymes such as cytochrome P450 oxidases may also be used to perform the conversion of L-tyrosine to L-DOPA. Such enzymes may exhibit oxidase activity on related BIA precursor compounds including L-DOPA and L-tyrosine.

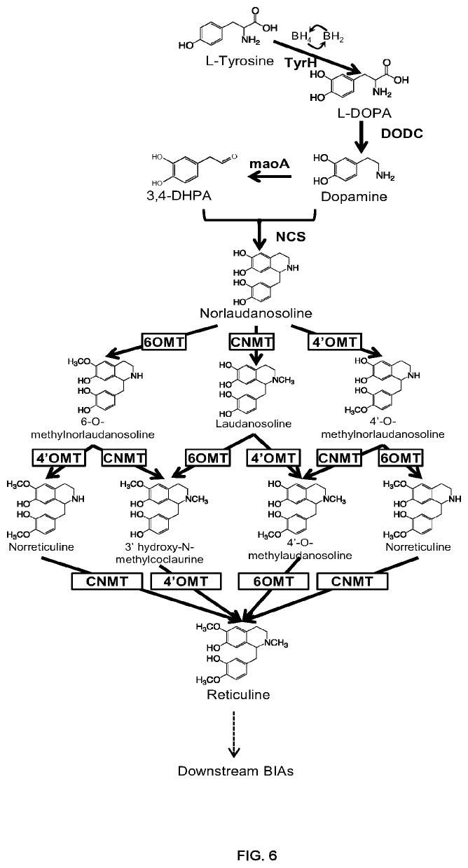

Additionally, norcoclaurine synthase catalyzes the reaction of 3,4-DHPA+dopamine→(S)-norlaudanosoline, as referenced in FIG. 6. In particular, FIG. 6 illustrates a biosynthetic scheme for conversion of L-tyrosine to reticuline via norlaudanosoline, in accordance with embodiments of the invention. FIG. 6 provides the use of the enzymes TyrH, tyrosine hydroxylase; DODC, DOPA decarboxylase; maoA, monoamine oxidase; NCS, norcoclaurine synthase; 6OMT, 6-O-methyltransferase; CNMT, coclaurine N-methyltransferase; 4′OMT, 3′hydroxy-N-methylcoclaurine 4′-O-methyltransferase. L-DOPA, L-3,4-dihydroxyphenylalanine; and 3,4-DHPA, 3,4-dihydroxyphenylacetaldehyde. Of the enzymes that are illustrated in FIG. 6, L-tyrosine is naturally synthesized in yeast. Other metabolites that are shown in FIG. 6 are not naturally produced in yeast.

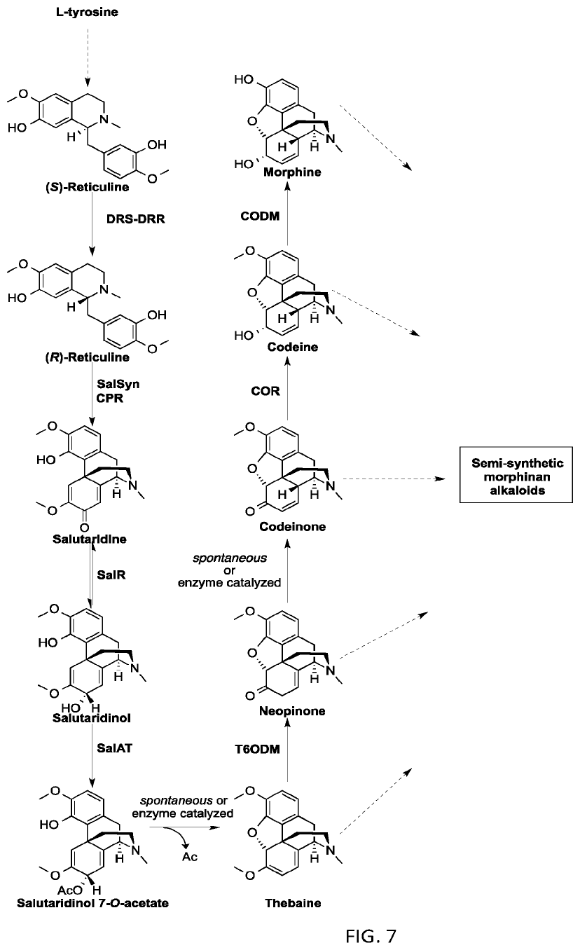

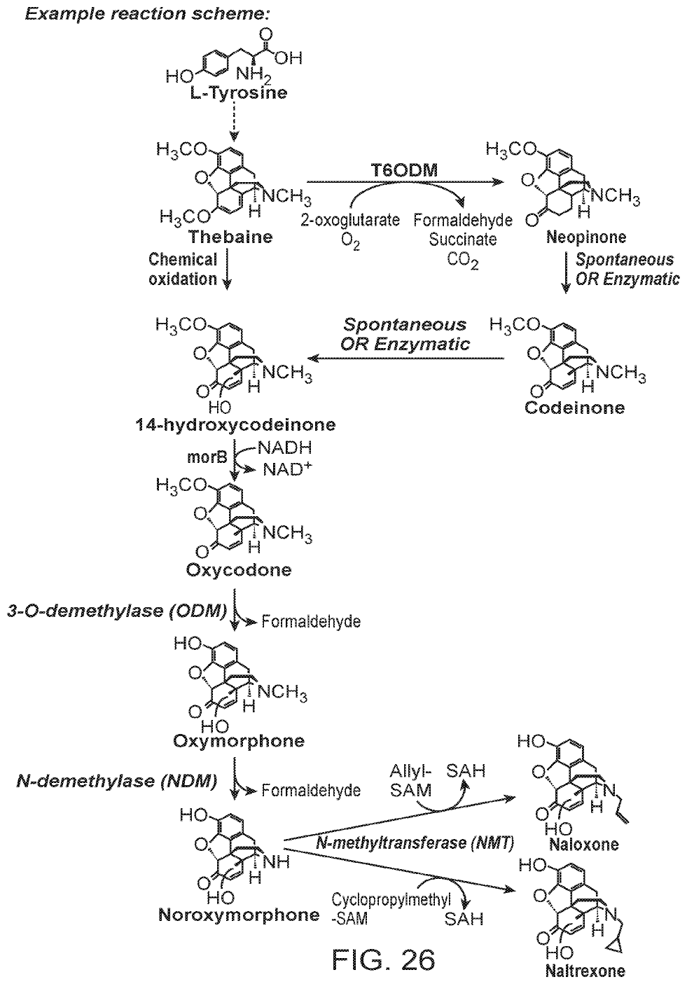

An engineered host cell may be modified to include constitutive expression of the NCS gene in the engineered host cell. Additionally or alternatively, the engineered host cell may be modified to synthetically regulate the expression of the NCS gene in the engineered host cell. In examples, the engineered host cell may be modified to incorporate a copy, copies, or additional copies, of the NCS gene. Additionally or alternatively, the engineered host cell may be modified to incorporate the introduction of a strong promoter element for the overexpression of the NCS gene within the engineered host cell. Additionally, the norcoclaurine synthase may have an N-terminal truncation. In some cases, the NCS gene may be codon optimized for expression in Saccharomyces cerevisiae. The NCS gene may be derived from Coptis japonica, Papaver somniferum, Papver bracteatum, Thalicitum flavum, Corydalis saxicola, or another species. In some examples, the NCS gene may be 80% similar to the naturally occurring gene.

[6OMT] In some examples, the engineered host cell may modify the expression of the enzyme norcoclaurine 6-O-methyltransferase. Norcoclaurine 6-O-methyltransferase is encoded by the 6OMT gene. In some examples, norcoclaurine 6-O-methyltransferase catalyzes the reaction of norcoclaurine→coclaurine, as referenced in FIG. 5. In other examples, norcoclaurine 6-O-methyltransferase catalyzes the reaction of norlaudanosoline→3′hydroxycoclaurine, as well as other reactions detailed herein, such as those provided in FIG. 6. Additionally, the engineered host cell may be modified to include constitutive expression of the 6OMT gene in the engineered host cell. Additionally or alternatively, the engineered host cell may be modified to synthetically regulate the expression of the 6OMT gene in the engineered host cell. In examples, the engineered host cell may be modified to incorporate a copy, copies, or additional copies, of the 6OMT gene. Additionally or alternatively, the engineered host cell may be modified to incorporate the introduction of a strong promoter element for the overexpression of the 6OMT gene within the engineered host cell. The 6OMT gene may be derived from P. somniferum, T. flavum, Coptis japonica, or another species. In some examples, the 6OMT gene may be 100% similar to the naturally occurring gene.

[CNMT] In some examples, the engineered host cell may modify the expression of the enzyme coclaurine-N-methyltransferase. Coclaurine-N-methyltransferase is encoded by the CNMT gene. In some examples, coclaurine-N-methyltransferase catalyzes the reaction of coclaurine→N-methylcoclaurine, as referenced in FIG. 5. In other examples, the coclaurine-N-methyltransferase enzyme may catalyze the reaction of 3′hydroxycoclaurine→3′hydroxy-N-methylcoclaurine. In other examples, coclaurine-N-methyltransferase may catalyze the reaction of noroxymorphone→naloxone, as referenced in FIG. 26. In other examples, coclaurine-N-methyltransferase may catalyze other reactions detailed herein, such as those provided in FIG. 6. Additionally, the engineered host cell may be modified to include constitutive expression of the CNMT gene in the engineered host cell. Additionally or alternatively, the engineered host cell may be modified to synthetically regulate the expression of the CNMT gene in the engineered host cell. In examples, the engineered host cell may be modified to incorporate a copy, copies, or additional copies, of the CNMT gene. Additionally or alternatively, the engineered host cell may be modified to incorporate the introduction of a strong promoter element for the overexpression of the CNMT gene within the engineered host cell. The CNMT gene may be derived from P. somniferum, T. flavum, Coptis japonica, or another species. In some examples, the CNMT gene may be 100% similar to the naturally occurring gene.

[4′OMT] In some examples, the engineered host cell may modify the expression of the enzyme 4′-O-methyltransferase. 4′-O-methyltransferase is encoded by the 4′OMT gene. In some examples, 4′-O-methyltransferase catalyzes the reaction of 3′-hydroxy-N-methylcoclaurine →reticuline, as referenced in FIG. 5. In other examples, 4′-O-methyltransferase catalyzes other reactions detailed herein, such as those provided in FIG. 6. Additionally, the engineered host cell may be modified to include constitutive expression of the 4′OMT gene in the engineered host cell. Additionally or alternatively, the engineered host cell may be modified to synthetically regulate the expression of the 4′OMT gene in the engineered host cell. In examples, the engineered host cell may be modified to incorporate a copy, copies, or additional copies, of the 4′OMT gene. Additionally or alternatively, the engineered host cell may be modified to incorporate the introduction of a strong promoter element for the overexpression of the 4′OMT gene within the engineered host cell. The 4′OMT gene may be derived from P. somniferum, T. flavum, Coptis japonica, or another species. In some examples, the 4′OMT gene may be 100% similar to the naturally occurring gene.

[CYP80B1] In some examples, the engineered host cell may modify the expression of the enzyme cytochrome P450 80B1. Cytochrome P450 80B1 is encoded by the CYP80B1 gene. In examples, cytochrome P450 80B1 catalyzes the reaction of N-methylcoclaurine→3′-hydroxy-N-methylcoclaurine, as referenced in FIG. 5. An engineered host cell may be modified to include constitutive expression of the cytochrome P450 80B1 gene in the engineered host cell. Additionally or alternatively, the engineered host cell may be modified to synthetically regulate the expression of the cytochrome P450 80B1 gene in the engineered host cell. In examples, the engineered host cell may be modified to incorporate a copy, copies, or additional copies, of the cytochrome P450 80B1 gene. Additionally or alternatively, the engineered host cell may be modified to incorporate the introduction of a strong promoter element for the overexpression of the cytochrome P450 80B1 gene within the engineered host cell. In some cases, the CYP80B1 gene may be codon optimized for expression in Saccharomyces cerevisiae. The cytochrome P450 80B1 gene may be derived from P. somniferum, E. californica, T. flavum, or another species. In some examples, the P450 80B1 gene may be 77% similar to the naturally occurring gene.

[FOL2] In some examples, the engineered host cell may modify the expression of the enzyme GTP cyclohydrolase. GTP cyclohydrolase is encoded by the FOL2 gene. In some examples, GTP cyclohydrolase catalyzes the reaction of GTP→dihydroneopterin triphosphate, as referenced in FIG. 1. The engineered host cell may be modified to include constitutive overexpression of the FOL2 gene in the engineered host cell. The engineered host cell may also be modified to include native regulation. Additionally or alternatively, the engineered host cell may be modified to synthetically regulate the expression of the FOL2 gene in the engineered host cell. In examples, the engineered host cell may be modified to incorporate a copy, copies, or additional copies, of the FOL2 gene. Additionally or alternatively, the engineered host cell may be modified to incorporate the introduction of a strong promoter element for the overexpression of the FOL2 gene within the engineered host cell. The FOL2 gene may be derived from Saccharomyces cerevisiae, Homo sapiens, Mus musculus, or another species. In some examples, the FOL2 gene may be 100% similar to the naturally occurring gene.

[PTPS] In some examples, the engineered host cell may modify the expression of the enzyme 6-pyruvoyl tetrahydrobiopterin (PTP) synthase. Pyruvoyl tetrahydrobiopterin synthase is encoded by the PTPS gene. In some examples, 6-pyruvoyl tetrahydrobiopterin synthase catalyzes the reaction of dihydroneopterin triphosphate→PTP, as referenced in FIG. 1. The engineered host cell may be modified to include constitutive expression of the PTPS gene in the engineered host cell. Additionally or alternatively, the engineered host cell may be modified to synthetically regulate the expression of the PTPS gene in the engineered host cell. In examples, the engineered host cell may be modified to incorporate a copy, copies, or additional copies, of the PTPS gene. Additionally or alternatively, the engineered host cell may be modified to incorporate the introduction of a strong promoter element for the overexpression of the PTPS gene within the engineered host cell. In some cases, the PTPS gene may be codon optimized for expression in Saccharomyces cerevisiae. The PTPS gene may be derived from Rattus norvegicus, Homo sapiens, Mus musculus, or another species. In some examples, the PTPS gene may be 80% similar to the naturally occurring gene.

[SepR] In some examples, the engineered host cell may modify the expression of the enzyme sepiapterin reductase. Sepiapterin reductase is encoded by the SepR gene. In some examples, sepiapterin reductase catalyzes the reaction of PTP→BH₄, as referenced in FIG. 1. The engineered host cell may be modified to include constitutive expression of the SepR gene in the engineered host cell. Additionally or alternatively, the engineered host cell may be modified to synthetically regulate the expression of the SepR gene in the engineered host cell. In examples, the engineered host cell may be modified to incorporate a copy, copies, or additional copies, of the SepR gene. Additionally or alternatively, the engineered host cell may be modified to incorporate the introduction of a strong promoter element for the overexpression of the SepR gene within the engineered host cell. In some cases, the SepR gene may be codon optimized for expression in Saccharomyces cerevisiae. The SepR gene may be derived from Rattus norvegicus, Homo sapiens, Mus musculus, or another species. In some examples, the SepR gene may be 72% similar to the naturally occurring gene.

[PCD] In some examples, the engineered host cell may modify the expression of the enzyme 4a-hydroxytetrahydrobiopterin (pterin-4α-carbinolamine) dehydratase. 4a-hydroxytetrahydrobiopterin dehydratase is encoded by the PCD gene. In some examples, 4a-hydroxytetrahydrobiopterin dehydratase catalyzes the reaction of 4a-hydroxytetrahydrobiopterin →H₂O+quinonoid dihydropteridine, as referenced in FIG. 1. The engineered host cell may be modified to include constitutive expression of the PCD gene in the engineered host cell. Additionally or alternatively, the engineered host cell may be modified to synthetically regulate the expression of the PCD gene in the engineered host cell. In examples, the engineered host cell may be modified to incorporate a copy, copies, or additional copies, of the PCD gene. Additionally or alternatively, the engineered host cell may be modified to incorporate the introduction of a strong promoter element for the overexpression of the PCD gene within the engineered host cell. In some cases, the PCD gene may be codon optimized for expression in Saccharomyces cerevisiae. The PCD gene may be derived from Rattus norvegicus, Homo sapiens, Mus musculus, or another species. In some examples, the PCD gene may be 79% similar to the naturally occurring gene.

[QDHPR] In some examples, the engineered host cell may modify the expression of the enzyme quinonoid dihydropteridine reductase. Quinonoid dihydropteridine reductase is encoded by the QDHPR gene. In some examples, quinonoid dihydropteridine reductase catalyzes the reaction of quinonoid dihydropteridine→BH₄, as referenced in FIG. 1. The engineered host cell may be modified to include constitutive expression of the QDHPR gene in the engineered host cell. Additionally or alternatively, the engineered host cell may be modified to synthetically regulate the expression of the QDHPR gene in the engineered host cell. In examples, the engineered host cell may be modified to incorporate a copy, copies, or additional copies, of the QDHPR gene. Additionally or alternatively, the engineered host cell may be modified to incorporate the introduction of a strong promoter element for the overexpression of the QDHPR gene within the engineered host cell. In some cases, the QDHPR gene may be codon optimized for expression in Saccharomyces cerevisiae. The QDHPR gene may be derived from Rattus norvegicus, Homo sapiens, Mus musculus, or another species. In some examples, the QDHPR gene may be 75% similar to the naturally occurring gene.

[DHFR] In some examples, the engineered host cell may modify the expression of the enzyme dihydrofolate reductase. Dihydrofolate reductase is encoded by the DHFR gene. In some examples, dihydrofolate reductase catalyzes the reaction of 7,8-dihydrobiopterin (BH₂)→5,6,7,8-tetrahydrobiopterin (BH₄), as referenced in FIG. 1. This reaction may be useful in recovering BH₄ as a co-substrate for the converstion of tyrosine to L-DOPA, as illustrated in FIG. 5. The engineered host cell may be modified to include constitutive expression of the DHFR gene in the engineered host cell. Additionally or alternatively, the engineered host cell may be modified to synthetically regulate the expression of the DHFR gene in the engineered host cell. In examples, the engineered host cell may be modified to incorporate a copy, copies, or additional copies, of the DHFR gene. Additionally or alternatively, the engineered host cell may be modified to incorporate the introduction of a strong promoter element for the overexpression of the DHFR gene within the engineered host cell. In some cases, the DHFR gene may be codon optimized for expression in Saccharomyces cerevisiae. The DHFR gene may be derived from Rattus norvegicus, Homo sapiens, or another species. In some examples, the DHFR gene may be 77% similar to the naturally occurring gene.

[CYP—COR] As discussed above with regard to epimerizing 1-BIAs, the engineered host cell may modify the expression of a BIA epimerase. The BIA epimerase is encoded by the CYP—COR gene (e.g., CYP82Y2-COR gene). The CYP—COR gene may also be referred to as the DRS-DRR gene. In some examples, the BIA epimerase catalyzes the conversion of (S)-1-BIA→ (R)-1-BIA, as referenced in FIG. 7. In particular, FIG. 7 illustrates a biosynthetic scheme for conversion of L-tyrosine to morphinan alkaloids, in accordance with embodiments of the invention. FIG. 7 provides the use of the enzymes CPR, cytochrome P450 reductase; CYP—COR, cytochrome P450 CYP82Y1-like codeinone reductase-like fusion; SalSyn, salutaridine synthase; SalR, salutaridine reductase; SalAT, salutaridinol 7-O-acetyltransferase; T6ODM, thebaine 6-O-demethylase; COR, codeinone reductase; and CODM, codeine-O-demethylase.

The engineered host cell may be modified to include constitutive expression of the CYP—COR gene in the engineered host cell. Additionally or alternatively, the engineered host cell may be modified to synthetically regulate the expression of the CYP—COR gene in the engineered host cell. In examples, the engineered host cell may be modified to incorporate a copy, copies, or additional copies, of the CYP—COR gene. Additionally or alternatively, the engineered host cell may be modified to incorporate the introduction of a strong promoter element for the overexpression of the CYP—COR gene within the engineered host cell. The CYP—COR gene may be derived from Papaver bracteatum, Papaver somniferum, Papaver setigerum, Chelidonium majus, or another species. In some examples, the CYP—COR gene may be 77% similar to the naturally occurring gene.

[CPR] In some examples, the engineered host cell may modify the expression of the enzyme cytochrome P450 reductase. The cytochrome P450 reductase is encoded by the CPR gene. In some examples, the cytochrome P450 reductase catalyzes the reaction of (R)-reticuline →salutaridine, as referenced in FIG. 7. Additionally, the cytochrome P450 reductase catalyzes other reactions such as those described in FIGs. throughout the application. The engineered host cell may be modified to include constitutive expression of the CPR gene in the engineered host cell. Additionally or alternatively, the engineered host cell may be modified to synthetically regulate the expression of the CPR gene in the engineered host cell. In examples, the engineered host cell may be modified to incorporate a copy, copies, or additional copies, of the CPR gene. Additionally or alternatively, the engineered host cell may be modified to incorporate the introduction of a strong promoter element for the overexpression of the CPR gene within the engineered host cell. The CPR gene may be derived from E. californica, P. somniferum, H. sapiens, S. cerevisiae, A. thaliana, or another species. In some examples, the CPR gene may be 100% similar to the naturally occurring gene.

[SalSyn] In some examples, the engineered host cell may modify the expression of the enzyme salutaridine synthase. The salutaridine synthase is encoded by the SalSyn gene. In some examples, the salutaridine synthase catalyzes the reaction of (R)-reticuline→salutaridine, as referenced in FIG. 7. The engineered host cell may be modified to include constitutive expression of the SalSyn gene in the engineered host cell. Additionally or alternatively, the engineered host cell may be modified to synthetically regulate the expression of the SalSyn gene in the engineered host cell. In examples, the engineered host cell may be modified to incorporate a copy, copies, or additional copies, of the SalSyn gene. Additionally or alternatively, the engineered host cell may be modified to incorporate the introduction of a strong promoter element for the overexpression of the SalSyn gene within the engineered host cell. In some cases, the SalSyn gene may be codon optimized for expression in Saccharomyces cerevisiae. In some examples the SalSyn may be modified at the N-terminus. The SalSyn gene may be derived from Papaver somniferum, Papaver spp, Chelidonium majus, or another species. In some examples, the SalSyn gene may be 78% similar to the naturally occurring gene.

[SalR] In some examples, the engineered host cell may modify the expression of the enzyme salutaridine reductase. Salutaridine reductase is encoded by the SalR gene. In some examples, salutaridine reductase reversibly catalyzes the reaction of salutaridinol→salutaridine, as referenced in FIG. 7. The engineered host cell may be modified to include constitutive expression of the SalR gene in the engineered host cell. Additionally or alternatively, the engineered host cell may be modified to synthetically regulate the expression of the SalR gene in the engineered host cell. In examples, the engineered host cell may be modified to incorporate a copy, copies, or additional copies, of the SalR gene. Additionally or alternatively, the engineered host cell may be modified to incorporate the introduction of a strong promoter element for the overexpression of the SalR gene within the engineered host cell. In some cases, the SalR gene may be codon optimized for expression in Saccharomyces cerevisiae. The SalR gene may be derived from Papaver somniferum, Papaver bracteatum, Papaver spp., Chelidonium majus, or another species. In some examples, the SalR gene may be 80-100% similar to the naturally occurring gene.

[SalAT] In some examples, the engineered host cell may modify the expression of the enzyme acetyl-CoA: salutaridinol 7-O-acetyltransferase. Acetyl-CoA: salutaridinol 7-O-acetyltransferase is encoded by the SalAT gene. In some examples, acetyl-CoA: salutaridinol 7-O-acetyltransferase catalyzes the reaction of acetyl-CoA+salutaridinol→CoA+7-O-acetylsalutaridinol, as referenced in FIG. 7. The engineered host cell may be modified to include constitutive expression of the SalAT gene in the engineered host cell. Additionally or alternatively, the engineered host cell may be modified to synthetically regulate the expression of the SalAT gene in the engineered host cell. In examples, the engineered host cell may be modified to incorporate a copy, copies, or additional copies, of the SalAT gene. Additionally or alternatively, the engineered host cell may be modified to incorporate the introduction of a strong promoter element for the overexpression of the SalAT gene within the engineered host cell. In some cases, the SalAT gene may be codon optimized for expression in Saccharomyces cerevisiae. The SalAT gene may be derived from Papaver somniferum, Papaver bracteatum, Papaver orientale, Papaver spp., or another species. In some examples, the SalAT gene may be 77-80% similar to the naturally occurring gene.

[T6ODM] In some examples, the engineered host cell may modify the expression of the enzyme thebaine 6-O-demethylase. Thebaine 6-O demethylase is encoded by the T6ODM gene. In some examples, thebaine 6-O-demethylase catalyzes the reaction of thebaine→neopinone, as referenced in FIG. 7. Once the neopinone has been produced, the neopinone may be converted to codeinone. The conversion of neopinone→codeinone may occur spontaneously. Alternatively, the conversion of neopinone→codeinone may occur as a result of a catalyzed reaction. In other examples, the T6ODM enzyme may catalyze the O-demethylation of substrates other than thebaine. For example, T6ODM may O-demethylate oripavine to produce morphinone. Alternatively, T6ODM may catalyze the O-demethylation of BIAs within the 1-benzylisoquinoline, protoberberine, or protopine classes such as papaverine, canadine, and allocryptopine, respectively. The engineered host cell may be modified to include constitutive expression of the T6ODM gene in the engineered host cell. Additionally or alternatively, the engineered host cell may be modified to synthetically regulate the expression of the T6ODM gene in the engineered host cell. In examples, the engineered host cell may be modified to incorporate a copy, copies, or additional copies, of the T6ODM gene. Additionally or alternatively, the engineered host cell may be modified to incorporate the introduction of a strong promoter element for the overexpression of the T6ODM gene within the engineered host cell. In some cases, the T6ODM gene may be codon optimized for expression in Saccharomyces cerevisiae. The T6ODM gene may be derived from Papaver somniferum, or another species. In some examples, the T6ODM gene may be 76.2% similar to the naturally occurring gene.

[COR] In some examples, the engineered host cell may modify the expression of the enzyme codeinone reductase. Codeinone reductase is encoded by the COR gene. In some examples, codeinone reductase catalyzes the reaction of codeinone to codeine, as referenced in FIG. 7. In some cases, codeinone reductase can catalyze the reaction of neopinone to neopine. In other examples, COR can catalyze the reduction of other morphinans including hydrocodone →dihydrocodeine, 14-hydroxycodeinone→14-hydroxycodeine, and hydromorphone→dihydromorphine. The engineered host cell may be modified to include constitutive expression of the COR gene in the engineered host cell. Additionally or alternatively, the engineered host cell may be modified to synthetically regulate the expression of the COR gene in the engineered host cell. In examples, the engineered host cell may be modified to incorporate a copy, copies, or additional copies, of the COR gene. Additionally or alternatively, the engineered host cell may be modified to incorporate the introduction of a strong promoter element for the overexpression of the COR gene within the engineered host cell. In some cases, the COR gene may be codon optimized for expression in Saccharomyces cerevisiae. Additionally or alternatively, the COR gene may be modified with the addition of targeting sequences for mitochondria, vacuole, endoplasmic reticulum, or a combination thereof. The COR gene may be derived from Papaver somniferum, or another species. In some examples, the COR gene may be 76-78% similar to the naturally occurring gene. In examples, the COR gene may be 76.8%, 77.0%, 77.3%, or 77.7% similar to the naturally occurring gene.

[CODM] In some examples, the engineered host cell may modify the expression of the enzyme codeine O-demethylase. Codeine O-demethylase is encoded by the CODM gene. In some examples, codeine O-demethylase catalyzes the reaction of codeine to morphine, as referenced in FIG. 7. Codeine O-demethylase can also catalyze the reaction of neopine to neomorphine. Codeine O-demethylase can also catalyze the reaction of thebaine to oripavine. In other examples, CODM may catalyze the O-demethylation of BIAs within the 1-benzylisoquinoline, aporphine, and protoberberine classes such as reticuline, isocorydine, and scoulerine, respectively. In other examples, the CODM enzyme may catalyze an O,O-demethylenation reaction to cleave the methylenedioxy bridge structures in protopines. The engineered host cell may be modified to include constitutive expression of the CODM gene in the engineered host cell. Additionally or alternatively, the engineered host cell may be modified to synthetically regulate the expression of the CODM gene in the engineered host cell. In examples, the engineered host cell may be modified to incorporate a copy, copies, or additional copies, of the CODM gene. Additionally or alternatively, the engineered host cell may be modified to incorporate the introduction of a strong promoter element for the overexpression of the CODM gene within the engineered host cell. In some cases, the CODM gene may be codon optimized for expression in Saccharomyces cerevisiae. Additionally or alternatively, the CODM gene may be modified with the addition of targeting sequences for mitochondria. The CODM gene may be derived from Papaver somniferum, Papaver spp., or another species. In some examples, the CODM gene may be 75% similar to the naturally occurring gene. In examples, the CODM gene may be 75.2% similar to the naturally occurring gene.

[BBE] In some examples, the engineered host cell may modify the expression of the enzyme berberine bridge enzyme. The berberine bridge enzyme is encoded by the BBE gene. In some examples, berberine bridge enzyme catalyzes the reaction of (S)-reticuline→(S)-scoulerine. The engineered host cell may be modified to include constitutive expression of the BBE gene in the engineered host cell. Additionally or alternatively, the engineered host cell may be modified to synthetically regulate the expression of the BBE gene in the engineered host cell. In examples, the engineered host cell may be modified to incorporate a copy, copies, or additional copies, of the BBE gene. Additionally or alternatively, the engineered host cell may be modified to incorporate the introduction of a strong promoter element for the overexpression of the BBE gene within the engineered host cell. The BBE gene may be derived from Papaver somniferum, Argemone mexicana, Eschscholzia californica, Berberis stolonifera, Thalictrum flavum subsp. glaucum, Coptis japonica, Papaver spp., or another species. In some examples, the BBE gene may be 99% similar to the naturally occurring gene.

[S90MT] In some examples, the engineered host cell may modify the expression of the enzyme S-adenosyl-L-methionine: (S)-scoulerine 9-O-methyltransferase. S-adenosyl-L-methionine: (S)-scoulerine 9-O-methyltransferase is encoded by the S9OMT gene. In some examples, S-adenosyl-L-methionine: (S)-scoulerine 9-O-methyltransferase catalyzes the reaction of S-adenosyl-L-methionine+ (S)-scoulerine→S-adenosyl-L-homocysteine+ (S)-tetrahydrocolumbamine. The engineered host cell may be modified to include constitutive expression of the S9OMT gene in the engineered host cell. Additionally or alternatively, the engineered host cell may be modified to synthetically regulate the expression of the S90MT gene in the engineered host cell. In examples, the engineered host cell may be modified to incorporate a copy, copies, or additional copies, of the S9OMT gene. Additionally or alternatively, the engineered host cell may be modified to incorporate the introduction of a strong promoter element for the overexpression of the S9OMT gene within the engineered host cell. In some cases, the S9OMT gene may be codon optimized for expression in Saccharomyces cerevisiae. The S9OMT gene may be derived from Thalictrum flavum subsp. glaucum, Coptis japonica, Coptis chinensis, Papaver somniferum, Thalictrum spp., Coptis spp., Papaver spp., or another species. In some examples, the S9OMT gene may be 100% similar to the naturally occurring gene. In examples, the S9OMT gene may be 80% similar to the naturally occurring gene.

[CAS] In some examples, the engineered host cell may modify the expression of the enzyme (S)-canadine synthase. (S)-canadine synthase is encoded by the CAS gene. In some examples, (S)-canadine synthase catalyzes the reaction of (S)-tetrahydrocolumbamine→(S)-canadine. The engineered host cell may be modified to express the CAS gene in the engineered host cell. The engineered host cell may be modified to include constitutive expression of the CAS gene in the engineered host cell. Additionally or alternatively, the engineered host cell may be modified to synthetically regulate the expression of the CAS gene in the engineered host cell. In examples, the engineered host cell may be modified to incorporate a copy, copies, or additional copies, of the CAS gene. Additionally or alternatively, the engineered host cell may be modified to incorporate the introduction of a strong promoter element for the overexpression of the CAS gene within the engineered host cell. The CAS gene may be derived from Thalictrum flavum subsp. glaucum, Coptis japonica, Thalictrum spp., Coptis spp., or another species. In some examples, the CAS gene may be 100%

[STOX] In some examples, the engineered host cell may modify the expression of the enzyme (S)-tetrahydroprotoberberine oxidase. (S)-tetrahydroprotoberberine oxidase is encoded by the STOX gene. In some examples, (S)-tetrahydroprotoberberine oxidase catalyzes the reaction of (S)-tetrahydroberberine+2 O₂→berberine+2H₂O₂. The engineered host cell may be modified to include constitutive expression of the STOX gene in the engineered host cell. Additionally or alternatively, the engineered host cell may be modified to synthetically regulate the expression of the STOX gene in the engineered host cell. In examples, the engineered host cell may be modified to incorporate a copy, copies, or additional copies, of the STOX gene. Additionally or alternatively, the engineered host cell may be modified to incorporate the introduction of a strong promoter element for the overexpression of the STOX gene within the engineered host cell. In some examples the STOX may be modified at the N-terminus. In some cases, the STOX gene may be codon optimized for expression in Saccharomyces cerevisiae. The STOX gene may be derived from Berberis wilsonae, Coptis japonica, Berberis spp., Coptis spp., or another species. In some examples, the STOX gene may be 78% similar to the naturally occurring gene.

[TNMT] In some examples, the engineered host cell may modify the expression of the enzyme tetrahydroprotoberberine-N-methyltransferase. Tetrahydroprotoberberine-N-methyltransferase is encoded by the TNMT gene. In some examples, tetrahydroprotoberberine-N-methyltransferase catalyzes the reaction of canadine→N-methylcanadine. In some examples, tetrahydroprotoberberine-N-methyltransferase catalyzes the reaction of noroxymorphone→naloxone, as referenced in FIG. 26.

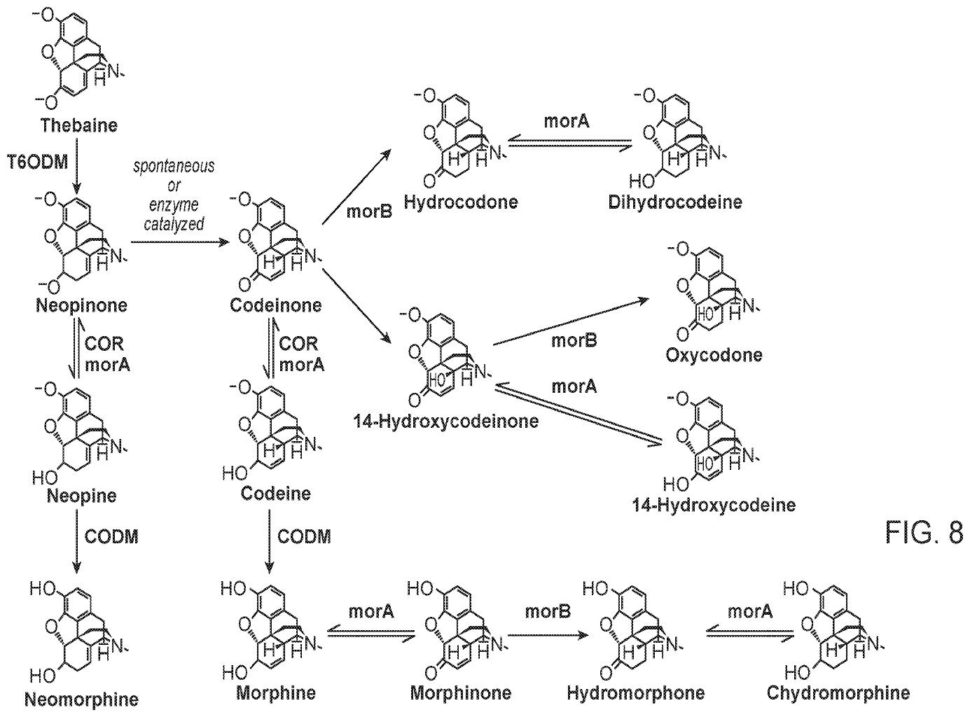

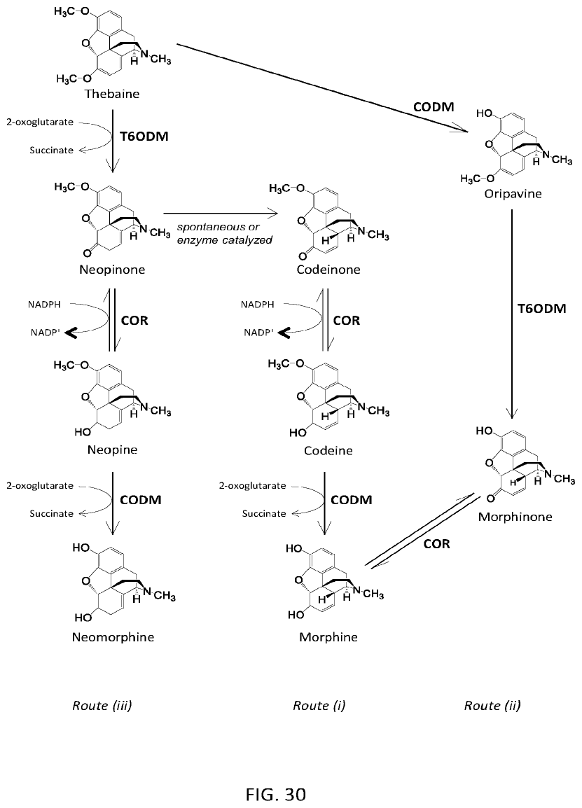

In other examples, tetrahydroprotoberberine-N-methyltransferase catalyzes the reaction of stylopine→cis-N-methylstylopine. The engineered host cell may be modified to include constitutive expression of the TNMT gene in the engineered host cell. Additionally or alternatively, the engineered host cell may be modified to synthetically regulate the expression of the TNMT gene in the engineered host cell. In examples, the engineered host cell may be modified to incorporate a copy, copies, or additional copies, of the TNMT gene. Additionally or alternatively, the engineered host cell may be modified to incorporate the introduction of a strong promoter element for the overexpression of the TNMT gene within the engineered host cell. In some cases, the TNMT gene may be codon optimized for expression in Saccharomyces cerevisiae. The TNMT gene may be derived from Papaver somniferum, Eschscholzia californica, Papaver bracteatum, Argemone mexicana, or another species. In some examples, the TNMT gene may be 100% similar to the naturally occurring gene. In examples, the TNMT gene may be 81% similar to the naturally occurring gene.

[CFS] In some examples, the engineered host cell may modify the expression of the enzyme cheilanthifoline synthase. Cheilanthifoline synthase is encoded by the CFS gene. In examples, cheilanthifoline synthase catalyzes the reaction of scoulerine→cheilanthifoline. An engineered host cell may be modified to include constitutive expression of the CFS gene in the engineered host cell. Additionally or alternatively, the engineered host cell may be modified to synthetically regulate the expression of the CFS gene in the engineered host cell. In examples, the engineered host cell may be modified to incorporate a copy, copies, or additional copies, of the CFS gene. Additionally or alternatively, the engineered host cell may be modified to incorporate the introduction of a strong promotor element for the overexpression of the CFS gene within the engineered host cell. The CFS gene may be derived from P. somniferum, E. californica, A. mexicana, or another species. In some examples, the CFS gene may be 77%, 78%, or 79% similar to the naturally occurring gene. Additionally, the CFS gene may be codon optimized for expression in Saccharomyces cerevisiae.

[STS] In some examples, the engineered host cell may modify the expression of the enzyme stylopine synthase. Stylopine synthase is encoded by the STS gene. In examples, stylopine synthase catalyzes the reaction of cheilanthifoline→stylopine. An engineered host cell may be modified to include constitutive expression of the STS gene in the engineered host cell. Additionally or alternatively, the engineered host cell may be modified to synthetically regulate the expression of the STS gene in the engineered host cell. In examples, the engineered host cell may be modified to incorporate a copy, copies, or additional copies, of the STS gene. Additionally or alternatively, the engineered host cell may be modified to incorporate the introduction of a strong promotor element for the overexpression of the STS gene within the engineered host cell. The STS gene may be derived from P. somniferum, E. californica, A. mexicana, or another species. In some examples, the STS gene may be 76%, 78%, or 79% similar to the naturally occurring gene. Additionally, the STS gene may be codon optimized for expression in Saccharomyces cerevisiae.

[MSH] In some examples, the engineered host cell may modify the expression of the enzyme cis-N-methylstylopine 14-hydroxylase. Cis-N-methylstylopine 14-hydroxylase is encoded by the MSH gene. In examples, cis-N-methylstylopine 14-hydroxylase catalyzes the reaction of cis-N-methylstylopine→protopine. An engineered host cell may be modified to include constitutive expression of the MSH gene in the engineered host cell. Additionally or alternatively, the engineered host cell may be modified to synthetically regulate the expression of the MSH gene in the engineered host cell. In examples, the engineered host cell may be modified to incorporate a copy, copies, or additional copies, of the MSH gene. Additionally or alternatively, the engineered host cell may be modified to incorporate the introduction of a strong promotor element for the overexpression of the MSH gene within the engineered host cell. The MSH gene may be derived from P. somniferum or another species. In some examples, the MSH gene may be 79% similar to the naturally occurring gene. Additionally, the MSH gene may be codon optimized for expression in Saccharomyces cerevisiae.

[P6H] In some examples, the engineered host cell may modify the expression of the enzyme protopine-6-hydroxylase. Protopine-6-hydroxylase is encoded by the P6H gene. In examples, protopine-6-hydroxylase catalyzes the reaction of Protopine→6-hydroxyprotopine. An engineered host cell may be modified to include constitutive expression of the P6H gene in the engineered host cell. Additionally or alternatively, the engineered host cell may be modified to synthetically regulate the expression of the P6H gene in the engineered host cell. In examples, the engineered host cell may be modified to incorporate a copy, copies, or additional copies, of the P6H gene. Additionally or alternatively, the engineered host cell may be modified to incorporate the introduction of a strong promotor element for the overexpression of the CFS gene within the engineered host cell. The P6H gene may be derived from P. somniferum, E. californica, or another species. In some examples, the P6H gene may be 79% similar to the naturally occurring gene. Additionally, the P6H gene may be codon optimized for expression in Saccharomyces cerevisiae.

[DBOX] In some examples, the engineered host cell may modify the expression of the enzyme dihydrobenzophenanthridine oxidase. Dihydrobenzophenanthridine oxidase is encoded by the DBOX gene. In examples, dihydrobenzophenanthridine oxidase catalyzes the reaction of dihydrosanguinarine→sanguinarine. An engineered host cell may be modified to include constitutive expression of the DBOX gene in the engineered host cell. Additionally or alternatively, the engineered host cell may be modified to synthetically regulate the expression of the DBOX gene in the engineered host cell. In examples, the engineered host cell may be modified to incorporate a copy, copies, or additional copies, of the DBOX gene. Additionally or alternatively, the engineered host cell may be modified to incorporate the introduction of a strong promotor element for the overexpression of the DBOX gene within the engineered host cell. The DBOX gene may be derived from P. somniferum or another species. In some examples, the DBOX gene may be 100% similar to the naturally occurring gene. Additionally, the DBOX gene may be codon optimized for expression in Saccharomyces cerevisiae.

[morA] In some examples, the engineered host cell may modify the expression of the enzyme morphine dehydrogenase. Morphine dehydrogenase is encoded by the morA gene. In some examples, morphine dehydrogenase catalyzes the reaction of morphine→morphinone, as referenced in FIG. 8. In other examples, morphine dehydrogenase catalyzes the reaction of codeinone→codeine, also as referenced in FIG. 8. FIG. 8 illustrates a biosynthetic scheme for production of semi-synthetic opiods, in accordance with embodiments of the invention. In particular, FIG. 8 illustrates extended transformations of thebaine in yeast by incorporating morA, morphine dehydrogenase; and morB, morphine reductase. FIG. 30 illustrates an additional transformation of thebaine, in accordance with embodiments of the invention.

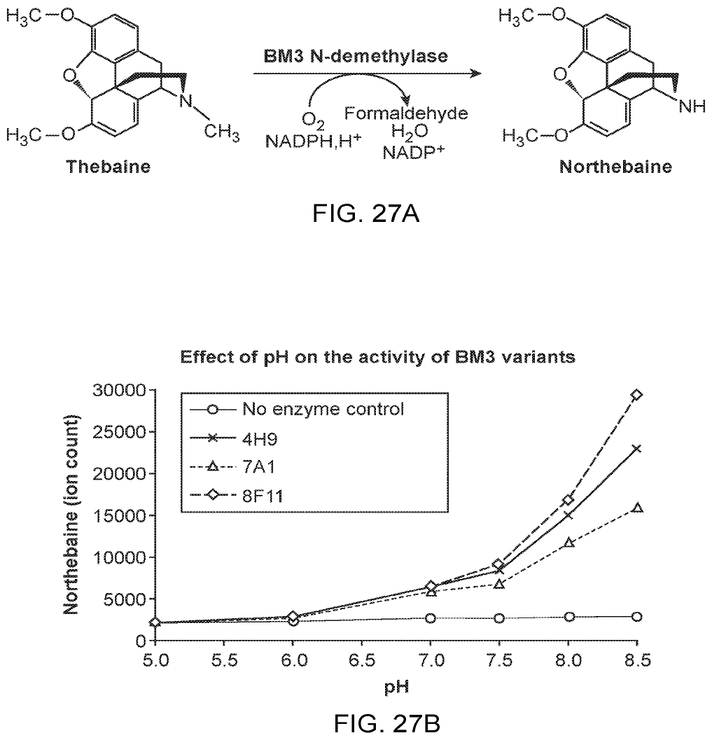

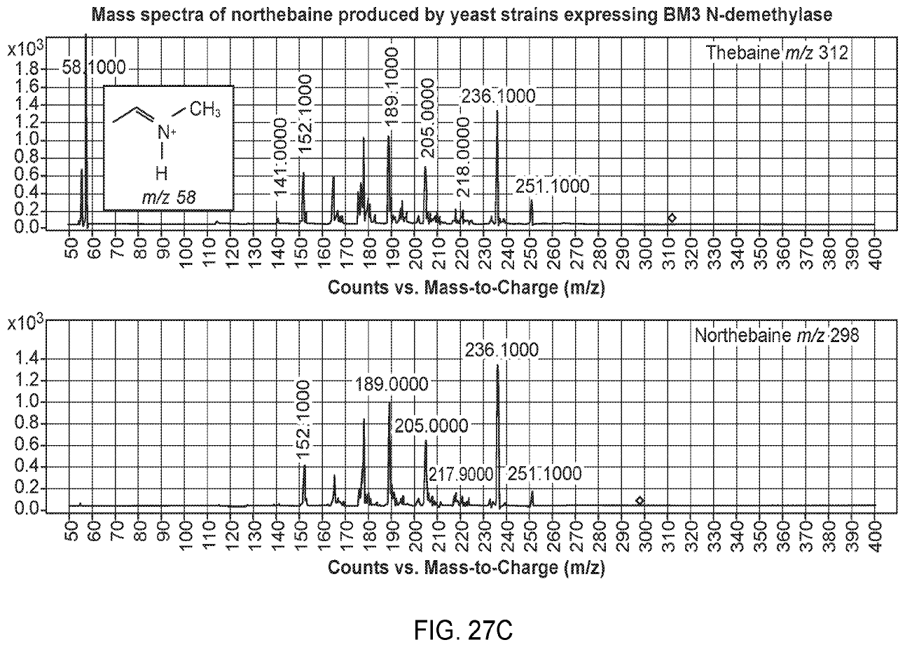

The engineered host cell may be modified to include constitutive expression of the morA gene in the engineered host cell. Additionally or alternatively, the engineered host cell may be modified to synthetically regulate the expression of the morA gene in the engineered host cell. In examples, the engineered host cell may be modified to incorporate a copy, copies, or additional copies, of the morA gene. Additionally or alternatively, the engineered host cell may be modified to incorporate the introduction of a strong promoter element for the overexpression of the morA gene within the engineered host cell. In some cases, the morA gene may be codon optimized for expression in Saccharomyces cerevisiae. The morA gene may be derived from Pseudomonas putida or another species. In some examples, the morA gene may be 73.7% similar to the naturally occurring gene.

[morB] In some examples, the engineered host cell may modify the expression of the enzyme morphinone reductase. Morphinone reductase is encoded by the morB gene. In some examples, morphinone reductase catalyzes the reaction of codeinone→hydrocodone, as referenced in FIG. 8. In other examples, morphinone reductase catalyzes the reaction of morphinone→hydromorphone, also as referenced in FIG. 8. In other examples, morphinone reductase catalyzes the reaction 14-hydroxycodeinone→oxycodone. The engineered host cell may be modified to include constitutive expression of the morB gene in the engineered host cell. Additionally or alternatively, the engineered host cell may be modified to synthetically regulate the expression of the morB gene in the engineered host cell. In examples, the engineered host cell may be modified to incorporate a copy, copies, or additional copies, of the morB gene. Additionally or alternatively, the engineered host cell may be modified to incorporate the introduction of a strong promoter element for the overexpression of the morB gene within the engineered host cell. In some cases, the morB gene may be codon optimized for expression in Saccharomyces cerevisiae. The morB gene may be derived from Pseudomonas putida or another species. In some examples, the morB gene may be 67.2% similar to the naturally occurring gene.

[CYP80A1] In some examples, the engineered host cell may express the enzyme berbamunine synthase. Berbamunine synthase is encoded by the gene for cytochrome P450 enzyme 80A1 (CYP80A1). In some examples, CYP80A1 catalyzes the reaction (S)—N-methylcoclaurine+(R)—N-methylcoclaurine→berbamunine. In other examples, CYP80A1 catalyzes the reaction (R)—N-methylcoclaurine+(R)—N-methylcoclaurine→guattegaumerine. In other examples, CYP80A1 catalyzes the reaction (R)—N-methylcoclaurine+(S)-coclaurine→2′norberbamunine. The engineered host cell may be modified to include constitutive expression of the CYP80A1 gene in the engineered host cell. Additionally or alternatively, the engineered host cell may be modified to synthetically regulate the expression of the CYP80A1 gene in the engineered host cell. In examples, the engineered host cell may be modified to incorporate a copy, copies, or additional copies, of the CYP80A1 gene. Additionally or alternatively, the engineered host cell may be modified to incorporate the introduction of a strong promoter element for the overexpression of the CYP80A1 gene within the engineered host cell. In some cases, the CYP80A1 gene may be codon optimized for expression in Saccharomyces cerevisiae. The CYP80A1 gene may be derived from Berberis stolonifera or another species. In some examples, the CYP80A1 gene may be 76% similar to the naturally occurring gene.

[PODA] In some example, the engineered host cell may express the enzyme protopine O-dealkylase. Protopine O-dealkylase is encoded by the gene PODA. In some examples, PODA catalyzes the O,O-demethylenation of protoberberines and protopines such as canadine, stylopine, berberine, cryptopine, allocryptopine, and protopine. In some examples, PODA catalyzes the O-demethylation of BIAs including tetrahydropapaverine, tetrahydropalmatine, and cryptopine. The engineered host cell may be modified to include constitutive expression of the PODA gene in the engineered host cell. Additionally or alternatively, the engineered host cell may be modified to synthetically regulate the expression of the PODA gene in the engineered host cell. In examples, the engineered host cell may be modified to incorporate a copy, copies, or additional copies, of the PODA gene. Additionally or alternatively, the engineered host cell may be modified to incorporate the introduction of a strong promoter element for the overexpression of the PODA gene within the engineered host cell. In some cases, the PODA gene may be codon optimized for expression in Saccharomyces cerevisiae. The PODA gene may be derived from Papaver somniferum or other species. In some examples, the PODA gene may be 70-100% similar to the naturally occurring gene.

[BM3] In some examples, the engineered host cell may express the enzyme BM3. BM3 is a Bacillus megaterium cytochrome P450 involved in fatty acid monooxygenation in its native host. In some cases BM3 N-demethylates an opioid to produce a nor-opioid, as referenced in FIG. 27. It is also readily expressed as an active heterologous enzyme in yeast and bacteria. BM3 has several advantages as a biosynthetic enzyme including that it is soluble, comes with a fused reductase partner protein, and can readily be engineered to accept new substrates. Additionally, Table 6 illustrates variants of BM3 N-demethylase.

Examples of the aforementioned genes can be expressed from a number of different platforms in the host cell, including plasmid (2μ, ARS/CEN), YAC, or genome. In addition, examples of the aforementioned gene sequences can either be native or codon optimized for expression in the desired heterologous host (e.g., Saccharomyces cerevisiae).

### EXAMPLES

The following examples are given for the purpose of illustrating various embodiments of the invention and are not meant to limit the invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Tyrosine Hydroxylase Mutants Improve Reticuline Production in Engineered Yeast Strains

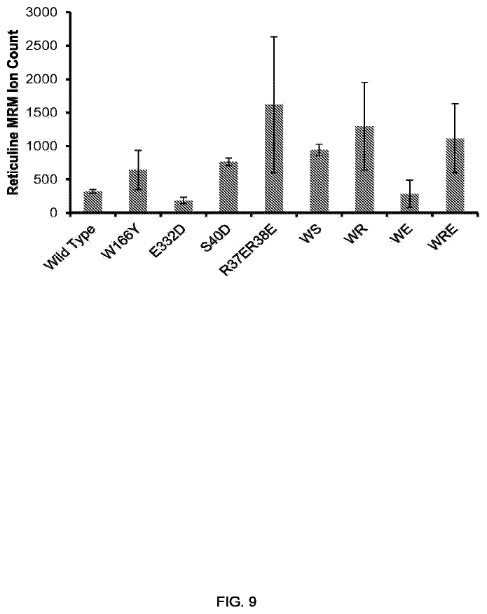

Tyrosine hydroxylase from R. norvegicus was yeast codon optimized, synthesized, and cloned into a low-copy plasmid. Single mutants (W166Y, E332D, S40D and R³⁷ER38E), double mutants (W166Y and E332D, W166Y and S40D, W166Y and R³⁷ER38E), and one triple mutant (W166Y, R³⁷ER38E, and E332D) were generated through site-directed mutagenesis. Each TyrH mutant was expressed from a low-copy plasmid with the GPD promoter in a yeast strain containing the following mutations to central metabolism (as described in U.S. Provisional Patent Application Ser. No. 61/899,496): ARO4^{FBR}, ΔZWF1, and GPD-TKL1 promoter replacement. In addition, the strain expressed a chromosomally integrated copy of DOPA decarboxylase (DODC) from P. putida, four chromosomally integrated genes from R. norvegicus that generate the cosubstrate tetrahydrobiopterin (pyruvoyl tetrahydropterin synthase, PTPS; sepiapterin reductase, SepR; pterin 4a-carbinolamine dehydratase, PCD; dihydropteridine reductase, QDHPR), norcoclaurine synthase (NCS) from C. japonica expressed from a low-copy plasmid with a GPD promoter, and five genes for the biosynthesis of reticuline from norcoclaurine (P. somniferum 6-O-methyltransferase, Ps6OMT; P. somniferum coclaurine N-methyltransferase, PsCNMT; E. californica cytochrome P450 80B1, EcCYP80B1; P. somniferum cytochrome P450 NADPH reductase, PsCPR; and P. somniferum 3′hydroxy-N-methylcoclaurine 4′-O-methyltransferase, Ps4′OMT). The strains harboring TyrH mutants were grown in selective defined media (YNB) lacking tyrosine with 2% dextrose for 96 hours, and the production of reticuline was measured in the media via LC-MS/MS in MRM mode with the transition 330 m/z to 137 m/z. FIG. 9 shows the results of this assay and demonstrates that TyrH mutants can improve reticuline production by as much as 5-fold when compared to wild-type TyrH. As such, FIG. 9 illustrates tyrosine hydroxylase mutants that improve reticuline production from sugar in engineered yeast strains, in accordance with embodiments of the invention.

### Example 2: Expression of DHFR Improves Tyrosine Hydroxylase Activity in Engineered Yeast Strains

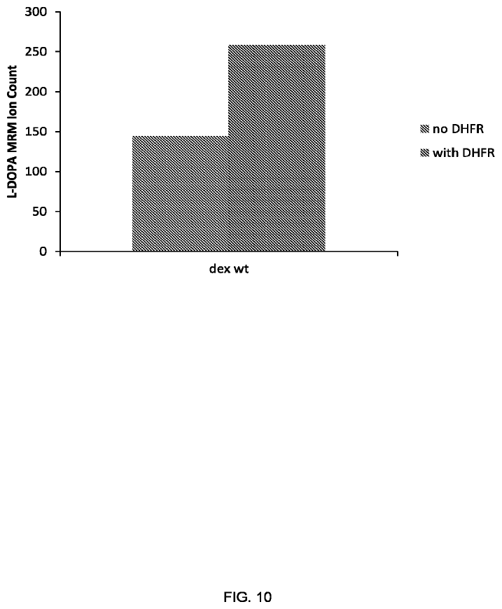

Dihydrofolate reductase (DHFR) from R. norvegicus was yeast codon optimized, synthesized, and cloned into a low-copy plasmid under the control of a GPD promoter. DHFR was coexpressed with wild-type RnTyrH (low-copy plasmid with a GPD promoter) in a yeast strain containing the following mutations to central metabolism (as described in U.S. Provisional Patent Application Ser. No. 61/899,496): ARO4^{FBR}, ΔZWF1, and GPD-TKL1 promoter replacement. In addition, the strain expressed four chromosomally integrated genes from R. norvegicus that generate the cosubstrate tetrahydrobiopterin (pyruvoyl tetrahydropterin synthase, PTPS; sepiapterin reductase, SepR; pterin 4a-carbinolamine dehydratase, PCD; dihydropteridine reductase, QDHPR). The strains expressing DHFR and wild-type RnTyrH were grown in selective defined media (YNB) lacking tyrosine with 2% dextrose for 96 hours, and the production of L-DOPA was measured in the media via LC-MS/MS in MRM mode with the transition 198 m/z to 152 m/z. Expression of DHFR with wild-type RnTyrH increases L-DOPA production by 1.8-fold, as illustrated in FIG. 10. As such, FIG. 10 illustrates coexpression of dihydrofolate reductase (DHFR) that improves L-DOPA production by tyrosine hydroxylase in engineered yeast strains, in accordance with embodiments of the invention.

### Example 3: Addition of Antioxidants to Growth Media Improve Tyrosine Hydroxylase Activity in Engineered Yeast Strains

A yeast strain containing the following mutations to central metabolism (as described in U.S. Provisional Patent Application Ser. No. 61/899,496): ARO4^{FBR}, ΔZWF1, and GPD-TKL1 promoter replacement and expressing four chromosomally integrated genes from R. norvegicus that generate the cosubstrate tetrahydrobiopterin (pyruvoyl tetrahydropterin synthase, PTPS; sepiapterin reductase, SepR; pterin 4a-carbinolamine dehydratase, PCD; dihydropteridine reductase, QDHPR) as well as wild-type RnTyrH from a low-copy plasmid under the control of the GPD promoter was grown in selective defined media (YNB) lacking tyrosine with 2% galactose and 2 mM ascorbic acid for 96 hours.

The production of L-DOPA was measured in the media via LC-MS/MS in MRM mode with the transition 198 m/z to 152 m/z. The addition of 2 mM ascorbic acid improves L-DOPA production with wild-type RnTyrH by 1.8-fold. In addition, the concentration BH₄ intermediates were measured with LC-MS/MS in MRM mode with the following transitions: B, 238 m/z to 178 m/z; BH2, 240 m/z to 165 m/z and BH4, 242 m/z to 166 m/z. The addition of ascorbic acid also increases BH₄ in the media, which indicates the oxidation of BH₄ to BH₂ is prevented.

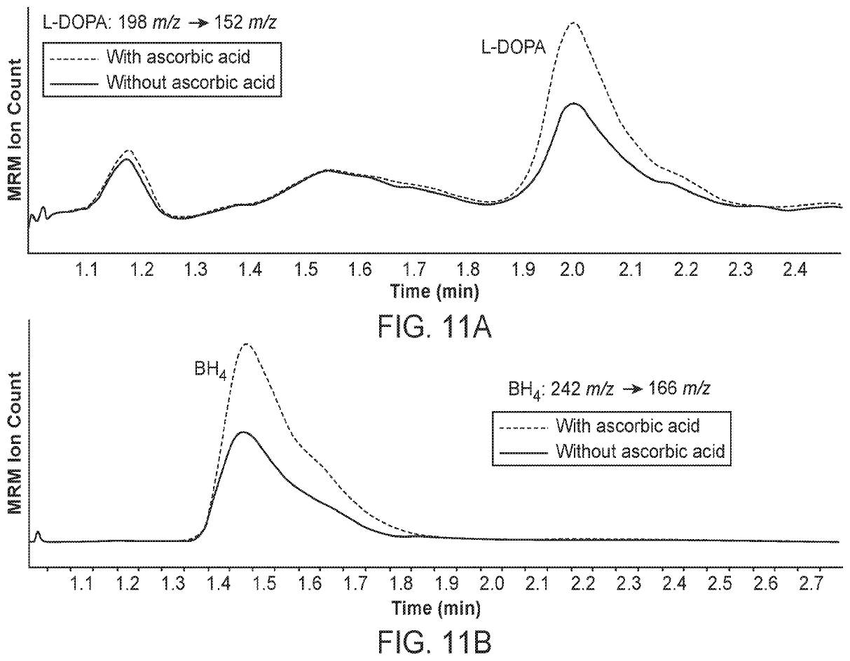

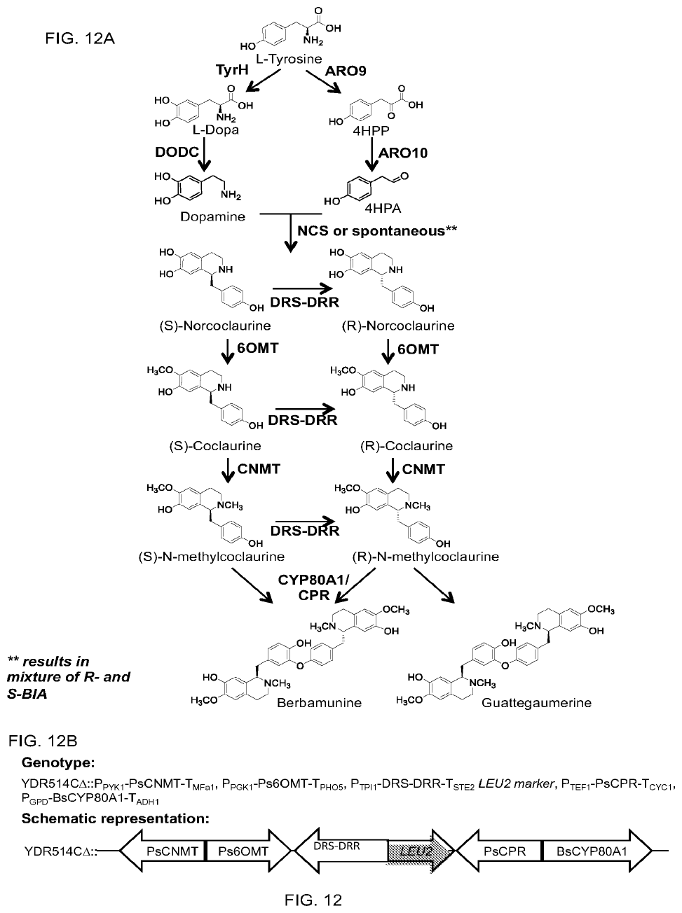

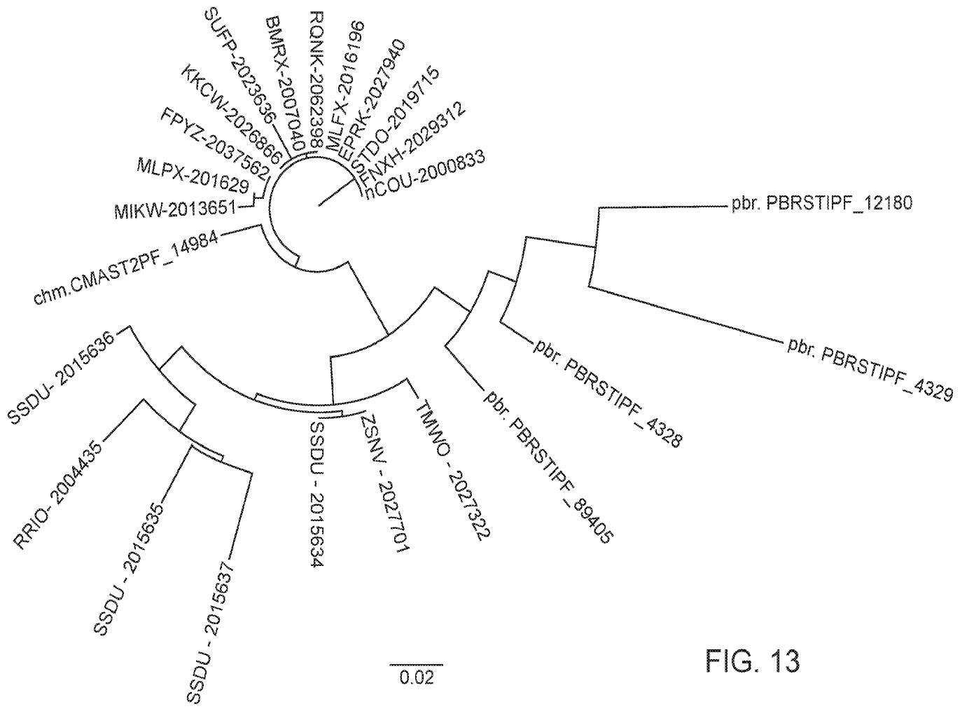

Accordingly, FIG. 11A illustrates addition of antioxidants to culture media that improves L-DOPA production by tyrosine hydroxylase in engineered yeast strains and (B) addition of antioxidants to culture media that increase BH₄ levels, in accordance with embodiments of the invention. In particular, FIG. 11A illustrate a wild-type RnTyrH (expressed from a low-copy plasmid under the control of a GPD promoter) was expressed in a yeast strain containing the following mutations to central metabolism (as described in U.S. Provisional Patent Application Ser. No. 61/899,496): ARO4^{FBR}, ΔZWF1, and GPD-TKL1 promoter replacement. In addition, the strain expressed four chromosomally integrated genes from R. norvegicus that generate the cosubstrate tetrahydrobiopterin (pyruvoyl tetrahydropterin synthase, PTPS; sepiapterin reductase, SepR; pterin 4a-carbinolamine dehydratase, PCD; dihydropteridine reductase, QDHPR). The strains expressing wild-type RnTyrH was grown in selective defined media (YNB) lacking tyrosine with 2% dextrose, with and without 2 mM ascorbic acid (aa) for 96 hours. The production of L-DOPA was measured in the media via LC-MS/MS in MRM mode with the transition 198 m/z to 152 m/z. Additionally, FIG. 11B illustrates, in the same strain described in FIG. 11A, the concentration of the BH₄ intermediate was measured in the media of strains grown with and without 2 mM ascorbic acid (aa) with LC-MS/MS in MRM mode with the following transition: BH₄, 242 m/z to 166 m/z.

### Example 4. Identification of an Epimerase Enzyme

To identify an epimerase enzyme suitable for performing the epimerization reactions of the methods disclosed herein, a cytochrome P450 oxidase 82Y1-like domain and a codeinone reductase-like domain were identified in a single open reading frame (CYP—COR) in publically available plant transcriptomes. The CYP—COR fusions were identified from a BLAST search of the 1000 Plants Project (Matasci, et al. 2014. Gigascience. 3:17) and PhytoMetaSyn (Facchini, et al. 2012. Trends Biotechnol. 30:127-31; Xiao, et al. 2013. J. Biotechnol. 166:122-34) transcriptomes using blastn with the query being the sequence of a previously published COR-silencing VIGS construct that resulted in reticuline accumulation (Wijekoon and Facchini. 2012. Plant J. 69:1052-63). Once one CYP—COR fusion sequence was observed as a hit, that sequence was translated and the amino acid sequence was used as the query for a second search of both databases with tblastn. A phylogenetic tree of the CYP—COR fusion enzymes identified from the databases is provided in FIG. 13. The sequences were identified from The 1000 Plants Project and PhytoMetaSyn transcriptome databases based on a bioinformatic search. Additionally, an example amino acid sequence is provided in FIG. 4, as discussed above. Additionally, Table 1 lists various examples of amino acid sequences identified for this CYP—COR enzyme, which come from various plants including Papaver somniferum (opium poppy), Papaver setigerum (poppy of Troy), Papaver bracteatum (Iranian poppy), and Chelidonium majus (greater celandine).

### Example 5. Epimerization of (S)-Reticuline to (R)-Reticuline in an Engineered Non-Plant Host Cell

Non-plant host cells were engineered to heterologously express enzymes described herein. For instance, yeast strains (Saccharomyces cerevisiae) were engineered to heterologously express the identified epimerases described in Example 4 and to verify their function in the context of this microbial host. The yeast-codon optimized DNA coding sequences for the partial amino acid sequences pbr.PBRST1PF_4328 and pbr.PBRSTIPF_89405 were synthesized in-frame with the yeast-codon optimized coding sequence for amino acids 1-40 of SSDU-2015634 (Table 1) to generate CYP-COR_4328 and CYP-COR_89405, respectively. These CYP—COR coding sequences were cloned into a low-copy plasmid harboring a URA3 selection marker and expressed from the TDH3 promoter. The plasmids were transformed into yeast strains that harbored an expression cassette for a cytochrome P450 reductase (P_{TEF1}-ATR1 or P_{TEF1}-PsCPRv2) integrated into the chromosome. These yeast strains harboring the two plasmids were grown in synthetic complete media with the appropriated drop out solution (-Ura-Trp). The yeast strains were fed (S)-reticuline and BIA metabolites were analyzed after 72 hours of growth by LC-MS/MS analysis.

### Example 6. Production of Salutaridine from (S)-Reticuline in an Engineered Yeast Cell

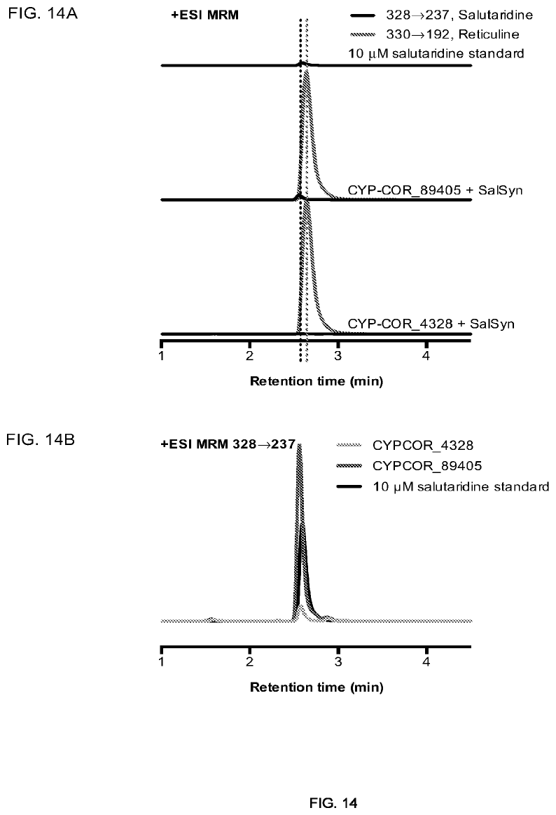

Yeast strains (Saccharomyces cerevisiae) were engineered to heterologously express the identified epimerases described in Example 4 and to verify their function in the context of this microbial host. The yeast-codon optimized DNA coding sequences for the partial amino acid sequences pbr.PBRST1PF_4328 and pbr.PBRST1PF_89405 were synthesized in-frame with the yeast-codon optimized coding sequence for amino acids 1-40 of SSDU-2015634 (Table 1) to generate CYP-COR_4328 and CYP-COR_89405, respectively. These CYP—COR coding sequences were cloned into a low-copy plasmid harboring a URA3 selection marker and expressed from the TDH3 promoter. The salutaridine synthase (SalSyn) coding sequence was cloned into a low-copy plasmid harboring a TRP1 selection marker and expressed from the TDH3 promoter. The plasmids were transformed into yeast strains that harbored an expression cassette for a cytochrome P450 reductase (P_{TEF1}-ATR1 or P_{TEF1}-PsCPRv2) integrated into the chromosome. These yeast strains harboring the two plasmids were grown in synthetic complete media with the appropriated drop out solution (-Ura-Trp). The yeast strains were fed (S)-reticuline and BIA metabolites were analyzed after 72 hours of growth by LC-MS/MS analysis. The analysis indicated that the engineered yeast cells were able to convert (S)-reticuline to (R)-reticuline, which was then acted on by salutaridine synthase to form salutaridine, a 4-ring promorphinan alkaloid (FIG. 7, FIG. 14A, FIG. 14B). Salutaridine synthase has been previously shown to act on (R)-reticuline and have no observable activity on (S)-reticuline (Gesell, et al. 2009. J. Biol. Chem. 284:24432-42).

As shown in FIG. 7, CYP—COR catalyzes the conversion of (S)-reticuline to (R)-reticuline, which is then acted on by salutaridine synthase to make the promorphinan alkaloid salutaridine. FIG. 14A illustrates chromatogram traces showing reticuline and salutaridine for two epimerase variants (CYP-COR_89405, CYP-COR_4328) and a standard. FIG. 14B illustrates the same chromatogram traces for salutaridine in FIG. 14A as replotted to demonstrate co-elution with the standard. In this experiment, the yeast contains two low-copy CEN/ARS plasmids with URA3 and TRP1 selective markers, TDH3 promoters, and the CYP—COR and SalSyn coding sequences. Yeast were grown from freshly transformed colonies in 3 mL selective media overnight, back-diluted into 3.5 mL media to OD 0.8, grown 7 hours, pelleted, and then resuspended into pH 7.4 HEPES buffer with 100 μM (S)-reticuline (Specs). After 16 hours on a spinner at 30° C., the yeast were pelleted and the buffer supernatant was analyzed by LC-MS/MS. Each trace is from a single sample representative of 2. Peaks are normalized such that the largest peak in all chromatograms is 100%.

### Example 7. Production of (R)-Reticuline from Racemic Norlaudanosoline in an Engineered Non-Plant Host Cell

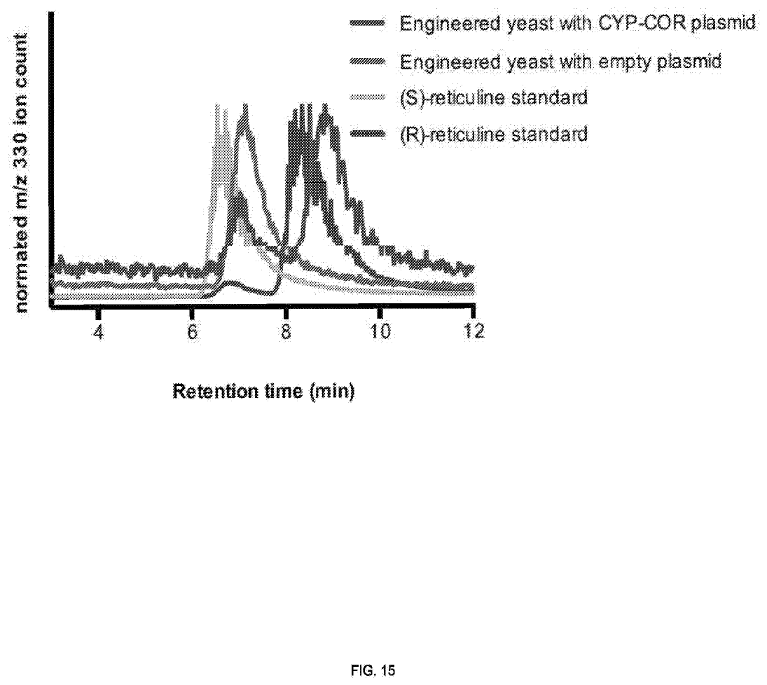

Yeast strains (Saccharomyces cerevisiae) were engineered to heterologously express the identified epimerases described in Example 4 and to verify their function in the context of this microbial host. The yeast-codon optimized DNA coding sequence CYP-COR_89405 described in Example 5 was cloned into a low-copy plasmid harboring a URA3 selection marker and expressed from the TDH3 promoter. This plasmid was transformed into yeast strains that harbored expression cassettes for a cytochrome P450 reductase (P_{TEF}1-ATR1 or P_{TEF}1-PsCPRv2) and three methyltransferases (Papaver somniferum norcoclaurine-6-O-methyltransferase, coclaurine N-methyltransferase, and 3′-hydroxy-N-methylcoclaurine 4′-O-methyltransferase, all expressed from P_{TEF1}) integrated into the chromosome. This yeast strain harboring the plasmid was grown in synthetic complete media with the appropriated drop out solution (-Ura). The yeast strain was fed racemic norlaudanosoline and BIA metabolites were analyzed after 72 hours of growth by LC-MS/MS analysis. For chiral characterization, reticuline was concentrated from yeast media by pelleting 5 mL yeast culture and adding 120 mg XAD-4 resin to 4 mL supernatant, incubating on rotator overnight at room temperature, and eluting with 0.5 mL methanol. The concentrate was fractionated by reverse-phase HPLC (Pursuit XRs-C18, 5 μm, 50 mm×10 mm) with isocratic 15% methanol with 0.1% formic acid over 6.5 min with a flow rate of 5 mL/min and injection volume of 40-50 μL. Peak-based fractions were collected at approximately 4.5 min. Fractions were pooled, freeze-dried, and resuspended in 0.5 mL isopropanol. Depending on concentration, 0.5-5 μL were injected onto a chiral column (Phenomenex Lux cellulose-1, 3 μm, 150 mm×2 mm) and separated with isocratic 72% N-hexane, 28% isopropanol, 0.1% diethylamine with a flow rate of 0.3 mL/min and detection by MS and 250 nm UV. MS detection was performed with an Agilent 6320 Ion Trap mass spectrometer with ESI source gas temperature 350° C., gas flow of 10 L/min, nebulizer pressure 40 PSI and isolation of m/z 330.1 with width 1.0. The retention time of reticuline peaks was compared to that of authentic (S)-reticuline and (R)-reticuline standards. The analysis indicated that the engineered yeast cells containing the CYP—COR plasmid were able to convert racemic norlaudanosoline to (R)-reticuline, while engineered yeast cells with an empty plasmid produced exclusively (S)-reticuline (FIG. 15).

### Example 8: Protein Engineering of Salutaridine Synthase to Improve its Processing and Activity when Expressed in a Microbial Host

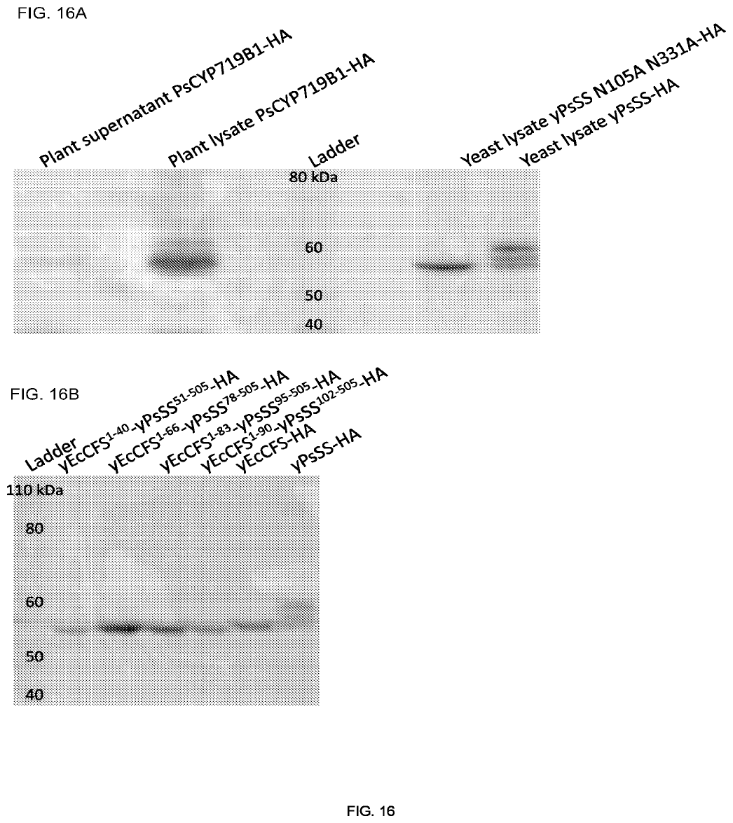

Heterologous proteins may be incorrectly processed when expressed in a recombinant host, for example, plant proteins such as cytochrome P450 enzymes expressed in microbial production hosts. For example, salutaridine synthase, which converts (R)-reticuline to salutaridine, undergoes N-linked glycosylation when heterologously expressed in yeast (FIG. 16A and FIG. 16B). The observed N-linked glycosylation pattern on salutaridine synthase is not observed when the enzyme is expressed in plants and is indicative of incorrect N-terminal sorting of the nascent SalSyn transcript, which reduces the activity of the enzyme in the heterologous microbial host. Thus, protein engineering directed at correcting N-terminal sorting of the nascent transcript and thereby removing the N-linked glycosylation pattern will result in improved activity of the salutaridine synthase enzyme in the recombinant production host.

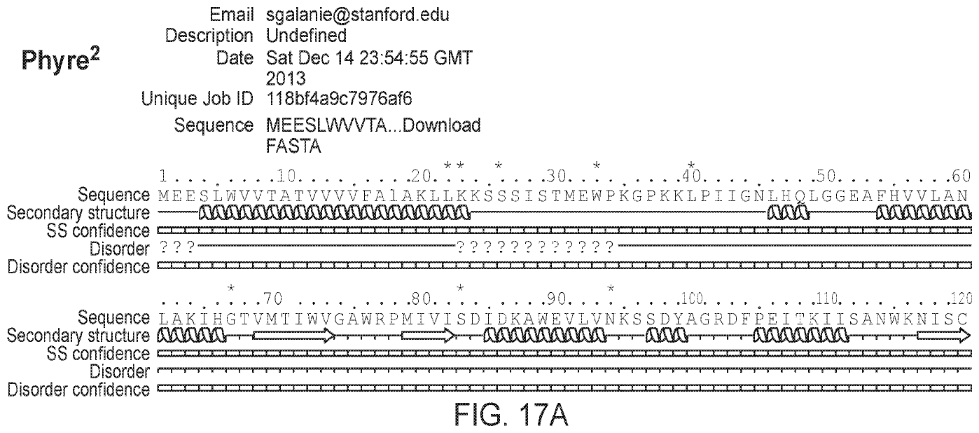

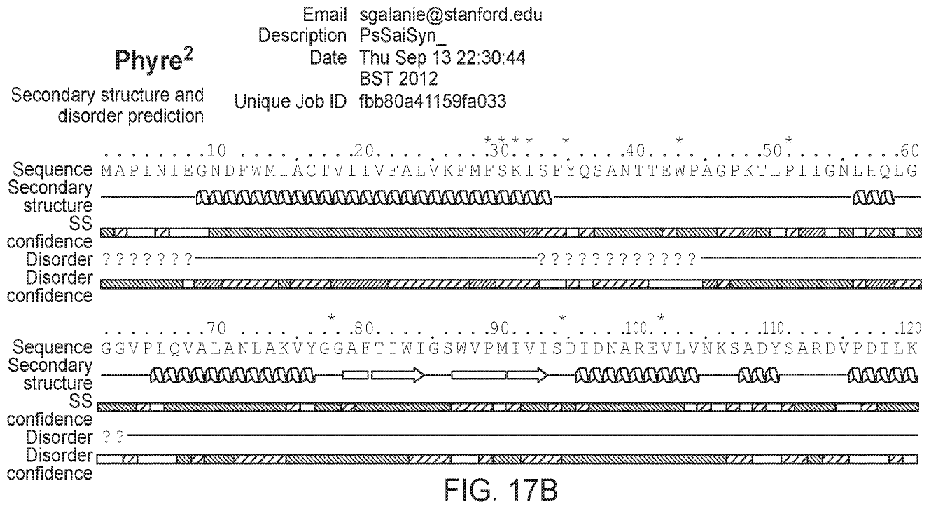

For example, N-terminal alpha-helices from cheilanthifoline synthase (CFS) were used to replace N-terminal alpha-helices from salutaridine synthase (SalSyn, FIG. 17). Junction points for these fusions were selected based on secondary structure motifs of CFS and SalSyn or based on amino acid alignments of CFS and SalSyn. The fusions were cloned by amplifying the N-terminal fragment from CFS and C-terminal fragment from SalSyn with 15-40 nucleotides of overlap with the other fragment, and then assembled with each other and a vector backbone by Gibson assembly to form the complete fusion open reading frame (Gibson, et al. 2009. Nat Methods. 6:343-5).

As another example, the coding sequence for the cytochrome P450 domain from salutaridine synthase was placed directly into the P450 encoding region of other stably expressed cytochrome P450s such as the BM3 enzyme. For example, the conserved cytochrome P450 domain of the salutaridine synthase and the cytochrome P450 domain from an engineered variant of the Bacillus megaterium P450 monooxygenase CYP102A1 (BM3, (Michener and Smolke. 2012. Metab. Eng. 14:306-16)) were identified by NCBI conserved domain search. Primers were designed to fuse the coding sequence of the first few amino acids of the BM3 to the coding sequence for the P450 domain of the salutaridine synthase, followed by the coding sequence for BM3 domains C-terminal to the P450 domain. As before, this construct was assembled via Gibson assembly.

The engineered salutaridine synthase protein fusions were analyzed by Western Blot analysis to confirm full-length expression and modification to or elimination of N-linked glycosylation patterns in yeast (FIG. 16A and FIG. 16B). The salutaridine synthase enzyme and protein fusions were C-terminally tagged with the human influenza hemagglutinin (HA) epitope and cloned into expression plasmids appropriate for yeast and plant expression. For yeast, the enzyme coding sequences were cloned into a low-copy yeast/E. coli shuttle vector harboring a URA3 selection marker and expressed from the TDH3 promoter. For plants, the sequences were cloned into an E. coli/Agrobacterium tumefaciens shuttle vector with kanamycin resistance and the Cauliflower mosaic virus (CaMV) 35S promoter with flanking 5′ and 3′-untranslated regions from Cowpea mosaic virus RNA-2 for transient plant expression via Agrobacterium tumefaciens-infiltration. Yeast engineered to express salutaridine synthase exhibited a banding pattern indicative of N-linked glycosylation. We confirmed that this pattern was due to N-linked glycosylation by performing site-directed mutagenesis on the glycosylation site. In contrast, plant expression of this enzyme did not result in a banding pattern indicative of N-linked glycosylation, as seen in FIG. 16A. Although the N-linked glycosylation sites were unmodified, the engineered salutaridine synthase protein fusions were not N-glycosylated when expressed in yeast, as seen in FIG. 16B. By Western blot, we demonstrated that the yeast-expressed fusion enzymes were present as a single band, similar to the expression observed for the plant-expressed parent enzyme, indicating that the mis-processing of the nascent protein in yeast that resulted in N-linked glycosylation was repaired by the engineered fusions.

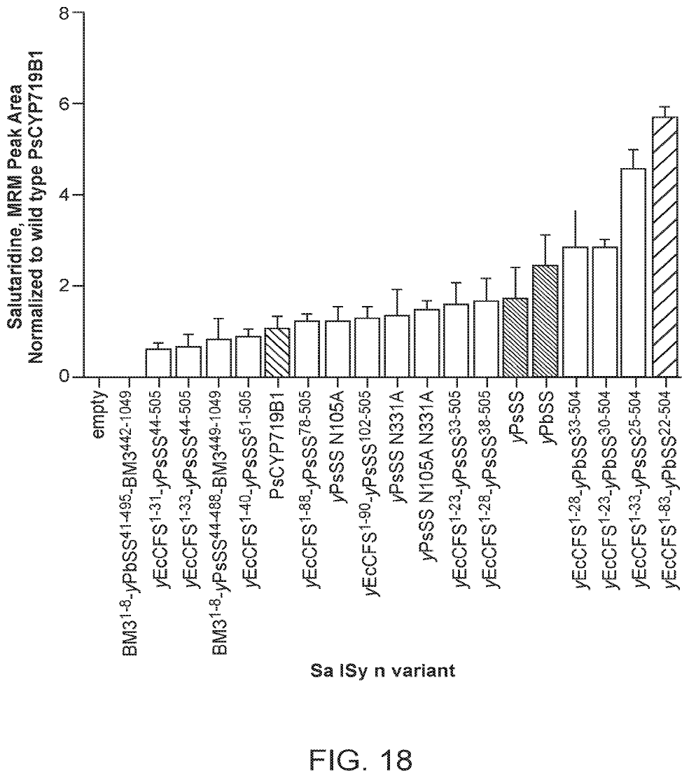

The engineered salutaridine synthase protein fusions were analyzed for improved enzyme activity when heterologously expressed in yeast. Coding sequences for salutaradine synthase and the engineered fusions were cloned into a low-copy plasmid harboring a URA3 selection marker and expressed from the TDH3 promoter. The yeast have P_{TEF1}-PsCPRv2 integrated into the TRP1 locus and contain a single low-copy plasmid with the URA3 selective marker and the salutaridine synthase coding sequence with the TDH3 promoter. Yeast were grown from freshly transformed colonies in 1 mL selective media (-Ura) overnight and back-diluted 1:20 into 0.5 mL selective media in 96-well plates with 10 ∝M (R)-reticuline (Toronto Research Chemicals). After 72-96 hours in the shaking incubator, the yeast were pelleted and the media supernatant was analyzed by LC-MS/MS. The analysis indicated that the engineered salutaradine synthase enzymes exhibited improved activity relative to that of the wild-type sequence when heterologously expressed in yeast (FIG. 18).

FIG. 18 illustrates salutaridine synthase codon-optimization and engineered fusions that improve activity in yeast, in accordance with embodiments of the invention. As seen in FIG. 18, a black bar indicates a native wild-type sequence for salutaridine synthase, PsCYP719B1. Grey bars with black borders are yeast codon-optimized variants from Papaver somniferum and a newly identified sequence from Papaver bracteatum. The diagonally patterned bar indicates the most improved engineered fusion, which is based on the P. bracteatum sequence. Error bars indicate the range of at least two biological replicates. Natural, synthetic codon-optimized, and/or protein engineered variants of salutaridine synthase from P. bracteatum, P. somniferum, or P. setigerum (or related plant) may be used in these engineered strains.

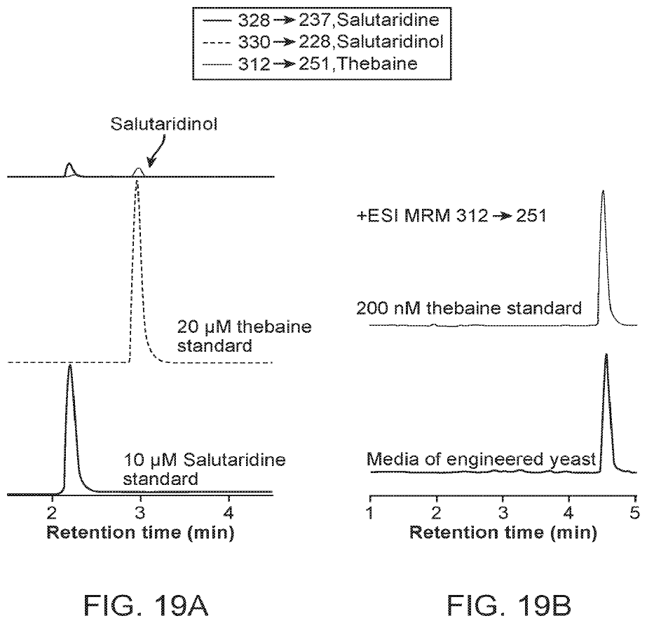

The engineered salutaridine synthase protein fusions can be used in the context of a biosynthetic pathway to increase production of downstream benzylisoquinoline alkaloid products. In one example, yeast were engineered to heterologously express yeast codon optimized genes encoding an engineered salutaridine synthase fusion, P. bracteatum salutaridine reductase, and P. somniferum salutaridinol 7-O-acetyltransferase. The three expression cassettes (P_{TDH3}-D94yPsSS, P_{TPI11}-yPbSalR, P_{TEF1}-yPsSalAT) were assembled into a yeast artificial chromosome (YAC) with a TRP1 section marker. The YAC was placed into yeast that harbored an expression cassette for a cytochrome P450 reductase (P_{TEF1}-ATR1 or P_{TEF1}-yPsCPRv2) integrated into the chromosome. The yeast strains were grown in synthetic complete media with the appropriated drop out solution (-Trp) and fed (R)-reticuline. BIA metabolites were analyzed after 96 hours of growth through LC-MS/MS analysis. The analysis indicates that yeast strains engineered with the engineered salutaridine synthase enzymes and other pathway enzymes produce the morphinan alkaloid thebaine, as illustrated in (A) of FIG. 19.

Accordingly, FIG. 19 illustrates (A) an LC/MS-MS analysis of small scale batch fermentation in which engineered yeast catalyze the conversion of (R)-reticuline to thebaine, in accordance with embodiments of the invention. As provided in (A) of FIG. 19, yeast strains are engineered to have a P_{TEF1}-ATR1 expression cassette integrated into the TRP1 locus and contain a single yeast artificial chromosome with the TRP1 selective marker and three expression cassettes: P_{TDH3}-yEcCFS¹⁻⁸³-yPsSS⁹⁵⁻⁵⁰⁵, P_{TPI1}-yPbSalR, and P_{TEF1}-yPsSalAT. Yeast were grown from freshly transformed colonies in 3 mL selective media overnight and back-diluted 1:20 into 0.5 mL selective media (-Trp) in culture tubes with 100 μM (R)-reticuline (Toronto Research Chemicals). After 72 hours in the shaking incubator, the yeast were pelleted and the media supernatant was analyzed by LC-MS/MS. Chromatogram traces show thebaine produced by this strain and salutaridinol and salutaridine accumulated, along with standards. These traces are representative of two samples.

### Example 9: Protein Engineering of Enzymes in the Downstream Morphinan Branch to Improve Production of Morphinan Products from a Heterologous Microbial Host

In one embodiment of the invention, pathway enzymes are engineered to exhibit increased activity to increase production of the BIA of interest. In this example, mutations were introduced into the open reading frame of a particular pathway enzyme by amplification with Mutazyme II (see Table 11). Sufficient template DNA was included in the amplification reaction to result in a mutation rate of 1-4 nucleotide substitutions per gene. The mutagenized library was cloned into the pYESIL vector by gap repair directly in yeast. In several instances, yeast strains selected for library expression contained integrated copies of genes that generate the substrate of the mutagenized enzyme. For example, a library of CODM variants was transformed into a strain with integrated copies of T6ODM and COR1.3 and fed thebaine in the culture medium. Expression of T6ODM and COR1.3 in these strains ensured that codeine and neopine would be available as substrates for each introduced CODM variant. Individual colonies were inoculated into 96-well plates and cultured 96 hours then assayed for production of their product by liquid chromatograph mass spectrometry (LC-MS). In the example of the CODM library, the products screened for were morphine and neomorphine. In each screen, variants with enhanced BIA production were sequenced and re-cloned for validation. Table 11 includes a summary of mutated enzyme variants identified through the screens that resulted in increased BIA production in yeast.

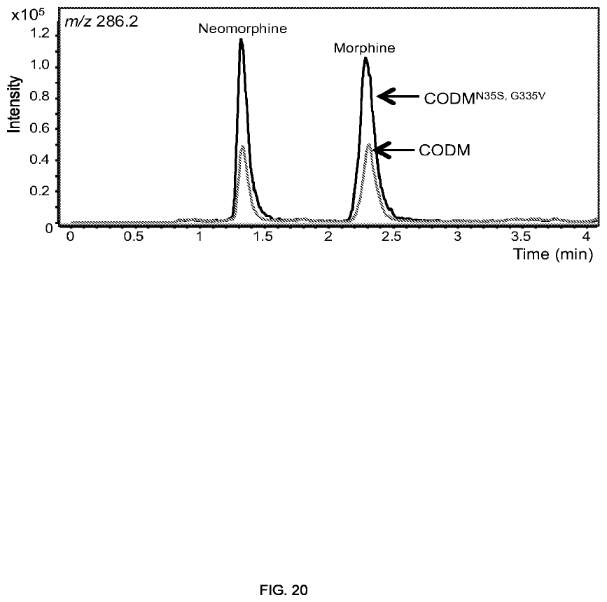

FIG. 20 shows data of the validated enhanced activity of one of these mutants. In particular, FIG. 20 illustrates generation of a CODM enzyme variant exhibiting enhanced activity in yeast through random mutagenesis and screening, in accordance with embodiments of the invention. A library of CODM variants was generated by mutagenizing the coding region by error-prone PCR. A variant identified by screening of this library, CODM^{N35S,G335V}, was re-cloned and expressed in a yeast strain harboring integrated copies of T6ODM and COR1.3. This strain and another control strain expressing wild-type CODM were cultured in liquid medium with 1 mM thebaine. After 96 hours the culture medium was analyzed for CODM activity by LC-MS. Variant CODM^{N35S,G335V} produced 1.4× more morphine and 2.6× more neomorphine than a strain expressing wild-type CODM.

### Example 10: Optimization of Expression and Growth Conditions to Improve Benzylisoquinoline Alkaloid Production from a Heterologous Microbial Host

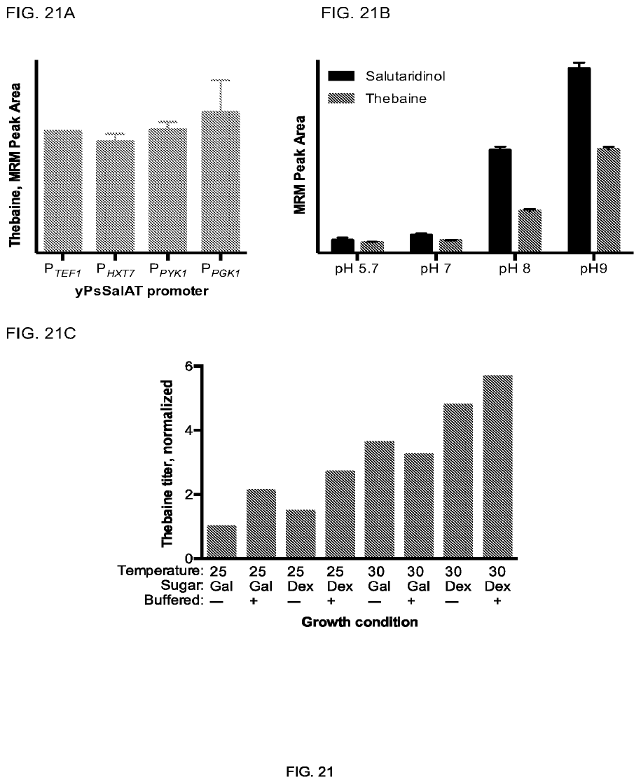

Bezylisoquinoline alkaloid production from an engineered microbial host can be further improved by optimizing the expression of pathway enzymes and growth conditions. In one example, the expression of salutaridinol 7-O-acetyltransferase was altered in yeast by expressing the enzyme from a series of different promoters. The yeast were engineered to heterologously express yeast codon-optimized genes encoding P. somniferum salutaridinol 7-O-acetyltransferase from different promoters (as provided in FIG. 21A). Two expression cassettes (P_{TPI1}-yPbSalR, P_{X}-yPsSalAT) were assembled into a yeast artificial chromosome (YAC) with a TRP1 section marker. The YAC was placed into yeast and cells were grown in synthetic complete media with the appropriated drop out solution (-Trp) and fed salutaridine. BIA metabolites were analyzed after 72 hours of growth by LC-MS/MS analysis. The optimization of pathway enzyme expression level can result in increased production of the morphinan alkaloid thebaine (as provided in FIG. 21A).

Optimization of strain cultivation conditions, including but not limited to sugar source, growth temperature, and pH, can be used to increase production of benzylisoquinoline alkaloids from engineered yeast strains (as provided in FIG. 21B and FIG. 21C). In one example, pH was varied to increase thebaine production from engineered yeast strains. Two expression cassettes (P_{TPI1}-yPbSalR, P_{TEF1}-yPsSalAT) were assembled into a yeast artificial chromosome (YAC) with a TRP1 section marker. The YAC was placed into yeast and cells were grown in synthetic complete media with the appropriated drop out solution (-Trp), resuspended in buffer at pH 5.7-9, and fed salutaridine. BIA metabolites were analyzed after 16 hours of incubation by LC-MS/MS analysis. Levels of the 4-ring promorphinan alkaloid salutaridinol and the 5-ring morphinan alkaloid thebaine increased as a function of increasing pH (as provided in (B) of FIG. 21).

In another example, temperature, sugar, and media buffer content were varied to increase thebaine production from engineered yeast strains. Three expression cassettes (P_{TDH3}-D94yPsSS, P_{TPI1}-yPbSalR, P_{TEF1}-yPsSalAT) were assembled into a yeast artificial chromosome (YAC) with a TRP1 section marker. The YAC was placed into yeast that harbored an expression cassette for a cytochrome P450 reductase (P_{TEF1}-ATR1 or P_{TEF1}-yPsCPRv2) integrated into the chromosome. The yeast strains were grown in synthetic complete media with the appropriated drop out solution (-Trp) and fed (R)-reticuline. BIA metabolites were analyzed after 72 hours of growth by LC-MS/MS analysis. The analysis indicates the microbial production of the morphinan alkaloid thebaine increases under certain cultivation conditions (buffered media with dextrose at 30° C., as provided in FIG. 21C).

Accordingly, FIG. 21A, FIG. 21B, and FIG. 21C illustrate fermentation optimization for conversion of (R)-reticuline to thebaine by engineered yeast, in accordance with embodiments of the invention. LC/MS-MS analysis of whole cell buffered assay of (A) SalAT promoter variants, (B) SalR and SalAT strain grown under different pH conditions, and (C) optimization of sugar source, growth temperature, and media buffer content. (A) Yeast strains engineered to contain a single yeast artificial chromosome with the TRP1 selective marker and two expression cassettes: P_{TPI1}-yPbSalR and P_{X}-yPsSalAT with varied SalAT promoters. Yeast were grown from freshly transformed colonies in 3 mL selective media overnight and back-diluted 1:20 into 0.5 mL media in culture tubes with 100 μM salutaridine (Specs). After 72 hours in the shaking incubator, the yeast were pelleted and the media supernatant was analyzed by LC-MS/MS. (B) Yeast strains engineered to contain a single yeast artificial chromosome with the TRP1 selective marker and two expression cassettes: P_{TPI1}-yPbSalR and P_{TEF1}-yPsSalAT. Yeast were grown from freshly transformed colonies in 3 mL selective media overnight, back-diluted into 3.5 mL media to OD 0.8, grown 7 hours, pelleted, and then resuspended into pH 5.7 MOPS, or pH 7, 8, or 9 Tris buffer with 10 μM salutaridine (Specs). After 16 hours on a spinner at 30° C., the yeast were pelleted and the buffer supernatant was analyzed by LC-MS/MS. Error bars represent the range of two samples. (C) Optimization of sugar source, growth temperature, and media buffer content. In this experiment, the yeast strains are engineered to have PTEFI-ATR1 integrated into the TRP1 locus and contain a single yeast artificial chromosome with the TRP1 selective marker and three expression cassettes: P_{TDH3}-yEcCFS¹⁻⁸³-yPsSS⁹⁵⁻⁵⁰⁵, P_{TPI1}-yPbSalR, and P_{TEF1}-yPsSalAT. Yeast were grown from freshly transformed colonies in 3 mL selective media overnight and back-diluted 1:20 into 0.5 mL media in culture tubes with 100 μM (R)-reticuline (Toronto Research Chemicals). After 72 hours in the shaking incubator, the yeast were pelleted and the media supernatant was analyzed by LC-MS/MS.

### Example 11: Yeast Engineered for the Production of Thebaine from an Early 1-Benzylisoquinoline Alkaloid Scaffold

Yeast strains can be engineered for the production of the morphinan alkaloid thebaine, or morphinan alkaloids derived from thebaine, from early 1-benzylisoquinoline alkaloids. As an example, the engineered yeast strains can produce the morphinan alkaloid products from racemic or (S)-norcoclaurine or racemic or (S)-norlaudanosoline (FIGS. 5, 6, and 7, and (B) of 23). Yeast strains are engineered to produce (S)-reticuline from (S)-norcoclaurine or racemic or (S)-norlaudanosoline by the integration of three or five expression cassettes into the yeast genome. To produce (S)-reticuline from racemic or (S)-norlaudanosoline, the integrated expression cassettes encode Papaver somniferum norcoclaurine 6-O-methyltransferase (Ps6OMT, EC 2.1.1.128), 4′-O-methyltransferase (Ps4′OMT, EC 2.1.1.116), and coclaurine-N-methyltransferase (CNMT, EC 2.1.1.140), each with a TEF1 promoter (Hawkins and Smolke. 2008. Nat. Chem. Biol. 4:564-73). To produce (S)-reticuline from racemic or (S)-norcoclaurine, the strain further harbors integrated expression cassettes for yeast codon-optimized Eschscholzia californica N-methylcoclaurine 3′-hydroxylase (yEcCYP80B1, EC 1.14.13.71) and ATR1 or yPsCPRv2 cytochrome P450 reductase expressed from the TDH3 or TEF1 promoter (CPR, EC 1.6.2.4). These strains are further engineered to incorporate epimerization-catalyzing enzymes (e.g., CYP—COR), salutaridine synthase, salutaridine reductase, and salutaridinol acetyltransferase to convert racemic or (S)-norcoclaurine or racemic or (S)-norlaudanosoline to the morphinan alkaloid thebaine, or morphinan alkaloids derived from thebaine (FIG. 7). As an alternative to expression of an epimerization-catalyzing enzyme, 6OMT, 4′OMT, CNMT, and/or CYP80B1 may be engineered such that rac-reticuline is produced from rac-norcoclaurine or rac-norlaudanosoline.

In one example, a yeast strain was engineered to convert rac-norlaudanosoline to thebaine. The yeast strain harbors integrated expression cassettes encoding Ps6OMT, Ps4′OMT, CNMT, and yPsCPRv2, each with a TEF1 promoter. Four expression cassettes (P_{TDH3}-yEcCFS)¹⁻⁸³-yPsSS⁹⁵⁻⁵⁰⁵, P_{TPI1}-yPbSalR, P_{TEF1}-yPsSalAT, P_{HXT7}—CYP-COR_89405) were assembled into a yeast artificial chromosome (YAC) with a TRP1 selective marker in this strain. The yeast strain harboring the YAC and integrated cassettes was grown in synthetic complete media with the appropriated drop out solution (-Trp) and 1 mM rac-norlaudanosoline substrate. After 96 hours of growth, the media was analyzed for BIA metabolites by LC-MS/MS analysis. Nearly 200 nM thebaine was detected ((B) of FIG. 19). Other engineered salutaridine synthase variants may also be used in this strain (FIG. 18, Example 8).

### Example 12: Platform Yeast Strains Engineered for the Production of Reticuline from L-Tyrosine

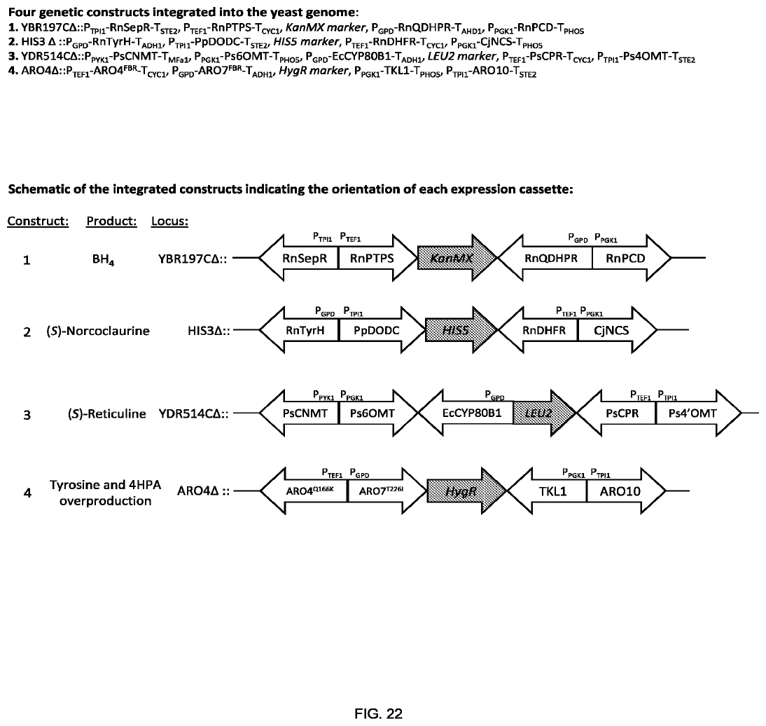

A platform yeast strain that produces the key branch point BIA intermediate (S)-reticuline from L-tyrosine was constructed (FIG. 5). Specifically, four multi-gene expression constructs were integrated into the genome of a yeast strain. The composition of the four constructs is indicated in FIG. 22. Each construct is comprised of 4 or 5 genes expressed from strong constitutive promoters. Genes are positioned at each locus as complete expression cassettes comprising a promoter, gene open reading frame, and terminator as specified in the annotations above the schematic. The schematic shows the orientation of each expression cassette by the direction of the arrow representing a given gene. Selectable markers are italicized in the annotation and represented by grey arrows in the schematic. Each selection marker is flanked by loxP sites to allow removal of the marker from the locus. Additionally, each construct has a selectable marker flanked by loxP sites so that it can be removed by Cre recombinase.

In the first integration construct, four heterologous genes from Rattus norvegicus are integrated into the YBR197C locus together with a G418 selection marker (KanMX). RnPTPS, RnSepR, RnPCD, and RnQDHPR are required to synthesize and regenerate tetrahydrobiopterin (BH₄) from the yeast endogenous folate synthesis pathway. Each gene is codon optimized for expression in yeast.

In the second integration construct, four heterologous genes are integrated into the HIS3 locus together with the HIS5 selection marker. Rattus norvegicus tyrosine hydroxylase (RnTyrH) converts tyrosine to L-DOPA using the cosubstrate BH₄ generated by the preceding integration construct. The RnTyrH gene can be any of the wild-type or improved mutants which confer enhanced activity (e.g., W166Y, R³⁷E, and R³⁸E, Example 1). A second Rattus norvegicus gene, RnDHFR, encodes an enzyme that reduces dihydrobiopterin (an oxidation product of BH₄) to BH₄, in this way increasing the availability of this cosubstrate. Also included in the third construct is PpDODC from Pseudomonas putida, an enzyme that converts L-DOPA to dopamine. The fourth enzyme is CjNCS from Coptis japonica, which condenses 4-HPA and dopamine to make norcoclaurine. Each gene is codon optimized for expression in yeast.

In the third integration construct, five heterologous genes from plants and the LEU2 selection marker are integrated into the locus YDR514C. Ps6OMT, Ps4′OMT, and PsCNMT are methyltransferases from Papaver somniferum and are expressed as native plant nucleotide sequences. A fourth P. somniferum gene, yPsCPRv2, is codon optimized for yeast and encodes a reductase that supports the activity of a cytochrome P450 from Eschscholzia californica, EcCYP80A1. EcCYP80A1 is expressed as its native plant nucleotide sequence. The enzymes encoded in this construct perform two O-methylations, an N-methylation, and a hydroxylation to produce reticuline from the norcoclaurine produced by the preceding integration construct.

In the final integration construct, additional copies of Saccharomyces cerevisiae endogenous genes ARO4^{Q66K}, ARO7^{T226I}, TKL1, and ARO10 are integrated into the ARO4 locus together with a hygromycin resistance selection marker. ARO4^{Q166K} and ARO7^{T226I} are feedback-resistant mutants of ARO4 and ARO10 which each encode a single base pair substitution relative to the wild-type sequence. TKL1 and ARO10 are identical to the native yeast genes, but are expressed behind strong promoters. Aro4p and Aro7p are enzymes in the biosynthesis of aromatic amino acids including tyrosine. Removing feedback inhibition from these enzymes results in upregulation of endogenous tyrosine biosynthesis. Overexpression of Tkl1p upregulates the pentose phosphate pathway resulting in enhanced supply of erythrose 4-phosphate (E4P), a precursor for tyrosine. Overexpression of Aro10p increases the production of 4-HPA.

Platform yeast strains can be constructed with any number of the four expression cassettes. Specifically, platform yeast strains were constructed with integration constructs 1˜4 and integration constructs 1-3. In the latter strain in which the tyrosine over-production construct (construct 4) is excluded, additional tyrosine may be supplied in the culture medium to support the biosynthesis of reticuline. Additional genetic modifications may be incorporated into the platform strains to support production of downstream BIAs and increased flux to BIA biosynthesis.

The yeast strains were grown in synthetic complete media with the appropriated amino acid drop out solution at 25 and 30° C. BIA metabolites in the media supernatant were analyzed after 48 and 96 hours of growth by LC-MS/MS analysis.

### Example 13: Yeast Engineered for the Production of Thebaine and Other Morphinan Alkaloids from L-Tyrosine

Yeast strains can be engineered for the production of the morphinan alkaloid thebaine, or morphinan alkaloids derived from thebaine, from early precursors such as tyrosine. As an example, the platform yeast strains described in Example 12 can be further engineered to produce the morphinan alkaloid products from L-tyrosine (FIG. 7).

The platform yeast strain producing (S)-reticuline from L-tyrosine (see description in Example 12) was further engineered to incorporate epimerization-catalyzing enzymes, such as the newly identified CYP—COR, salutaridine synthase, salutaridine reductase, and salutaridinol acetyltransferase to convert the biosynthesized (S)-reticuline to the morphinan alkaloid thebaine, or morphinan alkaloids derived from thebaine (FIG. 7). Three expression cassettes (P_{TDH3}-yEcCFS¹⁻²⁶-yPbSS³³⁻⁵⁰⁴, P_{TPI1}-yPbSalR, P_{TEF1}-yPsSalAT) were assembled into a yeast artificial chromosome (YAC) with a TRP1 selective marker directly in the platform yeast strain. Other engineered salutaridine synthase variants may also be incorporated into the YAC (FIG. 18, Example 8). The resulting yeast strain was also transformed with a low-copy CEN/ARS plasmid with a URA3 selective marker, TDH3 promoter, and a CYP—COR coding sequence.

The yeast strains harboring the YAC, low-copy plasmid, and integrated cassettes were grown in synthetic complete media with the appropriated drop out solution (-Ura-Trp) at 25 and 30° C. After 96 hours of growth, the media was analyzed for BIA metabolites by LC-MS/MS analysis. Further culture optimization with respect to temperature, carbon source, pH condition, and media composition was performed to improve BIA production.

Additional genetic modifications can be introduced into the yeast strains to produce morphinan alkaloids derived from thebaine (FIG. 7). In one example, the expression cassettes P_{ADH1}-T6ODM-T_{ADH1}, P_{HXT7}-COR-T_{PGK1}, and P_{TEF1}-CODM-T_{CYC1} were directly assembled and integrated into the trp1 locus of the thebaine-producing yeast strain (Thodey et al., 2014). In another example, these yeast strains can be further engineered to produce additional morphine alkaloids by directly assembling the expression cassettes P_{GPD}-morA-T_{CYC1}, P_{PGK1}-morB-T_{PHO5} and integrating this construct into the ura3 locus on the chromosome (Thodey et al., 2014).

### Example 14: O-Demethylation of Opioid Molecules

For high throughput screening of demethylation reactions a purpald assay was used. For example, demethylation catalyzed by 2-oxoglutarate dependent dioxygenases produces formaldehyde as a product as shown in the generalized chemical equation: [substrate]+2-oxoglutarate+O2 [product]+formaldehyde+succinate+CO2. Purpald reagent in alkaline conditions undergoes a color change in the presence of formaldehyde that can be quantified to concentrations as low as 1 nM with a spectrophotometer at 510 nm.

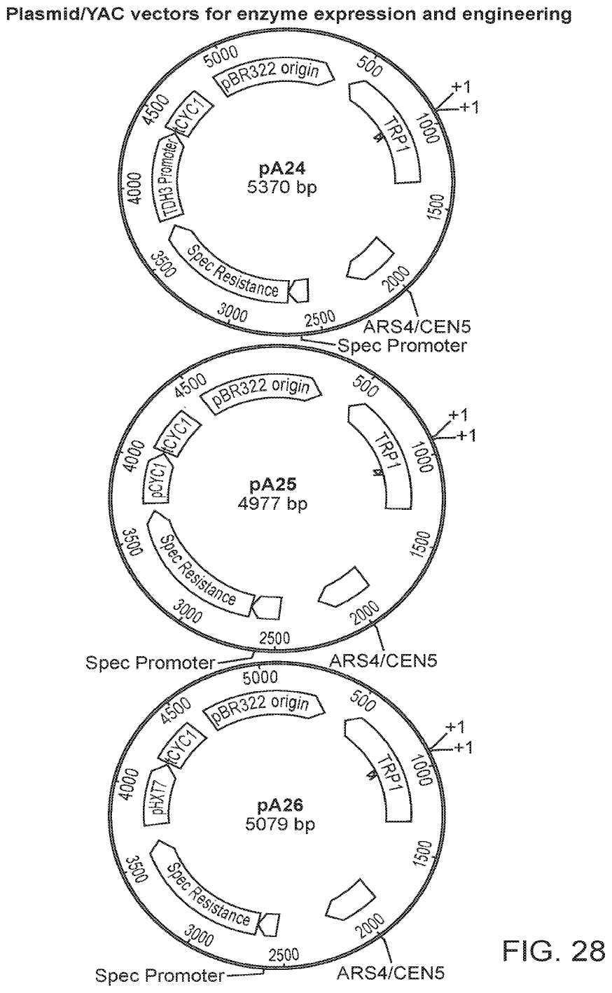

An important step in the production of nor-opioid compounds is the O-demethylation of molecules such as oxycodone (see FIG. 23). To identify enzymes capable of performing this step, sequences from Table 3 were subjected to codon optimization for expression in S. cerevisiae, and ordered as synthetic genes (from Integrated DNA Technologies). Codon optimized sequences were cloned into expression vectors pA24, pA25, or pA26 (or similar vectors), shown in FIG. 28, which harbor promoter sequences of varying strength, by gap repair in the Cen.PK2 yeast host strain, according to standard molecular biology procedures. Individual colonies were isolated and verified by PCR and sequencing (ELIM biopharmaceuticals).

FIG. 28 illustrates plasmid/YAC vectors for enzyme expression and engineering, in accordance with embodiments of the invention. Candidate and engineered enzymes were cloned into these vectors for expression in S. cerevisiae strains. Examples of pA24, pA25, and pA26 sequences are provided in Table 7.

Strains expressing putative O-demethylase enzymes were then tested for basal levels of activity on the various substrates listed in FIG. 23. To detect activity, cell cultures were grown in selective medium (as described above), lysed by glass bead disruption, and incubated with substrates in the presence of redox molecules and other cofactors (such as NADH, NADPH, and iron, at different concentrations depending on the enzyme requirements). O-demethylation of substrates, including but not limited to those listed in FIG. 23, was then detected by analysis via LC-MS of experimental and control samples (such as quantified amounts of oxycodone and oxymorphone, for example).

To identify engineered enzymes with improved O-demethylation activity, sequences encoding enzymes listed in Table 3 were subjected to random mutagenesis and then screened via a high-throughput colorimetric assay. Initial libraries were generated by error-prone PCR using Mutazyme II (Agilent Technologies), and variants were cloned into the pA24 (or similar) vector by gap repair in the Cen.PK2 screening host. Strains expressing mutated enzymes, with either individual mutations or combinations of mutations introduced by gene shuffling or other methods, were grown in selective medium in 96-well plate format under varying fermentation conditions (different media components, pH, and temperature, for example), pelleted, and lysed by glass bead disruption. Lysates were incubated with substrates (listed in FIG. 23) and assayed for formaldehyde production in the purpald assay. Enzymes with improved O-demethylation activity were verified by directly measuring O-demethylated product (oxymorphone, for example) formation in the culture medium by LC-MS.

### Example 15: N-Demethylation of Opioid Molecules

N-demethylase activity removes the N-methyl group present in opioid substrate molecules (such as oxymorphone) and produces a nor-opioid compound (such as noroxymorphone), an important intermediate in the ultimate biosynthesis of nal-opioids. To identify enzymes capable of performing this step, sequences from Table 4 were subjected to codon optimization for expression in S. cerevisiae, and ordered as synthetic genes (from Integrated DNA Technologies). Codon optimized sequences were cloned into expression vectors pA24, pA25, or pA26 (or similar vectors), shown in FIG. 28, which harbor promoter sequences of varying strength, by gap repair in the Cen.PK2 yeast host strain, according to standard molecular biology procedures. Individual colonies were isolated and verified by PCR and sequencing (ELIM biopharmaceuticals).

Strains expressing putative O-demethylase enzymes were then tested for basal levels of activity on the various substrates listed in FIG. 24. To detect activity, cell cultures were grown in selective medium (as described above), lysed by glass bead disruption, and incubated with substrates in the presence of redox molecules and other cofactors (NADH, NADPH, and iron, for example). N-demethylation of substrates was then detected by analysis via LC-MS of experimental and control samples.

To identify engineered enzymes with improved N-demethylation activity, sequences encoding enzymes listed in Table 4 were subjected to random mutagenesis and then screened via a high-throughput colorimetric assay. Initial libraries were generated by error-prone PCR using Mutazyme II (Agilent Technologies), and variants were cloned into the pA24 (or similar) vector by gap repair in the Cen.PK2 screening host. Strains expressing mutated enzymes, with either individual mutations or combinations of mutations introduced by gene shuffling or other methods, were grown in selective medium in 96-well plate format under varying fermentation conditions (different media components, pH, and temperature, for example), pelleted, and lysed by glass bead disruption. Lysates were incubated with substrates (listed in FIG. 24) and assayed for formaldehyde production in the purpald assay. Enzymes with improved N-demethylation activity were verified by direct measurement of N-demethylated product (noroxymorphone, for example) formation in the culture medium by LC-MS.

### Example 16: Modification of Nor-Opioid Compounds to Generate Nal-Opioids

Nor-opioid molecules can be modified at the exposed nitrogen to generate nal-opioids (see FIG. 25), an important class of pharmacotherapies for combating opioid addiction and opioid-associated side effects. To identify enzymes capable of modifying nor-opioid molecules, sequences from Table 5 were subjected to codon optimization for expression in S. cerevisiae, and ordered as synthetic genes (from Integrated DNA Technologies). Codon optimized sequences were cloned into expression vectors pA24, pA25, or pA26 (or similar vectors), shown in FIG. 28, which harbor promoter sequences of varying strength, by gap repair in the Cen.PK2 yeast host strain, according to standard molecular biology procedures. Individual colonies were isolated and verified by PCR and sequencing (ELIM biopharmaceuticals).

Strains expressing putative modifying enzymes were then tested for basal levels of activity on the various substrates listed in FIG. 25. To detect activity, cell cultures were grown in selective medium (as described above), lysed by glass bead disruption, and incubated with substrates in the presence of redox molecules and other cofactors (NADH, NADPH, and iron, for example). N-methylation of substrates was tested using S-adenosylmethionine (SAM) as the cosubstrate, and then additional modifying activity of enzymes was tested using SAM analogues (see “Cosubstrates” in FIG. 25). Modification of BIA substrates was detected via LC-MS of experimental and control samples.

To identify engineered enzymes with improved BIA modifying activity, sequences encoding enzymes listed in Table 5 were subjected to random mutagenesis and then screened via a high-throughput colorimetric assay. Initial libraries were generated by error-prone PCR using Mutazyme II (Agilent Technologies), and variants were cloned into the pA24 (or similar) vector by gap repair in the Cen.PK2 screening host. Strains expressing mutated enzymes, with either individual mutations or combinations of mutations introduced by gene shuffling or other methods, were grown in selective medium in 96-well plate format under varying fermentation conditions (different media components, pH, and temperature, for example), pelleted, and lysed by glass bead disruption. To detect N-methylation activity in a high-throughput screen, lysates were incubated with substrates (such as noroxymorphone) in the presence of a BM3 variant with demethylating activity, and assayed for formaldehyde production in the purpald assay (for indirect measurement of methylation). In this case, formaldehyde formation can only result from the activity of BM3 on a substrate that has been N-methylated by an enzyme of interest. Enzymes with improved modifying activity were additionally tested for activity in cell lysates using various SAM analogues as cosubstrates (see FIG. 25), and verified by direct measurement of product formation by LC-MS. The best variant enzymes were selected for the efficient bioconversion of substrate molecules to nal-opioid compounds.

### Example 17: Demethylase Activity of BM3 Enzyme on Opioid Molecules

BM3 is a Bacillus megaterium cytochrome P450 involved in fatty acid monooxygenation in its native host. It is also readily expressed as an active heterologous enzyme in yeast and bacteria. BM3 has several advantages as a biosynthetic enzyme including that it is soluble, comes with a fused reductase partner protein, and can readily be engineered to accept new substrates. Several known BM3 variants have specific alanine substitutions which allow the rigid morphinan pentacyclic structure to access the active site. These variants were expressed in yeast and observed to N-demethylate thebaine to northebaine.

Specifically, BM3 variants 4H9, 7A1, and 8F11 (listed in Table 6) were integrated into the genomes of individual yeast strains (CEN.PK2) and incubated in citric acid-phosphate buffer (pH 5.0, 6.0, 7.0) and Tris-HCl buffer (pH 7.5, 8.0, 8.5) with 100 μM thebaine for 20 hours. A genetic construct which was identical except for the exclusion of the BM3 open reading frame was integrated to generate a no-enzyme control strain. The cells expressing BM3 produced northebaine at all tested pH levels above 7.0. The northebaine generated by the yeast strains was quantified by liquid chromatography mass spectrometry. The mass spectrum of northebaine (m/z 298) lacked the m/z 58 product ion consistent with a demethylated nitrogen (see FIG. 27C).

FIG. 27A, FIG. 27B and FIG. 27C illustrate the functional expression of BM3 variants, in accordance with embodiments of the invention. FIG. 27A shows the reaction mediated by the BM3 N-demethylase enzyme. FIG. 27B illustrates the functional expression of BM3 variants with thebaine N-demethylase activity in yeast. BM3 variants 4H9, 7A1, and 8F11 were integrated into the genomes of individual yeast strains (CEN.PK2) and incubated in citric acid-phosphate buffer (pH 5.0, 6.0, 7.0) and Tris-HCl buffer (pH 7.5, 8.0, 8.5) with 100 μM thebaine for 20 hours. A genetic construct which was identical except for the exclusion of the BM3 open reading frame was integrated to generate a no-enzyme control strain. The cells expressing BM3 produced northebaine at all tested pH levels above 7.0. The northebaine generated by the yeast strains was quantified by liquid chromatography mass spectrometry. The mass spectrum of northebaine (m/z 298) lacked the m/z 58 product ion consistent with a demethylated nitrogen (see FIG. 27C).

### Example 18: Biological Production of O-Demethylated Opioid Molecules

Enzymes described in Example 14 and listed in Table 3, that displayed O-demethylase activity on BIA molecules (such as those listed in Table 2), were incorporated into a microbial strain (either Saccharomyces cerevisiae or Escherichia coli) which biosynthesizes morphinan alkaloids de novo. The complete BIA biosynthetic pathway uses tyrosine produced by the host cell and/or supplemented in the culture medium. Two molecules of tyrosine are modified and condensed to form the first benzylisoquinoline structure which may be either norcoclaurine or norlaudanosoline. The benzylisoquinoline is further modified to form (S)-reticuline and then stereochemically inverted by the activity of an epimerase enzyme to yield (R)-reticuline. (R)-reticuline undergoes a carbon-carbon coupling reaction to form the first promorphinan, salutaridine, and is further modified before undergoing an oxygen-carbon coupling reaction to arrive at the first morphinan alkaloid structure, thebaine (see FIG. 26). Table 2 lists enzymes and activities in the complete pathway.

FIG. 26 illustrates a biosynthesis scheme in a microbial cell, in accordance with embodiments of the invention. Tyrosine produced endogenously by the cell and/or supplied in the culture medium is converted to oxycodone (broken arrows represent multiple enzymatic steps). The oxycodone is then 3-O-demethylated to oxymorphone and N-demethylated to noroxymorphone. Finally, an N-methyltransferase accepts allyl and cyclopropylmethyl carbon moieties from SAM analogues to produce naloxone and naltrexone, respectively.

To detect O-demethylase activity in strains producing morphinan alkaloid molecules (see FIG. 26), cells expressing candidate enzymes, either from plasmid vectors or chromosomally-integrated cassettes, were propagated by fermentation and cell supernatants were collected to analyze the total opioid profile (as described above). O-demethylation of opioid molecules in strains harboring the complete BIA pathway was detected by LC-MS (as described above). Specifically, the conversion of oxycodone to oxymorphone was detected. To detect O-demethylation activity via biocatalysis, strains were cultured in selective medium and then lysed by glass bead disruption. Cell lysates were supplied exogenously with opioid substrates (see FIG. 23), and other cofactors necessary for enzyme function. O-demethylation of opioid molecules was detected by LC-MS.

### Example 19: Biological Production of N-Demethylated Opioid Molecules

Enzymes described in Example 15 and listed in Table 4, that displayed N-demethylase activity on BIA molecules (such as those listed in Table 2), were incorporated into a microbial strain (either Saccharomyces cerevisiae or Escherichia coli) which biosynthesizes morphinan alkaloids de novo. The complete BIA biosynthetic pathway uses tyrosine produced by the host cell and/or supplemented in the culture medium. Two molecules of tyrosine are modified and condensed to form the first benzylisoquinoline structure which may be either norcoclaurine or norlaudanosoline. The benzylisoquinoline is further modified to form (S)-reticuline and then stereochemically inverted by the activity of an epimerase enzyme to yield (R)-reticuline. (R)-reticuline undergoes a carbon-carbon coupling reaction to form the first promorphinan, salutaridine, and is further modified before undergoing an oxygen-carbon coupling reaction to arrive at the first morphinan alkaloid structure, thebaine (see FIG. 26). Table 2 lists enzymes and activities in the complete pathway.

To detect N-demethylase activity in strains producing morphinan alkaloid molecules (see FIG. 26), cells expressing candidate enzymes, either from plasmid vectors or chromosomally-integrated cassettes, were propagated by fermentation and cell supernatants were collected to analyze the total opioid profile (as described above). N-demethylation of opioid molecules in strains harboring the complete BIA pathway was detected by LC-MS (as described above). Specifically, the conversion of oxymorphone to noroxymorphone was detected. To detect N-demethylation activity via biocatalysis, strains were cultured in selective medium and then lysed by glass bead disruption. Cell lysates were supplied exogenously with opioid substrates (see FIG. 24), and other cofactors necessary for enzyme function. N-demethylation of opioid molecules was detected by LC-MS.

### Example 20: Biological Production of Nal-Opioid Compounds

Enzymes described in Example 16 and listed in Table 5, that displayed N-methylase activity on BIA molecules (such as those listed in Table 2), were incorporated into a microbial strain (either Saccharomyces cerevisiae or Escherichia coli) which biosynthesizes morphinan alkaloids de novo. FIG. 26 shows an example of the complete reaction scheme from the precursor molecule thebaine to the final nal-opioid compounds naloxone and naltrexone. These strains additionally express enzymes from Examples 1 and 2 and Tables 1 and 2, that are responsible for generating nor-opioid compounds from the complete BIA pathway. N-methylase enzymes were also expressed in a microbial strain (either Cen.PK2 for S. cerevisiae or BL21 for E. coli, for example) lacking the biosynthetic pathway, to generate a strain that is capable of biocatalysis of several different exogenously-supplied substrate molecules. The complete BIA biosynthetic pathway uses tyrosine produced by the host cell and/or supplemented in the culture medium. Two molecules of tyrosine are modified and condensed to form the first benzylisoquinoline structure which may be either norcoclaurine or norlaudanosoline. The benzylisoquinoline is further modified to form (S)-reticuline and then stereochemically inverted by the activity of an epimerase enzyme to yield (R)-reticuline. (R)-reticuline undergoes a carbon-carbon coupling reaction to form the first promorphinan, salutaridine, and is further modified before undergoing an oxygen-carbon coupling reaction to arrive at the first morphinan alkaloid structure, thebaine (see FIG. 26). Table 2 lists enzymes and activities in the complete pathway.

To detect N-modifying activity in strains with the complete BIA pathway to nor-opioids (see FIG. 26), cells expressing candidate enzymes were propagated by fermentation (as described above) and incubated with SAM or SAM analogs, such as those listed in FIG. 25. Enzymatic modification of nor-opioid or other BIA molecules in strains harboring the complete BIA pathway was detected in supernatants by LC-MS (as described above). To detect N-modifying activity via biocatalysis, strains were cultured in selective medium and then lysed by glass bead disruption. Cell lysates were supplied exogenously with SAM or SAM analogs, and other cofactors necessary for enzyme function. Specifically, the conversion of noroxymorphone to naloxone and naltrexone (using the SAM analogs allyl-SAM or cyclopropane-SAM, as shown in FIG. 25) was detected. Modification of nor-opioid or other BIA molecules was detected by LC-MS. To detect N-modifying activity by biocatalysis in a strain that does not have the complete BIA pathway, Cen.PK2 strains expressing enzymes described in Example 16 were grown in selective medium and lysed by glass bead disruption. Cell lysates were supplied exogenously with SAM or SAM analogs, cofactors necessary for enzyme function, and nor-opioid molecules such as those listed in FIG. 25 and Table 2. Modification of these compounds was detected by LC-MS.

### Example 21: O-Demethylase Activity of CODM on Opioid Molecules

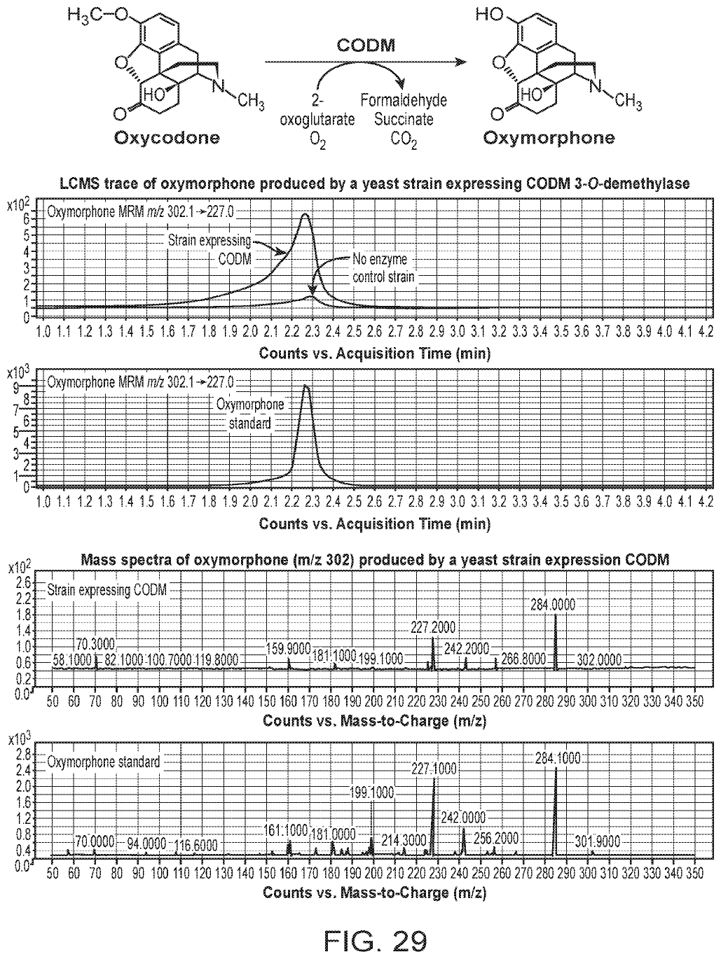

FIG. 29 illustrates the functional expression of CODM, in accordance with embodiments of the invention. In particular, FIG. 29 illustrates the functional expression of CODM with oxycodone 3-O-demethylase activity in yeast. The yeast codon-optimized CODM gene was integrated into the genome of yeast strain W303 and cultured in synthetic complete media for 16 hours. The parent W303 strain was also cultured in synthetic complete media for 16 hours as a no-enzyme control. The cells were pelleted and washed with 1 mL breaking buffer (100 mM Tris-HCl pH 7.5, 10% glycerol, 14 mM 2-mercaptoethanol, 1× protease inhibitor). Cells were resuspended in 200 μL breaking buffer and lysed by glass bead disruption. The crude cell lysates were incubated with 10 mM ascorbic acid, 0.5 mM iron (II) sulfate, 0.1 mM oxycodone as substrate and 10 mM 2-oxoglutarate as cosubstrate in a total volume of 100 μL. 4 mM DTT was also added as a reducing agent to keep iron in the Fe²⁺ state. The reaction was incubated at 30° C. for 3 h and quenched by diluting it 1:1 in ethanol with 0.1% acetic acid. The oxymorphone generated by the yeast strain expressing CODM was detected by LC-MS. The mass-charge ratio (m/z 302), retention time, and mass spectrum of oxymorphone produced by the yeast strain matched that of a purchased oxymorphone standard (see FIG. 29).

TABLE 2
Enzyme list

| | | | Source | Genbank |
|---|---|---|---|---|
| Enzyme | Abbrev | Catalyzed Reactions | organisms | # |
| 3-deoxy-d-arabinose- | ARO4, | erythrose-4-phosphate + | Saccharomyces | CAA85212.1 |
| heptulosonate-7- | DHAP | PEP → DHAP (EC 2.5.1.54) | cerevisiae | |
| phosphate synthase | synthase | | | |
| Chorismate mutase | ARO7 | chorismate → prephenate | Saccharomyces | NP_015385.1 |
| | | (EC 5.4.99.5) | cerevisiae | |
| Phenylpyruvate | ARO10 | hydroxyphenylpyruvate → | Saccharomyces | NP_010668.3 |
| decarboxylase | | 4HPA (EC 4.1.1.80) | cerevisiae | |
| Aromatic | ARO9 | hydroxyphenylpyruvate + | Saccharomyces | AEC14313.1 |
| aminotransferase | | glutamate → tyrosine + | cerevisiae | |
| | | alpha-ketogluterate (EC | | |
| | | 2.6.1.57) | | |
| Transketolase | TKL1 | fructose-6-phosphate + | Saccharomyces | NP_015399.1 |
| | | glyceraldehyde-3- | cerevisiae | |
| | | phosphate ↔ xylulose-5- | | |
| | | phosphate + erythrose-4- | | |
| | | phosphate (EC 2.2.1.1) | | |
| Glucose-6-phosphate | ZWF1 | glucose-6-phosphate → 6- | Saccharomyces | CAA96146.1 |
| dehydrogenase | | phosphogluconolactone | cerevisiae | |
| | | (EC 1.1.1.49) | | |
| Alcohol dehydrogenase | ADH2-7, | 4HPA → tyrosol (EC | Saccharomyces | NP_014032.1, |
| | SFA1 | 1.1.1.90) | cerevisiae | AAT93007.1, |
| | | | | NP_011258.2, |
| | | | | NP_009703.3, |
| | | | | NP_014051.3, |
| | | | | NP_010030.1, |
| | | | | NP_010113.1 |
| Aldehyde oxidase | ALD2-6 | 4HPA → | Saccharomyces | NP_013893.1, |
| | | hydroxyphenylacetic acid | cerevisiae | NP_013892.1, |
| | | (EC 1.2.1.39) | | NP_015019.1, |
| | | | | NP_010996.2, |
| | | | | NP_015264.1 |
| Tyrosinase | TYR | tyrosine → L-DOPA, L- | Ralstonia | NP_518458.1, |
| | | DOPA → dopaquinone (EC | solanacearum, | AJ223816, |
| | | 1.14.18.1) | Agaricus | |
| | | | bisporus | |
| Tyrosine hydroxylase | TyrH | tyrosine → L-DOPA (EC | Homo | NM012740, |
| | | 1.14.16.2) | sapiens, | NM000240, |
| | | | Rattus | |
| | | | norvegicus, | |
| | | | Mus | |
| | | | musculus | |
| GTP cyclohydrolase | FOL2 | GTP → dihydroneopterin | Saccharomyces | CAA97297.1, |
| | | triphosphate (EC 3.5.4.16) | cerevisiae, | NP_001019195.1, |
| | | | Homo | NP_032128.1 |
| | | | sapiens, | |
| | | | Mus | |
| | | | musculus | |
| 6-pyruvoyl | PTPS | dihydroneopterin | Rattus | AAH59140.1, |
| tetrahydrobiopterin | | triphosphate → PTP (EC | norvegicus, | BAA04224.1, |
| (PTP) synthase | | 4.2.3.12) | Homo | AAH29013.1 |
| | | | sapiens, | |
| | | | Mus | |
| | | | musculus | |
| Sepiapterin reductase | SepR | PTP → BH4 (EC 1.1.1.153) | Rattus | NP_062054.1, |
| | | | norvegicus, | NP_003115.1, |
| | | | Homo | NP_035597.2 |
| | | | sapiens, | |
| | | | Mus | |
| | | | musculus | |
| 4a- | PCD | 4a- | Rattus | NP_001007602.1, |
| hydroxytetrahydrobiopterin | | hydroxytetrahydrobiopterin → | norvegicus, | AAB25581.1, |
| (pterin-4α- | | H2O + quinoid | Homo | NP_079549.1 |
| carbinolamine) | | dihydropteridine (EC | sapiens, | |
| dehydratase | | 4.2.1.96) | Mus | |
| | | | musculus | |
| Quinoid | QDHPR | quinoid dihydropteridine → | Rattus | AAH72536.1, |
| dihydropteridine | | BH4 (EC 1.5.1.34) | norvegicus, | NP_000311.2, |
| reductase | | | Homo | AAH02107.1 |
| | | | sapiens, | |
| | | | Mus | |
| | | | musculus | |
| L-DOPA decarboxylase | DODC | L-DOPA → dopamine (EC | Pseudomonas | AE015451.1, |
| | | 4.1.1.28) | putida, | NP_001257782.1 |
| | | | Rattus | |
| | | | norvegicus | |
| Tyrosine/DOPA | TYDC | L-DOPA → dopamine (EC | Papaver | AAA97535.1, |
| decarboxylase | | 4.1.1.28) | somniferum | CAB56038.1 |
| Monoamine oxidase | MAO | dopamine → 3,4-DHPA (EC | E. coli, Homo | J03792, |
| | | 1.4.3.4) | sapiens, | D2367, |
| | | | Micrococcus | AB010716.1 |
| | | | luteus | |
| Dihydrofolate | DHFR | 7,8-Dihydrobiopterin → | Rattus | AF318150.1 |
| reductase | | 5,6,7,8- | norvegicus, | |
| | | Tetrahydrobiopterin (BH4) | Homo | |
| | | EC 1.5.1.3 | sapiens | |
| Norcoclaurine 6-O- | 6OMT | Norcoclaurine | P. | AY268894 |
| methyltransferase | | → coclaurine | somniferum | AY610507 |
| | | Norlaudanosoline | T. flavum | D29811 |
| | | → 3′hydroxycoclaurine | Coptis | |
| | | EC 2.1.1.128 | japonica* | |
| Coclaurine-N- | CNMT | Coclaurine → N- | P. | AY217336 |
| methyltransferase | | methylcoclaurine | somniferum | AY610508 |
| | | 3′hydroxycoclaurine | T. flavum | AB061863 |
| | | → 3′-hydroxy-N- | Coptis | |
| | | methylcoclaurine | japonica* | |
| | | EC 2.1.1.140 | | |
| 4′-O-methyltransferase | 4'OMT | 3′-hydroxy-N- | P. | AY217333, |
| | | methylcoclaurine | somniferum | AY217334 |
| | | → Reticuline EC 2.1.1.116 | T. flavum | AY610510 |
| | | | Coptis | D29812 |
| | | | japonica* | |
| Norcoclaurine synthase | NCS | 4HPA + dopamine → S- | Coptis | BAF45337-1, |
| | | norcoclaurine (EC 4.2.1.78) | japonica, | ACI45396.1, |
| | | 3,4-DHPA + dopamine → S- | Papaver | ACO90258.1, |
| | | norlaudanosoline | somniferum, | ACO90247.1, |
| | | | Papver | AEB71889.1 |
| | | | bracteatum, | |
| | | | Thalicitum | |
| | | | flavum, | |
| | | | Corydalis | |
| | | | saxicola | |
| Cytochrome P450 80B1 | CYP80B1 | N-methylcoclaurine → 3'- | P. | AAF61400.1, |
| | | hydroxy-N-methylcoclaurine | somniferum, | AAC39453.1, |
| | | | E. californica, | AA1120767.1 |
| | | | T. flavum | |
| Cheilanthifoline synthase | CFS | Scoulerine | P. | GU325749 |
| | | □ cheilanthifoline | somniferum | AB434654 |
| | | EC 1.14.21.2 | E. californica | EF451152 |
| | | | A. mexicana | |
| Stylopine synthase | STS | Cheilanthifoline | P. | GU325750 |
| | | □ stylopine | somniferum | AB126257 |
| | | EC 1.14.21.1 | E. californica | EF451151 |
| | | | A. mexicana | |
| Tetrahydroprotoberberine-N- | TNMT | Stylopine □ cis-N- | P. | DQ028579 |
| methyltransferase | | methylstylopine | somniferum | EU882977 |
| | | EC 2.1.1.122 | E. californica | EU882994 |
| | | | P. | HQ116698 |
| | | | bracteatum | |
| | | | A. mexicana | |
| Cis-N-methylstylopine 14- | MSH | cis-N-methylstylopine | P. | KC154003 |
| hydroxylase | | □ protopine | somniferum | |
| | | EC 1.14.13.37 | | |
| Protopine-6-hyd roxylase | P6H | Protopine → 6- | E. californica | AB598834 |
| | | hydroxyprotopine | P. | AGC92397 |
| | | EC 1.14.13.55 | somniferum | |
| Dihydrobenzophenanthridine | DBOX | Dihydrosanguinarine | P. | [not in |
| oxidase | | □ sanguinarine EC | somniferum | genbank] |
| | | 1.5.3.12 | | |
| (S)-tetrahydroprotoberberine | STOX | (S)-tetrahydroberberine + 2 | Berberis | HQ116697, |
| oxidase | | O₂ = berberine + 2H₂O₂ | wilsonae, | AB564543 |
| | | EC 1.3.3.8 | Coptis | |
| | | | japonica, | |
| | | | Berberis | |
| | | | spp, Coptis | |
| | | | spp | |
| S-adenosyl-L-methionine: | S9OMT | S-adenosyl-L-methionine + | Thalictrum | AY610512, |
| (S)-scoulerine 9-O- | | (S)-scoulerine = S-adenosyl- | flavum | D29809, |
| methyltransferase | | L-homocysteine + (S)- | subsp. | EU980450, |
| | | tetrahydrocolumbamine | glaucum, | JN185323 |
| | | EC 2.1.1.117 | Coptis | |
| | | | japonica, | |
| | | | Coptis | |
| | | | chinensis, | |
| | | | Papaver | |
| | | | somniferum, | |
| | | | Thalictrum | |
| | | | spp, Coptis | |
| | | | spp, | |
| | | | Papaver spp | |
| (S)- | CAS | (S)-tetrahydrocolumbamine + | Thalictrum | AY610513, |
| tetrahydrocolumbamine, | | NADPH + H+ + O2 = (S)- | flavum | AB026122, |
| NADPH:oxygen oxidoreductase | | canadine + NADP+ + 2H2O | subsp. | AB374407, |
| (methylenedioxy-bridge- | | EC 1.14.21.5 | glaucum, | AB374408 |
| forming), also known as (S)- | | | Coptis | |
| canadine synthase | | | japonica, | |
| | | | Thalictrum | |
| | | | spp, Coptis | |
| | | | spp | |
| (S)-reticuline:oxygen | BBE | (S)-reticuline + O2 = (S)- | Papaver | AF025430, |
| oxidoreductase (methylene- | | scoulerine + H2O2 | somniferum, | EU881889, |
| bridge-forming), also known | | EC 1.21.3.3 | Argemone | EU881890, |
| as berberine bridge enzyme | | | mexicana, | S65550 |
| | | | Eschscholzia | AF005655, |
| | | | californica, | AF049347, |
| | | | Berberis | AY610511, |
| | | | stolonifera, | AB747097 |
| | | | Thalictrum | |
| | | | flavum | |
| | | | subsp. | |
| | | | glaucum, | |
| | | | Coptis | |
| | | | japonica, | |
| | | | Papaver | |
| | | | spp, | |
| | | | Eschscholzia | |
| | | | spp, | |
| | | | Berberis | |
| | | | spp, | |
| | | | Thalictrum | |
| | | | spp, Coptis | |
| | | | spp | |
| NADPH:hemoprotein | ATR1, | NADPH + H+ + n oxidized | Arabidopsis | CAB58576.1, |
| oxidoreductase, also known | CPR | hemoprotein = NADP+ + | thaliana, | CAB58575.1, |
| as cytochrome P450 | | n reduced hemoprotein EC | Eschscholzia | AAC05021.1, |
| reductase | | 1.6.2.4 | californica, | AAC05022.1, |
| | | | Papaver | NM118585, |
| | | | somniferum, | many |
| | | | Homo | others |
| | | | sapiens, | (Ref |
| | | | Saccharomyces | PMID |
| | | | cerevisiae, | 19931102) |
| | | | Papaver | |
| | | | bracteatum, | |
| | | | Papaver | |
| | | | spp, all | |
| | | | plants | |
| salutaridinol:NADP+ 7- | SalR | salutaridinol + NADP+ = | Papaver | DQ316261, |
| oxidoreductase, also known | | salutaridine + NADPH + H+ | somniferum, | EF184229 |
| as salutaridine reductase | | EC 1.1.1.248 | Papaver | (Ref |
| | | | bracteatum, | PMID |
| | | | Papaver spp | 22424601) |
| | | | Chelidonium | |
| | | | majus | |
| acetyl-CoA:salutaridinol 7-O- | SalAT | acetyl-CoA + salutaridinol = | Papaver | AF339913, |
| acetyltransferase, also known | | CoA + 7-O- | somniferum, | FJ200355, |
| as salutaridinol 7-O- | | acetylsalutaridinol | Papaver | FJ200358, |
| acetyltransferase | | EC 2.3.1.150 | bracteatum, | FJ200356, |
| | | | Papaver | JQ659008 |
| | | | orientate, | |
| | | | Papaver spp | |
| (R)-reticuline, NADPH:oxygen | SalSyn | (R)-reticuline + NADPH + H+ + | Papaver | EF451150 |
| oxidoreductase (C-C phenol- | | O2 = salutaridine + NADP+ + | somniferum, | (Ref |
| coupling), also known as | | 2H2O | Papaver spp | PMID |
| salutaridine synthase | | EC 1.14.21.4 | Chelidonium | 22424601) |
| | | | majus | |
| 1-benzylisoquinoline alkaloid | CYP-COR | (S)-reticuline → (R)-reticuline | Papaver | P0DKI7.1, |
| epimerase (cytochrome P450 | or DRS- | (S)-1-benzylisoquinoline → | bracteatum, | AKO60175.1, |
| 82Y1-like codeinone | DRR | (R)-1-benzylisoquinoline | Papaver | AKO60180.1, |
| reductase-like) | | EC 1.5.1.27 | somniferum, | AKO60179.1, |
| | | | Papaver | AKO60175.1 |
| | | | setigerum, | |
| | | | Chelidonium | |
| | | | majus | |
| Cytochrome P450, family 2, | CYP2D6 | Promiscuous oxidase, can | Homo | BC067432 |
| subfamily D, polypeptide 6 | | perform | sapiens | |
| | | (R)-reticuline + NADPH + H+ + | | |
| | | O2 = salutaridine + NADP+ + | | |
| | | 2H2O among other | | |
| | | reactions | | |
| | | EC 1.14.14.1 | | |
| Thebaine 6-O demethylase | T6ODM | thebaine → □ neopinone EC | Papaver | GQ500139.1 |
| | | 1.14.11.31 | somniferium, | |
| | | | Papaver | |
| | | | spp. | |
| Codeinone reductase | COR | codeinone → □ codeine EC | Papaver | AF108432.1 |
| | | 1.1.1.247, | somniferium, | AF108433.1 |
| | | neopinone → □ neopine | Papaver | AF108434.1 |
| | | | spp. | AF108435.1 |
| Codeine O-demethylase | CODM | codeine → □ morphine EC | Papaver | GQ500141.1 |
| | | 1.14.11.32, | somniferium, | |
| | | neopine → □ neomorphine | Papaver | |
| | | | spp. | |
| Morphine dehydrogenase | morA | morphine → □ morphinone EC | Pseudomonas | M94775.1 |
| | | 1.1.1.218, | putida | |
| | | codeinone → □ codeine EC | | |
| | | 1.1.1.247 | | |
| Morphinone reductase | morB | codeinone → □ hydrocodone | Pseudomonas | U37350.1 |
| | | morphinone | putida | |
| | | □ □ hydromorphone EC 1.3.1.- | | |
| Reticuline N- | RNMT | reticuline→tembetarine | Papaver | KX369612.1 |
| methyltransferase | | | somniferum, | |
| | | | Papaver | |
| | | | spp. | |
| Papaverine 7-O-demethylase | P7OMT | papaverine→pacodine | Papaver | KT159979.1 |
| | | | somniferum, | |
| | | | Papaver | |
| | | | spp. | |
| 3-O-demethylase | 3ODM | oxycodone→oxymorphone | Papaver | |
| | | hydrocodone→hydromorphone | somniferum, | |
| | | dihydrocodeine→ | Papaver | |
| | | dihydromorphine | bracteatum, | |
| | | 14-hydroxycodeine→14- | Papaver | |
| | | hydroxymorphine | rhoeas, | |
| | | codeinone→morphinone | Papaver | |
| | | 14-hydroxycodeinone→14- | spp. | |
| | | hydroxymorphinone | | |
| N-demethylase | NDM | Codeine→Norcodeine | Bacillus | |
| | | Morphine→Normorphine | megaterium, | |
| | | Oxycodone→Noroxycodone | Homo | |
| | | Oxymorphone→ | sapiens, | |
| | | Noroxymorphone | Papaver | |
| | | Thebaine→Northebaine | somniferum, | |
| | | Oripavine→Nororipavine | Papaver | |
| | | Hydrocodone→ | spp., | |
| | | Norhydrocodone | Chelidonium | |
| | | Hydromorphone→ | majus, | |
| | | Norhydromorphone | Stylophorum | |
| | | Dihydrocodeine→ | diphyllum, | |
| | | Nordihydrocodeine | Nigella | |
| | | Dihydromorphine→ | sativa, | |
| | | Nordihydromorphine | Hydrastis | |
| | | 14-hydroxycodeine→Nor-14- | canadensis, | |
| | | hydroxycodeine | Glaucium | |
| | | 14-hydroxymorphine→Nor- | flavum, | |
| | | 14-hydroxymorphine | Eschscholzia | |
| | | Codeinone→Norcodeinone | californica, | |
| | | Morphinone→Normorphinone | Menispermum | |
| | | 14-hydroxycodeinone→Nor- | canadense, | |
| | | 14-hydroxycodeinone | Papaver | |
| | | 14- | bracteatum | |
| | | hydroxymorphinone→Nor- | | |
| | | 14-hydroxymorphinone | | |
| N-methyltransferase | NMT | Norcodeine→ codeine | Papaver | |
| | | Normorphine→morphine | spp., | |
| | | Noroxycodone→oxycodone | Chelidonium | |
| | | Noroxymorphone→ | majus, | |
| | | noroxymorphone | Thalictrum | |
| | | Northebaine→thebaine | flavum, | |
| | | Nororipavine→oripavine | Coptis | |
| | | Norhydrocodone→hydrocodone | japonica, | |
| | | Norhydromorphone→ | Papaver | |
| | | Hydromorphone | somniferum, | |
| | | Nordihyd rocodeine→ | Eschscholzia | |
| | | Dihydrocodeine | californica, | |
| | | Nordihyd romorphine→ | Papaver | |
| | | Dihydromorphine | bracteatum, | |
| | | Nor-14-hydroxycodeine→ | Argenome | |
| | | 14-hydroxycodeine | mexicana, | |
| | | Nor-14-hydroxymorphine→ | Glaucium | |
| | | 14-hydroxymorphine | flavum, | |
| | | Norcodeineone→ | Sanguinaria | |
| | | Codeineone | canadensis, | |
| | | Normorphinone→ | Corydalis | |
| | | Morphinone | chelanthifolia, | |
| | | Nor-14-hydroxy-codeinone→ | Nigella | |
| | | 14-hydroxycodeinone | sativa, | |
| | | Nor-14-hydroxy- | Jeffersonia | |
| | | morphinone→ 14- | diphylla, | |
| | | hydroxymorphinone | Berberis | |
| | | | thunbergii, | |
| | | | Mahonia | |
| | | | aguifolium, | |
| | | | Menispermum | |
| | | | canadense, | |
| | | | Tinospora | |
| | | | cordifolia, | |
| | | | Cissampelos | |
| | | | mucronata, | |
| | | | Cocculus | |
| | | | trilobus | |
| N-allyltransferase | NAT | Norcodeine→N-allyl- | Papaver | |
| | | norcodeine | spp., | |
| | | Normorphine→N-allyl- | Chelidonium | |
| | | normorphine | majus, | |
| | | Noroxycodone→N-allyl- | Thalictrum | |
| | | noroxycodone | flavum, | |
| | | Noroxymorphone→N-allyl- | Coptis | |
| | | nornoroxymorphone | japonica, | |
| | | Northebaine→N-allyl- | Papaver | |
| | | northebaine | somniferum, | |
| | | Nororipavine→N-allyl- | Eschscholzia | |
| | | nororipavine | californica, | |
| | | Norhydrocodone→N-allyl- | Papaver | |
| | | norhydrocodone | bracteatum, | |
| | | Norhydromorphone→ N- | Argenome | |
| | | allyl-norhydromorphone | mexicana, | |
| | | Nordihydrocodeine→ N-allyl- | Glaucium | |
| | | nordihydrocodeine | flavum, | |
| | | Nordihydromorphine→ N- | Sanguinaria | |
| | | allyl-nordihydromorphine | canadensis, | |
| | | Nor-14-hydroxycodeine→ N- | Corydalis | |
| | | allyl-nor-14-hydroxycodeine | chelanthifolia, | |
| | | Nor-14-hydroxymorphine→ | Nigella | |
| | | N-allyl-nor-14- | sativa, | |
| | | hydroxymorphine | Jeffersonia | |
| | | Norcodeineone→ N-allyl- | diphylla, | |
| | | norcodeineone | Berberis | |
| | | Normorphinone→ N-allyl- | thunbergii, | |
| | | normorphinone | Mahonia | |
| | | Nor-14-hydroxy-codeinone→ | aguifolium, | |
| | | N-allyl-nor-14- | Menispermum | |
| | | hydroxycodeinone | canadense, | |
| | | Nor-14-hydroxy- | Tinospora | |
| | | morphinone→ N-allyl-nor- | cordifolia, | |
| | | 14-hydroxymorphinone | Cissampelos | |
| | | | mucronata, | |
| | | | Cocculus | |
| | | | trilobus | |
| N- | NCPMT | Norcodeine→N- | Papaver | |
| cyclopropylmethyltransferase | | (Cyclopropylmethyl)norcodeine | spp., | |
| | | Normorphine→N- | Chelidonium | |
| | | (Cyclopropylmethyl)normorphine | majus, | |
| | | Noroxycodone→N- | Thalictrum | |
| | | (Cyclopropylmethyl)noroxycodone | flavum, | |
| | | Noroxymorphone→N- | Coptis | |
| | | (Cyclopropylmethyl)nornoroxymorphone | japonica, | |
| | | Northebaine→N- | Papaver | |
| | | (Cyclopropylmethyl)northebaine | somniferum, | |
| | | Nororipavine→N- | Eschscholzia | |
| | | (Cyclopropylmethyl)nororipavine | californica, | |
| | | Norhydrocodone→N- | Papaver | |
| | | (Cyclopropylmethyl)norhydrocodone | bracteatum, | |
| | | Nordihydrocodeine→ N- | Argenome | |
| | | (Cyclopropylmethyl)nordihydrocodeine | mexicana, | |
| | | Nordihydromorphine→ N- | Glaucium | |
| | | (Cyclopropylmethyl)nordihydromorphine | flavum, | |
| | | Nor-14-hydroxycodeine→ N- | Sanguinaria | |
| | | (Cyclopropylmethyl)nor-14- | canadensis, | |
| | | hydroxycodeine | Corydalis | |
| | | Nor-14-hydroxymorphine→ | chelanthifolia, | |
| | | N-(Cyclopropylmethyl)nor- | Nigella | |
| | | 14-hydroxymorphine | sativa, | |
| | | Norcodeineone→ N- | Jeffersonia | |
| | | (Cyclopropylmethyl)norcodeineone | diphylla, | |
| | | Normorphinone→ N- | Berberis | |
| | | (Cyclopropylmethyl)normorphinone | thunbergii, | |
| | | Nor-14-hydroxy-codeinone→ | Mahonia | |
| | | N-(Cyclopropylmethyl)nor- | aguifolium, | |
| | | 14-hydroxycodeinone | Menispermum | |
| | | Nor-14-hydroxy- | canadense, | |
| | | morphinone→ N- | Tinospora | |
| | | (Cyclopropylmethyl)nor-14- | cordifolia, | |
| | | hydroxymorphinone | Cissampelos | |
| | | | mucronata, | |
| | | | Cocculus | |
| | | | trilobus | |

TABLE 3
O-demethylase candidate enzymes

| | | SEQ ID |
|---|---|---|
| Name | Sequence | NO: |
| T6ODM | MEKAKLMKLGNGMEIPSVQELAKLTLAEIPSRYVCANENLLLPMGA | 16 |
| | SVINDHETIPVIDIENLLSPEPIIGKLELDRLHFACKEWGFFQVVNHGV | |
| | DASLVDSVKSEIQGFFNLSMDEKTKYEQEDGDVEGFGQGFIESEDQT | |
| | LDWADIFMMFTLPLHLRKPHLFSKLPVPLRETIESYSSEMKKLSMVL | |
| | FNKMEKALQVQAAEIKGMSEVFIDGTQAMRMNYYPPCPQPNLAIGL | |
| | TSHSDFGGLTILLQINEVEGLQIKREGTWISVKPLPNAFVVNVGDILEI | |
| | MTNGIYHSVDHRAVVNSTNERLSIATFHDPSLESVIGPISSLITPETPAL | |
| | FKSGSTYGDLVEECKTRKLDGKSFLDSMRI | |
| CODM | METPILIKLGNGLSIPSVQELAKLTLAEIPSRYTCTGESPLNNIGASVT | 17 |
| | DDETVPVIDLQNLLSPEPVVGKLELDKLHSACKEWGFFQLVNHGVD | |
| | ALLMDNIKSEIKGFFNLPMNEKTKYGQQDGDFEGFGQPYIESEDQRL | |
| | DWTEVFSMLSLPLHLRKPHLFPELPLPFRETLESYLSKMKKLSTVVFE | |
| | MLEKSLQLVEIKGMTDLFEDGLQTMRMNYYPPCPRPELVLGLTSHS | |
| | DFSGLTILLQLNEVEGLQIRKEERWISIKPLPDAFIVNVGDILEIMTNGI | |
| | YRSVEHRAVVNSTKERLSIATFHDSKLESEIGPISSLVTPETPALFKRG | |
| | RYEDILKENLSRKLDGKSFLDYMRM | |
| PsP7ODM | MEKAKLMKLGNGLSIPSVQELAELTFAEVPSRYVCTNDENLLLMTM | 18 |
| | GASEIDDETVPVIDLQNLLSPEPAIGKSELDWLHYSCKEWGFFQLVN | |
| | HGVDALLVDHVKSEIHSFFNLPLNEKTKYGQRDGDVEGFGQAFLVS | |
| | ENQKLDWADMFFINTLPLHLRKPHLFPNLPLPLRETIESYSSEMKKLS | |
| | MVLFEMMGKAIEVIDIKEAITEMFEDGMQSMRMNYYPPCPQPERVI | |
| | GITPHSDFDGLTILLQLNEVEGLQIRKEDKWISIKPLPDAFIVNVGDIW | |
| | EIMTNGVHRSVDHRGVINSTKERLSIATFHSPKLELEIGPISSLIRPETP | |
| | AVFKSAGRFEDLLKEGLSRKLDGKSFLDCMRM | |
| PsoDIOX1 | MEKAKLMKLGNGMEIPSVQELAKLTLAEIPSRYVCANENLLLPMGA | 19 |
| | SVINDHETIPVIDIENLLSPEPIIGKLELDRLHFACKEWGFFQVVNHGV | |
| | DASLVDSVKSEIQGFFNLSMDEKTKYEQEDGDVEGFGQGFIESEDQT | |
| | LDWADIFMMFTLPLHLRKPHLFSKLPVPLRETIESYSSEMKKLSMVL | |
| | FNKMEKALQVQAAEIKGMSEVFIDGTQAMRMNYYPPCPQPNLAIGL | |
| | TSHSDFGGLTILLQINEVEGLQIKREGTWISVKPLPNAFVVNVGDILEI | |
| | MTNGIYHSVD | |
| PsoDIOX2 | METAKLMKLGNGMSIPSVQELAKLTLAEIPSRYICTVENLQLPVGAS | 20 |
| | VIDDHETVPVIDIENLISSEPVTEKLELDRLHSACKEWGFFQVVNHGV | |
| | DTSLVDNVKSDIQGFFNLSMNEKIKYGQKDGDVEGFGQAFVASEDQ | |
| | TLDWADIFMILTLPLHLRKPHLFSKLPLPLRETIESYSSEMKKLSMVL | |
| | FEKMEKALQVQAVEIKEISEVFKDMTQVMRMNYYPPCPQPELAIGL | |
| | TPHSDFGGLTILLQLNEVEGLQIKNEGRWISVKPLPNAFVVNVGDVL | |
| | EIMTNGMYRSVDHRAVVNSTKERLSIATFHDPNLESEIGPISSLITPNT | |
| | PALFRSGSTYGELVEEFHSRKLDGKSFLDSMRM | |
| PbrDIOX2 | METPKSIKLGGSLLVPSVQELAQQSFAEVPARYVRDDLEPLTDLSGV | 21 |
| | SMIDQTIPVIDLQKLQSPVPIIRELESEKLHSACKEWGFFQVVNHGVDI | |
| | LLVEKTKSEIKDFFNLPMDEKKKFWQEEGDIQGFGQAFVQSEDQKL | |
| | DWADIFLMVTLPRHTRNPRLFPKLPLPLRNTMDSYSSKLSKLASTLIE | |
| | MMGKALHMETSVLAELFEDGRQTMRINYYPPCPQPKDVIGLTPHSD | |
| | GGGLTILLQLNEVDGLQIRKEKIWIPIKPLPNAFVVNIGNILEIMTNGI | |
| | YRSVEHRATIHSTKERLSVAAFHNPKVGVEIGPIVSMITPESPALFRTI | |
| | EYDDYGKKYFSRKLDGKSSLDFMRIGEGDEENKAT | |
| PbrDIOX3 | METPKLIKLGGSLLVPSVLELTKQSPAEVPARYIRNDLEPMTDLSSAS | 22 |
| | LTDQTIPVIDLQNLLSPEPELELEKLHSGCKEWGFFQVMNHGVDILLV | |
| | EKVKSEIQGFFNLPIDEKNKFWQEEGDLEGYGKAFVHSEDEKLDWA | |
| | DMFFILTQPQYMRKPRVFPKLPLRLRETIESYSLELSKLGLTLLDLMG | |
| | KALQIETGVMSELFEDGRQTMRMNYYPPCPQPEHVIGLTPHSDGGA | |
| | LTILLQLNQVDGLQIRKEEIWVPIKPLPNAFVVNIGDILEIMSNGVYRS | |
| | VEHRATINSSKERLSVAIFQSPKHGTEIGPILSMITPEAPALFKTIPYED | |
| | YLRKFFSRKLGGKSFVDSMRIGESDEDNNTA | |
| PbrDIOX4 | METQKQENFGASLSVPNVQELAKQSPEQVPDRYIRSDQDSSTNISCPS | 23 |
| | MTDQIPVIDLQSLLSPDPIIGELELERLHSACKEWGFFQVVNHGVDNL | |
| | LVEKVKSEIQGFFNLPMDEKKKFWQEEGDFEGFGQAFVFSEDQKLD | |
| | WGDVFFILTQPQHMRKPRLFPKLPLPFRKTIESYSLETNKLSMTLLEL | |
| | MEKALKIETGVMTELFEGGIQRMRMTYYPPCPQPKHVIGLTPHSDPD | |
| | ALTILLQLNEVDGLQIRKEKIWVPIKPLSNAFVVNIGDILEIMSNGIYR | |
| | SVEHRATVNSTKERLSVATFHSPRKDTEIGPILITPETPALFRTSGFED | |
| | YFRKFFAHKLNGKSFLSSIRIGETDEGNNAT | |
| PbrDIOX5 | MEAPKLIMLGGSLFVPSVQELAKQSLAEVPVRYVRDDQDTLGNNINI | 24 |
| | TPMSMIDQSIPVIDLEKLLSPEPIVGELELERLHSACKEWGFFQVVNH | |
| | GVDSLLVEKVKSEIEGFFKLPMDEKTKFWQEEGDIEGFGQVFVHSQD | |
| | QKLDWGDMFLMQTLPRHTRKPRLFPNLPLPLRQTIESYSSELSKLVL | |
| | TLVDLMGKALQMESGVLTELFENGIQRMRMNYYPPCPQPEQVIGLT | |
| | PHSDVGGLTILLQLNEVDGLQIKKDKVWVPIKPLANAFVVNVGDAL | |
| | EIMSNGIYRSVEHRATINSTKERLSIATFHNPRADREIGPIPSMISPETP | |
| | ALFKTTGYEEYFKKFFSRKLEGKSFLDSLRIREGDEHCGRLDVKGPC | |
| | N | |
| PbrDIOX6 | MEIPNPIKIGSSLLVPSVQELAKQSFAEVPARYIRNDVDPLITKLSDVS | 25 |
| | LIDQTVPVIDLQKLLSPEPIVGELELERLHSACKEWGFFQVVNHGVD | |
| | NLLVEKVKSEIQGFFNLPMEEKKKFWQEEGDFEGFGQMFVQSEEQK | |
| | LDWGDMFFILTQPQHMRKPRLFSKLPLPLRETIESYSLELIKLGLTIIK | |
| | LMEKALQIDAGVMAELFEDGIHTMRMNYYPPCPQPEHVIGLTPHSD | |
| | GGGLTILLQLNEVDGLQIRRENIWVPIKPLPNAFVVNIGDILEILSNGI | |
| | YRSVEHRSTVNATKERLSVATFQNPKQESVIGPNMITPERPALFRKIV | |
| | YKDYMKKLFSRKLDGKSFLDSLRIGEGDERP | |
| PbrDIOX8 | METLKTVKPGGSLFIPNGQELAKQSLEEVYVGNDQDTMLLIGQTIPVI | 26 |
| | DLQKLLSPEPITGDMELDKLHSACKEWGFFQVVNHGVDILLVEKVK | |
| | SEVHDFFNIPMDEKKPFWQEEGDLEGFGQVFITSEDQQLDWGDMFF | |
| | MVTLPKHMRKPRLFLKLPLPLRETIESYSLKLSKLGVTLVELMGKAL | |
| | QMEDRIMSELFDDGRQTMRMNYYPPCPQPEQVIGLTPHSDPGGLTIL | |
| | LELNEVNGLIRKENIWVPIIPLPNAFIVNIGDILEIMSNGIYHSVEHRAT | |
| | INSTKERLSVAMFNSPKVDTEIGPIHSMITPETPALFRTIGYDEYLKIFF | |
| | SRKLDGKSLLESMKI | |
| PbrDIOX10 | MEAPKLIMLGGSLFVPSVQELAKQSLAEVPVRYVRDDQDTLGNNINI | 27 |
| | TPMSMIDQSIPVIDLEKLLSPEPIVGELELERLHSACKEWGFFQVVNH | |
| | GVDSLLVEKVKSEIEGFFELPVDEKKKFWQEEGDIEGFGQIFVHSEDQ | |
| | KLDWADMFYMLTLPPNMRKPRLFPNLPLPLRQTIDSYSSELSKLVLT | |
| | LVDLMGKALQMESGVLTELFENGIQRMRMNYYPPCPQPEQVIGLTP | |
| | HSDVGGLTILLQLNEVDGLQIKKDKIWVPIKPLRNAFVVNVGDALEI | |
| | MSNGIYRSVEHRATINSTKERLSIATFHNPRADREIGPIPSMISPETPAL | |
| | FKTTGYEEYFKKFFSRKLEGKSFLDSLRIGEGDEHCGRLXVKGXCN | |
| PbrDIOX11 | METPKLMKLGGSLFVPSVQELAKQSLAEVPARYVRDDRDMVGNIIN | 28 |
| | VTPMSMIDQSIPVIDLEKLLSPDLIVGELELERLHSACKEWGFFQVVN | |
| | HGVDSLLVEKVKSEIEGFFELPMDEKKKFWQEEGDAEGFAQFFVQS | |
| | EDQKLDYSGDMFFMLNLPQHMRKPRLFLKLPLPLRETIESYSLKLSK | |
| | LGVTLVELMGKALQMEDRIMSELFDDGRQTMRMNYYPPCPQPEQVI | |
| | GLTPHSDPGGLTILLELNEVNGLIRKENIWVPIIPLPNAFIVNIGDILEI | |
| | MSNGIYHSVEHRATINSTKERLSVAMFNSPKVDTEIGPIHSMITPETPA | |
| | LFRTIGYDEYLKIFFSRKLDGKSLLESMKI | |
| PbrDIOX13 | METPKLRDFGSFLPVPSVQELAKQVLTEIPPRYIRTDLEALNKLSCAS | 29 |
| | NTDQTVPIIDMQCLLSAEPEMELEKLHSACKEWGFFRVVNHGVDNL | |
| | ESVKSEIESFLNLPVNAKNKYGQKQGDDQGFGSRFVLSEEQKLDWG | |
| | DFFYMVTRPLYLRKPHLFPELPLPLRETIESYSSEVSKLAMALFEMM | |
| | GKALKIETGVMTEIFEGGMQAMRMNYYPPCPRPDLVIGLNAHSDFG | |
| | GLTILLQLNEVEGLEIRNKGEWVSVKPLANAFVVNVGDVMEILTNGI | |
| | YHSVEHRATINSSKERLSVATFHYPKLETGIGPLPCMITPKTPALFGRI | |
| | ERYELLLRKYYARKLNGKSTLDCMRIGNGFEDDNTA | |
| PbrDIOX18 | MEAPKLIMLGGSLFVPSVQELAKQSLAEVPARYVRDDQDTLGNNINI | 30 |
| | TPMSMIDQSIPVIDLEKLLSPEPIVGELELERLHSACKEWGFFQVVNH | |
| | GVDSLLVEKVKSEIEGFFELPVDEKKKFWQEEGDIEGFGQIFVHSEDQ | |
| | KLDWADMFYMLTLPPNMRKPRLFPNLPLPLRQTIDSYSSELSKLVLT | |
| | LVDLMGKALQMESGVLTELFENGIQRMRMNYYPPCPQPEQVIGLTP | |
| | HSEVGGLTILLQLNEVDGLQIRKEKIWVPIKPLSNAFIVNIGDILEIMS | |
| | NGIYRSVEHRATVNSTKERLSVATFHSPRKDTEIGPILITPETPALFRTS | |
| | GFEDYFRKFFAHKLNGKSFLSSIRIGETDEGNNAT | |
| PbrDIOX19 | MSMIDQSIPVIDLEKLLSPEPIVGELELERLHSACKEWGFFQVVNHGV | 31 |
| | DSLLVEKVKSEIEGFFELPVDEKKKFWQEEGDIEGFGQIFVHSEDQKL | |
| | DWADMFYMLTLPPNMRKPRLFPNLPLPLRQTIDSYSSELSKLVLTLV | |
| | DLMGKALQMESGVLTELFENGIQRMRMNYYPPCPQPEQVIGLTPHS | |
| | DVGGLTILLQLNEVDGLQIRKEKIWVPIKPLSNAFIVNIGDILEIMSNGI | |
| | YHSVEHRATINSTKERLSVAMFNSPKVDTEIGPIHSMITPETPALFRTI | |
| | GYDEYLKIFFSRKLDGKSLLESMKI | |
| PbrDIOX21 | METPKLVKSSGSSLFLSTSVQELAKQSLPEVPARYIRTNLEPLSNVSG | 32 |
| | DSQSVPVIDLQKLLSSEPIIGELELDKLHSACKEWGFFQVVNHGVDN | |
| | LVMEKIKTEIQGFFNLSLDEKQKFWKKEGDAEGFGQNFIESEDQKLD | |
| | WGDTFGMFTLPIHMRNPRLFPELPLPLRETIESYSLDVRKLALALIGL | |
| | MEKALKIKTSAMSELFEDGGQAMRMNYYPPCPQPEHVIGLTPHSDA | |
| | GGLTILLQLNEVDGLQIKKDKIWVPIKPLPNAFVVNIGDILEIMTNGIY | |
| | RSVEHRATINSSKERLSVAAFHSPKGDTLIGPMVSLITPETPALFRTIG | |
| | YQDYMKKFMSRKLDGKSLVNSMRIGEGDEDK | |
| PbrDIOX- | METPTLMKLGNGLSVPSVQELAKATLAEIPSRYICTDENLLTMGAST | 33 |
| ZSNV- | TDNETVPVIDLQNLLSPEPVIGMLELDRLHSACKEWGFFQLVNHGVD | |
| 2004018 | ALLVDNEVQGFFNLPMDEKTKYGQKDGDDEGFGQFFVISEDQKLD | |
| | WADVFYMSTLPLHSRKPHLFPELPLPLRETMESYSSEMKKLSMVLFD | |
| | MMGKALQVVEIKGITELFEDGAQQIRMNYYPPCPQPELVFGLTSHSD | |
| | FDGLTILLQLGEVEGLQIKKEERWISIKPLPDAFIVNVGDILEIMTNGI | |
| | YRSVDHRAVVNSIKERLTIATFHDPRLEAEIGPISSLITPETPALFKRGV | |
| | FEDLLKEMFLRKLDGKSFLDCMRM | |
| PrhDIOX- | GNGLSVPSVQELAKQTLAEIPSRYICTDENPLITGASVVDDETVPVIN | 34 |
| MVTX- | LQNLLSPEPVIGKLELDKLHSACKEWGFFQVVNHGVNDSLVDSVKS | |
| 2001522 | EIEGFFNLPANEKLKYGQKDGDVEGFGQHFVVSEDQKLDWADVFY | |
| | MVTLPVRLRKPHLFPELPLPLRDTLDSYSSELNKLSMVLLEMMEKAL | |
| | KLVECKGITDFFEDGFQQMRMNYYPPCPRPELVTGLTSHSDFGGLTI | |
| | LLQLNDVEGLQIKKEERWISIKPLPNAFIVNIGDVLEIMSNGIYRSVDH | |
| | RAVINSTKVRMSVATFHDPRLEAVIGPISSLITPETPALFKRGVFEDLL | |
| | KEMFLRKLDGKSFLDCMRI | |
| PseDIOX- | LMKLANGMSVPIVQELAKLTVGEIPSRYICTDGNLLTMGASVIDYET | 35 |
| JSVC- | VPVIDLQNLQSREPVIEKLELDRLHSACKEWGFFQLLNHGVDASLMD | |
| 2005842 | NVRSEIRGFFNLPISDKMKYGQKDGDEEGFGQHFIVSEDQKLDWVD | |
| | AFMMFTLPLHSRNPRLTPEFPQPLRETVESYSSEMKKLSVLLFELME | |
| | KALQVKGITEMFEDGLQSIRMNYYPPCPRPELAIGLTSHSDFDGLTIL | |
| | LQLNEVEGLQIKKEERWISIKPLPNAFIVNVGDVLEVMTNGIYRSVD | |
| | HRAVVNSTKERLSIATFHDPELESEIGPIASLITPETPALFKRGRFKDLL | |
| | KENLSTKLDGKSFLDCIRM | |
| CYP2D6 | MGLEALVPLAVIVAIFLLLVDLMHRRQRWAARYSPGPLPLPGLGNLL | 36 |
| | HVDFQNTPYCFDQLRRRFGDVFSLQLAWTPVVVLNGLAAVREALVT | |
| | HGEDTADRPPVPITQILGFGPRSQGVFLARYGPAWREQRRFSVSTLR | |
| | NLGLGKKSLEQWVTEEAACLCAAFANHSGRPFRPNGLLDKAVSNVI | |
| | ASLTCGRRFEYDDPRFLRLLDLAQEGLKEESGFLREVLNAVPVLLHIP | |
| | ALAGKVLRFQKAFLTQLDELLTEHRMTWDPAQPPRDLTEAFLAEME | |
| | KAKGNPESSFNDENLRIVVADLFSAGMVTTSTTLAWGLLLMILHPDV | |
| | QRRVQQEIDDVIGQVRRPEMGDQAHMPYTTAVIHEVQRFGDIVPLG | |
| | VTHMTSRDIEVQGFRIPKGTTLITNLSSVLKDEAVWEKPFRFHPEHFL | |
| | DAQGHFVKPEAFLPFSAGRRACLGEPLARMELFLFFTSLLQHFSFSVP | |
| | TGQPRPSHHGVFAFLVTPSPYELCAVPR | |

TABLE 4
N-demethylase candidate enzymes

| | | SEQ ID |
|---|---|---|
| Name | Sequence | NO: |
| BM3 | MTIKEMPQPKTFGELKNLPLLNTDKPVQALMKIADELGEIFKFEAPG | 37 |
| | RVTRYLSSQRLIKEACDESRFDKNLSQAAKFARDFAGDGLVTSWTH | |
| | EKNWKKAHNILLPSFSQQAMKGYHAMMVDIAVQLVQKWERLNA | |
| | DEHIEVSEDMTRLTLDTIGLCGFNYRFNSFYRDQPHPFIISMVRAAD | |
| | EVMNKLQRANPDDPAYDENKRQFQEDIKVMNDLVDKIIADRKARG | |
| | EQSDDLLTQMLNGKDPETGEPLDDGNIRYQIITFLIAGHETTSGLLSF | |
| | ALYFLVKNPHVLQKVAEEAARVLVDPVPSYKQVKQLKYVGMVLN | |
| | EALRLWPTAPAFSLYAKEDTVLGGEYPLEKGDEVMVLIPQLHRDKT | |
| | VWGDDVEEFRPERFENPSAIPQHAFKPFGNGQRACIGQQFALHEAT | |
| | LVLGMMLKHFDFEDHTNYELDIKETLTLKPKGFVVKAKSKKIPLGG | |
| | IPSPSTEQSAKKVRKKAENAHNTPLLVLYGSNMGTAEGTARDLADI | |
| | AMSKGFAPQVATLDSHAGNLPREGAVLIVTASYNGHPPDNAKQFV | |
| | DWLDQASADEVKGVRYSVFGCGDKNWATTYQKVPAFIDETLAAK | |
| | GAENIADRGEADASDDFEGTYEEWREHMWSDVAAYFNLDIENSED | |
| | NKSTLSLQFVDSAADMPLAKMHGAFSTNVVASKELQQPGSARSTR | |
| | HLEIELPKEASYQEGDHLGVIPRNYEGIVNRVTARFGLDASQQIRLE | |
| | AEEEKLAHLPLAKTVSVEELLQYVELQDPVTRTQLRAMAAKTVCPP | |
| | HKVELEALLEKQAYKEQVLAKRLTMLELLEKYPACEMKFSEFIALL | |
| | PSIRPRYYSISSSPRVDEKQASITVSVVSGEAWSGYGEYKGIASNYLA | |
| | ELQEGDTITCFISTPQSEFTLPKDPETPLIMVGPGTGVAPFRGFVQAR | |
| | KQLKEQGQSLGEAHLYFGCRSPHEDYLYQEELENAQSEGHTLHTAF | |
| | SRMPNQPKTYVQHVMEQDGKKLIELLDQGAHFYICGDGSQMAPAV | |
| | EATLMKSYADVHQVSEADARLWLQQLEEKGRYAKDVWAG | |
| CYP3A4-1 | MALIPDLAMETWLLLAVSLVLLYLYGTHSHGLFKKLGIPGPTPLPFL | 38 |
| | GNILSYHKGFCMFDMECHKKYGKVWGFYDGQQPVLAITDPDMIKT | |
| | VLVKECYSVFTNRRPFGPVGFMKSAISIAEDEEWKRLRSLLSPTFTS | |
| | GKLKEMVPIIAQYGDVLVRNLRREAETGKPVTLKDVFGAYSMDVIT | |
| | STSFGVNIDSLNNPQDPFVENTKKLLRFDFLDPFFLSITVFPFLIPILEV | |
| | LNICVFPREVTNFLRKSVKRMKESRLEDTQKHRVDFLQLMIDSQNS | |
| | KETESHKALSDLELVAQSIIFIFAGYETTSSVLSFIMYELATHPDVQQ | |
| | KLQEEIDAVLPNKAPPTYDTVLQMEYLDMVVNETLRLFPIAMRLER | |
| | VCKKDVEINGMFIPKGVVVMIPSYALHRDPKYWTEPEKFLPERFSK | |
| | KNKDNIDPYIYTPFGSGPRNCIGMRFALMNMKLALIRVLQNFSFKPC | |
| | KETQIPLKLSLGGLLQPEKPVVLKVESRDGTVSGA | |
| CYP3A4-2 | MALIPDLAMETWLLLAVSLVLLYLYGTHSHGLFKKLGIPGPTPLPFL | 39 |
| | GNILSYHKGFCMFDMECHKKYGKVWGFYDGQQPVLAITDPDMIKT | |
| | VLVKECYSVFTNRRPFGPVGFMKSAISIAEDEEWKRLRSLLSPTFTS | |
| | GKLKEMVPIIAQYGDVLVRNLRREAETGKPVTLKDVFGAYSMDVIT | |
| | STSFGVNIDSLNNPQDPFVENTKKLLRFDFLDPFFLSIIFPFLIPILEVL | |
| | NICVFPREVTNFLRKSVKRMKESRLEDTQKHRVDFLQLMIDSQNSK | |
| | ETESHKALSDLELVAQSIIFIFAGYETTSSVLSFIMYELATHPDVQQK | |
| | LQEEIDAVLPNKAPPTYDTVLQMEYLDMVVNETLRLFPIAMRLERV | |
| | CKKDVEINGMFIPKGVVVMIPSYALHRDPKYWTEPEKFLPERFSKK | |
| | NKDNIDPYIYTPFGSGPRNCIGMRFALMNMKLALIRVLQNFSFKPCK | |
| | ETQIPLKLSLGGLLQPEKPVVLKVESRDGTVSGA | |
| McaCYP82-4 | MIMMFIDYYSSWLPQTLLLQSILLAVSLVIFINLFLTRRRSYSSKSHT | 40 |
| | NIIHPPKAAGALPVIGHLYTLFRGLSAGVPLYRQLDAMADRYGPAFI | |
| | IHLGVYPTLVVTCRELAKECFTTNDQTFATRPSTCAGKYIGYNYAFF | |
| | GFAPYGPYWREARKIATVELLSNYRLDSLRHVREAEVGRNVDELY | |
| | ALHASSSTNKQNMMKIDMKQWFDQVTLNVILMMVVGKRCVTTGG | |
| | NEEEVRVVKVLHEFFKHLGTLSVSDVVPYVEWMDLDGNIGRMKST | |
| | AKELDCILGRWLEEHRRERRSDFMDAMLAMVEGIKIPYYDSDTVIK | |
| | AICLNLLNAGSDTLGITMTWALSLLLNNRHVLKKVKDELDVHVGK | |
| | NRQVEELDVKNLVYLHAVVKETLRLFPPAPLGVPHEAMEDCVVGG | |
| | FHVAKGTRLVVNVWKLHRDPSVWSDPLAFKPERFLDNNTVDVRG | |
| | QHFQLLPFGSGRRGCPGITFALQVAHLTLARLLHGFEWDTPDGAPV | |
| | DMSEVSVLTTAKKNPVEVLFTPRLPAEVYTQN | |
| NsaCYP82-4 | MLSIHDSTMVFLQLQAICGIFGFIFIITWWTRWKSSNKMKAPEVAGA | 41 |
| | WPVIGHLHLLGGGRPLYQLLGDMSDKYGPAFTLRMGIQKALVVSS | |
| | WEVAKECLTTNDRALATRPSSAGGKYMGYNNALIPFSPYGPYWRD | |
| | MRKIATLELLSNHRLEELKHVREMEINTCISDMYKLCQVEDGVEIKP | |
| | ISVDLSQWFADLTFNVVVMMITGKRYIGSTDAGDMNEIRHFQAALV | |
| | KFMRLLRISLLVDVFPVLQWINYGGFKGVMKSTARDIDSVLENWLQ | |
| | EHQRKRLSPDFNGNHDFIDVMISTLEGTEFSDYDHNTIIKAISMAMV | |
| | VGGTDTTTTTLIWAISLLLNNPNAMKKVQEELEIHVGKERNVDGSD | |
| | IQHLVYLQAVVKETLRLYPPVPLSVMHQAMEDCVIGSYNIQAGTRV | |
| | LFNLWKLHRDSSVWSDPLEFRPERFLTSHVDVDVRGQHFELIPFGSG | |
| | RRSCPGISFALQVIHLTIARLFHGFNLTTPGNSSVDMSEISGATLSKV | |
| | TPLEVLVTPRLSSKLYN | |
| HcaCYP82- | MDSLLQLQIIGALAALIFTYKLLKVICRSPMTDGMEAPEPPGAWPIIG | 42 |
| 10 | HLHLLGGQDPIARTLGVMTDKYGPILKLRLGVHTGLVVSNWELAK | |
| | ECFTTNDRVLASRPMGAAGKYLGYNYAIFGLAPHGPYWSEVRKIV | |
| | LRELLSNQSLEKLKHVRISEINTCLKNLFSLNNGNTPIKVDMKQWFE | |
| | RPMFNVVTMMIAGKRYFSMENDNEAMNFRKVATEFMYLTGVFVV | |
| | SDALPYLEWLDLQGHVSAMKRTAKELDIHVGKWLEEHRRAKLLGE | |
| | TKNEDDFVDVLLTILPEDLKDNQTYIHDRDTIIKATALALFLAASDT | |
| | TAITLTWALSLILNNPDVLKRAQDELDKHVGKEKLVKESDIINLVYL | |
| | QAIIKETLRLYPAAPLLLPHEAMEDCTVGGYHVPKGTRIFVNIWKLQ | |
| | RDPRVWFDPNEFRPERFLTTHANVDFKGQHFEYIPFSSGRRVCPGIT | |
| | FSTQIMHLTLAHLLHEFNIVTPTKSNAGVDMTESLGITMPKATPLEV | |
| | LLTPRLPSNLYNQYRD | |
| EcaCYP82-7 | MNLLIFFQFLLQFQVLVGLSVLLAFSYYLWVSKNPKINKFKGKGAL | 43 |
| | LAPQAAGAWPIVGHLPQLVGPKPLFRILGAMADNYGPIFMLRFGVH | |
| | PTVVVSSWEMTKECFTTNDRHLASRPSNAASQYLIYEVYALFGFSL | |
| | YGSSYWRDARKIATLELLSHRRLELLKHVPYTEIDTCIKQLHRLWT | |
| | KNNKNQNNPELKVEMNQFFTDLTMNVILKLVVGKRFFNVDDAAD | |
| | HEKEEARKIQGTIFEFFKLTEGSVSAGALPLLNWLDLNGQKRAMKR | |
| | TAKKMDSIAEKLLDEHRQKRLSKEGVKGTHDHNDFMDVLLSILDA | |
| | DQGDYSHHPFNYSRDHVIKATTLSMILSSMSISVSLSWALSLLLNNR | |
| | HVLKKAQDELDMNVGKDRQVEEGDIKNLVYLQAIVKETFRMYPA | |
| | NPLLLPHEAIEDCKIGGFNVPAGTRVVVNAWKLQHDPRVWSNPSEF | |
| | KPERFLNDQAAKVVDVRGQNFEYLPFGSGRRVCPGISFSLQTIHMSL | |
| | ARLVQAFELGTPSNERIDMTEGSGLTMPKTTPLHVLLNPRLPLPLYE | |
| GflCYP82-8 | MELINSLEIQPITISILALLTVSILLYKIIWNHGSRKNNKSNKNNRKTS | 44 |
| | SSAGVVEIPGAWPIIGHLHLFNGSEQMFHKLGSLADQYGPAPFFIRF | |
| | GSRKYVVVSNWELVKTCFTAQSQIFVSRPPMLAMNILFFPKDSLSYI | |
| | QHGDHWRELRKISSTKLLSSHRVETQKHLIASEVDYCFKQLYKLSN | |
| | NGEFTLVRLNTWCEDMALNVHVRMIAGMKNYVAAPGSGEYGGQ | |
| | ARRYRKALEEALDLLNQFTITDVVPWLGWLDHFRDVVGRMKRCG | |
| | AELDSIFATWVEEHRVKRASGKGGDVEPDFIDLCWESMEQLPGNDP | |
| | ATVIKLMCKEHIFNGSGTSSLTLAWILSLIMNNPYVIKKAREELEKH | |
| | VGNHRQVEESDLPNLLYIQAIIKEGMRLYTPGPFIDRNTTEDYEING | |
| | VHIPAGTCLYVNLWKIHRDPNVYEDPLEFKPERFLKNNSDLDLKGQ | |
| | NYQLLPFGAGRRICPGVSLALPLMYLTVSRLIHGFDMKLPKGVEKA | |
| | DMTAHGGVINQRAYPLEVLLKPRLTFQQA | |
| SdiCYP82-3 | MTIGALALLSFIYFLRVSVIKRTKYTNTAVTATNKLENDEDEANHSK | 45 |
| | RVVAPPEVAGAWPILGHLPQLVGLKQPLFRVLGDMADKYGPIFIVR | |
| | FGMYPTLVVSSWEMAKECFTTNDRVLASRPASASGKYLTYNYAMF | |
| | GFTNGPYWREIRKISMLELLSHRRVELLKHVPSTEIDSSIKQLYHLW | |
| | VENQNQNKQGDHQVKVDMSQLLRDLTLNIVLKLVVGKRLFNNND | |
| | MDHEQDEAARKLQKTMVELIKVAGASVASDALPFLGWLDVDGLK | |
| | RTMKRIAKEIDVIAERWLQEHRQKKLTSNDKGGSNNIQGGGGDND | |
| | FMDVMLSILDDDSNFFINYNRDTVIKATSLTMILAGSDTTTLSLTWA | |
| | LTLLATNPGALRKAQDELDTKVGRDRQVDERDIKNLVYLQAIVKET | |
| | LRMYPAAPLAIPHEATQDCIVGGYHVTAGTRVWVNLWKLQRDPH | |
| | AWPNPSEFRPERFLAVENDCKQQGTCDGEAANMDFRGQHFEYMPF | |
| | GSGRRMCPGINFAIQIIHMTLARLLHSFELRVPEEEVIDMAEDSGLTI | |
| | SKVTPLELLLTPRLPLPLYI | |
| SdiCYP82-6 | FCQFQGIVGILLAFLTFLYYLWRASITGLRTKPKHNDFKVTKAAPEA | 46 |
| | DGAWPIVGHFAQFIGPRPLFRILGDMADKYGSIFMVRFGMYPTLVV | |
| | SSWEMAKECFTTNDRFLASRPASAAGKYLTYDFAMLSFSFYGPYW | |
| | REIRKISMLELLSHRRVELLKHVPSTEIDSSIKQLYHLWVENQNQNK | |
| | QGDHQVKVDMSQLLRDLTLNIVLKLVVGKRLFNNNDMDHEQDEA | |
| | ARKLQKTMVELIKVAGASVASDALPFLGWLDVDGLKRTMKRIAKE | |
| | IDVIAERWLQEHRQKKLTSNDKGGSNNIQGGGGDNDFMDVMLSIL | |
| | DDDSNFFINYNRDTVIKATSLTMILAGSDTTTLSLTWALTLLATYPL | |
| | CALRKAQDELDTKVGRDRQVDERDIKNLVYLQAIVKETLRMYPAA | |
| | PLAIPHEATQDCIVGGYHVTAGTRVWVNLWKLQRDPHAWPNPSEF | |
| | RPERFLAVENDCKQQGTCDGEAANMDFRGQHFEYMPFGSGRRMC | |
| | PGINFAIQIIHMTLARLLHSFELRVPEEEVIDMAEDSGLTISKVTPLEL | |
| | LLTPRLPLPLYI | |
| CmaCYP82-6 | MDLFIFFSRFQYIVGLLAFLTFFYYLWRVSITGTRIKTNQNIMNGTN | 47 |
| | MMAPEAAGAWPIVGHLPQLVGPQPLFKILGDMADKYGSIFMVRFG | |
| | MHPTLVVSSWEMAKECFTTNDKFLASRPTSAGGKYLTYDFAMFGF | |
| | SFYGPYWREIRKISTLELLSHRRVELLKHVPYTEIGGSIKQLYKLWM | |
| | ETQNQNKQRDDHQVKVDMSQVFGYLTLNTVLKLVVGKGLFNNND | |
| | MNHEQEEGRKLHETVLEFFKLAGVSVASDALPFLGWLDVDGQKRS | |
| | MKRIAKEMDLIAERWLQEHRQKRLTSNNKASSGHDDFMSVLLSILD | |
| | DDSNFFNYNRDTVIKATSLNLILAASDTTSVSLTWVLSLLVTNPGAL | |
| | KKVQDELDTKVGRNRHVEERDIEKLVYLQATVKETLRMYPAGPLS | |
| | VPHEATQDCTVGGYQVTAGTRLVVNVWKLQRDPRVWPNPSEFKP | |
| | ERFLPDGCEVGCGEAANMDFRGQHFEYIPFGSGRRMCPGIDFAIQII | |
| | HMTLACLLHAFEFQVPSSLDKHLVPAVIDMSEGSGLTMPKVTPLEV | |
| | LLNPRLPLPLYEL | |
| EcaCYP82-5 | MEKPILLQLQPGILGLLALMCFLYYVIKVSLSTRNCNQLVRHPPEAA | 48 |
| | GSWPIVGHLPQLVGSGKPLFRVLGDMADKFGPIFMVRFGVHPTLVV | |
| | SSWEMAKECFTSNDKFLASRPPSAASIYMAYDHAMLGFSSYGPYW | |
| | REIRKISTLHLLSHRRLELLKHVPHLEIHNFIKGLYGIWKDHQKQQQ | |
| | QPTARDDQDSVMLEMSQLFGYLTLNIVLSLVVGKRVCNYHADGHL | |
| | DDGEEAGQGQKLHQTITDFFKLSGVSVASDALPFLGLFDLDGQKKI | |
| | MKRVAKEMDFVAERWLQDKKSSLLLSSKSNNKQNEAGEGDVDDF | |
| | MDVLMSTLPDDDDSFFTKYSRDTVIKANSLSMVVAGSDTTSVSLT | |
| | WALSLLLNNIQVLRKAQDELDTKVGRDRHVEEKDIDNLVYLQAIV | |
| | KETLRMYPAGPLSVPHEAIEDCNVGGYHIKTGTRLLVNIWKLQRDP | |
| | RVWSNPSEFRPERFLDNQSNGTLLDFRGQHFEYIPFGSGRRMCPGV | |
| | NLATPILHMTLARLLQSFDLTTPSSSPVDMTEGSGLTMPKVTPLKVL | |
| | LTPRLPLPLYDY | |
| PbrCYP82-5 | MDVAIIVDHHYLQPFVSIAGLLALLSFFYCIWVFIIRPRIIKSNLDERK | 49 |
| | LSPSSPPEVAGAWPIVGHLPQLIGSTPLFKILADMSNKYGPIFMVRFG | |
| | MYPTLVVSSWEMSKECFTTNDRLFATRPPSAAGKYLTKALFAFSVY | |
| | GPYWREIRKISTIHLLSLRRLELLKHGRYLEIDKCMKRLFEYWMEH | |
| | HKNIISTTSSVKVNMSQVFAELSLNVVLKIIVGKTLFIKNGNEDYTKE | |
| | EEEGQKLHKTILKFMELAGVSVASDVLPFLGWLDVDGQKKQMKR | |
| | VYKEMNLIASKWLGEHRERKRLQIIQKRGAARGSNYDDGNDFMDV | |
| | LMSILDEENDDLFFGYSRDTVIKSTCLQLIVAASDTTSLAMTWALSL | |
| | LLTNPNVLQKAQDELDTKVGRDRIIEEHDIECLVYLQAIVKETLRLY | |
| | PPAPLSLPHEAMEDCTVGGYQVKAGTRLVVNLWKLQRDPRVWSN | |
| | PLEFKPERFLPQSDGGFGGEEARMDFRGQHFEYTPFGSGRRICPGIDF | |
| | FLQTVHMALARLLQAFDFNTAGGLVIDMVEGPGLTMPKVTPLEVH | |
| | LNP | |
| | RLPVTLY | |
| PbrCYP82-6 | MQVDWPNILQKYYPIITCSLLTLLSFYYIWVSITKPSRNSKTKLPPPE | 50 |
| | VAGSWPIVGHLPQLVGSTPLFKILANMSDKYGPIFMVRFGMHPTLV | |
| | VSSWEMSKECFTTNDKFLASRPPSASAKYLGYDNAMFVFSDYGPY | |
| | WREIRKISTLQLLTHKRLDSLKNIPYLEINSCVKTLYTRWAKTQSQIK | |
| | QNVGGAADDFVKVDMTEMFGHLNLNVVLRLVVGKPIFIQKDNAD | |
| | EDYTKDGHNKEELGQKLHKTIIEFFELAGASVASDVLPYLGWLDVD | |
| | GQKKRMKKIAMEMDLFAQKWLEEHRQKGINHDNENDFMAVLISV | |
| | LGEGKDDHIFGYSRDTVIKATCLTLIVAATDTTLVSLTWALSLLLTN | |
| | PRVLSKAQDELDTVVGKERNVEDRDVNHLVYLQAVIKETLRLYPPS | |
| | PLAVPHEAIENCNVGGYEVKARTRLLVNLWKIHRDPRVWSNPLEFK | |
| | PERFLPKLDGGTGEASKLDFKGQDFVYTPFGSGRRMCPGINFASQTL | |
| | HMTLARLLHAFDFDIESNGLVIDMTEGSGLTMPKVTPLQVHLRPRL | |
| | PATLY | |
| McaCYP82-4 | MIMMFIDYYSSWLPQTLLLQSILLAVSLVIFINLFLTRRRSYSSKSHT | 51 |
| | NIIHPPKAAGALPVIGHLYTLFRGLSAGVPLYRQLDAMADRYGPAFI | |
| | IHLGVYPTLVVTCRELAKECFTTNDQTFATRPSTCAGKYIGYNYAFF | |
| | GFAPYGPYWREARKIATVELLSNYRLDSLRHVREAEVGRNVDELY | |
| | ALHASSSTNKQNMMKIDMKQWFDQVTLNVILMMVVGKRCVTTGG | |
| | NEEEVRVVKVLHEFFKHLGTLSVSDVVPYVEWMDLDGNIGRMKST | |
| | AKELDCILGRWLEEHRRERRSDFMDAMLAMVEGIKIPYYDSDTVIK | |
| | AICLNLLNAGSDTLGITMTWALSLLLNNRHVLKKVKDELDVHVGK | |
| | NRQVEELDVKNLVYLHAVVKETLRLFPPAPLGVPHEAMEDCVVGG | |
| | FHVAKGTRLVVNVWKLHRDPSVWSDPLAFKPERFLDNNTVDVRG | |
| | QHFQLLPFGSGRRGCPGITFALQVAHLTLARLLHGFEWDTPDGAPV | |
| | DMSEVSVLTTAKKNPVEVLFTPRLPAEVYTQN | |
| NsaCYP82-4 | MLSIHDSTMVFLQLQAICGIFGFIFIITWWTRWKSSNKMKAPEVAGA | 52 |
| | WPVIGHLHLLGGGRPLYQLLGDMSDKYGPAFTLRMGIQKALVVSS | |
| | WEVAKECLTTNDRALATRPSSAGGKYMGYNNALIPFSPYGPYWRD | |
| | MRKIATLELLSNHRLEELKHVREMEINTCISDMYKLCQVEDGVEIKP | |
| | ISVDLSQWFADLTFNVVVMMITGKRYIGSTDAGDMNEIRHFQAALV | |
| | KFMRLLRISLLVDVFPVLQWINYGGFKGVMKSTARDIDSVLENWLQ | |
| | EHQRKRLSPDFNGNHDFIDVMISTLEGTEFSDYDHNTIIKAISMAMV | |
| | VGGTDTTTTTLIWAISLLLNNPNAMKKVQEELEIHVGKERNVDGSD | |
| | IQHLVYLQAVVKETLRLYPPVPLSVMHQAMEDCVIGSYNIQAGTRV | |
| | LFNLWKLHRDSSVWSDPLEFRPERFLTSHVDVDVRGQHFELIPFGSG | |
| | RRSCPGISFALQVIHLTIARLFHGFNLTTPGNSSVDMSEISGATLSKV | |
| | TPLEVLVTPRLSSKLYN | |
| HcaCYP82- | MDSLLQLQIIGALAALIFTYKLLKVICRSPMTDGMEAPEPPGAWPIIG | 53 |
| 10 | HLHLLGGQDPIARTLGVMTDKYGPILKLRLGVHTGLVVSNWELAK | |
| | ECFTTNDRVLASRPMGAAGKYLGYNYAIFGLAPHGPYWSEVRKIV | |
| | LRELLSNQSLEKLKHVRISEINTCLKNLFSLNNGNTPIKVDMKQWFE | |
| | RPMFNVVTMMIAGKRYFSMENDNEAMNFRKVATEFMYLTGVFVV | |
| | SDALPYLEWLDLQGHVSAMKRTAKELDIHVGKWLEEHRRAKLLGE | |
| | TKNEDDFVDVLLTILPEDLKDNQTYIHDRDTIIKATALALFLAASDT | |
| | TAITLTWALSLILNNPDVLKRAQDELDKHVGKEKLVKESDIINLVYL | |
| | QAIIKETLRLYPAAPLLLPHEAMEDCTVGGYHVPKGTRIFVNIWKLQ | |
| | RDPRVWFDPNEFRPERFLTTHANVDFKGQHFEYIPFSSGRRVCPGIT | |
| | FSTQIMHLTLAHLLHEFNIVTPTKSNAGVDMTESLGITMPKATPLEV | |
| | LLTPRLPSNLYNQYRD | |
| EcaCYP82-7 | MNLLIFFQFLLQFQVLVGLSVLLAFSYYLWVSKNPKINKFKGKGAL | 54 |
| | LAPQAAGAWPIVGHLPQLVGPKPLFRILGAMADNYGPIFMLRFGVH | |
| | PTVVVSSWEMTKECFTTNDRHLASRPSNAASQYLIYEVYALFGFSL | |
| | YGSSYWRDARKIATLELLSHRRLELLKHVPYTEIDTCIKQLHRLWT | |
| | KNNKNQNNPELKVEMNQFFTDLTMNVILKLVVGKRFFNVDDAAD | |
| | HEKEEARKIQGTIFEFFKLTEGSVSAGALPLLNWLDLNGQKRAMKR | |
| | TAKKMDSIAEKLLDEHRQKRLSKEGVKGTHDHNDFMDVLLSILDA | |
| | DQGDYSHHPFNYSRDHVIKATTLSMILSSMSISVSLSWALSLLLNNR | |
| | HVLKKAQDELDMNVGKDRQVEEGDIKNLVYLQAIVKETFRMYPA | |
| | NPLLLPHEAIEDCKIGGFNVPAGTRVVVNAWKLQHDPRVWSNPSEF | |
| | KPERFLNDQAAKVVDVRGQNFEYLPFGSGRRVCPGISFSLQTIHMSL | |
| | ARLVQAFELGTPSNERIDMTEGSGLTMPKTTPLHVLLNPRLPLPLYE | |
| GflCYP82-8 | MELINSLEIQPITISILALLTVSILLYKIIWNHGSRKNNKSNKNNRKTS | 55 |
| | SSAGVVEIPGAWPIIGHLHLFNGSEQMFHKLGSLADQYGPAPFFIRF | |
| | GSRKYVVVSNWELVKTCFTAQSQIFVSRPPMLAMNILFFPKDSLSYI | |
| | QHGDHWRELRKISSTKLLSSHRVETQKHLIASEVDYCFKQLYKLSN | |
| | NGEFTLVRLNTWCEDMALNVHVRMIAGMKNYVAAPGSGEYGGQ | |
| | ARRYRKALEEALDLLNQFTITDVVPWLGWLDHFRDVVGRMKRCG | |
| | AELDSIFATWVEEHRVKRASGKGGDVEPDFIDLCWESMEQLPGNDP | |
| | ATVIKLMCKEHIFNGSGTSSLTLAWILSLIMNNPYVIKKAREELEKH | |
| | VGNHRQVEESDLPNLLYIQAIIKEGMRLYTPGPFIDRNTTEDYEING | |
| | VHIPAGTCLYVNLWKIHRDPNVYEDPLEFKPERFLKNNSDLDLKGQ | |
| | NYQLLPFGAGRRICPGVSLALPLMYLTVSRLIHGFDMKLPKGVEKA | |
| | DMTAHGGVINQRAYPLEVLLKPRLTFQQA | |
| SdiCYP82-3 | MTIGALALLSFIYFLRVSVIKRTKYTNTAVTATNKLENDEDEANHSK | 56 |
| | RVVAPPEVAGAWPILGHLPQLVGLKQPLFRVLGDMADKYGPIFIVR | |
| | FGMYPTLVVSSWEMAKECFTTNDRVLASRPASASGKYLTYNYAMF | |
| | GFTNGPYWREIRKISMLELLSHRRVELLKHVPSTEIDSSIKQLYHLW | |
| | VENQNQNKQGDHQVKVDMSQLLRDLTLNIVLKLVVGKRLFNNND | |
| | MDHEQDEAARKLQKTMVELIKVAGASVASDALPFLGWLDVDGLK | |
| | RTMKRIAKEIDVIAERWLQEHRQKKLTSNDKGGSNNIQGGGGDND | |
| | FMDVMLSILDDDSNFFINYNRDTVIKATSLTMILAGSDTTTLSLTWA | |
| | LTLLATNPGALRKAQDELDTKVGRDRQVDERDIKNLVYLQAIVKET | |
| | LRMYPAAPLAIPHEATQDCIVGGYHVTAGTRVWVNLWKLQRDPH | |
| | AWPNPSEFRPERFLAVENDCKQQGTCDGEAANMDFRGQHFEYMPF | |
| | GSGRRMCPGINFAIQIIHMTLARLLHSFELRVPEEEVIDMAEDSGLTI | |
| | SKVTPLELLLTPRLPLPLYI | |
| SdiCYP82-6 | FCQFQGIVGILLAFLTFLYYLWRASITGLRTKPKHNDFKVTKAAPEA | 57 |
| | DGAWPIVGHFAQFIGPRPLFRILGDMADKYGSIFMVRFGMYPTLVV | |
| | SSWEMAKECFTTNDRFLASRPASAAGKYLTYDFAMLSFSFYGPYW | |
| | REIRKISMLELLSHRRVELLKHVPSTEIDSSIKQLYHLWVENQNQNK | |
| | QGDHQVKVDMSQLLRDLTLNIVLKLVVGKRLFNNNDMDHEQDEA | |
| | ARKLQKTMVELIKVAGASVASDALPFLGWLDVDGLKRTMKRIAKE | |
| | IDVIAERWLQEHRQKKLTSNDKGGSNNIQGGGGDNDFMDVMLSIL | |
| | DDDSNFFINYNRDTVIKATSLTMILAGSDTTTLSLTWALTLLATYPL | |
| | CALRKAQDELDTKVGRDRQVDERDIKNLVYLQAIVKETLRMYPAA | |
| | PLAIPHEATQDCIVGGYHVTAGTRVWVNLWKLQRDPHAWPNPSEF | |
| | RPERFLAVENDCKQQGTCDGEAANMDFRGQHFEYMPFGSGRRMC | |
| | PGINFAIQIIHMTLARLLHSFELRVPEEEVIDMAEDSGLTISKVTPLEL | |
| | LLTPRLPLPLYI | |
| CmaCYP82-6 | MDLFIFFSRFQYIVGLLAFLTFFYYLWRVSITGTRIKTNQNIMNGTN | 58 |
| | MMAPEAAGAWPIVGHLPQLVGPQPLFKILGDMADKYGSIFMVRFG | |
| | MHPTLVVSSWEMAKECFTTNDKFLASRPTSAGGKYLTYDFAMFGF | |
| | SFYGPYWREIRKISTLELLSHRRVELLKHVPYTEIGGSIKQLYKLWM | |
| | ETQNQNKQRDDHQVKVDMSQVFGYLTLNTVLKLVVGKGLFNNND | |
| | MNHEQEEGRKLHETVLEFFKLAGVSVASDALPFLGWLDVDGQKRS | |
| | MKRIAKEMDLIAERWLQEHRQKRLTSNNKASSGHDDFMSVLLSILD | |
| | DDSNFFNYNRDTVIKATSLNLILAASDTTSVSLTWVLSLLVTNPGAL | |
| | KKVQDELDTKVGRNRHVEERDIEKLVYLQATVKETLRMYPAGPLS | |
| | VPHEATQDCTVGGYQVTAGTRLVVNVWKLQRDPRVWPNPSEFKP | |
| | ERFLPDGCEVGCGEAANMDFRGQHFEYIPFGSGRRMCPGIDFAIQII | |
| | HMTLACLLHAFEFQVPSSLDKHLVPAVIDMSEGSGLTMPKVTPLEV | |
| | LLNPRLPLPLYEL | |
| EcaCYP82-5 | MEKPILLQLQPGILGLLALMCFLYYVIKVSLSTRNCNQLVRHPPEAA | 59 |
| | GSWPIVGHLPQLVGSGKPLFRVLGDMADKFGPIFMVRFGVHPTLVV | |
| | SSWEMAKECFTSNDKFLASRPPSAASIYMAYDHAMLGFSSYGPYW | |
| | REIRKISTLHLLSHRRLELLKHVPHLEIHNFIKGLYGIWKDHQKQQQ | |
| | QPTARDDQDSVMLEMSQLFGYLTLNIVLSLVVGKRVCNYHADGHL | |
| | DDGEEAGQGQKLHQTITDFFKLSGVSVASDALPFLGLFDLDGQKKI | |
| | MKRVAKEMDFVAERWLQDKKSSLLLSSKSNNKQNEAGEGDVDDF | |
| | MDVLMSTLPDDDDSFFTKYSRDTVIKANSLSMVVAGSDTTSVSLT | |
| | WALSLLLNNIQVLRKAQDELDTKVGRDRHVEEKDIDNLVYLQAIV | |
| | KETLRMYPAGPLSVPHEAIEDCNVGGYHIKTGTRLLVNIWKLQRDP | |
| | RVWSNPSEFRPERFLDNQSNGTLLDFRGQHFEYIPFGSGRRMCPGV | |
| | NLATPILHMTLARLLQSFDLTTPSSSPVDMTEGSGLTMPKVTPLKVL | |
| | LTPRLPLPLYDY | |
| PbrCYP82-5 | MDVAIIVDHHYLQPFVSIAGLLALLSFFYCIWVFIIRPRIIKSNLDERK | 60 |
| | LSPSSPPEVAGAWPIVGHLPQLIGSTPLFKILADMSNKYGPIFMVRFG | |
| | MYPTLVVSSWEMSKECFTTNDRLFATRPPSAAGKYLTKALFAFSVY | |
| | GPYWREIRKISTIHLLSLRRLELLKHGRYLEIDKCMKRLFEYWMEH | |
| | HKNIISTTSSVKVNMSQVFAELSLNVVLKIIVGKTLFIKNGNEDYTKE | |
| | EEEGQKLHKTILKFMELAGVSVASDVLPFLGWLDVDGQKKQMKR | |
| | VYKEMNLIASKWLGEHRERKRLQIIQKRGAARGSNYDDGNDFMDV | |
| | LMSILDEENDDLFFGYSRDTVIKSTCLQLIVAASDTTSLAMTWALSL | |
| | LLTNPNVLQKAQDELDTKVGRDRIIEEHDIECLVYLQAIVKETLRLY | |
| | PPAPLSLPHEAMEDCTVGGYQVKAGTRLVVNLWKLQRDPRVWSN | |
| | PLEFKPERFLPQSDGGFGGEEARMDFRGQHFEYTPFGSGRRICPGIDF | |
| | FLQTVHMALARLLQAFDFNTAGGLVIDMVEGPGLTMPKVTPLEVH | |
| | LNPRLPVTLY | |
| PbrCYP82-6 | MQVDWPNILQKYYPIITCSLLTLLSFYYIWVSITKPSRNSKTKLPPPE | 61 |
| | VAGSWPIVGHLPQLVGSTPLFKILANMSDKYGPIFMVRFGMHPTLV | |
| | VSSWEMSKECFTTNDKFLASRPPSASAKYLGYDNAMFVFSDYGPY | |
| | WREIRKISTLQLLTHKRLDSLKNIPYLEINSCVKTLYTRWAKTQSQIK | |
| | QNVGGAADDFVKVDMTEMFGHLNLNVVLRLVVGKPIFIQKDNAD | |
| | EDYTKDGHNKEELGQKLHKTIIEFFELAGASVASDVLPYLGWLDVD | |
| | GQKKRMKKIAMEMDLFAQKWLEEHRQKGINHDNENDFMAVLISV | |
| | LGEGKDDHIFGYSRDTVIKATCLTLIVAATDTTLVSLTWALSLLLTN | |
| | PRVLSKAQDELDTVVGKERNVEDRDVNHLVYLQAVIKETLRLYPPS | |
| | PLAVPHEAIENCNVGGYEVKARTRLLVNLWKIHRDPRVWSNPLEFK | |
| | PERFLPKLDGGTGEASKLDFKGQDFVYTPFGSGRRMCPGINFASQTL | |
| | HMTLARLLHAFDFDIESNGLVIDMTEGSGLTMPKVTPLQVHLRPRL | |
| | PATLY | |
| PbrCYP82-7 | MMDLAMFIDQYFSLAKIAGLLALLSFFYYLWISTLWSPRNPKLSSVS | 62 |
| | PPEVAGAWPILGHLPQLLGSRPLFKILADMSDNYGPIFMVRFGMHPT | |
| | LVVSSWEMAKECFTTNDRFLAGRPSGAANKYLTFALFGFSTYGPY | |
| | WREIRKIATLHLLSHRRLELLKHVPDLEVTNCMKHLHRRWIDSQNQ | |
| | IKQNDAAAGSVKVDMGRVFGELTLNVVLKLVAGKSIFFKNDNTRQ | |
| | YDSKDGHNKEEEEGKKLHKTIIDFYSLAGASVASDVLPFLGWLDVD | |
| | GQKKRMKRVAKDMDFIAAKWLEEHRHQKRQTVLSSSATLGSSNH | |
| | DDAKDFMDVLMSILDGENDDLFFGYSRDTVIKTTCLQLIAAAADTT | |
| | SVTMTWALALLITNPTILRKAQDELDTKVGKDRNIEERDINDLVYL | |
| | QAIVKETLRMYPAGPLNVPHEAIADCNIGGYEVRAGTRLLVNLWK | |
| | MHRDPRVWSNPSEFKPERFLPQLDGGSGGEAANLDFRGQDFEYLPF | |
| | SAGRRMCPGIDFSLQTLHMTLARLLHGFDFNNDSAGIIIDMEEGSGL | |
| | TMPKLTPLEIYLCPRLPAKLY | |

TABLE 5
N-methyl transferase and N-modifying candidate enzymes

| | | SEQ ID |
|---|---|---|
| Name | Sequence | NO: |
| TfCNMT | MAVEGKQVAPKKAIIVELLKKLELGLVPDDEIKKLIRIQLGRRLQWG | 63 |
| | CKSTYEEQIAQLVNLTHSLRQMKIATEVETLDDQMYEVPIDFLKIMN | |
| | GSNLKGSCCYFKNDSTTLDEAEIAMLELYCERAQIKDGHSVLDLGCG | |
| | QGALTLYVAQKYKNSRVTAVTNSVSQKEFIEEESRKRNLSNVEVLL | |
| | ADITTHKMPDTYDRILVVELFEHMKNYELLLRKIKEWMAKDGLLFV | |
| | EHICHKTFAYHYEPIDEDDWFTEYVFPAGTMIIPSASFFLYFQDDVSV | |
| | VNHWTLSGKHFSRTNEEWLKRLDANVELIKPMFVTITGQCRQEAMK | |
| | LINYWRGFCLSGMEMFGYNNGEEWMASHVLFKKK | |
| CjCNMT | MAVEAKQTKKAAIVELLKQLELGLVPYDDIKQLIRRELARRLQWGY | 64 |
| | KPTYEEQIAEIQNLTHSLRQMKIATEVETLDSQLYEIPIEFLKIMNGSN | |
| | LKGSCCYFKEDSTTLDEAEIAMLDLYCERAQIQDGQSVLDLGCGQG | |
| | ALTLHVAQKYKNCRVTAVTNSVSQKEYIEEESRRRNLLNVEVKLAD | |
| | ITTHEMAETYDRILVIELFEHMKNYELLLRKISEWISKDGLLFLEHICH | |
| | KTFAYHYEPLDDDDWFTEYVFPAGTMIIPSASFFLYFQDDVSVVNH | |
| | WTLSGKHFSRTNEEWLKRLDANLDVIKPMFETLMGNEEEAVKLINY | |
| | WRGFCLSGMEMFGYNNGEEWMASHVLFKKK | |
| PsCNMT | MQLKAKEELLRNMELGLIPDQEIRQLIRVELEKRLQWGYKETHEEQL | 65 |
| | SQLLDLVHSLKGMKMATEMENLDLKLYEAPMEFLKIQHGSNMKQS | |
| | AGYYTDESTTLDEAEIAMLDLYMERAQIKDGQSVLDLGCGLGAVAL | |
| | FGANKFKKCQFTGVTSSVEQKDYIEGKCKELKLTNVKVLLADITTYE | |
| | TEERFDRIFAVELIEHMKNYQLLLKKISEWMKDDGLLFVEHVCHKTL | |
| | AYHYEPVDAEDWYTNYIFPAGTLTLSSASMLLYFQDDVSVVNQWTL | |
| | SGKHYSRSHEEWLKNMDKNIVEFKEIMRSITKTEKEAIKLLNFWRIFC | |
| | MCGAELFGYKNGEEWMLTHLLFKKK | |
| PsTNMT | MGSIDEVKKESAGETLGRLLKGEIKDEELKKLIKFQFEKRLQWGYKS | 66 |
| | SHQEQLSFNLDFIKSLKKMEMSGEIETMNKETYELPSEFLEAVFGKT | |
| | VKQSMCYFTHESATIDEAEEAAHELYCERAQIKDGQTVLDIGCGQG | |
| | GLVLYIAQKYKNCHVTGLTNSKAQVNYLLKQAEKLGLTNVDAILAD | |
| | VTQYESDKTYDRLLMIEAIEHMKNLQLFMKKLSTWMTKESLLFVDH | |
| | VCHKTFAHFFEAVDEDDWYSGFIFPPGCATILAANSLLYFQDDVSVV | |
| | DHWVVNGMHMARSVDIWRKALDKNMEAAKEILLPGLGGSHETVN | |
| | GVVTHIRTFCMGGYEQFSMNNGDEWMVAQLLFKKK | |
| EcTNMT | MGSSAGEIMGRLMKGEIEDEELKKLIRHQWDRRIEWGYKPTHEKQL | 67 |
| | AFNLDFIKGLKEMVMSGEIDTMNKETYELPTAFLEAVFGKTVKQSC | |
| | CYFKDENSTIDEAEEAAHELYCERAQIKDGQTVLDIGCGQGGLVLYI | |
| | AEKYKNCHVTGLTNSKAQANYIEQQAEKLELTNVDVIFADVTKFDT | |
| | DKTYDRILVVETIEHMKNIQLFMKKLSTWMTEDSLLFVDHISHKTFN | |
| | HNFEALDEDDWYSGFIFPKGCVTILSSSTLLYFQDDVSALDHWVVNG | |
| | MHMARSVEAWRKKLDETIEAAREILEPGLGSKEAVNQVITHIRTFCI | |
| | GGYEQFSYNNGEEWMITQILFKKK | |
| PsRNMT | MSTTMETTKISQQDDLWKNMELGQISDEEVRRLMKIGIEKRIKWGT | 68 |
| | KPTQQEQLAQLLDFNKSLRGMKMATEIDTLENHKIYETPESFNQIIGG | |
| | KESAGLFTDETTTTMEEANTKMMDLYCERAGLKDGHTILDLGCGA | |
| | GLLVLHLAKKYKKSKITGITNTSSHKEYILKQCKNLNLSNVEIILADV | |
| | TKVDIESTFDRVFVIGLIEHMKNFELFLRKISKWMKDDGLLLLEHLC | |
| | HKSFSDHWEPLSEDDWYAKNFFPSGTLVIPSATCLLYFQEDVTVIDH | |
| | WILSGNNFARSNEVILKRIDGKIEEVKDIFMSFYGIGREEAVKLINWW | |
| | RLLCITANELFKYNNGEEWLISQLLFKKKLMTCI | |
| TfPNMT | METKQTKKEAVANLIKRIEHGEVSDEEIRGMMKIQVQKRLKWGYKP | 69 |
| | THEQQLAQLVTFAQSLKGMEMAEEVDTLDAELYEIPLPFLHIMCGKT | |
| | LKFSPGYFKDESTTLDESEVYMMDLYCERAQIKDGQSILDLGCGHGS | |
| | LTLHVAQKYRGCKVTGITNSVSQKEFIMDQCKKLDLSNVEIILEDVT | |
| | KFETEITYDRIFAVALIEHMKNYELFLKKVSTWIAQYGLLFVEHHCH | |
| | KVFAYQYEPLDEDDWYTEYIFPSGTLVMSSSSILLYFQEDVSVVNHW | |
| | TLSGKHPSLGFKQWLKRLDDNIDEVKEIFESFYGSKEKAMKFITYWR | |
| | VFCIAHSQMYSTNNGEEWMLSQVLFKKK | |
| PbrTNMT1 | MGSIDEVKKESAGETLGRLLKGEIKDEELKKLIKFQFEKRLQWGYKS | 70 |
| | SHQEQLSFNLDFIKSLKKMEMSGEIETMNKETYELPSEFLEAVFGKT | |
| | VKQSMCYFKHESATIDEAEEAAHELYCERAQIKDGQTVLDIGCGQG | |
| | GLVLYIARKYKKCHVTGLTNSKAQVNYLLKQAEKLGLTNVDAILAD | |
| | VTQYESDKTYDRLLMIEAIEHMKNLQLFMKKLSTWMTEESLLFVDH | |
| | VCHKTFAHFFEAVDEDDWYSGFIFPPGCATILAANSLLYFQDDVSVV | |
| | DHWVVNGMHMARSVDIWRKALDKNMEAAKEILLPGLGGSHEAVN | |
| | GVVTHIRTFCMGGYEQFSMNDGDEWMVAQLLFKKK | |
| PbrTNMT2 | MGSIEEVKKESAEETLGRLLRGEINDEELKKLIKYQLEKRLQWGYKS | 71 |
| | SHQEQLSFNLDFINSLKKMGMSGQVEAFTNEVYELPTECFEAAYGKS | |
| | MKLSGCYFKHESSTIDEAEEASHELYCERAQIKDGQTVLDIGCGQGG | |
| | LVLYVAQKYKNCHVTGLTNSKEQVNYILKQAEKLGLRNVDVILAD | |
| | VTQYESDKTYDRILVIGVVEHMKNMQLFIKKLSTWMAEDSLLFVDH | |
| | SCHKTFNHFFEALDEDDWYSGYIFPPGCATFLSADSLLYFQDDVSVV | |
| | DHWVVNGMHFARTVDAWRKKLDKNMEAVKEILLPGLGGNHEAVN | |
| | GVITHIRTCCVGGYVQFSLNDGDEWMNAQLLFKKK | |
| AmeNMT1 | MCLFFAEKMGLMAEANNQQQLKKEDLLKNMELGLIPDEEIRKLIRV | 72 |
| | QLEKRLNWGYKSTHEQQLSQLLHLVHSLKKMKIATEMENLDLKLY | |
| | EAPFSFVQIQHGSTIKESSGLFKDESTTLDEAEIAMLDLYTKRAKIEDG | |
| | QSVLDLGCGLGAVTLYVAQKFKNCYVTGITSSVEQKDFIEGRCKELK | |
| | LSNVKVILADITTYETEEKYNRIFAVELIEHMKNYELLLRKISEWMKQ | |
| | DGLLFIEHVCHKTLAYHYEPLDEEDWYTNYIFPAGTLTLSSATLLLYF | |
| | QDDVAVVDQWTLSGKHYSRSHEEWLKRIDGNIEEVKEIMKSITKSEE | |
| | EAKKLLNFWRIFCMCGAELFGYKNGEEWMMTHILFKKK | |
| GflNMT1 | MDLMATSKQVKKKEELLKNMELGLVPDEEIRRLIRIELEKRLKWGY | 73 |
| | KPTHQQQLAQLLDLVHSLKKMKIATEMESLDLKLYEAPFSFVQIKHG | |
| | STIKESSSYFKDESMTLDEAEIAMLDLYVERAQIEDGQSVLDLGCGL | |
| | GAVTLHVAKKYKNCHVTGLTNSVEQKDFIEGKCKELNLSNVKVILA | |
| | DVTSHEMEDKFDRIFAVELIEHMKNYELLLRRISKWMKDDGLLFIEH | |
| | VCHKTFAYHYEPIDEDDWYTEYIFPAGTLTLSSASLLLYFQDDVSVV | |
| | NHWTLSGKHYSRSHEEWLKRIDGNMDAVKEIMKSITKTEEEAVKLI | |
| | NFWRIFCMCGAELFGYKDGEEWMMSHVLFKKKQLLQQC | |
| EcaNMT1 | MVDLKVEKEELLKSMELGLVPDEDIRKHIRSQLEKRLKWGYKPNHE | 74 |
| | QQLAQLLDVIHSLKKMKISKEYESFDLRLYEAPFDFHKIQLGTHLKES | |
| | CSYYKDESTTLDEAEGAMLDLYTQKAKIEDGQSILDLGCGVGAVTL | |
| | FVANKYKNCKVTGITSCQWQKDFIENKCKELNLTNVRVIIGDVTAYE | |
| | MEETFDRIFAIELIEHMKNYELLLRKISKWMKDDGLLFIEHVCHKILA | |
| | YPYEPIDEEDWFTEYIFPGGTLTLSSASLLLYFQDDVSVVEHSSLNGK | |
| | HYSRSHGEWLKNIDANIDEVKGIMRSITKTEEEAVRLVNFWRIFCMC | |
| | GIELFGYNNGEEWMVSHILLKKK | |
| EcaNMT2 | MAADLVVKKWNNKKELIDEMELGLVGDEEIRELIRNDLEKRLKWG | 75 |
| | YKSNHEQQLAQLLHFVHSLRGMKIAADEVESFNIKVYEAPFSFNKIQ | |
| | LGSSLKESSCYYKHDETTLDEGEIAMMELYTEKAQIKDGQSVLDLGC | |
| | GLGSLTLYVANKYPNCKVTGTTASLWHKDFIESKCKEQELTNVKIVL | |
| | GDATTHEMEERFDRILAIGLIEHLKNYGLLLGRISKWLKDDGFLFIQH | |
| | VCHKTLAYPLVPVDEEDWIGEYIFPGGTLTMPSASLLLYFQDELSVV | |
| | DHSTLNGKHFSRTHEEWLKNIDAKIDEVKEILKSVTKTEEEVVRLTN | |
| | FWRIFCMFGVEMFGYNEGEEWMLSQILFKKK | |
| CmaNMT4 | MASGKVVDLLKRLDSGLVSDEELRRVIRFELERRLKWGYKPTHEQQ | 76 |
| | LAELLNLAHATKQMEIATKIDTLNSTMYEVPNSFLEIQLGSTLKESCL | |
| | YFKDESTTVDEAEIAMMDLYLERAQIKDGQIILDLGCGLGALAFHIA | |
| | QKYTNCNVTSVTNSVKQKEFIEEKCKILNVSNVKVILTDICTLEMEAT | |
| | FDRIFAIGLIEHMKNYELLLRKFSAWMKQDGLLFIEHLCHKTLGYHN | |
| | EPIDEDDWYTAYFFPAGTLTFIPSSFLLYFQDDVSVVNHWTLSGKHFS | |
| | RSNEEWLKRMDNKIDEVKEIYKAAASETKDDDIMKLIRLWRFLSISA | |
| | AEMFGYKDGEEWMISQVLFKKK | |
| EcNMT3 | MASLVEEGSFVNNKESVKERVSELVKRLKNGLVSDEELRKLMRVEL | 77 |
| | EKRLEWGYKSTHEQQLSQLIDLAHSMKKMEIAMEIDALNSTVYEVP | |
| | LSFLQIIHGTTIKESCLYFKDESTTVDEAEIAMMDLYLERAQIKDGQSI | |
| | LDLGCGLGGFSFHIASKFTGCNITAVTNSVKQKEFIEEKCKTLNVPNI | |
| | KVILADICTTEIENVFDRIIAIGLIEHMKNYELLLKKFSKWMTQDGLLF | |
| | IEHLCHKTFGYHNEPLDEDDWYTTYFFPAGTLTFIPSSFLLYFQDDVS | |
| | VVDHWTLNGKHFARSNEEWLKRMDEKMDEVKQIFRSNLKSENEVT | |
| | KTIGEWRFLSMSAAEMFGYNNGEEWMVSQLLFKKK | |
| GflNMT5 | MGSNETNGELKTKEMVPDLLKRLESGLVADEELRKLIRFELERRLK | 78 |
| | WGYKPTHEQQLAELLKLAHSTKQMKIATETDSLNSTMYEVPIPFLQL | |
| | QFGSAIKESCCYFKDESTTLDEAEVAMMDLYLERTQIKDGQSILDLG | |
| | CGLGALAFHIVQKYPNCNVLAITNSVEQKEFIEEKCKIRKVENVKVS | |
| | LADICTLEMKTTFDRIFAIGLLEHMKNYQLLLKKFSNWMKQDGLLFI | |
| | EHLCHKTLAYHYEPLDEDDWYTEYFFPAGTLTIISSSFLLYFQDDVSI | |
| | VNHWSLSGKHFSRSNEEWLKRMDMKIDEVKEILEAAFENKDHDITK | |
| | LINHWRFLAINATEMFGYNNGEEWMVSQVLFKKK | |
| ScaNMT1 | MASDHEVSNKELKKKKEVITELLKRLESGLVSDEELRGLIRFELERRL | 79 |
| | RWGYKPTHEQQLAQLLNLAHSMKQMKIATEIDALNSTMYEVPIPFL | |
| | QIQLGSTLKESCCYFKDESTTVDEAEIAMMDLYLERAQIKDGQSILDL | |
| | GCGLGALAFHIAQKYTNCNITAITNSVRQKEFIEEKCKILNVSNVKVS | |
| | LADICTLEMEATFDRIFAIGLIEHMKNYELLLKKFSEWMKQDGLIFIE | |
| | HLCHKTLAYHYEPLDEDDWYTEYFFPAGTLTLISSSFLLYFQDDVSV | |
| | VDHWTLSGKHFSRSNEEWLKRMDEKIDEVKEIFESVSDSKDDDVTK | |
| | LINHWRFFCISSAEMFGYNNGEEWMISQVLFKKK | |
| CchNMT3 | MIKKSKIMAFSDHHHEVVKNHSKKEMIADLLKRLEAGLVPDEEMRN | 80 |
| | LFRFELERRLQWGYKSIHQEQLSQLLKLAHSTKEMTIVAEMDALNSS | |
| | MYELPISFLQIQLGSNLKQSSLYFKDELTTVDEAEVAIMDLYLERAQI | |
| | EDGQSILDLGCGLGAFSFHVARKYTNCNITAVTNSLTQKEFIEKKSKI | |
| | LNIQNVKVIFADVTTVEMETTFDRVFAIGLIEHMQNYELFLKKLSKW | |
| | MKQDGLLFIEHFCHKTLAYHYKPIDEDDWFTNLLYPNGTVISSSLLL | |
| | YFQDDVSVVDHWSLSGKHFSRASEESLKRMDAKMDEMKEIFESITD | |
| | SKEEAMKLINQWRIFCISCAEMFGYNNGEEWMTSHFLFKKKL | |
| CchNMT6 | MGSSTASDHEMVIMENDSKNKQVVIADLLKRLVGGLVPDEEMRNM | 81 |
| | FRFELEKRLKWGYKSTHQQQLSQLLNLVELNKGIAKIAPEMDALNS | |
| | AMYEVPIPYLKLMLGSTLKQSCLYFKDESTTLDEAEIEMMDLYLERA | |
| | DIQDGQSILDLGCGLGGLGFHIAQKYISCNITALTNSLTQKEFIEEKCK | |
| | TLNIPNVKVILADVTTVEIETTFDRLFAIGLVEHMENYELFLRKLSKW | |
| | MKQDGLLFIEHLCHKTLAYHYKPIDEDDWYSNLLYPTGTLTSASFLL | |
| | YFQDDLSVVDHWSLSGKHFSRATEEWLKMIDANMDKIREIYESVTE | |
| | SKEEATRSINQWRIFCISCAEMFGYNDGEEWMISHFLFKNKKQIE | |
| CchNMT1 | MATSDQEVKTSKMEMIADLLKRLEAGLVPDDEIRSLIRVELERRLKW | 82 |
| | GYKSTHQEQLDQLLNLAHSIKKMKIASTEMDGLTSTMYEVPISLVQI | |
| | QLGSHLKESCLYFKDETTTVDEAEIAMMDLYLERAQIKDGQSILDLG | |
| | CGLGAVSFHIAQKYTSCNITAVTNSVRQKEFIEEKSKTLNVPNVKVL | |
| | LADITTLEMEHTFDRLFAISLIEHMENYELLLRKLSEWMKQDGLLFIE | |
| | HLCHKTLSYHFEPMDEDDWYTNLLFPAGTLTLVSASFLLYFQDDLS | |
| | VVNQWVMSGKHFSRANEEWLKNMDAKMDEMREIFESITDSEEEVV | |
| | KLINHWRIFCISSAEMFAYNDGEEWMNSHVLFKKKKQIQ | |
| CchNMT2 | MAGSGANKEMIADLLKRLEVGLVPDEEIRSLIRFQLKRRLKWGYKT | 83 |
| | THQEQLEQLLSLAHSIRKMKIATEMDALNSTMYEVPISFMQIVFGSTL | |
| | KESCLYFKDEATTVNEAEIAMMDLYLERAQIKDGQSILDLGCGMGS | |
| | LCFHIARKYTNCNITAVTNSVSQKEFIEEKSKTLNLPNVKVILADITTL | |
| | EMDDTYDCLFAIGLIEHMKNYELLLRKLSNWMKQDSLLFIDHVCHK | |
| | TLAYHYEPIDEDDWYTNLLFPAGTLTLVSASFLLYFQDDLSLVDHWS | |
| | MSGKHFSRTNKEWLKNIDGKMDKIREIVKSITDSEEEVVKLINHWR | |
| | MLCINSSEMFGFNDGEEWMNSHVLFKKKKQI | |
| ScaNMT2 | MEMIADLLKRLEAGLVPDDEIRSLIRVELERRLKWGYKSTHQEQLDQ | 84 |
| | LLNLAHSIKKMKIASTEMDGLTSTMYEVPISLVQIQLGSHLKESCLYF | |
| | KDETTTVDEAEIAMMDLYLERAQIKDGQSILDLGCGLGSVCFHIARK | |
| | YTSCNITAVTNSVSQKEFIEEKSKTLNVPNVKVLLADITTLEMDDTFD | |
| | CLFAIGLIEHMENYELLLRKLSDWMKQDGLLFIDHVCHKTLSYHFEP | |
| | MDEDDWYTNLLFPAGTLTLVSASFLLYFQDDLSLVDHWSMSGKHFS | |
| | RTNKEWLKNIDGKMDKIREIVKSITDSEEEVVKLINHWRMLCINSSE | |
| | MFGFNDGEEWMNSHVLFKKKKQI | |
| PbrNMT2 | MCTTMDTTKISQQDDLWKNMELGLISDEEVRRLMKIETEKRIKWGT | 85 |
| | KPTQQEQLAQLLDFNKSLRGMKMATEVHALENHKIYEIPDSFNQIIG | |
| | GKESAGLFTDEATTTIEEANTKMMDLYCERAGLKDGQTILDIGCGA | |
| | GLLVLHLAKKYKNCKITGVTNTSWHKEHILEQCKNLNLSNVEVILA | |
| | DVTTVDIERTFDRVFVIGLIEHMKNFELFLRKISKWMKDDGLLFLEH | |
| | LCHKSFSDHWEPLSEDDWYAKNFFPSGTLVIPSATCLLYFQEDVTVK | |
| | DHWLLSGNNFARSNEAILKRIDSKIEEVKDIFMSFYGIGEEEAVKLIN | |
| | WWRLLCITANELFKYNNGEEWLISQLLFKKKLMTCI | |
| PbrNMT1 | MVKGDQFQTTTMEETKISQENDLWTNMELGLIPDEEVRRLMKIEIEK | 86 |
| | RIEWGMKPTQHQQLAQLLDFTKSLRGMKMATELDKLDSKLYETPHS | |
| | FNQIVNGSTLKESSGLYTDVTTTMDEASIKMMDLYCERANIKDGQTI | |
| | LDLGCGPGPLVLHIAKKYSNCKITGVTNAFSQREYILEECKKLSLSNV | |
| | EIILADVTSLDLETTFDRVFVIGFIEHMKNFELFLRKISKWMKDDAVL | |
| | FLEHFCHKSFSYHGEPLSEDDWYAKNFFAPGTLVIPSATCLLYFQEDL | |
| | AVIDHWFLSGNHFARTNEEMLKGIDGKIEEIKDIFMSFYGINEAEAVK | |
| | LINWWRLFCITGAEMFSYNNGEEWFISQLLFKKK | |
| EcaNMT4 | MALEQEDSMSVPERNEGVADLIKRMELGLVNDEEIRRLMRIQIENRL | 87 |
| | KWGYKPTHDQQLAQHLHFINSLKEMKMATEMDSLDSQVYESPNSF | |
| | QQIMCGRSMKESAGLFMDDVTTVEEAHIRMMDLYCDKATFEDGQK | |
| | ILDLGCGHGSVVLHVAQKYKGCQVTGVTNSSAQKQYILEQCKKLDL | |
| | SNVEIILADVTTLEMEEKFDRVIIIGLIEHMKNFKLFFQKVSKWMKEG | |
| | GLLFLENYFHKDFAYHCEKIDEDDWYDGYIFPPGSLLMPSASTLLYF | |
| | QEDLTVADHWVLPGTHFAKTFEEFLKKIDLRIEEVREIFEAFYGISKE | |
| | EAMKLSNYWRNFCISAMEIFNYNNGQEWMISHLLYTKK | |
| CmaNMT5 | METGKNNQNMKTTIDDLWNQMMLGIVPDKEIRRLMKIELKKRLDW | 88 |
| | GYRPTHQQQLSQLLDFAKGLCNYCWTALRCMKMSAEFDTLDSKVY | |
| | ETPKSFQQIMCGTTIKESSGLFMNESTTLDQAQISMLDLYFDKAKIKD | |
| | GQSILDLGCGHGALILYLAQKYQNCNITGVTNSLSQKEFIVEKCKKL | |
| | GLSNVEILLADVTKLEMEDMFDRVFVIGLIEHMKNFELFLRKISEWM | |
| | KPDGLLFLEHYCHKSFAHQWEPIDEEDWFSKYIFPPGTVIIPSASFLLY | |
| | FQEDVKVIDHWTLSGNHFARTQEEWLKGIDGHIDEVEKTFESFYGIS | |
| | KEEAVKLINFWRVFCLSGVEMFGYNNGEEWMISHLLFKKK | |
| GflNMT4 | MTMEANNAKKEAIENLWEQMMMGLVPDHEITRLMKSELQKRLNW | 89 |
| | GYKPTHQQQISQLLDFAKSLRRMEMSLDFDNLELDTKMYETPESFQL | |
| | IMSGTTLKESSGLFTDETATLDQTQIRMMDLYLEKAKIKDGQSILDL | |
| | GCGHGALILHVAQKYRNCNVTGVTNSIAQKEFIFKQCKKLGLSNVE | |
| | MVLADVTKCEMKATFDHIFVIGLIEHMKNFELFLRKVSEWMKSDGL | |
| | LFMEHYCHKSFAYQWEPMDDDDLFSKYVFPPGSAIIPSASFLLYFQD | |
| | DLTVVDHWTLSGNHFARTHQEWLKRIDSQSDEIKGIFESFYGISKEEA | |
| | VKLINYWRVFCLFGVEMFGYNNGEEWMISHLLFKKK | |
| CchNMT5 | MEVVATSSARNPKKEIVDLWKRMELGLIPDEEIRDLMKIGLEKRLK | 90 |
| | WGYKPTHEQQLSQLLHFAKSLRSMKMASEMETLDDQMYETPTAFQ | |
| | QLMCGSTIKESAGFFKDESTTLDEAEIKMLDLYCEKARIEDGQKILDL | |
| | GCGHGAVMLHIAQKYKNCNVTGVTNSISQQQFIVQRSKELNLSNVN | |
| | MILADVTMLEMDATYDRIFIIGLIEHMKNFELFLRKISKWITKEGLLF | |
| | LEHYCHKTFAYQCEPVDEDDWYNMFIFPPGTLILPSASFLLYFQDDLI | |
| | VVDRWTLNGNHYARTQEEWLKRIDANVDGVKQMFESVCDGNKEE | |
| | AVKLMNFWRIFCISGAEMLAYNNGEEWMISHYLFKKRN | |
| NsNMT2 | MEATQITKKQGVAELIKRIENGQVPDEEITRMMKIQIQKRLKLGYKS | 91 |
| | THEQQLAQLLHFVHSLQKMEMAEEVDTLDSELYEIPLPFLHIMCGKA | |
| | LKFSPGYFKDESTTLDESEVNMLDLYCERAQIEDGQTILDLGCGHGS | |
| | LTLHVAKKYRGCKVTGITNSVSQKDFIMEECKKLNLSNVEIILEDVT | |
| | KFETGTTYDRIFAVALIEHMKNYELFLKKVSAWMAQDGLLFVEHHC | |
| | HKVFAYKYEPIDDDDWYTEYIFPTGTLVMSSSSILLYFQEDVSVVNH | |
| | WTLSGKHPSLGFKQWLKRIDDNIDEIKEIFESFYGSKEKATKFITYWR | |
| | VFCIAHSEMYATNGGEEWMLSQVLFKRK | |
| ScaNMT5 | MGGVADLLKKMELGLVPEEEIRRLMRIIIEKRLEWGYKPTHAEQLDH | 92 |
| | LTNFIQCLRGMKMADEIDALDAKMYEIPLPFMQTICGSTLKFSPGYF | |
| | KDESTTLDESEIHMMDLYCERAEVKDGHSILDLGCGHGGFVLHVAQ | |
| | KYKNSIVTGVTNSVAEKEFIMTQCKKLCLSNVEIILADVTKFEPETTY | |
| | DRVFAIALIEHMKNYELVLEKLSKWVAQDGFLFVEHHCHKVFPYKY | |
| | EPLDEDDWYTEYIFPGGTIVLPSASILLYFQKDVSVVNHWSLNGKHP | |
| | ARGFKEWLKRLDENMDAVKAIFEPFYGSKEEAMKWITYWRVFCITH | |
| | SEMYAYNNGEEWMLSQVLFKRK | |
| JdiNMT1 | MSKGVAKLVERMELGLVSDDEVRRLMRILIEKRLKWGYKPTHEEQL | 93 |
| | TYLTNFIQGLKGMKIAEEIDALDAKMYEIPIAFMQILCGYSLKFSPGFF | |
| | EDESTTLDESETIMMDLYCERAQVQDGQSILDLGCGHGGFVLHVAQ | |
| | KYKNCKVTGVTNSVSETEYIMEQCKKLGLSNVEIIIADVTKFEPEVTY | |
| | DRVFAIALIEHMKNYELVLQKLSKWVAQDGFLFVDHHCHKVFPYK | |
| | YEPIDEDDWYTQYIFPGGTLVLPSASILLYFQEDVSIVNHWTLSGNHP | |
| | ARGFKEWLKRLDDNMDEIKAIFEPFYGSKEEAMKWITYWRVFCITH | |
| | SEMYAYNGGEEWMISQVLFKRK | |
| BthNMT1 | MEVKQAGKEGVTELLVKRMELGLVPEEEIRRLMRIQIQKRLDWGYK | 94 |
| | PTHEEQLAHLTKFIQNIRGMKMADEIDALDAKMYEIPLPFLQTICGKT | |
| | LKFSPGYFKDESTTLDESETLMMDLYCERAQVKDGQSILDLGCGHG | |
| | GFVLHLAQKYRNSVVTGVTNSVSETEYIKEQCKKLGLSNVEIIIADVT | |
| | KFEPEVTYDRVFAIALIEHMKNYALVLNKISKWVAQDGYLFVEHHC | |
| | HKVFPYKYEPLDEDDWYTNYIFPGGTLILPSASILLYFQEDVTVLNH | |
| | WSLSGKHPSRGFIEWLKRLDENIDVIMGIFEPFYGSKEEATKWINYW | |
| | RVFCMTHSEMYAYGNGEEWMLSQVLLKRK | |
| MaqNMT3 | MELGLVPEKEIRRLMRIQIQKRLEWGYKPTHEEQLAHLTKFIQNIRG | 95 |
| | MKMADEIDALDAKMYEIPLPFLQTICGKTLKFSPGYFKDESTTLDESE | |
| | TLMMDLYCERAQVKDGQSILDLGCGHGGFVLHLAQKYRNSIVTGV | |
| | TNSVSETEYIKEQCKKLGLSNVEIIIADVTKFEPEVTYDRVFAIALIEH | |
| | MKNYALVLNKISKWVAQDGYLFVEHHCHKVFPYKYEPLDEDDWY | |
| | TNYIFPGGTLILPSASILLYFQEDVTVLNHWSLSGKHPSRGFIEWLKRL | |
| | DENIDVIMGIFEPFYGSKEEATKWINYWRVFCITHSEMYAYGNGEEW | |
| | MLSQVLLKRK | |
| McaNMT4 | MDKANERELKRAELFKKLEDDLVTYDEIKQVMRTELAKRLEWGYK | 96 |
| | PTHQQQLAHLLDFAHALEGMKIANEVETLASEVYETPLPFXEIVLGP | |
| | AKKXSSCLFEDESTTLEQAEIAMLDLYFERAQIRXGMSVLDLGCGXG | |
| | SVGLHIARKYKNCXVTCITNSISQKQYIENQCKLYNLSNVKIILADIV | |
| | AHDTDDTFDVVLVIGVIEHMKNYALLLNKISKWMAKDGLLFVEHLC | |
| | HKTFPYHFEPLDEDDWYSNFVFPTGTLTMPSVSFLLYFQADVSILNH | |
| | WILSGKNFSRTXEEFLKRIDANVDAIKDGLKPSLGSEGVAKLISYWR | |
| | GFCLTGMEMFGYNNGEEWMVSQVLFKNK | |
| TcoNMT3 | MEDNNNLLQEEMNVVELLQRPELGLVPDEKIRKLTRLQLQKRLKW | 97 |
| | GYKPTHEAQLSHLFQFIHSLPSLNMESEDENPKSWLYETPTSFLQLLY | |
| | GDCIKESDTYYKEDTATLEEAVINMLELYCERARITEGLSVLDLGCG | |
| | YGALTLHVAQKYKSCKVTGVTSSISQKQYIMEKCKKLNLTNVEIILA | |
| | DVATIEIEAASYDRIFALGIFEHVNDYKLFLGKLSKWMKQDGLLFVE | |
| | YLCHKTFPYQNKPLDKGDKWYNEYVFPSGGLIIPSASFILYFQNDVS | |
| | VVRQWTQGGQHSARTFEELLKRIDGNIDKIKEIFIESYGSKEDAVRFI | |
| | NYWRVFLITGVEMFSYNDGEEWMGAHFLFKKKFIMQE | |
| CmuNMT4 | MEVKQSKGDELRSRVAELLERPELGLVPDEEIRRLAKARLEKRLKW | 98 |
| | GYKATHGEQLSSLLQFVESLPSLNMASEDDSPKAWLYETPTSFLQLI | |
| | YGDIIKESGSYYKDESTTLEEAMIHNMNLCCERANIKEGQSVVDLGC | |
| | GYGAFILHVAQKYKTCRVTGITSSISQKHYIMEQCKKLNLSNVEVILA | |
| | DVATIKLDATFDRVFAAGMFEHVNDYKSFLRKITNWMKPDGRLFVE | |
| | HLCNKTFPYQNKPLDDGDNWGEYVFPSGGLIIPSASLLLYFQEDVSIV | |
| | NHWTFSGKHAANKFEELLKRIDAKIDAIKRIFNECYGSKDSIRFINYW | |
| | RVFLITAAEMFGYNNGEEWMGVHLLFKKK | |
| CtrNMT2 | GLKSSVAELLERPELGLVPDGEIRKLTKTRLAKRLEWGYKATHEDQL | 99 |
| | SHLLRFIHSLPSLNMASEDDSPKAWLYETPTSFLQLIYGDIIKESGTYY | |
| | KDESSTLEEAIIHNMDLCCERARIKEGQSVLDLGCGYGAFTLHVAQK | |
| | YKSCSVTGITSSISQKDYIMEQCKKLNLSNVEVILADVATIKMNTTFD | |
| | RVFALGMFEHINDYKLFLRRISNWMKHDGLLFVEHLCNKTFAYQNK | |
| | PLDDGDDWFNEYVFPSAGLIIPSASLLLYFQEDVSIVHHWTFSGKHA | |
| | AYKFEELLERIDAKIEAIKEIFIECYGSKEDAIRFINYWRVFLITAAEMF | |
| | AYRDGEEWMGSHVLFKKK | |
| CmuNMT5 | MEAKQHESNNNIDEELKNRVNIGEQEERPGFEDEEIRRLAKAQLAKR | 100 |
| | LKWGYKPTHEQQLSHLLQFLQSLPSLNMASEDESSKAWLYETPTSFL | |
| | QLLFGNVIKFSGYYYKHESSTFEESMIHNMDLCCERANIKEGQNVID | |
| | LGCGYGAFVLHVAQKYKSCSVTGITCSITQKHHIMEECKKLNLCNV | |
| | KVILADVATIELGTAFDRVFAFGMFEEINDYKLILRKISNWMKPDGLF | |
| | FVEHLCHKTLAYQNKLIDDQDWYEEYIFPSGGLIVPSASLLLYFQDD | |
| | LSVVYHWTYNGKHGARSFEKMLERTDANIDTIKDMFTEFYGSKEKA | |
| | IKFINYWRVFFITAAEMFAYNDGEEWMCSQLLFKKK | |
| CmuNMT8 | MEHKIEDIRKLKSRVEEQLERPELGLVKDEDIKTLAKAKLEKRLKWG | 101 |
| | YKPTYAEQLSNLLQFAQSLPSLKMENVDDQGSSKQWLYGVPSEFLQI | |
| | IYGGIIKMSGSYYEDESTTLEESMIKDMDSCCEKANVKEGHSVLDIG | |
| | CGYGSLIIHIAKKYRTCNVTGITNFVEQKQYIMEECKKLNLSNVEVIV | |
| | GDGTTINLNTTTFDRVFVTGMLEEINDYKLFLKSVSDWMKPDGLLL | |
| | VTHFCHKTFAYQNNKALDDEDWHNEYIFPSGNLIVPSASLLLYFQED | |
| | LSVVSHWATNGTHTGRTCKKLVERIDANIEKIKEIFSEFYGSKEDAIR | |
| | MINYWRVLCITGAEMYTCKDGEEWMDVYYLFKKK | |

TABLE 6
Variants of BM3 N-demethylase

| BM3 | | |
|---|---|---|
| variant | | SEQ ID NO: |
| | Genotype | |
| 8F11 | L437A | |
| 4H9 | L181A, T260A, L437A | |
| 8C7 | L75A, L181A | |
| 4H5 | L75A, M177A, L181A | |
| 7A1 | L75A, M177A, L181A, T260A | |
| | Amino Acid Sequence | |
| 8F11 | MTIKEMPQPKTFGELKNLPLLNTDKPVQALMKIADELGEIFKFEA | 102 |
| | PGRVTRYLSSQRLIKEACDESRFDKNLSQALKFARDFAGDGLVTS | |
| | WTHEKNWKKAHNILLPSFSQQAMKGYHAMMVDIAVQLVQKWE | |
| | RLNADEHIEVSEDMTRLTLDTIGLCGFNYRFNSFYRDQPHPFIISM | |
| | VRALDEVMNKLQRANPDDPAYDENKRQFQEDIKVMNDLVDKII | |
| | ADRKARGEQSDDLLTQMLNGKDPETGEPLDDGNIRYQIITFLIAG | |
| | HETTSGLLSFALYFLVKNPHVLQKVAEEAARVLVDPVPSYKQVK | |
| | QLKYVGMVLNEALRLWPTAPAFSLYAKEDTVLGGEYPLEKGDE | |
| | VMVLIPQLHRDKTVWGDDVEEFRPERFENPSAIPQHAFKPFGNG | |
| | QRACIGQQFALHEATLVLGMMLKHFDFEDHTNYELDIKETATLK | |
| | PKGFVVKAKSKKIPLGGIPSPSTEQSAKKVRKKAENAHNTPLLVL | |
| | YGSNMGTAEGTARDLADIAMSKGFAPQVATLDSHAGNLPREGA | |
| | VLIVTASYNGHPPDNAKQFVDWLDQASADEVKGVRYSVFGCGD | |
| | KNWATTYQKVPAFIDETLAAKGAENIADRGEADASDDFEGTYEE | |
| | WREHMWSDVAAYFNLDIENSEDNKSTLSLQFVDSAADMPLAKM | |
| | HGAFSTNVVASKELQQPGSARSTRHLEIELPKEASYQEGDHLGVI | |
| | PRNYEGIVNRVTARFGLDASQQIRLEAEEEKLAHLPLAKTVSVEE | |
| | LLQYVELQDPVTRTQLRAMAAKTVCPPHKVELEALLEKQAYKE | |
| | QVLAKRLTMLELLEKYPACEMKFSEFIALLPSIRPRYYSISSSPRV | |
| | DEKQASITVSVVSGEAWSGYGEYKGIASNYLAELQEGDTITCFIST | |
| | PQSEFTLPKDPETPLIMVGPGTGVAPFRGFVQARKQLKEQGQSLG | |
| | EAHLYFGCRSPHEDYLYQEELENAQSEGIITLHTAFSRMPNQPKT | |
| | YVQHVMEQDGKKLIELLDQGAHFYICGDGSQMAPAVEATLMKS | |
| | YADVHQVSEADARLWLQQLEEKGRYAKDVWAG | |
| 4H9 | MTIKEMPQPKTFGELKNLPLLNTDKPVQALMKIADELGEIFKFEA | 103 |
| | PGRVTRYLSSQRLIKEACDESRFDKNLSQALKFARDFAGDGLVTS | |
| | WTHEKNWKKAHNILLPSFSQQAMKGYHAMMVDIAVQLVQKWE | |
| | RLNADEHIEVSEDMTRLTLDTIGLCGFNYRFNSFYRDQPHPFIISM | |
| | VRAADEVMNKLQRANPDDPAYDENKRQFQEDIKVMNDLVDKII | |
| | ADRKARGEQSDDLLTQMLNGKDPETGEPLDDGNIRYQIIAFLIAG | |
| | HETTSGLLSFALYFLVKNPHVLQKVAEEAARVLVDPVPSYKQVK | |
| | QLKYVGMVLNEALRLWPTAPAFSLYAKEDTVLGGEYPLEKGDE | |
| | VMVLIPQLHRDKTVWGDDVEEFRPERFENPSAIPQHAFKPFGNG | |
| | QRACIGQQFALHEATLVLGMMLKHFDFEDHTNYELDIKETATLK | |
| | PKGFVVKAKSKKIPLGGIPSPSTEQSAKKVRKKAENAHNTPLLVL | |
| | YGSNMGTAEGTARDLADIAMSKGFAPQVATLDSHAGNLPREGA | |
| | VLIVTASYNGHPPDNAKQFVDWLDQASADEVKGVRYSVFGCGD | |
| | KNWATTYQKVPAFIDETLAAKGAENIADRGEADASDDFEGTYEE | |
| | WREHMWSDVAAYFNLDIENSEDNKSTLSLQFVDSAADMPLAKM | |
| | HGAFSTNVVASKELQQPGSARSTRHLEIELPKEASYQEGDHLGVI | |
| | PRNYEGIVNRVTARFGLDASQQIRLEAEEEKLAHLPLAKTVSVEE | |
| | LLQYVELQDPVTRTQLRAMAAKTVCPPHKVELEALLEKQAYKE | |
| | QVLAKRLTMLELLEKYPACEMKFSEFIALLPSIRPRYYSISSSPRV | |
| | DEKQASITVSVVSGEAWSGYGEYKGIASNYLAELQEGDTITCFIST | |
| | PQSEFTLPKDPETPLIMVGPGTGVAPFRGFVQARKQLKEQGQSLG | |
| | EAHLYFGCRSPHEDYLYQEELENAQSEGIITLHTAFSRMPNQPKT | |
| | YVQHVMEQDGKKLIELLDQGAHFYICGDGSQMAPAVEATLMKS | |
| | YADVHQVSEADARLWLQQLEEKGRYAKDVWAG | |
| 8C7 | MTIKEMPQPKTFGELKNLPLLNTDKPVQALMKIADELGEIFKFEA | 104 |
| | PGRVTRYLSSQRLIKEACDESRFDKNLSQAAKFARDFAGDGLVTS | |
| | WTHEKNWKKAHNILLPSFSQQAMKGYHAMMVDIAVQLVQKWE | |
| | RLNADEHIEVSEDMTRLTLDTIGLCGFNYRFNSFYRDQPHPFIISM | |
| | VRAADEVMNKLQRANPDDPAYDENKRQFQEDIKVMNDLVDKII | |
| | ADRKARGEQSDDLLTQMLNGKDPETGEPLDDGNIRYQIITFLIAG | |
| | HETTSGLLSFALYFLVKNPHVLQKVAEEAARVLVDPVPSYKQVK | |
| | QLKYVGMVLNEALRLWPTAPAFSLYAKEDTVLGGEYPLEKGDE | |
| | VMVLIPQLHRDKTVWGDDVEEFRPERFENPSAIPQHAFKPFGNG | |
| | QRACIGQQFALHEATLVLGMMLKHFDFEDHTNYELDIKETLTLK | |
| | PKGFVVKAKSKKIPLGGIPSPSTEQSAKKVRKKAENAHNTPLLVL | |
| | YGSNMGTAEGTARDLADIAMSKGFAPQVATLDSHAGNLPREGA | |
| | VLIVTASYNGHPPDNAKQFVDWLDQASADEVKGVRYSVFGCGD | |
| | KNWATTYQKVPAFIDETLAAKGAENIADRGEADASDDFEGTYEE | |
| | WREHMWSDVAAYFNLDIENSEDNKSTLSLQFVDSAADMPLAKM | |
| | HGAFSTNVVASKELQQPGSARSTRHLEIELPKEASYQEGDHLGVI | |
| | PRNYEGIVNRVTARFGLDASQQIRLEAEEEKLAHLPLAKTVSVEE | |
| | LLQYVELQDPVTRTQLRAMAAKTVCPPHKVELEALLEKQAYKE | |
| | QVLAKRLTMLELLEKYPACEMKFSEFIALLPSIRPRYYSISSSPRV | |
| | DEKQASITVSVVSGEAWSGYGEYKGIASNYLAELQEGDTITCFIST | |
| | PQSEFTLPKDPETPLIMVGPGTGVAPFRGFVQARKQLKEQGQSLG | |
| | EAHLYFGCRSPHEDYLYQEELENAQSEGIITLHTAFSRMPNQPKT | |
| | YVQHVMEQDGKKLIELLDQGAHFYICGDGSQMAPAVEATLMKS | |
| | YADVHQVSEADARLWLQQLEEKGRYAKDVWAG | |
| 4H5 | MTIKEMPQPKTFGELKNLPLLNTDKPVQALMKIADELGEIFKFEA | 105 |
| | PGRVTRYLSSQRLIKEACDESRFDKNLSQAAKFARDFAGDGLVTS | |
| | WTHEKNWKKAHNILLPSFSQQAMKGYHAMMVDIAVQLVQKWE | |
| | RLNADEHIEVSEDMTRLTLDTIGLCGFNYRFNSFYRDQPHPFIISA | |
| | VRAADEVMNKLQRANPDDPAYDENKRQFQEDIKVMNDLVDKII | |
| | ADRKARGEQSDDLLTQMLNGKDPETGEPLDDGNIRYQIITFLIAG | |
| | HETTSGLLSFALYFLVKNPHVLQKVAEEAARVLVDPVPSYKQVK | |
| | QLKYVGMVLNEALRLWPTAPAFSLYAKEDTVLGGEYPLEKGDE | |
| | VMVLIPQLHRDKTVWGDDVEEFRPERFENPSAIPQHAFKPFGNG | |
| | QRACIGQQFALHEATLVLGMMLKHFDFEDHTNYELDIKETLTLK | |
| | PKGFVVKAKSKKIPLGGIPSPSTEQSAKKVRKKAENAHNTPLLVL | |
| | YGSNMGTAEGTARDLADIAMSKGFAPQVATLDSHAGNLPREGA | |
| | VLIVTASYNGHPPDNAKQFVDWLDQASADEVKGVRYSVFGCGD | |
| | KNWATTYQKVPAFIDETLAAKGAENIADRGEADASDDFEGTYEE | |
| | WREHMWSDVAAYFNLDIENSEDNKSTLSLQFVDSAADMPLAKM | |
| | HGAFSTNVVASKELQQPGSARSTRHLEIELPKEASYQEGDHLGVI | |
| | PRNYEGIVNRVTARFGLDASQQIRLEAEEEKLAHLPLAKTVSVEE | |
| | LLQYVELQDPVTRTQLRAMAAKTVCPPHKVELEALLEKQAYKE | |
| | QVLAKRLTMLELLEKYPACEMKFSEFIALLPSIRPRYYSISSSPRV | |
| | DEKQASITVSVVSGEAWSGYGEYKGIASNYLAELQEGDTITCFIST | |
| | PQSEFTLPKDPETPLIMVGPGTGVAPFRGFVQARKQLKEQGQSLG | |
| | EAHLYFGCRSPHEDYLYQEELENAQSEGIITLHTAFSRMPNQPKT | |
| | YVQHVMEQDGKKLIELLDQGAHFYICGDGSQMAPAVEATLMKS | |
| | YADVHQVSEADARLWLQQLEEKGRYAKDVWAG | |
| | MTIKEMPQPKTFGELKNLPLLNTDKPVQALMKIADELGEIFKFEA | 106 |
| 7A1 | PGRVTRYLSSQRLIKEACDESRFDKNLSQAAKFARDFAGDGLVTS | |
| | WTHEKNWKKAHNILLPSFSQQAMKGYHAMMVDIAVQLVQKWE | |
| | RLNADEHIEVSEDMTRLTLDTIGLCGFNYRFNSFYRDQPHPFIISA | |
| | VRAADEVMNKLQRANPDDPAYDENKRQFQEDIKVMNDLVDKII | |
| | ADRKARGEQSDDLLTQMLNGKDPETGEPLDDGNIRYQIIAFLIAG | |
| | HETTSGLLSFALYFLVKNPHVLQKVAEEAARVLVDPVPSYKQVK | |
| | QLKYVGMVLNEALRLWPTAPAFSLYAKEDTVLGGEYPLEKGDE | |
| | VMVLIPQLHRDKTVWGDDVEEFRPERFENPSAIPQHAFKPFGNG | |
| | QRACIGQQFALHEATLVLGMMLKHFDFEDHTNYELDIKETLTLK | |
| | PKGFVVKAKSKKIPLGGIPSPSTEQSAKKVRKKAENAHNTPLLVL | |
| | YGSNMGTAEGTARDLADIAMSKGFAPQVATLDSHAGNLPREGA | |
| | VLIVTASYNGHPPDNAKQFVDWLDQASADEVKGVRYSVFGCGD | |
| | KNWATTYQKVPAFIDETLAAKGAENIADRGEADASDDFEGTYEE | |
| | WREHMWSDVAAYFNLDIENSEDNKSTLSLQFVDSAADMPLAKM | |
| | HGAFSTNVVASKELQQPGSARSTRHLEIELPKEASYQEGDHLGVI | |
| | PRNYEGIVNRVTARFGLDASQQIRLEAEEEKLAHLPLAKTVSVEE | |
| | LLQYVELQDPVTRTQLRAMAAKTVCPPHKVELEALLEKQAYKE | |
| | QVLAKRLTMLELLEKYPACEMKFSEFIALLPSIRPRYYSISSSPRV | |
| | DEKQASITVSVVSGEAWSGYGEYKGIASNYLAELQEGDTITCFIST | |
| | PQSEFTLPKDPETPLIMVGPGTGVAPFRGFVQARKQLKEQGQSLG | |
| | EAHLYFGCRSPHEDYLYQEELENAQSEGIITLHTAFSRMPNQPKT | |
| | YVQHVMEQDGKKLIELLDQGAHFYICGDGSQMAPAVEATLMKS | |
| | YADVHQVSEADARLWLQQLEEKGRYAKDVWAG | |
| | Nucleotide Sequence | |
| | ATGACCATCAAAGAAATGCCACAACCTAAGACTTTCGGTGAA | 107 |
| 8F11 | TTGAAGAATTTGCCTTTGTTGAACACCGATAAGCCAGTTCAAG | |
| | CTTTGATGAAGATTGCTGATGAATTGGGTGAAATCTTCAAGTT | |
| | TGAAGCTCCAGGTAGAGTCACTAGATACTTGTCATCTCAAAGA | |
| | TTGATCAAAGAAGCCTGCGACGAATCCAGATTTGATAAGAATT | |
| | TGTCTCAAGCTTTGAAGTTCGCTAGAGATTTTGCTGGTGATGG | |
| | TTTGGTTACTTCTTGGACTCACGAAAAGAATTGGAAGAAGGCC | |
| | CATAACATTTTGTTGCCATCTTTCTCACAACAAGCCATGAAGG | |
| | GTTATCATGCTATGATGGTTGATATCGCCGTTCAATTGGTTCA | |
| | AAAGTGGGAAAGATTGAACGCCGATGAACATATCGAAGTCTC | |
| | TGAAGATATGACCAGATTGACCTTGGATACCATTGGTTTGTGT | |
| | GGTTTCAACTACAGATTCAACTCCTTCTACAGAGATCAACCAC | |
| | ATCCATTCATCATCTCTATGGTTAGAGCTTTGGATGAAGTCAT | |
| | GAACAAATTGCAAAGAGCTAATCCAGACGATCCAGCTTATGA | |
| | CGAAAACAAGAGACAATTCCAAGAAGATATCAAGGTCATGAA | |
| | CGATTTGGTCGATAAGATTATCGCTGATAGAAAGGCTAGAGGT | |
| | GAACAATCTGATGATTTGTTGACCCAAATGTTGAACGGTAAGG | |
| | ATCCAGAAACTGGTGAACCATTGGATGATGGTAACATCAGAT | |
| | ACCAAATTATCACCTTCTTGATTGCTGGTCACGAAACTACATC | |
| | TGGTTTGTTGTCTTTTGCCTTGTACTTTTTGGTTAAGAACCCAC | |
| | ACGTCTTGCAAAAGGTTGCTGAAGAAGCTGCAAGAGTTTTGGT | |
| | TGATCCAGTTCCATCTTACAAGCAAGTCAAGCAATTGAAGTAC | |
| | GTTGGTATGGTTTTGAACGAAGCTTTGAGATTGTGGCCAACTG | |
| | CTCCAGCTTTTTCATTATACGCTAAAGAAGATACCGTCTTGGG | |
| | TGGTGAATATCCATTGGAAAAAGGTGATGAAGTTATGGTCTTG | |
| | ATCCCACAATTGCATAGAGATAAGACTGTTTGGGGTGATGATG | |
| | TCGAAGAATTCAGACCAGAAAGATTCGAAAACCCATCTGCTA | |
| | TTCCACAACATGCTTTTAAGCCATTTGGTAACGGTCAAAGAGC | |
| | TTGCATTGGTCAACAATTCGCTTTACATGAAGCTACCTTGGTTT | |
| | TGGGTATGATGTTGAAACACTTCGACTTCGAAGATCACACCAA | |
| | CTACGAATTGGATATCAAAGAAACCGCTACCTTGAAGCCAAA | |
| | GGGTTTTGTTGTTAAGGCTAAGTCCAAAAAGATTCCATTGGGT | |
| | GGTATTCCATCTCCATCTACTGAACAATCCGCTAAGAAGGTTA | |
| | GAAAGAAAGCTGAAAACGCTCATAACACACCTTTGTTGGTCTT | |
| | GTACGGTTCTAATATGGGTACTGCTGAAGGTACAGCAAGAGA | |
| | TTTGGCAGATATTGCTATGTCTAAAGGTTTCGCTCCACAAGTT | |
| | GCTACTTTGGATTCTCATGCTGGTAATTTGCCAAGAGAAGGTG | |
| | CTGTTTTGATAGTTACTGCTTCTTACAATGGTCACCCACCAGAT | |
| | AATGCTAAGCAATTCGTTGATTGGTTGGATCAAGCTTCAGCTG | |
| | ATGAAGTAAAAGGTGTTAGATACTCTGTTTTCGGTTGCGGTGA | |
| | CAAAAATTGGGCTACTACTTATCAAAAGGTTCCAGCCTTTATT | |
| | GACGAAACTTTGGCTGCTAAAGGTGCTGAAAACATTGCTGAC | |
| | AGAGGTGAAGCTGATGCCTCCGACGACTTCGAAGGTACTTAC | |
| | GAAGAATGGAGAGAACACATGTGGTCTGACGTTGCTGCTTACT | |
| | TCAACTTGGACATCGAAAACTCTGAAGACAACAAGTCCACTTT | |
| | GTCTTTGCAATTCGTTGACTCCGCTGCTGACATGCCATTGGCTA | |
| | AGATGCACGGTGCTTTCTCTACCAACGTCGTTGCCTCCAAGGA | |
| | ATTGCAACAACCAGGTTCTGCTAGATCTACTAGACACTTGGAA | |
| | ATCGAATTGCCAAAGGAAGCTTCCTACCAAGAAGGTGACCAC | |
| | TTGGGCGTTATTCCAAGAAACTACGAAGGTATCGTCAACAGA | |
| | GTTACTGCTAGATTCGGTTTGGATGCTTCTCAACAAATCAGAT | |
| | TAGAAGCTGAAGAAGAAAAGTTGGCTCACTTGCCATTAGCTA | |
| | AGACTGTCTCCGTTGAAGAATTGTTGCAATACGTCGAATTGCA | |
| | AGACCCAGTTACCAGAACCCAATTGAGAGCCATGGCTGCCAA | |
| | GACCGTCTGTCCACCACACAAGGTTGAATTGGAAGCCTTGTTG | |
| | GAAAAGCAAGCCTACAAGGAACAAGTTTTGGCTAAGAGATTG | |
| | ACCATGTTGGAATTGTTGGAAAAGTACCCAGCCTGCGAAATG | |
| | AAGTTCTCTGAATTTATCGCCTTGTTGCCATCTATCAGACCACG | |
| | TTACTACTCTATTTCTTCCTCTCCACGTGTTGACGAAAAGCAAG | |
| | CTTCTATTACTGTTTCCGTTGTCTCCGGTGAAGCTTGGTCCGGT | |
| | TACGGTGAATACAAGGGTATTGCTTCTAACTACTTGGCTGAAT | |
| | TGCAAGAAGGTGACACCATTACTTGTTTCATCTCTACTCCACA | |
| | ATCCGAATTTACTTTGCCAAAGGACCCAGAAACTCCATTGATC | |
| | ATGGTTGGTCCAGGTACTGGTGTCGCTCCATTCAGAGGTTTCG | |
| | TTCAAGCTAGAAAACAATTGAAGGAACAAGGTCAATCTTTGG | |
| | GTGAAGCTCACTTGTACTTCGGTTGTAGATCTCCACACGAAGA | |
| | CTACTTATACCAAGAAGAATTGGAAAACGCTCAATCCGAAGG | |
| | TATTATCACTTTGCACACCGCTTTCTCCAGAATGCCAAACCAA | |
| | CCAAAGACTTACGTCCAACACGTTATGGAACAAGACGGTAAG | |
| | AAGTTGATTGAATTGTTGGACCAAGGTGCTCACTTCTACATTT | |
| | GTGGTGATGGTTCTCAAATGGCTCCAGCCGTTGAAGCCACTTT | |
| | GATGAAGTCTTACGCTGATGTTCACCAAGTTTCCGAAGCCGAT | |
| | GCTAGATTATGGTTGCAACAATTGGAAGAAAAAGGTCGTTAC | |
| | GCTAAGGATGTCTGGGCCGGTTGA | |
| 4H9 | ATGACCATCAAAGAAATGCCACAACCTAAGACTTTCGGTGAA | 108 |
| | TTGAAGAATTTGCCTTTGTTGAACACCGATAAGCCAGTTCAAG | |
| | CTTTGATGAAGATTGCTGATGAATTGGGTGAAATCTTCAAGTT | |
| | TGAAGCTCCAGGTAGAGTCACTAGATACTTGTCATCTCAAAGA | |
| | TTGATCAAAGAAGCCTGCGACGAATCCAGATTTGATAAGAATT | |
| | TGTCTCAAGCTTTGAAGTTCGCTAGAGATTTTGCTGGTGATGG | |
| | TTTGGTTACTTCTTGGACTCACGAAAAGAATTGGAAGAAGGCC | |
| | CATAACATTTTGTTGCCATCTTTCTCACAACAAGCCATGAAGG | |
| | GTTATCATGCTATGATGGTTGATATCGCCGTTCAATTGGTTCA | |
| | AAAGTGGGAAAGATTGAACGCCGATGAACATATCGAAGTCTC | |
| | TGAAGATATGACCAGATTGACCTTGGATACCATTGGTTTGTGT | |
| | GGTTTCAACTACAGATTCAACTCCTTCTACAGAGATCAACCAC | |
| | ATCCATTCATCATCTCTATGGTTAGAGCTGCAGATGAAGTCAT | |
| | GAACAAATTGCAAAGAGCTAATCCAGACGATCCAGCTTATGA | |
| | CGAAAACAAGAGACAATTCCAAGAAGATATCAAGGTCATGAA | |
| | CGATTTGGTCGATAAGATTATCGCTGATAGAAAGGCTAGAGGT | |
| | GAACAATCTGATGATTTGTTGACCCAAATGTTGAACGGTAAGG | |
| | ATCCAGAAACTGGTGAACCATTGGATGATGGTAACATCAGAT | |
| | ACCAAATTATCGCTTTCTTGATTGCTGGTCACGAAACTACATC | |
| | TGGTTTGTTGTCTTTTGCCTTGTACTTTTTGGTTAAGAACCCAC | |
| | ACGTCTTGCAAAAGGTTGCTGAAGAAGCTGCAAGAGTTTTGGT | |
| | TGATCCAGTTCCATCTTACAAGCAAGTCAAGCAATTGAAGTAC | |
| | GTTGGTATGGTTTTGAACGAAGCTTTGAGATTGTGGCCAACTG | |
| | CTCCAGCTTTTTCATTATACGCTAAAGAAGATACCGTCTTGGG | |
| | TGGTGAATATCCATTGGAAAAAGGTGATGAAGTTATGGTCTTG | |
| | ATCCCACAATTGCATAGAGATAAGACTGTTTGGGGTGATGATG | |
| | TCGAAGAATTCAGACCAGAAAGATTCGAAAACCCATCTGCTA | |
| | TTCCACAACATGCTTTTAAGCCATTTGGTAACGGTCAAAGAGC | |
| | TTGCATTGGTCAACAATTCGCTTTACATGAAGCTACCTTGGTTT | |
| | TGGGTATGATGTTGAAACACTTCGACTTCGAAGATCACACCAA | |
| | CTACGAATTGGATATCAAAGAAACCGCTACCTTGAAGCCAAA | |
| | GGGTTTTGTTGTTAAGGCTAAGTCCAAAAAGATTCCATTGGGT | |
| | GGTATTCCATCTCCATCTACTGAACAATCCGCTAAGAAGGTTA | |
| | GAAAGAAAGCTGAAAACGCTCATAACACACCTTTGTTGGTCTT | |
| | GTACGGTTCTAATATGGGTACTGCTGAAGGTACAGCAAGAGA | |
| | TTTGGCAGATATTGCTATGTCTAAAGGTTTCGCTCCACAAGTT | |
| | GCTACTTTGGATTCTCATGCTGGTAATTTGCCAAGAGAAGGTG | |
| | CTGTTTTGATAGTTACTGCTTCTTACAATGGTCACCCACCAGAT | |
| | AATGCTAAGCAATTCGTTGATTGGTTGGATCAAGCTTCAGCTG | |
| | ATGAAGTAAAAGGTGTTAGATACTCTGTTTTCGGTTGCGGTGA | |
| | CAAAAATTGGGCTACTACTTATCAAAAGGTTCCAGCCTTTATT | |
| | GACGAAACTTTGGCTGCTAAAGGTGCTGAAAACATTGCTGAC | |
| | AGAGGTGAAGCTGATGCCTCCGACGACTTCGAAGGTACTTAC | |
| | GAAGAATGGAGAGAACACATGTGGTCTGACGTTGCTGCTTACT | |
| | TCAACTTGGACATCGAAAACTCTGAAGACAACAAGTCCACTTT | |
| | GTCTTTGCAATTCGTTGACTCCGCTGCTGACATGCCATTGGCTA | |
| | AGATGCACGGTGCTTTCTCTACCAACGTCGTTGCCTCCAAGGA | |
| | ATTGCAACAACCAGGTTCTGCTAGATCTACTAGACACTTGGAA | |
| | ATCGAATTGCCAAAGGAAGCTTCCTACCAAGAAGGTGACCAC | |
| | TTGGGCGTTATTCCAAGAAACTACGAAGGTATCGTCAACAGA | |
| | GTTACTGCTAGATTCGGTTTGGATGCTTCTCAACAAATCAGAT | |
| | TAGAAGCTGAAGAAGAAAAGTTGGCTCACTTGCCATTAGCTA | |
| | AGACTGTCTCCGTTGAAGAATTGTTGCAATACGTCGAATTGCA | |
| | AGACCCAGTTACCAGAACCCAATTGAGAGCCATGGCTGCCAA | |
| | GACCGTCTGTCCACCACACAAGGTTGAATTGGAAGCCTTGTTG | |
| | GAAAAGCAAGCCTACAAGGAACAAGTTTTGGCTAAGAGATTG | |
| | ACCATGTTGGAATTGTTGGAAAAGTACCCAGCCTGCGAAATG | |
| | AAGTTCTCTGAATTTATCGCCTTGTTGCCATCTATCAGACCACG | |
| | TTACTACTCTATTTCTTCCTCTCCACGTGTTGACGAAAAGCAAG | |
| | CTTCTATTACTGTTTCCGTTGTCTCCGGTGAAGCTTGGTCCGGT | |
| | TACGGTGAATACAAGGGTATTGCTTCTAACTACTTGGCTGAAT | |
| | TGCAAGAAGGTGACACCATTACTTGTTTCATCTCTACTCCACA | |
| | ATCCGAATTTACTTTGCCAAAGGACCCAGAAACTCCATTGATC | |
| | ATGGTTGGTCCAGGTACTGGTGTCGCTCCATTCAGAGGTTTCG | |
| | TTCAAGCTAGAAAACAATTGAAGGAACAAGGTCAATCTTTGG | |
| | GTGAAGCTCACTTGTACTTCGGTTGTAGATCTCCACACGAAGA | |
| | CTACTTATACCAAGAAGAATTGGAAAACGCTCAATCCGAAGG | |
| | TATTATCACTTTGCACACCGCTTTCTCCAGAATGCCAAACCAA | |
| | CCAAAGACTTACGTCCAACACGTTATGGAACAAGACGGTAAG | |
| | AAGTTGATTGAATTGTTGGACCAAGGTGCTCACTTCTACATTT | |
| | GTGGTGATGGTTCTCAAATGGCTCCAGCCGTTGAAGCCACTTT | |
| | GATGAAGTCTTACGCTGATGTTCACCAAGTTTCCGAAGCCGAT | |
| | GCTAGATTATGGTTGCAACAATTGGAAGAAAAAGGTCGTTAC | |
| | GCTAAGGATGTCTGGGCCGGTTGA | |
| 8C7 | ATGACCATCAAAGAAATGCCACAACCTAAGACTTTCGGTGAA | 109 |
| | TTGAAGAATTTGCCTTTGTTGAACACCGATAAGCCAGTTCAAG | |
| | CTTTGATGAAGATTGCTGATGAATTGGGTGAAATCTTCAAGTT | |
| | TGAAGCTCCAGGTAGAGTCACTAGATACTTGTCATCTCAAAGA | |
| | TTGATCAAAGAAGCCTGCGACGAATCCAGATTTGATAAGAATT | |
| | TGTCTCAAGCTGCTAAGTTCGCTAGAGATTTTGCTGGTGATGG | |
| | TTTGGTTACTTCTTGGACTCACGAAAAGAATTGGAAGAAGGCC | |
| | CATAACATTTTGTTGCCATCTTTCTCACAACAAGCCATGAAGG | |
| | GTTATCATGCTATGATGGTTGATATCGCCGTTCAATTGGTTCA | |
| | AAAGTGGGAAAGATTGAACGCCGATGAACATATCGAAGTCTC | |
| | TGAAGATATGACCAGATTGACCTTGGATACCATTGGTTTGTGT | |
| | GGTTTCAACTACAGATTCAACTCCTTCTACAGAGATCAACCAC | |
| | ATCCATTCATCATCTCTATGGTTAGAGCTGCAGATGAAGTCAT | |
| | GAACAAATTGCAAAGAGCTAATCCAGACGATCCAGCTTATGA | |
| | CGAAAACAAGAGACAATTCCAAGAAGATATCAAGGTCATGAA | |
| | CGATTTGGTCGATAAGATTATCGCTGATAGAAAGGCTAGAGGT | |
| | GAACAATCTGATGATTTGTTGACCCAAATGTTGAACGGTAAGG | |
| | ATCCAGAAACTGGTGAACCATTGGATGATGGTAACATCAGAT | |
| | ACCAAATTATCACCTTCTTGATTGCTGGTCACGAAACTACATC | |
| | TGGTTTGTTGTCTTTTGCCTTGTACTTTTTGGTTAAGAACCCAC | |
| | ACGTCTTGCAAAAGGTTGCTGAAGAAGCTGCAAGAGTTTTGGT | |
| | TGATCCAGTTCCATCTTACAAGCAAGTCAAGCAATTGAAGTAC | |
| | GTTGGTATGGTTTTGAACGAAGCTTTGAGATTGTGGCCAACTG | |
| | CTCCAGCTTTTTCATTATACGCTAAAGAAGATACCGTCTTGGG | |
| | TGGTGAATATCCATTGGAAAAAGGTGATGAAGTTATGGTCTTG | |
| | ATCCCACAATTGCATAGAGATAAGACTGTTTGGGGTGATGATG | |
| | TCGAAGAATTCAGACCAGAAAGATTCGAAAACCCATCTGCTA | |
| | TTCCACAACATGCTTTTAAGCCATTTGGTAACGGTCAAAGAGC | |
| | TTGCATTGGTCAACAATTCGCTTTACATGAAGCTACCTTGGTTT | |
| | TGGGTATGATGTTGAAACACTTCGACTTCGAAGATCACACCAA | |
| | CTACGAATTGGATATCAAAGAAACCTTGACCTTGAAGCCAAA | |
| | GGGTTTTGTTGTTAAGGCTAAGTCCAAAAAGATTCCATTGGGT | |
| | GGTATTCCATCTCCATCTACTGAACAATCCGCTAAGAAGGTTA | |
| | GAAAGAAAGCTGAAAACGCTCATAACACACCTTTGTTGGTCTT | |
| | GTACGGTTCTAATATGGGTACTGCTGAAGGTACAGCAAGAGA | |
| | TTTGGCAGATATTGCTATGTCTAAAGGTTTCGCTCCACAAGTT | |
| | GCTACTTTGGATTCTCATGCTGGTAATTTGCCAAGAGAAGGTG | |
| | CTGTTTTGATAGTTACTGCTTCTTACAATGGTCACCCACCAGAT | |
| | AATGCTAAGCAATTCGTTGATTGGTTGGATCAAGCTTCAGCTG | |
| | ATGAAGTAAAAGGTGTTAGATACTCTGTTTTCGGTTGCGGTGA | |
| | CAAAAATTGGGCTACTACTTATCAAAAGGTTCCAGCCTTTATT | |
| | GACGAAACTTTGGCTGCTAAAGGTGCTGAAAACATTGCTGAC | |
| | AGAGGTGAAGCTGATGCCTCCGACGACTTCGAAGGTACTTAC | |
| | GAAGAATGGAGAGAACACATGTGGTCTGACGTTGCTGCTTACT | |
| | TCAACTTGGACATCGAAAACTCTGAAGACAACAAGTCCACTTT | |
| | GTCTTTGCAATTCGTTGACTCCGCTGCTGACATGCCATTGGCTA | |
| | AGATGCACGGTGCTTTCTCTACCAACGTCGTTGCCTCCAAGGA | |
| | ATTGCAACAACCAGGTTCTGCTAGATCTACTAGACACTTGGAA | |
| | ATCGAATTGCCAAAGGAAGCTTCCTACCAAGAAGGTGACCAC | |
| | TTGGGCGTTATTCCAAGAAACTACGAAGGTATCGTCAACAGA | |
| | GTTACTGCTAGATTCGGTTTGGATGCTTCTCAACAAATCAGAT | |
| | TAGAAGCTGAAGAAGAAAAGTTGGCTCACTTGCCATTAGCTA | |
| | AGACTGTCTCCGTTGAAGAATTGTTGCAATACGTCGAATTGCA | |
| | AGACCCAGTTACCAGAACCCAATTGAGAGCCATGGCTGCCAA | |
| | GACCGTCTGTCCACCACACAAGGTTGAATTGGAAGCCTTGTTG | |
| | GAAAAGCAAGCCTACAAGGAACAAGTTTTGGCTAAGAGATTG | |
| | ACCATGTTGGAATTGTTGGAAAAGTACCCAGCCTGCGAAATG | |
| | AAGTTCTCTGAATTTATCGCCTTGTTGCCATCTATCAGACCACG | |
| | TTACTACTCTATTTCTTCCTCTCCACGTGTTGACGAAAAGCAAG | |
| | CTTCTATTACTGTTTCCGTTGTCTCCGGTGAAGCTTGGTCCGGT | |
| | TACGGTGAATACAAGGGTATTGCTTCTAACTACTTGGCTGAAT | |
| | TGCAAGAAGGTGACACCATTACTTGTTTCATCTCTACTCCACA | |
| | ATCCGAATTTACTTTGCCAAAGGACCCAGAAACTCCATTGATC | |
| | ATGGTTGGTCCAGGTACTGGTGTCGCTCCATTCAGAGGTTTCG | |
| | TTCAAGCTAGAAAACAATTGAAGGAACAAGGTCAATCTTTGG | |
| | GTGAAGCTCACTTGTACTTCGGTTGTAGATCTCCACACGAAGA | |
| | CTACTTATACCAAGAAGAATTGGAAAACGCTCAATCCGAAGG | |
| | TATTATCACTTTGCACACCGCTTTCTCCAGAATGCCAAACCAA | |
| | CCAAAGACTTACGTCCAACACGTTATGGAACAAGACGGTAAG | |
| | AAGTTGATTGAATTGTTGGACCAAGGTGCTCACTTCTACATTT | |
| | GTGGTGATGGTTCTCAAATGGCTCCAGCCGTTGAAGCCACTTT | |
| | GATGAAGTCTTACGCTGATGTTCACCAAGTTTCCGAAGCCGAT | |
| | GCTAGATTATGGTTGCAACAATTGGAAGAAAAAGGTCGTTAC | |
| | GCTAAGGATGTCTGGGCCGGTTGA | |
| 4H5 | ATGACCATCAAAGAAATGCCACAACCTAAGACTTTCGGTGAA | 110 |
| | TTGAAGAATTTGCCTTTGTTGAACACCGATAAGCCAGTTCAAG | |
| | CTTTGATGAAGATTGCTGATGAATTGGGTGAAATCTTCAAGTT | |
| | TGAAGCTCCAGGTAGAGTCACTAGATACTTGTCATCTCAAAGA | |
| | TTGATCAAAGAAGCCTGCGACGAATCCAGATTTGATAAGAATT | |
| | TGTCTCAAGCTGCTAAGTTCGCTAGAGATTTTGCTGGTGATGG | |
| | TTTGGTTACTTCTTGGACTCACGAAAAGAATTGGAAGAAGGCC | |
| | CATAACATTTTGTTGCCATCTTTCTCACAACAAGCCATGAAGG | |
| | GTTATCATGCTATGATGGTTGATATCGCCGTTCAATTGGTTCA | |
| | AAAGTGGGAAAGATTGAACGCCGATGAACATATCGAAGTCTC | |
| | TGAAGATATGACCAGATTGACCTTGGATACCATTGGTTTGTGT | |
| | GGTTTCAACTACAGATTCAACTCCTTCTACAGAGATCAACCAC | |
| | ATCCATTCATCATCTCTGCTGTTAGAGCTGCAGATGAAGTCAT | |
| | GAACAAATTGCAAAGAGCTAATCCAGACGATCCAGCTTATGA | |
| | CGAAAACAAGAGACAATTCCAAGAAGATATCAAGGTCATGAA | |
| | CGATTTGGTCGATAAGATTATCGCTGATAGAAAGGCTAGAGGT | |
| | GAACAATCTGATGATTTGTTGACCCAAATGTTGAACGGTAAGG | |
| | ATCCAGAAACTGGTGAACCATTGGATGATGGTAACATCAGAT | |
| | ACCAAATTATCACCTTCTTGATTGCTGGTCACGAAACTACATC | |
| | TGGTTTGTTGTCTTTTGCCTTGTACTTTTTGGTTAAGAACCCAC | |
| | ACGTCTTGCAAAAGGTTGCTGAAGAAGCTGCAAGAGTTTTGGT | |
| | TGATCCAGTTCCATCTTACAAGCAAGTCAAGCAATTGAAGTAC | |
| | GTTGGTATGGTTTTGAACGAAGCTTTGAGATTGTGGCCAACTG | |
| | CTCCAGCTTTTTCATTATACGCTAAAGAAGATACCGTCTTGGG | |
| | TGGTGAATATCCATTGGAAAAAGGTGATGAAGTTATGGTCTTG | |
| | ATCCCACAATTGCATAGAGATAAGACTGTTTGGGGTGATGATG | |
| | TCGAAGAATTCAGACCAGAAAGATTCGAAAACCCATCTGCTA | |
| | TTCCACAACATGCTTTTAAGCCATTTGGTAACGGTCAAAGAGC | |
| | TTGCATTGGTCAACAATTCGCTTTACATGAAGCTACCTTGGTTT | |
| | TGGGTATGATGTTGAAACACTTCGACTTCGAAGATCACACCAA | |
| | CTACGAATTGGATATCAAAGAAACCTTGACCTTGAAGCCAAA | |
| | GGGTTTTGTTGTTAAGGCTAAGTCCAAAAAGATTCCATTGGGT | |
| | GGTATTCCATCTCCATCTACTGAACAATCCGCTAAGAAGGTTA | |
| | GAAAGAAAGCTGAAAACGCTCATAACACACCTTTGTTGGTCTT | |
| | GTACGGTTCTAATATGGGTACTGCTGAAGGTACAGCAAGAGA | |
| | TTTGGCAGATATTGCTATGTCTAAAGGTTTCGCTCCACAAGTT | |
| | GCTACTTTGGATTCTCATGCTGGTAATTTGCCAAGAGAAGGTG | |
| | CTGTTTTGATAGTTACTGCTTCTTACAATGGTCACCCACCAGAT | |
| | AATGCTAAGCAATTCGTTGATTGGTTGGATCAAGCTTCAGCTG | |
| | ATGAAGTAAAAGGTGTTAGATACTCTGTTTTCGGTTGCGGTGA | |
| | CAAAAATTGGGCTACTACTTATCAAAAGGTTCCAGCCTTTATT | |
| | GACGAAACTTTGGCTGCTAAAGGTGCTGAAAACATTGCTGAC | |
| | AGAGGTGAAGCTGATGCCTCCGACGACTTCGAAGGTACTTAC | |
| | GAAGAATGGAGAGAACACATGTGGTCTGACGTTGCTGCTTACT | |
| | TCAACTTGGACATCGAAAACTCTGAAGACAACAAGTCCACTTT | |
| | GTCTTTGCAATTCGTTGACTCCGCTGCTGACATGCCATTGGCTA | |
| | AGATGCACGGTGCTTTCTCTACCAACGTCGTTGCCTCCAAGGA | |
| | ATTGCAACAACCAGGTTCTGCTAGATCTACTAGACACTTGGAA | |
| | ATCGAATTGCCAAAGGAAGCTTCCTACCAAGAAGGTGACCAC | |
| | TTGGGCGTTATTCCAAGAAACTACGAAGGTATCGTCAACAGA | |
| | GTTACTGCTAGATTCGGTTTGGATGCTTCTCAACAAATCAGAT | |
| | TAGAAGCTGAAGAAGAAAAGTTGGCTCACTTGCCATTAGCTA | |
| | AGACTGTCTCCGTTGAAGAATTGTTGCAATACGTCGAATTGCA | |
| | AGACCCAGTTACCAGAACCCAATTGAGAGCCATGGCTGCCAA | |
| | GACCGTCTGTCCACCACACAAGGTTGAATTGGAAGCCTTGTTG | |
| | GAAAAGCAAGCCTACAAGGAACAAGTTTTGGCTAAGAGATTG | |
| | ACCATGTTGGAATTGTTGGAAAAGTACCCAGCCTGCGAAATG | |
| | AAGTTCTCTGAATTTATCGCCTTGTTGCCATCTATCAGACCACG | |
| | TTACTACTCTATTTCTTCCTCTCCACGTGTTGACGAAAAGCAAG | |
| | CTTCTATTACTGTTTCCGTTGTCTCCGGTGAAGCTTGGTCCGGT | |
| | TACGGTGAATACAAGGGTATTGCTTCTAACTACTTGGCTGAAT | |
| | TGCAAGAAGGTGACACCATTACTTGTTTCATCTCTACTCCACA | |
| | ATCCGAATTTACTTTGCCAAAGGACCCAGAAACTCCATTGATC | |
| | ATGGTTGGTCCAGGTACTGGTGTCGCTCCATTCAGAGGTTTCG | |
| | TTCAAGCTAGAAAACAATTGAAGGAACAAGGTCAATCTTTGG | |
| | GTGAAGCTCACTTGTACTTCGGTTGTAGATCTCCACACGAAGA | |
| | CTACTTATACCAAGAAGAATTGGAAAACGCTCAATCCGAAGG | |
| | TATTATCACTTTGCACACCGCTTTCTCCAGAATGCCAAACCAA | |
| | CCAAAGACTTACGTCCAACACGTTATGGAACAAGACGGTAAG | |
| | AAGTTGATTGAATTGTTGGACCAAGGTGCTCACTTCTACATTT | |
| | GTGGTGATGGTTCTCAAATGGCTCCAGCCGTTGAAGCCACTTT | |
| | GATGAAGTCTTACGCTGATGTTCACCAAGTTTCCGAAGCCGAT | |
| | GCTAGATTATGGTTGCAACAATTGGAAGAAAAAGGTCGTTAC | |
| | GCTAAGGATGTCTGGGCCGGTTGA | |
| 7A1 | ATGACCATCAAAGAAATGCCACAACCTAAGACTTTCGGTGAA | 111 |
| | TTGAAGAATTTGCCTTTGTTGAACACCGATAAGCCAGTTCAAG | |
| | CTTTGATGAAGATTGCTGATGAATTGGGTGAAATCTTCAAGTT | |
| | TGAAGCTCCAGGTAGAGTCACTAGATACTTGTCATCTCAAAGA | |
| | TTGATCAAAGAAGCCTGCGACGAATCCAGATTTGATAAGAATT | |
| | TGTCTCAAGCTGCTAAGTTCGCTAGAGATTTTGCTGGTGATGG | |
| | TTTGGTTACTTCTTGGACTCACGAAAAGAATTGGAAGAAGGCC | |
| | CATAACATTTTGTTGCCATCTTTCTCACAACAAGCCATGAAGG | |
| | GTTATCATGCTATGATGGTTGATATCGCCGTTCAATTGGTTCA | |
| | AAAGTGGGAAAGATTGAACGCCGATGAACATATCGAAGTCTC | |
| | TGAAGATATGACCAGATTGACCTTGGATACCATTGGTTTGTGT | |
| | GGTTTCAACTACAGATTCAACTCCTTCTACAGAGATCAACCAC | |
| | ATCCATTCATCATCTCTGCTGTTAGAGCTGCAGATGAAGTCAT | |
| | GAACAAATTGCAAAGAGCTAATCCAGACGATCCAGCTTATGA | |
| | CGAAAACAAGAGACAATTCCAAGAAGATATCAAGGTCATGAA | |
| | CGATTTGGTCGATAAGATTATCGCTGATAGAAAGGCTAGAGGT | |
| | GAACAATCTGATGATTTGTTGACCCAAATGTTGAACGGTAAGG | |
| | ATCCAGAAACTGGTGAACCATTGGATGATGGTAACATCAGAT | |
| | ACCAAATTATCGCTTTCTTGATTGCTGGTCACGAAACTACATC | |
| | TGGTTTGTTGTCTTTTGCCTTGTACTTTTTGGTTAAGAACCCAC | |
| | ACGTCTTGCAAAAGGTTGCTGAAGAAGCTGCAAGAGTTTTGGT | |
| | TGATCCAGTTCCATCTTACAAGCAAGTCAAGCAATTGAAGTAC | |
| | GTTGGTATGGTTTTGAACGAAGCTTTGAGATTGTGGCCAACTG | |
| | CTCCAGCTTTTTCATTATACGCTAAAGAAGATACCGTCTTGGG | |
| | TGGTGAATATCCATTGGAAAAAGGTGATGAAGTTATGGTCTTG | |
| | ATCCCACAATTGCATAGAGATAAGACTGTTTGGGGTGATGATG | |
| | TCGAAGAATTCAGACCAGAAAGATTCGAAAACCCATCTGCTA | |
| | TTCCACAACATGCTTTTAAGCCATTTGGTAACGGTCAAAGAGC | |
| | TTGCATTGGTCAACAATTCGCTTTACATGAAGCTACCTTGGTTT | |
| | TGGGTATGATGTTGAAACACTTCGACTTCGAAGATCACACCAA | |
| | CTACGAATTGGATATCAAAGAAACCTTGACCTTGAAGCCAAA | |
| | GGGTTTTGTTGTTAAGGCTAAGTCCAAAAAGATTCCATTGGGT | |
| | GGTATTCCATCTCCATCTACTGAACAATCCGCTAAGAAGGTTA | |
| | GAAAGAAAGCTGAAAACGCTCATAACACACCTTTGTTGGTCTT | |
| | GTACGGTTCTAATATGGGTACTGCTGAAGGTACAGCAAGAGA | |
| | TTTGGCAGATATTGCTATGTCTAAAGGTTTCGCTCCACAAGTT | |
| | GCTACTTTGGATTCTCATGCTGGTAATTTGCCAAGAGAAGGTG | |
| | CTGTTTTGATAGTTACTGCTTCTTACAATGGTCACCCACCAGAT | |
| | AATGCTAAGCAATTCGTTGATTGGTTGGATCAAGCTTCAGCTG | |
| | ATGAAGTAAAAGGTGTTAGATACTCTGTTTTCGGTTGCGGTGA | |
| | CAAAAATTGGGCTACTACTTATCAAAAGGTTCCAGCCTTTATT | |
| | GACGAAACTTTGGCTGCTAAAGGTGCTGAAAACATTGCTGAC | |
| | AGAGGTGAAGCTGATGCCTCCGACGACTTCGAAGGTACTTAC | |
| | GAAGAATGGAGAGAACACATGTGGTCTGACGTTGCTGCTTACT | |
| | TCAACTTGGACATCGAAAACTCTGAAGACAACAAGTCCACTTT | |
| | GTCTTTGCAATTCGTTGACTCCGCTGCTGACATGCCATTGGCTA | |
| | AGATGCACGGTGCTTTCTCTACCAACGTCGTTGCCTCCAAGGA | |
| | ATTGCAACAACCAGGTTCTGCTAGATCTACTAGACACTTGGAA | |
| | ATCGAATTGCCAAAGGAAGCTTCCTACCAAGAAGGTGACCAC | |
| | TTGGGCGTTATTCCAAGAAACTACGAAGGTATCGTCAACAGA | |
| | GTTACTGCTAGATTCGGTTTGGATGCTTCTCAACAAATCAGAT | |
| | TAGAAGCTGAAGAAGAAAAGTTGGCTCACTTGCCATTAGCTA | |
| | AGACTGTCTCCGTTGAAGAATTGTTGCAATACGTCGAATTGCA | |
| | AGACCCAGTTACCAGAACCCAATTGAGAGCCATGGCTGCCAA | |
| | GACCGTCTGTCCACCACACAAGGTTGAATTGGAAGCCTTGTTG | |
| | GAAAAGCAAGCCTACAAGGAACAAGTTTTGGCTAAGAGATTG | |
| | ACCATGTTGGAATTGTTGGAAAAGTACCCAGCCTGCGAAATG | |
| | AAGTTCTCTGAATTTATCGCCTTGTTGCCATCTATCAGACCACG | |
| | TTACTACTCTATTTCTTCCTCTCCACGTGTTGACGAAAAGCAAG | |
| | CTTCTATTACTGTTTCCGTTGTCTCCGGTGAAGCTTGGTCCGGT | |
| | TACGGTGAATACAAGGGTATTGCTTCTAACTACTTGGCTGAAT | |
| | TGCAAGAAGGTGACACCATTACTTGTTTCATCTCTACTCCACA | |
| | ATCCGAATTTACTTTGCCAAAGGACCCAGAAACTCCATTGATC | |
| | ATGGTTGGTCCAGGTACTGGTGTCGCTCCATTCAGAGGTTTCG | |
| | TTCAAGCTAGAAAACAATTGAAGGAACAAGGTCAATCTTTGG | |
| | GTGAAGCTCACTTGTACTTCGGTTGTAGATCTCCACACGAAGA | |
| | CTACTTATACCAAGAAGAATTGGAAAACGCTCAATCCGAAGG | |
| | TATTATCACTTTGCACACCGCTTTCTCCAGAATGCCAAACCAA | |
| | CCAAAGACTTACGTCCAACACGTTATGGAACAAGACGGTAAG | |
| | AAGTTGATTGAATTGTTGGACCAAGGTGCTCACTTCTACATTT | |
| | GTGGTGATGGTTCTCAAATGGCTCCAGCCGTTGAAGCCACTTT | |
| | GATGAAGTCTTACGCTGATGTTCACCAAGTTTCCGAAGCCGAT | |
| | GCTAGATTATGGTTGCAACAATTGGAAGAAAAAGGTCGTTAC | |
| | GCTAAGGATGTCTGGGCCGGTTGA | |

TABLE 7
pA24, pA25, and pA26 sequences

| | | |
|---|---|---|
| pA24 | cctcgccgcagttaattaaagtcagtgagcgaggaagcgcgtaactataacggtcctaaggtagcg | SEQ ID |
| Se- | aatcctgatgcggtattttctccttacgcatctgtgcggtatttcacaccgcatagatcggcaagtgca | NO: 112 |
| quence | caaacaatacttaaataaatactactcagtaataacctatttcttagcatttttgacgaaatttgctattttgt | |
| | tagagtcttttacaccatttgtctccacacctccgcttacatcaacaccaataacgccatttaatctaagc | |
| | gcatcaccaacattttctggcgtcagtccaccagctaacataaaatgtaagctttcggggctctcttgc | |
| | cttccaacccagtcagaaatcgagttccaatccaaaagttcacctgtcccacctgcttctgaatcaaa | |
| | caagggaataaacgaatgaggtttctgtgaagctgcactgagtagtatgttgcagtcttttggaaatac | |
| | gagtcttttaataactggcaaaccgaggaactcttggtattcttgccacgactcatctccatgcagtgg | |
| | agccaatcaattcttgcggtcaactttggacgatatcaatgccgtaatcattgaccagagccaaaaca | |
| | tcctccttaagttgattacgaaacacgccaaccaagtatttcggagtgcctgaactatttttatatgctttt | |
| | acaagacttgaaattttccttgcaataaccgggtcaattgttctctttctattgggcacacatataatacc | |
| | cagcaagtcagcatcggaatctagagcacattctgcggcctctgtgctctgcaagccgcaaactttc | |
| | accaatggaccagaactacctgtgaaattaataacagacatactccaagctgcctttgtgtgcttaatc | |
| | acgtatactcacgtgctcaatagtcaccaatgccctccctcttggccctctccttttcttttttcgaccga | |
| | attaattcttaatcggcaaaaaaagaaaagctccggatcaagattgtacgtaaggtgacaagctatttt | |
| | tcaataaagaatatcttccactactgccatctggcgtcataactgcaaagtacacatatattacgatgct | |
| | gttctattaaatgcttcctatattatatatatagtaatgtcgtgatctatggtgcactctcagtacaatctgc | |
| | tctgatgccgcatagttaagccagccccgacacccgccaacacccgctgacgcgccctgacggg | |
| | cttgtctgctcccggcatccgcttacagacaagctgtgaccgtctccgggagctgcatgtgtcagag | |
| | gttttcaccgtcatcaccgaaacgcgcgagacgaaagggcctcgtgatacgcctatttttataggtta | |
| | atgtcatgataataatggtttcttagacggatcgcttgcctgtaacttacacgcgcctcgtatcttttaat | |
| | gatggaataatttgggaatttactctgtgtttatttatttttatgttttgtatttggattttagaaagtaaataaa | |
| | gaaggtagaagagttacggaatgaagaaaaaaaaataaacaaaggtttaaaaaatttcaacaaaaa | |
| | gcgtactttacatatatatttattagacaagaaaagcagattaaatagatatacattcgattaacgataa | |
| | gtaaaatgtaaaatcacaggattttcgtgtgtggtcttctacacagacaaggtgaaacaattcggcatt | |
| | aatacctgagagcaggaagagcaagataaaaggtagtatttgttggcgatccccctagagtcttttac | |
| | atcttcggaaaacaaaaactattttttctttaatttctttttttactttctatttttaatttatatatttatattaaaa | |
| | aatttaaattataattatttttatagcacgtgatgaaaaggacccaggtggcacttttcggggaaatgtg | |
| | cgcggaacccctatttgtttatttttctaaatacattcaaatatgtatccgctcatgagacaataaccctg | |
| | ataaatgcttcaataatattgaaaaaggaagagtatgagtattcaacatttccgtgtcgcccttattccct | |
| | tttttgcggcattttgccttcctgtttttgctcacccagaaacgctggtgaaagtaaaagatgctgaaga | |
| | tcagttgggacgcgtagtctagaccagccaggacagaaatgcctcgacttcgctgctacccaaggt | |
| | tgccgggtgacgcacaccgtggaaacggatgaaggcacgaacccagtggacataagcctgttcg | |
| | gttcgtaagctgtaatgcaagtagcgtatgcgctcacgcaactggtccagaaccttgaccgaacgca | |
| | gcggtggtaacggcgcagtggcggttttcatggcttgttatgactgtttttttggggtacagtctatgcc | |
| | tcgggcatccaagcagcaagcgcgttacgccgtgggtcgatgtttgatgttatggagcagcaacga | |
| | tgttacgcagcagggcagtcgccctaaaacaaagttaaacattatgagggaagcggtgatcgccga | |
| | agtatcgactcaactatcagaggtagttggcgccatcgagcgccatctcgaaccgacgttgctggc | |
| | cgtacatttgtacggctccgcagtggatggcggcctgaagccacacagtgatattgatttgctggtta | |
| | cggtgaccgtaaggcttgatgaaacaacgcggcgagctttgatcaacgaccttttggaaacttcggc | |
| | ttcccctggagagagcgagattctccgcgctgtagaagtcaccattgttgtgcacgacgacatcattc | |
| | cgtggcgttatccagctaagcgcgaactgcaatttggagaatggcagcgcaatgacattcttgcagg | |
| | tatcttcgagccagccacgatcgacattgatctggctatcttgctgacaaaagcaagagaacatagc | |
| | gttgccttggtaggtccagcggcggaggaactctttgatccggttcctgaacaggatctatttgaggc | |
| | gctaaatgaaaccttaacgctatggaactcgccgcccgactgggctggcgatgagcgaaatgtagt | |
| | gcttacgttgtcccgcatttggtacagcgcagtaaccggcaaaatcgcgccgaaggatgtcgctgc | |
| | cggctgggcaatggagcgcctgccggcccagtatcagcccgtcatacttgaagctagacaggctt | |
| | atcttggacaagaagaagatcgcttggcctcgcgcgcagatcagttggaagaatttgtccactacgt | |
| | gaaaggcgagatcaccaaggtagtcggcaaataaccctcgagcattcaaggcgccttgattatttga | |
| | cgtggtttgatggcctccacgcacgttgtgatatgtagatgattcagttcgagtttatcattatcaatact | |
| | gccatttcaaagaatacgtaaataattaatagtagtgattttcctaactttatttagtcaaaaaattagcctt | |
| | ttaattctgctgtaacccgtacatgcccaaaatagggggcgggttacacagaatatataacatcgtag | |
| | gtgtctgggtgaacagtttattcctggcatccactaaatataatggagcccgctttttaagctggcatcc | |
| | agaaaaaaaaagaatcccagcaccaaaatattgttttcttcaccaaccatcagttcataggtccattct | |
| | cttagcgcaactacagagaacaggggcacaaacaggcaaaaaacgggcacaacctcaatggagt | |
| | gatgcaacctgcctggagtaaatgatgacacaaggcaattgacccacgcatgtatctatctcattttct | |
| | tacaccttctattaccttctgctctctctgatttggaaaaagctgaaaaaaaaggttgaaaccagttccc | |
| | tgaaattattcccctacttgactaataagtatataaagacggtaggtattgattgtaattctgtaaatctatt | |
| | tcttaaacttcttaaattctacttttatagttagtcttttttttagttttaaaacaccaagaacttagtttcgaat | |
| | aaacacacataaacaaacaaaacaggccccttttcctttgtcgatatcatgtaattagttatgtcacgct | |
| | tacattcacgccctcctcccacatccgctctaaccgaaaaggaaggagttagacaacctgaagtcta | |
| | ggtccctatttattttttttaatagttatgttagtattaagaacgttatttatatttcaaatttttcttttttttctgta | |
| | caaacgcgtgtacgcatgtaacattatactgaaaaccttgcttgagaaggttttgggacgctcgaagg | |
| | ctttaatttgtaatcattatcactttacgggtcctttccggtgatccgacaggttacggggcggcgacct | |
| | cgcgggttttcgctatttatgaaaattttccggtttaaggcgtttccgttcttcttcgtcataacttaatgttt | |
| | ttatttaaaatacctcgcgagtggcaacactgaaaatacccatggagcggcgtaaccgtcgcacag | |
| | gatctaggtgaagatcctttttgataatctcatgaccaaaatcccttaacgtgagttttcgttccactgag | |
| | cgtcagaccccgtagaaaagatcaaaggatcttcttgagatcctttttttctgcgcgtaatctgctgctt | |
| | gcaaacaaaaaaaccaccgctaccagcggtggtttgtttgccggatcaagagctaccaactctttttc | |
| | cgaaggtaactggcttcagcagagcgcagataccaaatactgtccttctagtgtagccgtagttagg | |
| | ccaccacttcaagaactctgtagcaccgcctacatacctcgctctgctaatcctgttaccagtggctg | |
| | ctgccagtggcgataagtcgtgtcttaccgggttggactcaagacgatagttaccggataaggcgc | |
| | agcggtcgggctgaacggggggttcgtgcacacagcccagcttggagcgaacgacctacaccga | |
| | actgagatacctacagcgtgagctatgagaaagcgccacgcttcccgaagggagaaaggcggac | |
| | aggtatccggtaagcggcagggtcggaacaggagagcgcacgagggagcttccagggggaaa | |
| | cgcctggtatctttatagtcctgtcgggtttcgccacctctgacttgagcgtcgatttttgtgatgctcgt | |
| | caggggggcggagcctatggaaaaacgccagcaacgcggcagtggaacgtgcattatgaattag | |
| | ttacgctagggataacagggtaatatagaacccgaacgaccgagcgcagcggcggccgcgctga | |
| | taccgccgc | |
| pA25 | aacgacctacaccgaactgagatacctacagcgtgagctatgagaaagcgccacgcttcccgaag | SEQ ID |
| sequence | ggagaaaggcggacaggtatccggtaagcggcagggtcggaacaggagagcgcacgaggga | NO: 113 |
| | gcttccagggggaaacgcctggtatctttatagtcctgtcgggtttcgccacctctgacttgagcgtc | |
| | gatttttgtgatgctcgtcaggggggcggagcctatggaaaaacgccagcaacgcggcagtggaa | |
| | cgtgcattatgaattagttacgctagggataacagggtaatatagaacccgaacgaccgagcgcag | |
| | cggcggccgcgctgataccgccgccctcgccgcagttaattaaagtcagtgagcgaggaagcgc | |
| | gtaactataacggtcctaaggtagcgaatcctgatgcggtattttctccttacgcatctgtgcggtattt | |
| | cacaccgcatagatcggcaagtgcacaaacaatacttaaataaatactactcagtaataacctatttct | |
| | tagcatttttgacgaaatttgctattttgttagagtcttttacaccatttgtctccacacctccgcttacatca | |
| | acaccaataacgccatttaatctaagcgcatcaccaacattttctggcgtcagtccaccagctaacat | |
| | aaaatgtaagctttcggggctctcttgccttccaacccagtcagaaatcgagttccaatccaaaagttc | |
| | acctgtcccacctgcttctgaatcaaacaagggaataaacgaatgaggtttctgtgaagctgcactga | |
| | gtagtatgttgcagtcttttggaaatacgagtcttttaataactggcaaaccgaggaactcttggtattct | |
| | tgccacgactcatctccatgcagtggagccaatcaattcttgcggtcaactttggacgatatcaatgc | |
| | cgtaatcattgaccagagccaaaacatcctccttaagttgattacgaaacacgccaaccaagtatttc | |
| | ggagtgcctgaactatttttatatgcttttacaagacttgaaattttccttgcaataaccgggtcaattgtt | |
| | ctctttctattgggcacacatataatacccagcaagtcagcatcggaatctagagcacattctgcggc | |
| | ctctgtgctctgcaagccgcaaactttcaccaatggaccagaactacctgtgaaattaataacagaca | |
| | tactccaagctgcctttgtgtgcttaatcacgtatactcacgtgctcaatagtcaccaatgccctccctc | |
| | ttggccctctccttttcttttttcgaccgaattaattcttaatcggcaaaaaaagaaaagctccggatcaa | |
| | gattgtacgtaaggtgacaagctatttttcaataaagaatatcttccactactgccatctggcgtcataa | |
| | ctgcaaagtacacatatattacgatgctgttctattaaatgcttcctatattatatatatagtaatgtcgtga | |
| | tctatggtgcactctcagtacaatctgctctgatgccgcatagttaagccagccccgacacccgcca | |
| | acacccgctgacgcgccctgacgggcttgtctgctcccggcatccgcttacagacaagctgtgacc | |
| | gtctccgggagctgcatgtgtcagaggttttcaccgtcatcaccgaaacgcgcgagacgaaaggg | |
| | cctcgtgatacgcctatttttataggttaatgtcatgataataatggtttcttagacggatcgcttgcctgt | |
| | aacttacacgcgcctcgtatcttttaatgatggaataatttgggaatttactctgtgtttatttatttttatgtt | |
| | ttgtatttggattttagaaagtaaataaagaaggtagaagagttacggaatgaagaaaaaaaaataaa | |
| | caaaggtttaaaaaatttcaacaaaaagcgtactttacatatatatttattagacaagaaaagcagatta | |
| | aatagatatacattcgattaacgataagtaaaatgtaaaatcacaggattttcgtgtgtggtcttctacac | |
| | agacaaggtgaaacaattcggcattaatacctgagagcaggaagagcaagataaaaggtagtattt | |
| | gttggcgatccccctagagtcttttacatcttcggaaaacaaaaactattttttctttaatttctttttttacttt | |
| | ctatttttaatttatatatttatattaaaaaatttaaattataattatttttatagcacgtgatgaaaaggaccc | |
| | aggtggcacttttcggggaaatgtgcgcggaacccctatttgtttatttttctaaatacattcaaatatgt | |
| | atccgctcatgagacaataaccctgataaatgcttcaataatattgaaaaaggaagagtatgagtattc | |
| | aacatttccgtgtcgcccttattcccttttttgcggcattttgccttcctgtttttgctcacccagaaacgct | |
| | ggtgaaagtaaaagatgctgaagatcagttgggacgcgtagtctagaccagccaggacagaaatg | |
| | cctcgacttcgctgctacccaaggttgccgggtgacgcacaccgtggaaacggatgaaggcacga | |
| | acccagtggacataagcctgttcggttcgtaagctgtaatgcaagtagcgtatgcgctcacgcaact | |
| | ggtccagaaccttgaccgaacgcagcggtggtaacggcgcagtggcggttttcatggcttgttatga | |
| | ctgtttttttggggtacagtctatgcctcgggcatccaagcagcaagcgcgttacgccgtgggtcgat | |
| | gtttgatgttatggagcagcaacgatgttacgcagcagggcagtcgccctaaaacaaagttaaacat | |
| | tatgagggaagcggtgatcgccgaagtatcgactcaactatcagaggtagttggcgccatcgagcg | |
| | ccatctcgaaccgacgttgctggccgtacatttgtacggctccgcagtggatggcggcctgaagcc | |
| | acacagtgatattgatttgctggttacggtgaccgtaaggcttgatgaaacaacgcggcgagctttga | |
| | tcaacgaccttttggaaacttcggcttcccctggagagagcgagattctccgcgctgtagaagtcac | |
| | cattgttgtgcacgacgacatcattccgtggcgttatccagctaagcgcgaactgcaatttggagaat | |
| | ggcagcgcaatgacattcttgcaggtatcttcgagccagccacgatcgacattgatctggctatcttg | |
| | ctgacaaaagcaagagaacatagcgttgccttggtaggtccagcggcggaggaactctttgatccg | |
| | gttcctgaacaggatctatttgaggcgctaaatgaaaccttaacgctatggaactcgccgcccgact | |
| | gggctggcgatgagcgaaatgtagtgcttacgttgtcccgcatttggtacagcgcagtaaccggca | |
| | aaatcgcgccgaaggatgtcgctgccggctgggcaatggagcgcctgccggcccagtatcagcc | |
| | cgtcatacttgaagctagacaggcttatcttggacaagaagaagatcgcttggcctcgcgcgcagat | |
| | cagttggaagaatttgtccactacgtgaaaggcgagatcaccaaggtagtcggcaaataaccctcg | |
| | agcattcaaggcgccttgattatttgacgtggtttgatggcctccacgcacgttgtgatatgtagatga | |
| | gagcgttggttggtggatcaagcccacgcgtaggcaatcctcgagcagatccgccaggcgtgtata | |
| | tatagcgtggatggccaggcaactttagtgctgacacatacaggcatatatatatgtgtgcgacaaca | |
| | catgatcatatggcatgcatgtgctctgtatgtatataaaactcttgttttcttcttttctctaaatattctttc | |
| | cttatacattaggacctttgcagcataaattactatacttctatagacacacaaacacaaatacacacac | |
| | taaattaataacaggccccttttcctttgtcgatatcatgtaattagttatgtcacgcttacattcacgccc | |
| | tccccccacatccgctctaaccgaaaaggaaggagttagacaacctgaagtctaggtccctatttatt | |
| | tttttatagttatgttagtattaagaacgttatttatatttcaaatttttcttttttttctgtacaaacgcgtgtac | |
| | gcatgtaacattatactgaaaaccttgcttgagaaggttttgggacgctcgaaggctttaatttgtaatc | |
| | attatcactttacgggtcctttccggtgatccgacaggttacggggcggcgacctcgcgggttttcgc | |
| | tatttatgaaaattttccggtttaaggcgtttccgttcttcttcgtcataacttaatgtttttatttaaaatacct | |
| | cgcgagtggcaacactgaaaatacccatggagcggcgtaaccgtcgcacaggatctaggtgaag | |
| | atcctttttgataatctcatgaccaaaatcccttaacgtgagttttcgttccactgagcgtcagaccccgt | |
| | agaaaagatcaaaggatcttcttgagatcctttttttctgcgcgtaatctgctgcttgcaaacaaaaaaa | |
| | ccaccgctaccagcggtggtttgtttgccggatcaagagctaccaactctttttccgaaggtaactgg | |
| | cttcagcagagcgcagataccaaatactgtccttctagtgtagccgtagttaggccaccacttcaaga | |
| | actctgtagcaccgcctacatacctcgctctgctaatcctgttaccagtggctgctgccagtggcgat | |
| | aagtcgtgtcttaccgggttggactcaagacgatagttaccggataaggcgcagcggtcgggctga | |
| | acggggggttcgtgcacacagcccagcttggagcg | |
| pA26 | acgacctacaccgaactgagatacctacagcgtgagctatgagaaagcgccacgcttcccgaagg | SEQ ID |
| sequence | gagaaaggcggacaggtatccggtaagcggcagggtcggaacaggagagcgcacgagggag | NO: 114 |
| | cttccagggggaaacgcctggtatctttatagtcctgtcgggtttcgccacctctgacttgagcgtcga | |
| | tttttgtgatgctcgtcaggggggcggagcctatggaaaaacgccagcaacgcggcagtggaacg | |
| | tgcattatgaattagttacgctagggataacagggtaatatagaacccgaacgaccgagcgcagcg | |
| | gcggccgcgctgataccgccgccctcgccgcagttaattaaagtcagtgagcgaggaagcgcgta | |
| | actataacggtcctaaggtagcgaatcctgatgcggtattttctccttacgcatctgtgcggtatttcac | |
| | accgcatagatcggcaagtgcacaaacaatacttaaataaatactactcagtaataacctatttcttag | |
| | catttttgacgaaatttgctattttgttagagtcttttacaccatttgtctccacacctccgcttacatcaac | |
| | accaataacgccatttaatctaagcgcatcaccaacattttctggcgtcagtccaccagctaacataa | |
| | aatgtaagctttcggggctctcttgccttccaacccagtcagaaatcgagttccaatccaaaagttcac | |
| | ctgtcccacctgcttctgaatcaaacaagggaataaacgaatgaggtttctgtgaagctgcactgagt | |
| | agtatgttgcagtcttttggaaatacgagtcttttaataactggcaaaccgaggaactcttggtattcttg | |
| | ccacgactcatctccatgcagtggagccaatcaattcttgcggtcaactttggacgatatcaatgccg | |
| | taatcattgaccagagccaaaacatcctccttaagttgattacgaaacacgccaaccaagtatttcgg | |
| | agtgcctgaactatttttatatgcttttacaagacttgaaattttccttgcaataaccgggtcaattgttctc | |
| | tttctattgggcacacatataatacccagcaagtcagcatcggaatctagagcacattctgcggcctct | |
| | gtgctctgcaagccgcaaactttcaccaatggaccagaactacctgtgaaattaataacagacatact | |
| | ccaagctgcctttgtgtgcttaatcacgtatactcacgtgctcaatagtcaccaatgccctccctcttgg | |
| | ccctctccttttcttttttcgaccgaattaattcttaatcggcaaaaaaagaaaagctccggatcaagatt | |
| | gtacgtaaggtgacaagctatttttcaataaagaatatcttccactactgccatctggcgtcataactgc | |
| | aaagtacacatatattacgatgctgttctattaaatgcttcctatattatatatatagtaatgtcgtgatctat | |
| | ggtgcactctcagtacaatctgctctgatgccgcatagttaagccagccccgacacccgccaacac | |
| | ccgctgacgcgccctgacgggcttgtctgctcccggcatccgcttacagacaagctgtgaccgtct | |
| | ccgggagctgcatgtgtcagaggttttcaccgtcatcaccgaaacgcgcgagacgaaagggcctc | |
| | gtgatacgcctatttttataggttaatgtcatgataataatggtttcttagacggatcgcttgcctgtaact | |
| | tacacgcgcctcgtatcttttaatgatggaataatttgggaatttactctgtgtttatttatttttatgttttgta | |
| | tttggattttagaaagtaaataaagaaggtagaagagttacggaatgaagaaaaaaaaataaacaaa | |
| | ggtttaaaaaatttcaacaaaaagcgtactttacatatatatttattagacaagaaaagcagattaaata | |
| | gatatacattcgattaacgataagtaaaatgtaaaatcacaggattttcgtgtgtggtcttctacacaga | |
| | caaggtgaaacaattcggcattaatacctgagagcaggaagagcaagataaaaggtagtatttgttg | |
| | gcgatccccctagagtcttttacatcttcggaaaacaaaaactattttttctttaatttctttttttactttctat | |
| | ttttaatttatatatttatattaaaaaatttaaattataattatttttatagcacgtgatgaaaaggacccagg | |
| | tggcacttttcggggaaatgtgcgcggaacccctatttgtttatttttctaaatacattcaaatatgtatcc | |
| | gctcatgagacaataaccctgataaatgcttcaataatattgaaaaaggaagagtatgagtattcaac | |
| | atttccgtgtcgcccttattcccttttttgcggcattttgccttcctgtttttgctcacccagaaacgctggt | |
| | gaaagtaaaagatgctgaagatcagttgggacgcgtagtctagaccagccaggacagaaatgcct | |
| | cgacttcgctgctacccaaggttgccgggtgacgcacaccgtggaaacggatgaaggcacgaac | |
| | ccagtggacataagcctgttcggttcgtaagctgtaatgcaagtagcgtatgcgctcacgcaactgg | |
| | tccagaaccttgaccgaacgcagcggtggtaacggcgcagtggcggttttcatggcttgttatgact | |
| | gtttttttggggtacagtctatgcctcgggcatccaagcagcaagcgcgttacgccgtgggtcgatgt | |
| | ttgatgttatggagcagcaacgatgttacgcagcagggcagtcgccctaaaacaaagttaaacatta | |
| | tgagggaagcggtgatcgccgaagtatcgactcaactatcagaggtagttggcgccatcgagcgc | |
| | catctcgaaccgacgttgctggccgtacatttgtacggctccgcagtggatggcggcctgaagcca | |
| | cacagtgatattgatttgctggttacggtgaccgtaaggcttgatgaaacaacgcggcgagctttgat | |
| | caacgaccttttggaaacttcggcttcccctggagagagcgagattctccgcgctgtagaagtcacc | |
| | attgttgtgcacgacgacatcattccgtggcgttatccagctaagcgcgaactgcaatttggagaatg | |
| | gcagcgcaatgacattcttgcaggtatcttcgagccagccacgatcgacattgatctggctatcttgct | |
| | gacaaaagcaagagaacatagcgttgccttggtaggtccagcggcggaggaactctttgatccggt | |
| | tcctgaacaggatctatttgaggcgctaaatgaaaccttaacgctatggaactcgccgcccgactgg | |
| | gctggcgatgagcgaaatgtagtgcttacgttgtcccgcatttggtacagcgcagtaaccggcaaaa | |
| | tcgcgccgaaggatgtcgctgccggctgggcaatggagcgcctgccggcccagtatcagcccgt | |
| | catacttgaagctagacaggcttatcttggacaagaagaagatcgcttggcctcgcgcgcagatca | |
| | gttggaagaatttgtccactacgtgaaaggcgagatcaccaaggtagtcggcaaataaccctcgag | |
| | cattcaaggcgccttgattatttgacgtggtttgatggcctccacgcacgttgtgatatgtagatgactc | |
| | gtaggaacaatttcgggcccctgcgtgttcttctgaggttcatcttttacatttgcttctgctggataatttt | |
| | cagaggcaacaaggaaaaattagatggcaaaaagtcgtctttcaaggaaaaatccccaccatctttc | |
| | gagatcccctgtaacttattggcaactgaaagaatgaaaaggaggaaaatacaaaatatactagaac | |
| | tgaaaaaaaaaaagtataaatagagacgatatatgccaatacttcacaatgttcgaatctattcttcattt | |
| | gcagctattgtaaaataataaaacatcaagaacaaacaagctcaacttgtcttttctaagaacaaaga | |
| | ataaacacaaaaacaaaaagtttttttaattttaatcaaaaaacaggccccttttcctttgtcgatatcatg | |
| | taattagttatgtcacgcttacattcacgccctccccccacatccgctctaaccgaaaaggaaggagt | |
| | tagacaacctgaagtctaggtccctatttatttttttatagttatgttagtattaagaacgttatttatatttca | |
| | aatttttcttttttttctgtacaaacgcgtgtacgcatgtaacattatactgaaaaccttgcttgagaaggtt | |
| | ttgggacgctcgaaggctttaatttgtaatcattatcactttacgggtcctttccggtgatccgacaggtt | |
| | acggggcggcgacctcgcgggttttcgctatttatgaaaattttccggtttaaggcgtttccgttcttctt | |
| | cgtcataacttaatgtttttatttaaaatacctcgcgagtggcaacactgaaaatacccatggagcggc | |
| | gtaaccgtcgcacaggatctaggtgaagatcctttttgataatctcatgaccaaaatcccttaacgtga | |
| | gttttcgttccactgagcgtcagaccccgtagaaaagatcaaaggatcttcttgagatcctttttttctgc | |
| | gcgtaatctgctgcttgcaaacaaaaaaaccaccgctaccagcggtggtttgtttgccggatcaaga | |
| | gctaccaactctttttccgaaggtaactggcttcagcagagcgcagataccaaatactgtccttctagt | |
| | gtagccgtagttaggccaccacttcaagaactctgtagcaccgcctacatacctcgctctgctaatcc | |
| | tgttaccagtggctgctgccagtggcgataagtcgtgtcttaccgggttggactcaagacgatagtta | |
| | ccggataaggcgcagcggtcgggctgaacggggggttcgtgcacacagcccagcttggagcga | |

TABLE 8
Tailoring enzymes

| Reaction | | |
|---|---|---|
| Catalyzed | Enzyme | Species |
| Carbon-carbon | Berberine bridge enzyme (BBE) | Ps, Ec, Cj, |
| coupling | | Bs, Tf |
| | Salutaridine synthase (SalSyn) | Ps |
| | Coryluberine synthase (CorSyn) | Cj |
| Oxidation | Tetrahydroprotoberberine oxidase (STOX) | Cj, Am, Bw |
| | Dihydrobenzophenanthridine oxidase | Ps |
| | (DBOX) | |
| | Methylstylopine hydroxylase (MSH) | Ps |
| | Protopine 6-hydroxylase (PGH) | Ps, Ec |
| Methylenedioxy | Stylopine synthase (StySyn) | Ps, Ec, Am |
| bridge | Cheilanthifoline synthase (CheSyn) | Ps, Ec, Am |
| formation | Canadine synthase (CAS) | Tf, Cc |
| O-methylation | Norcoclaurine 6-O-methyltransferase | Ps, Tf, |
| | (6OMT) | Cj, Pb |
| | 3′hydroxy-N-methylcoclaurine 4′-O- | Ps, Tf, |
| | methytransferase (4′OMT) | Cj, Cc |
| | Reticuline 7-O-methyltransferase (7OMT) | Ps, Ec |
| | Scoulerine 9-O-methyltransferase (9OMT) | Ps, Tf, |
| | | Cj, Cc |
| N-methylation | Coclaurine N-methyltransferase (CNMT) | Ps, Tf, Cj |
| | Tetrahydrprotoberberine N- | Ps, Ec, Pb |
| | methyltransferase (TMNT) | |
| O- | Thebaine demethylase (T6ODM) | Ps |
| demethylation | Codeine demethylase (CODM) | Ps, Ga |
| Reduction | Salutaridine reductase (SalR) | Ps, Pb, Ga |
| | Codeinone reductase (COR) | Ps |
| | Sanguinarine reductase (SanR) | Ec |
| Acetylation | Salutaridine acetyltransferase (SalAT) | Ps |

TABLE 9
Comparison of impurities that may be present in concentrate of
poppy straw and clarified yeast culture medium.

| | | Concentrate | |
|---|---|---|---|
| | | of Poppy | Clarified Yeast Culture |
| Impurities | | Straw | Medium |
| Inorganic | Sodium | ✓ | ✓ |
| | Magnesium | ✓ | ✓ |
| | Silicon | ✓ | X (not in culture medium) |
| | Phosphorus | ✓ | ✓ |
| | Sulfur | ✓ | ✓ |
| | Chlorine | ✓ | ✓ |
| | Potassium | ✓ | ✓ |
| | Calcium | ✓ | ✓ |
| | Copper | ✓ | ✓ |
| | Zinc | ✓ | ✓ |
| | Molybdenum | ✓ | ✓ (sodium molybdenite in |
| | | | medium) |
| | Iron | ✓ | ✓ |
| | Manganese | ✓ | ✓ |
| | Ammonium | ✓ | ✓ |
| | Boron | ✓ | ✓ |
| Organic | Polysaccharides (starch, cellulose, xylan) | ✓ | X (yeast fed simple sugars) |
| | Lignin (p-courmaryl, coniferyl, sinapyl | ✓ | X |
| | alcohols) | ✓ | |
| | Pigments (chlorophyll, anthiocyanins, | ✓ | X |
| | carotenoids) | | |
| | Flavonoids | ✓ | X |
| | Phenanthreoids | ✓ | X |
| | Latex, gum, and wax | ✓ | X |
| | Rubisco | ✓ | X |
| | Meconic acid | ✓ | X |
| | Pseudomorphine | ✓ | X |
| | Narceine | ✓ | X |
| | Thebaol | ✓ | X |
| Other | Pesticides | ✓ | X |
| | Pollen | ✓ | X |

TABLE 10
Distinct groups of molecules present in clarified yeast culture medium (CYCM).
Unlike concentrate of poppy straw (CPS), yeast host strains may be engineered to produce
molecules of a predetermined class of alkaloids (i.e., only one biosynthesis pathway per strain)
such that other classes of alkaloids are not present. Therefore, the CYCM may contain molecules
within a single biosynthesis pathway including a subset of molecules spanning one or two
columns, whereas the CPS may contain a subset of molecules across many columns.

| | Protoberberine | | | | |
|---|---|---|---|---|---|
| 1- | and | | | | |
| Benzylisoquinoline | Phthalideisoquinoline | Morphinan | Isopavine | Aporphine | BisBIA |
| Tetrahydro- | Scoulerine | Salutaridine | Pavine | Magnoflorine | Dauricine |
| papaverine | Stylopine | Salutaridnol | Caryachine | Coryluberine | Berbamunine |
| Dihydro- | Cis-N- | Salutaridine-7-O- | Bisnor- | Aparinorphine | Ligensinine |
| papverine | methylstylopine | acetate | argemonine | Boidine | Fangchinoline |
| Papaverine | Protopine | Thebaine | Isonor- | | Tetradrine |
| | Dihydro- | Codeinone | aremonine | | Curine |
| | sanguinarine | Oripavine | | | Cepharanthine |
| | Tetrahydro- | Morphinone | | | Berbamine |
| | columbarine | Neopinone | | | |
| | N-methylcanadine | Neopine | | | |
| | Noscapine | Codeine | | | |
| | Berberine | Morphine | | | |
| | | Neomorphine | | | |
| | | Hydrocodone | | | |
| | | Oxycodone | | | |
| | | 14- | | | |
| | | hydroxycodeinone | | | |
| | | 14-hydroxycodeine | | | |
| | | Dihydromorphine | | | |
| | | Dihydrocodeine | | | |

TABLE 11
Impurities that may be present in chemical synthesis
preparations of compounds

| Compound | Impurities |
|---|---|
| Buprenorphine | 15,16 -Dehydrobuprenorphine, 17,18- |
| | Dehydrobuprenorphine, 18,19-demethylbuprenorphine, |
| | 19,19′-Ethylbuprenorphine, 2,2′-Bisbuprenorphine, 3- |
| | Deshydroxybuprenorphine, 3-O-Methylbuprenorphine, 3- |
| | O-Methyl-N-cyanonorbuprenorphine, 3-O-Methyl-N- |
| | methylnorbuprenorphine, 6-O-Desmethylbuprenorphine, |
| | Buprenorphine N-oxide, N-But-3-enylnorbuprenorphine, |
| | N-But-3-enylnormethylbuprenorphine, N- |
| | Butylnorbuprenorphine, N-Methylbuprenorphine, |
| | Norbuprenorphine, Tetramethylfuran buprenorphine |
| Oxymorphone | 1-Bromooxymorphone, 6-Beta oxymorphol, 10-Alpha- |
| | hydroxyoxymorphone, 10-Ketooxymorphone, 2,2- |
| | Bisoxymorphone, Noroxymorphone, Oxymorphone N- |
| | oxide, 10-Hydroxyoxymorphone, 4- |
| | Hydroxyoxymorphone, 8-Hydroxyoxymorphone, |
| | Hydromorphinol. |
| Naltrexone | 10-Hydroxynaltrexone, 10-Ketonaltrexone, 14-Hydroxy- |
| | 17-cyclopropylmethylnormorphinone, 2,2′-Bisnaltrexone, |
| | 3-Cyclopropylmethylnaltrexone, 3-O-Methylnaltrexone, |
| | 8-Hydroxynaltrexone, N-(3-Butenyl)-noroxymorphone, |
| | Naltrexone aldol dimer, N-Formyl-noroxymorphone |
| Naloxone | 10-Alpha-hydroxynaloxone, 10-Beta-hydroxynaloxone, |
| | 10-Ketonaloxone, 3-O-Allylnaloxone, 7,8- |
| | Didehydronaloxone, 2,2′-Bisnaloxone, Naloxone N- |
| | oxide |
| Nalbuphine | Beta-epimer of nalbuphine, 2,2′-Bisnalbuphine, 6- |
| | Ketonalbuphine, 10-Ketonalbuphine, Alpha- |
| | noroxymorphol, N-(Cyclobutylcarbonyl)-alpha- |
| | noroxymorphol, N-Formyl-6-alpha-noroxymophol. |

While preferred embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

What is claimed is:

1. A nal-opioid compound does not contain one or more detectable impurities associated with chemical synthesis.

2. The nal-opioid compound of claim 1, wherein the nal-opioid is buprenorphine and the one or more impurities associated with chemical synthesis include 15,16-Dehydrobuprenorphine, 17,18-Dehydrobuprenorphine, 18,19-demethylbuprenorphine, 19,19′-Ethylbuprenorphine, 2,2′-Bisbuprenorphine, 3-Deshydroxybuprenorphine, 3-O-Methylbuprenorphine, 3-O-Methyl-N-cyanonorbuprenorphine, 3-O-Methyl-N-methylnorbuprenorphine, 6-O-Desmethylbuprenorphine, Buprenorphine N-oxide, N-But-3-enylnorbuprenorphine, N-But-3-enylnormethylbuprenorphine, N-Butylnorbuprenorphine, N-Methylbuprenorphine, Norbuprenorphine, and Tetramethylfuran buprenorphine.

3. The nal-opioid compound of claim 1, wherein the nal-opioid is oxymorphone and the one or more impurities associated with chemical synthesis include 1-Bromooxymorphone, 6-Beta oxymorphol, 10-Alpha-hydroxyoxymorphone, 10-Ketooxymorphone, 2,2-Bisoxymorphone, Noroxymorphone, Oxymorphone N-oxide, 10-Hydroxyoxymorphone, 4-Hydroxyoxymorphone, 8-Hydroxyoxymorphone, and Hydromorphinol.

4. The nal-opioid compound of claim 1, wherein the nal-opioid is naltrexone and the one or more impurities associated with chemical synthesis include 10-, 10-Ketonaltrexone, 14-Hydroxy-17-cyclopropylmethylnormorphinone, 2,2′-Bisnaltrexone, 3-Cyclopropylmethylnaltrexone, 3-O-Methylnaltrexone, 8-Hydroxynaltrexone, N-(3-Butenyl)-noroxymorphone, Naltrexone aldol dimer, and N-Formyl-noroxymorphone.

5. The nal-opioid compound of claim 1, wherein the nal-opioid is naloxone and the one or more impurities associated with chemical synthesis include 10-Alpha-hydroxynaloxone, 10-Beta-hydroxynaloxone, 10-Ketonaloxone, 3-O-Allylnaloxone, 7,8-Didehydronaloxone, 2,2′-Bisnaloxone, and Naloxone N-oxide.

6. The nal-opioid compound of claim 1, wherein the nal-opioid is nalbuphine and the one or more impurities associated with chemical synthesis include Beta-epimer of nalbuphine, 2,2′-Bisnalbuphine, 6-Ketonalbuphine, 10-Ketonalbuphine, Alpha-noroxymorphol, N-(Cyclobutylcarbonyl)-alpha-noroxymorphol, and N-Formyl-6-alpha-noroxymophol.

7. A nal-opioid compound that contains less of one or more of the following impurities: 15,16-Dehydrobuprenorphine, 17,18-Dehydrobuprenorphine, 18,19-demethylbuprenorphine, 19,19′-Ethylbuprenorphine, 2,2′-Bisbuprenorphine, 3-Dehydroxybuprenorphine, 3-O-Methylbuprenorphine, 3-O-Methyl-N-cyanonorbuprenorphine, 3-O-Methyl-N-methylnorbuprenorphine, 6-O-Desmethylbuprenorphine, Buprenorphine N-oxide, N-But-3-enylnorbuprenorphine, N-But-3-enylnormethylbuprenorphine, N-Butylnorbuprenorphine, N-Methylbuprenorphine, Norbuprenorphine, Tetramethylfuran buprenorphine, 1-Bromooxymorphone, 6-Beta oxymorphol, 10-Alpha-hydroxyoxymorphone, 10-Ketooxymorphone, 2,2-Bisoxymorphone, Noroxymorphone, Oxymorphone N-oxide, 10-Hydroxyoxymorphone, 4-Hydroxyoxymorphone, 8-Hydroxyoxymorphone, Hydromorphinol, 10-Hydroxynaltrexone, 10-Ketonaltrexone, 14-Hydroxy-17-cyclopropylmethylnormorphinone, 2,2′-Bisnaltrexone, 3-Cyclopropylmethylnaltrexone, 3-O-Methylnaltrexone, 8-Hydroxynaltrexone, N-(3-Butenyl)-noroxymorphone, Naltrexone aldol dimer, N-Formyl-noroxymorphone, 10-Alpha-hydroxynaloxone, 10-Beta-hydroxynaloxone, 10-Ketonaloxone, 3-O-Allylnaloxone, 7,8-Didehydronaloxone, 2,2′-Bisnaloxone, Naloxone N-oxide, Beta-epimer of nalbuphine, 2,2′-Bisnalbuphine, 6-Ketonalbuphine, 10-Ketonalbuphine, Alpha-noroxymorphol, N-(Cyclobutylcarbonyl)-alpha-noroxymorphol, or N-Formyl-6-alpha-noroxymophol; as compared to a nal-opioid produced by chemical synthesis.

8. The nal-opioid compound of claim 7, wherein the nal-opioid is a buprenorphine compound and it contains less of one or more of the following impurities: 15,16-Dehydrobuprenorphine, 17,18-Dehydrobuprenorphine, 18,19-demethylbuprenorphine, 19,19′-Ethylbuprenorphine, 2,2′-Bisbuprenorphine, 3-Deshydroxybuprenorphine, 3-O-Methylbuprenorphine, 3-O-Methyl-N-cyanonorbuprenorphine, 3-O-Methyl-N-methylnorbuprenorphine, 6-O-Desmethylbuprenorphine, Buprenorphine N-oxide, N-But-3-enylnorbuprenorphine, N-But-3-enylnormethylbuprenorphine, N-Butylnorbuprenorphine, N-Methylbuprenorphine, Norbuprenorphine, or Tetramethylfuran buprenorphine; as compared to chemically synthesized buprenorphinc.

9. The nal-opioid compound of claim 7, wherein the nal-opioid is oxymorphone and it contains less of one or more of the following impurities: 1-Bromooxymorphone, 6-Beta oxymorphol, 10-Alpha-hydroxyoxymorphone, 10-Ketooxymorphone, 2,2-Bisoxymorphone, Noroxymorphone, Oxymorphone N-oxide, 10-Hydroxyoxymorphone, 4-Hydroxyoxymorphone, 8-Hydroxyoxymorphone, or Hydromorphinol; as compared to chemically synthesized oxymorphone.

10. The nal-opioid compound of claim 7, wherein the nal-opioid is naltrexone and it contains less of one or more of the following impurities: 10-Hydroxynaltrexone, 10-Ketonaltrexone, 14-Hydroxy-17-cyclopropylmethylnormorphinone, 2,2′-Bisnaltrexone, 3-Cyclopropylmethylnaltrexone, 3-O-Methylnaltrexone, 8-Hydroxynaltrexone, N-(3-Butenyl)-noroxymorphone, Naltrexone aldol dimer, or N-Formyl-noroxymorphone; as compared to chemically synthesized naltrexone.

11. The nal-opioid compound of claim 7, wherein the nal-opioid is naloxone and it contains less of one or more of the following impurities: 10-Alpha-hydroxynaloxone, 10-Beta-hydroxynaloxone, 10-Ketonaloxone, 3-O-Allylnaloxone, 7,8-Didehydronaloxone, 2,2′-Bisnaloxone, or Naloxone N-oxide; as compared to chemically synthesized naloxone.

12. The nal-opioid compound of claim 7, wherein the nal-opioid is nalbuphine and it contains less of one or more of the following impurities: Beta-epimer of nalbuphine, 2,2′-Bisnalbuphine, 6-Ketonalbuphine, 10-Ketonalbuphine, Alpha-noroxymorphol, N-(Cyclobutylcarbonyl)-alpha-noroxymorphol, or N-Formyl-6-alpha-noroxymorphol; as compared to chemically synthesized nalbuphine.


SEQUENCE LISTING (117 sequences)

SEQ ID NO: 1 - protein, 901 aa, Papaver somniferum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 2 - protein, 919 aa, Papaver somniferum
  SITE 209: note = variant - Any amino acid
  SITE 226: note = variant - Any amino acid
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTXLLLPQLAWRQPWKLYYXTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 3 - protein, 732 aa, Papaver somniferum
  SITE 725: note = variant - Any amino acid
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRXVSKPWLH

SEQ ID NO: 4 - protein, 764 aa, Papaver somniferum
  SITE 6: note = variant - Any amino acid
MRWHRXIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 5 - protein, 900 aa, Papaver somniferum
  source 1..900: note = subsp. setigerum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 6 - protein, 724 aa, Papaver somniferum
  source 1..724: note = subsp. setigerum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQALYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEE

SEQ ID NO: 7 - protein, 889 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 8 - protein, 889 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSCKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 9 - protein, 848 aa, Papaver bracteatum
SSPASSTETAVLCHQRQQSCALPISGLLHIFMNKNGLIHVTLGNMADKYGPIFSFPTGSHRILVVSSWEMVKECFTGNNDTAFSNRPIPLAFKTIFYACRGIDSYGLSSVPYGKYWRELRKVCVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGAGTDTTKLTIIWTLSLLLNNPNVLAKAKQEVDAHFETKKRSTNEASVVVDFDDIGNLVYIQAIIKESMRLYPVSPVVERLSSEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 10 - protein, 847 aa, Papaver bracteatum
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSHGQKKMVDVRGKNYELLPFGAGRRICPGISFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 11 - protein, 845 aa, Papaver bracteatum
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLNFRHLIISQVDTSFNKLYDLSNKKKNTTTDSGTVRMDDWLAQLSFNVIGRIVCGFQTHTETSATSSVERFTEAIDEASRFMSIATVSDTFPWLGWIDQLTGLTRKMKHYGKKLDLVVESIIEDHRQNRRISGTKQGDDFIDICLSIMEQPQIIPGNNDPPRQIPIKSIVLDMIGGGTDTTKLTTTWTLSLLLNNPHVLEKAREEVDAHFGTKRRPTNDDAVMVEFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 12 - protein, 869 aa, Papaver bracteatum
VALRKKILKNYYSSSSSTATAVSHQWPKASRALPLIDLLHVFFNKTDLMHVTLGNMADKFGPIFSFPTGSHRTLVVSSWEKAKECFTGNNDIVFSGRPLPLAFKLIFYAGGIDSYGISQVPYGKKWRELRNICVHNILSNQQLLKFRHLMISQVDNSFNKLYEVCNSNKDEGDSATSTTAAGIVRMDDWLGKLAFDVIARIVCGFQSQTETSTTSSMERFTEAMDEASRFMSVTAVSDTVPWLGWIDQLTGLKRNMKHCGKKLNLVVKSIIEDHRQKRRLSSTKKGDENIIDEDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 13 - protein, 858 aa, Papaver bracteatum
  SITE 822: note = variant - Any amino acid
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVXKFSAYAIVWSLFFGHRICITLYSFLIRNVAYICITY

SEQ ID NO: 14 - protein, 716 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRQVFLMQIRLIYICTYQQVHLNIYFQINEFVLCDMYRNLKLEY

SEQ ID NO: 15 - protein, 864 aa, Chelidonium majus
LNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAFLKAIEVGYRYFDTAAAYETEEFLGEAIAEALQLGLIKSRDELFITSKLWPCDAHPDLVVPALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 16 - protein, 364 aa, Papaver somniferum
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVDHRAVVNSTNERLSIATFHDPSLESVIGPISSLITPETPALFKSGSTYGDLVEECKTRKLDGKSFLDSMRI

SEQ ID NO: 17 - protein, 360 aa, Papaver somniferum
METPILIKLGNGLSIPSVQELAKLTLAEIPSRYTCTGESPLNNIGASVTDDETVPVIDLQNLLSPEPVVGKLELDKLHSACKEWGFFQLVNHGVDALLMDNIKSEIKGFFNLPMNEKTKYGQQDGDFEGFGQPYIESEDQRLDWTEVFSMLSLPLHLRKPHLFPELPLPFRETLESYLSKMKKLSTVVFEMLEKSLQLVEIKGMTDLFEDGLQTMRMNYYPPCPRPELVLGLTSHSDFSGLTILLQLNEVEGLQIRKEERWISIKPLPDAFIVNVGDILEIMTNGIYRSVEHRAVVNSTKERLSIATFHDSKLESEIGPISSLVTPETPALFKRGRYEDILKENLSRKLDGKSFLDYMRM

SEQ ID NO: 18 - protein, 364 aa, Papaver somniferum
MEKAKLMKLGNGLSIPSVQELAELTFAEVPSRYVCTNDENLLLMTMGASEIDDETVPVIDLQNLLSPEPAIGKSELDWLHYSCKEWGFFQLVNHGVDALLVDHVKSEIHSFFNLPLNEKTKYGQRDGDVEGFGQAFLVSENQKLDWADMFFINTLPLHLRKPHLFPNLPLPLRETIESYSSEMKKLSMVLFEMMGKAIEVIDIKEAITEMFEDGMQSMRMNYYPPCPQPERVIGITPHSDFDGLTILLQLNEVEGLQIRKEDKWISIKPLPDAFIVNVGDIWEIMTNGVHRSVDHRGVINSTKERLSIATFHSPKLELEIGPISSLIRPETPAVFKSAGRFEDLLKEGLSRKLDGKSFLDCMRM

SEQ ID NO: 19 - protein, 294 aa, Papaver somniferum
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVD

SEQ ID NO: 20 - protein, 364 aa, Papaver somniferum
METAKLMKLGNGMSIPSVQELAKLTLAEIPSRYICTVENLQLPVGASVIDDHETVPVIDIENLISSEPVTEKLELDRLHSACKEWGFFQVVNHGVDTSLVDNVKSDIQGFFNLSMNEKIKYGQKDGDVEGFGQAFVASEDQTLDWADIFMILTLPLHLRKPHLFSKLPLPLRETIESYSSEMKKLSMVLFEKMEKALQVQAVEIKEISEVFKDMTQVMRMNYYPPCPQPELAIGLTPHSDFGGLTILLQLNEVEGLQIKNEGRWISVKPLPNAFVVNVGDVLEIMTNGMYRSVDHRAVVNSTKERLSIATFHDPNLESEIGPISSLITPNTPALFRSGSTYGELVEEFHSRKLDGKSFLDSMRM

SEQ ID NO: 21 - protein, 369 aa, Papaver bracteatum
METPKSIKLGGSLLVPSVQELAQQSFAEVPARYVRDDLEPLTDLSGVSMIDQTIPVIDLQKLQSPVPIIRELESEKLHSACKEWGFFQVVNHGVDILLVEKTKSEIKDFFNLPMDEKKKFWQEEGDIQGFGQAFVQSEDQKLDWADIFLMVTLPRHTRNPRLFPKLPLPLRNTMDSYSSKLSKLASTLIEMMGKALHMETSVLAELFEDGRQTMRINYYPPCPQPKDVIGLTPHSDGGGLTILLQLNEVDGLQIRKEKIWIPIKPLPNAFVVNIGNILEIMTNGIYRSVEHRATIHSTKERLSVAAFHNPKVGVEIGPIVSMITPESPALFRTIEYDDYGKKYFSRKLDGKSSLDFMRIGEGDEENKAT

SEQ ID NO: 22 - protein, 366 aa, Papaver bracteatum
METPKLIKLGGSLLVPSVLELTKQSPAEVPARYIRNDLEPMTDLSSASLTDQTIPVIDLQNLLSPEPELELEKLHSGCKEWGFFQVMNHGVDILLVEKVKSEIQGFFNLPIDEKNKFWQEEGDLEGYGKAFVHSEDEKLDWADMFFILTQPQYMRKPRVFPKLPLRLRETIESYSLELSKLGLTLLDLMGKALQIETGVMSELFEDGRQTMRMNYYPPCPQPEHVIGLTPHSDGGALTILLQLNQVDGLQIRKEEIWVPIKPLPNAFVVNIGDILEIMSNGVYRSVEHRATINSSKERLSVAIFQSPKHGTEIGPILSMITPEAPALFKTIPYEDYLRKFFSRKLGGKSFVDSMRIGESDEDNNTA

SEQ ID NO: 23 - protein, 366 aa, Papaver bracteatum
METQKQENFGASLSVPNVQELAKQSPEQVPDRYIRSDQDSSTNISCPSMTDQIPVIDLQSLLSPDPIIGELELERLHSACKEWGFFQVVNHGVDNLLVEKVKSEIQGFFNLPMDEKKKFWQEEGDFEGFGQAFVFSEDQKLDWGDVFFILTQPQHMRKPRLFPKLPLPFRKTIESYSLETNKLSMTLLELMEKALKIETGVMTELFEGGIQRMRMTYYPPCPQPKHVIGLTPHSDPDALTILLQLNEVDGLQIRKEKIWVPIKPLSNAFVVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 24 - protein, 379 aa, Papaver bracteatum
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFKLPMDEKTKFWQEEGDIEGFGQVFVHSQDQKLDWGDMFLMQTLPRHTRKPRLFPNLPLPLRQTIESYSSELSKLVLTLVDLMGKALQMESGVLTELFENGIQRMRMNYYPPCPQPEQVIGLTPHSDVGGLTILLQLNEVDGLQIKKDKVWVPIKPLANAFVVNVGDALEIMSNGIYRSVEHRATINSTKERLSIATFHNPRADREIGPIPSMISPETPALFKTTGYEEYFKKFFSRKLEGKSFLDSLRIREGDEHCGRLDVKGPCN

SEQ ID NO: 25 - protein, 365 aa, Papaver bracteatum
MEIPNPIKIGSSLLVPSVQELAKQSFAEVPARYIRNDVDPLITKLSDVSLIDQTVPVIDLQKLLSPEPIVGELELERLHSACKEWGFFQVVNHGVDNLLVEKVKSEIQGFFNLPMEEKKKFWQEEGDFEGFGQMFVQSEEQKLDWGDMFFILTQPQHMRKPRLFSKLPLPLRETIESYSLELIKLGLTIIKLMEKALQIDAGVMAELFEDGIHTMRMNYYPPCPQPEHVIGLTPHSDGGGLTILLQLNEVDGLQIRRENIWVPIKPLPNAFVVNIGDILEILSNGIYRSVEHRSTVNATKERLSVATFQNPKQESVIGPNMITPERPALFRKIVYKDYMKKLFSRKLDGKSFLDSLRIGEGDERP

SEQ ID NO: 26 - protein, 349 aa, Papaver bracteatum
METLKTVKPGGSLFIPNGQELAKQSLEEVYVGNDQDTMLLIGQTIPVIDLQKLLSPEPITGDMELDKLHSACKEWGFFQVVNHGVDILLVEKVKSEVHDFFNIPMDEKKPFWQEEGDLEGFGQVFITSEDQQLDWGDMFFMVTLPKHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 27 - protein, 379 aa, Papaver bracteatum
  SITE 373: note = variant - Any amino acid
  SITE 377: note = variant - Any amino acid
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIKKDKIWVPIKPLRNAFVVNVGDALEIMSNGIYRSVEHRATINSTKERLSIATFHNPRADREIGPIPSMISPETPALFKTTGYEEYFKKFFSRKLEGKSFLDSLRIGEGDEHCGRLXVKGXCN

SEQ ID NO: 28 - protein, 362 aa, Papaver bracteatum
METPKLMKLGGSLFVPSVQELAKQSLAEVPARYVRDDRDMVGNIINVTPMSMIDQSIPVIDLEKLLSPDLIVGELELERLHSACKEWGFFQVVNHGVDSLLVEKVKSEIEGFFELPMDEKKKFWQEEGDAEGFAQFFVQSEDQKLDYSGDMFFMLNLPQHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 29 - protein, 365 aa, Papaver bracteatum
METPKLRDFGSFLPVPSVQELAKQVLTEIPPRYIRTDLEALNKLSCASNTDQTVPIIDMQCLLSAEPEMELEKLHSACKEWGFFRVVNHGVDNLESVKSEIESFLNLPVNAKNKYGQKQGDDQGFGSRFVLSEEQKLDWGDFFYMVTRPLYLRKPHLFPELPLPLRETIESYSSEVSKLAMALFEMMGKALKIETGVMTEIFEGGMQAMRMNYYPPCPRPDLVIGLNAHSDFGGLTILLQLNEVEGLEIRNKGEWVSVKPLANAFVVNVGDVMEILTNGIYHSVEHRATINSSKERLSVATFHYPKLETGIGPLPCMITPKTPALFGRIERYELLLRKYYARKLNGKSTLDCMRIGNGFEDDNTA

SEQ ID NO: 30 - protein, 370 aa, Papaver bracteatum
MEAPKLIMLGGSLFVPSVQELAKQSLAEVPARYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSEVGGLTILLQLNEVDGLQIRKEKIWVPIKPLSNAFIVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 31 - protein, 313 aa, Papaver bracteatum
MSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIRKEKIWVPIKPLSNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 32 - protein, 365 aa, Papaver bracteatum
METPKLVKSSGSSLFLSTSVQELAKQSLPEVPARYIRTNLEPLSNVSGDSQSVPVIDLQKLLSSEPIIGELELDKLHSACKEWGFFQVVNHGVDNLVMEKIKTEIQGFFNLSLDEKQKFWKKEGDAEGFGQNFIESEDQKLDWGDTFGMFTLPIHMRNPRLFPELPLPLRETIESYSLDVRKLALALIGLMEKALKIKTSAMSELFEDGGQAMRMNYYPPCPQPEHVIGLTPHSDAGGLTILLQLNEVDGLQIKKDKIWVPIKPLPNAFVVNIGDILEIMTNGIYRSVEHRATINSSKERLSVAAFHSPKGDTLIGPMVSLITPETPALFRTIGYQDYMKKFMSRKLDGKSLVNSMRIGEGDEDK

SEQ ID NO: 33 - protein, 356 aa, Papaver bracteatum
METPTLMKLGNGLSVPSVQELAKATLAEIPSRYICTDENLLTMGASTTDNETVPVIDLQNLLSPEPVIGMLELDRLHSACKEWGFFQLVNHGVDALLVDNEVQGFFNLPMDEKTKYGQKDGDDEGFGQFFVISEDQKLDWADVFYMSTLPLHSRKPHLFPELPLPLRETMESYSSEMKKLSMVLFDMMGKALQVVEIKGITELFEDGAQQIRMNYYPPCPQPELVFGLTSHSDFDGLTILLQLGEVEGLQIKKEERWISIKPLPDAFIVNVGDILEIMTNGIYRSVDHRAVVNSIKERLTIATFHDPRLEAEIGPISSLITPETPALFKRGVFEDLLKEMFLRKLDGKSFLDCMRM

SEQ ID NO: 34 - protein, 350 aa, Papaver rhoeas
GNGLSVPSVQELAKQTLAEIPSRYICTDENPLITGASVVDDETVPVINLQNLLSPEPVIGKLELDKLHSACKEWGFFQVVNHGVNDSLVDSVKSEIEGFFNLPANEKLKYGQKDGDVEGFGQHFVVSEDQKLDWADVFYMVTLPVRLRKPHLFPELPLPLRDTLDSYSSELNKLSMVLLEMMEKALKLVECKGITDFFEDGFQQMRMNYYPPCPRPELVTGLTSHSDFGGLTILLQLNDVEGLQIKKEERWISIKPLPNAFIVNIGDVLEIMSNGIYRSVDHRAVINSTKVRMSVATFHDPRLEAVIGPISSLITPETPALFKRGVFEDLLKEMFLRKLDGKSFLDCMRI

SEQ ID NO: 35 - protein, 351 aa, Papaver somniferum
  source 1..351: note = subsp. setigerum
LMKLANGMSVPIVQELAKLTVGEIPSRYICTDGNLLTMGASVIDYETVPVIDLQNLQSREPVIEKLELDRLHSACKEWGFFQLLNHGVDASLMDNVRSEIRGFFNLPISDKMKYGQKDGDEEGFGQHFIVSEDQKLDWVDAFMMFTLPLHSRNPRLTPEFPQPLRETVESYSSEMKKLSVLLFELMEKALQVKGITEMFEDGLQSIRMNYYPPCPRPELAIGLTSHSDFDGLTILLQLNEVEGLQIKKEERWISIKPLPNAFIVNVGDVLEVMTNGIYRSVDHRAVVNSTKERLSIATFHDPELESEIGPIASLITPETPALFKRGRFKDLLKENLSTKLDGKSFLDCIRM

SEQ ID NO: 36 - protein, 497 aa, Homo sapiens
MGLEALVPLAVIVAIFLLLVDLMHRRQRWAARYSPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLRIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 37 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 38 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 39 - protein, 502 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSIIFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 40 - protein, 534 aa, Menispermum canadense
MIMMFIDYYSSWLPQTLLLQSILLAVSLVIFINLFLTRRRSYSSKSHTNIIHPPKAAGALPVIGHLYTLFRGLSAGVPLYRQLDAMADRYGPAFIIHLGVYPTLVVTCRELAKECFTTNDQTFATRPSTCAGKYIGYNYAFFGFAPYGPYWREARKIATVELLSNYRLDSLRHVREAEVGRNVDELYALHASSSTNKQNMMKIDMKQWFDQVTLNVILMMVVGKRCVTTGGNEEEVRVVKVLHEFFKHLGTLSVSDVVPYVEWMDLDGNIGRMKSTAKELDCILGRWLEEHRRERRSDFMDAMLAMVEGIKIPYYDSDTVIKAICLNLLNAGSDTLGITMTWALSLLLNNRHVLKKVKDELDVHVGKNRQVEELDVKNLVYLHAVVKETLRLFPPAPLGVPHEAMEDCVVGGFHVAKGTRLVVNVWKLHRDPSVWSDPLAFKPERFLDNNTVDVRGQHFQLLPFGSGRRGCPGITFALQVAHLTLARLLHGFEWDTPDGAPVDMSEVSVLTTAKKNPVEVLFTPRLPAEVYTQN

SEQ ID NO: 41 - protein, 527 aa, Nigella sativa
MLSIHDSTMVFLQLQAICGIFGFIFIITWWTRWKSSNKMKAPEVAGAWPVIGHLHLLGGGRPLYQLLGDMSDKYGPAFTLRMGIQKALVVSSWEVAKECLTTNDRALATRPSSAGGKYMGYNNALIPFSPYGPYWRDMRKIATLELLSNHRLEELKHVREMEINTCISDMYKLCQVEDGVEIKPISVDLSQWFADLTFNVVVMMITGKRYIGSTDAGDMNEIRHFQAALVKFMRLLRISLLVDVFPVLQWINYGGFKGVMKSTARDIDSVLENWLQEHQRKRLSPDFNGNHDFIDVMISTLEGTEFSDYDHNTIIKAISMAMVVGGTDTTTTTLIWAISLLLNNPNAMKKVQEELEIHVGKERNVDGSDIQHLVYLQAVVKETLRLYPPVPLSVMHQAMEDCVIGSYNIQAGTRVLFNLWKLHRDSSVWSDPLEFRPERFLTSHVDVDVRGQHFELIPFGSGRRSCPGISFALQVIHLTIARLFHGFNLTTPGNSSVDMSEISGATLSKVTPLEVLVTPRLSSKLYN

SEQ ID NO: 42 - protein, 525 aa, Hydrastis canadensis
MDSLLQLQIIGALAALIFTYKLLKVICRSPMTDGMEAPEPPGAWPIIGHLHLLGGQDPIARTLGVMTDKYGPILKLRLGVHTGLVVSNWELAKECFTTNDRVLASRPMGAAGKYLGYNYAIFGLAPHGPYWSEVRKIVLRELLSNQSLEKLKHVRISEINTCLKNLFSLNNGNTPIKVDMKQWFERPMFNVVTMMIAGKRYFSMENDNEAMNFRKVATEFMYLTGVFVVSDALPYLEWLDLQGHVSAMKRTAKELDIHVGKWLEEHRRAKLLGETKNEDDFVDVLLTILPEDLKDNQTYIHDRDTIIKATALALFLAASDTTAITLTWALSLILNNPDVLKRAQDELDKHVGKEKLVKESDIINLVYLQAIIKETLRLYPAAPLLLPHEAMEDCTVGGYHVPKGTRIFVNIWKLQRDPRVWFDPNEFRPERFLTTHANVDFKGQHFEYIPFSSGRRVCPGITFSTQIMHLTLAHLLHEFNIVTPTKSNAGVDMTESLGITMPKATPLEVLLTPRLPSNLYNQYRD

SEQ ID NO: 43 - protein, 550 aa, Eschscholzia californica
MNLLIFFQFLLQFQVLVGLSVLLAFSYYLWVSKNPKINKFKGKGALLAPQAAGAWPIVGHLPQLVGPKPLFRILGAMADNYGPIFMLRFGVHPTVVVSSWEMTKECFTTNDRHLASRPSNAASQYLIYEVYALFGFSLYGSSYWRDARKIATLELLSHRRLELLKHVPYTEIDTCIKQLHRLWTKNNKNQNNPELKVEMNQFFTDLTMNVILKLVVGKRFFNVDDAADHEKEEARKIQGTIFEFFKLTEGSVSAGALPLLNWLDLNGQKRAMKRTAKKMDSIAEKLLDEHRQKRLSKEGVKGTHDHNDFMDVLLSILDADQGDYSHHPFNYSRDHVIKATTLSMILSSMSISVSLSWALSLLLNNRHVLKKAQDELDMNVGKDRQVEEGDIKNLVYLQAIVKETFRMYPANPLLLPHEAIEDCKIGGFNVPAGTRVVVNAWKLQHDPRVWSNPSEFKPERFLNDQAAKVVDVRGQNFEYLPFGSGRRVCPGISFSLQTIHMSLARLVQAFELGTPSNERIDMTEGSGLTMPKTTPLHVLLNPRLPLPLYE

SEQ ID NO: 44 - protein, 537 aa, Glaucium flavum
MELINSLEIQPITISILALLTVSILLYKIIWNHGSRKNNKSNKNNRKTSSSAGVVEIPGAWPIIGHLHLFNGSEQMFHKLGSLADQYGPAPFFIRFGSRKYVVVSNWELVKTCFTAQSQIFVSRPPMLAMNILFFPKDSLSYIQHGDHWRELRKISSTKLLSSHRVETQKHLIASEVDYCFKQLYKLSNNGEFTLVRLNTWCEDMALNVHVRMIAGMKNYVAAPGSGEYGGQARRYRKALEEALDLLNQFTITDVVPWLGWLDHFRDVVGRMKRCGAELDSIFATWVEEHRVKRASGKGGDVEPDFIDLCWESMEQLPGNDPATVIKLMCKEHIFNGSGTSSLTLAWILSLIMNNPYVIKKAREELEKHVGNHRQVEESDLPNLLYIQAIIKEGMRLYTPGPFIDRNTTEDYEINGVHIPAGTCLYVNLWKIHRDPNVYEDPLEFKPERFLKNNSDLDLKGQNYQLLPFGAGRRICPGVSLALPLMYLTVSRLIHGFDMKLPKGVEKADMTAHGGVINQRAYPLEVLLKPRLTFQQA

SEQ ID NO: 45 - protein, 568 aa, Stylophorum diphyllum
MTIGALALLSFIYFLRVSVIKRTKYTNTAVTATNKLENDEDEANHSKRVVAPPEVAGAWPILGHLPQLVGLKQPLFRVLGDMADKYGPIFIVRFGMYPTLVVSSWEMAKECFTTNDRVLASRPASASGKYLTYNYAMFGFTNGPYWREIRKISMLELLSHRRVELLKHVPSTEIDSSIKQLYHLWVENQNQNKQGDHQVKVDMSQLLRDLTLNIVLKLVVGKRLFNNNDMDHEQDEAARKLQKTMVELIKVAGASVASDALPFLGWLDVDGLKRTMKRIAKEIDVIAERWLQEHRQKKLTSNDKGGSNNIQGGGGDNDFMDVMLSILDDDSNFFINYNRDTVIKATSLTMILAGSDTTTLSLTWALTLLATNPGALRKAQDELDTKVGRDRQVDERDIKNLVYLQAIVKETLRMYPAAPLAIPHEATQDCIVGGYHVTAGTRVWVNLWKLQRDPHAWPNPSEFRPERFLAVENDCKQQGTCDGEAANMDFRGQHFEYMPFGSGRRMCPGINFAIQIIHMTLARLLHSFELRVPEEEVIDMAEDSGLTISKVTPLELLLTPRLPLPLYI

SEQ ID NO: 46 - protein, 561 aa, Stylophorum diphyllum
FCQFQGIVGILLAFLTFLYYLWRASITGLRTKPKHNDFKVTKAAPEADGAWPIVGHFAQFIGPRPLFRILGDMADKYGSIFMVRFGMYPTLVVSSWEMAKECFTTNDRFLASRPASAAGKYLTYDFAMLSFSFYGPYWREIRKISMLELLSHRRVELLKHVPSTEIDSSIKQLYHLWVENQNQNKQGDHQVKVDMSQLLRDLTLNIVLKLVVGKRLFNNNDMDHEQDEAARKLQKTMVELIKVAGASVASDALPFLGWLDVDGLKRTMKRIAKEIDVIAERWLQEHRQKKLTSNDKGGSNNIQGGGGDNDFMDVMLSILDDDSNFFINYNRDTVIKATSLTMILAGSDTTTLSLTWALTLLATYPLCALRKAQDELDTKVGRDRQVDERDIKNLVYLQAIVKETLRMYPAAPLAIPHEATQDCIVGGYHVTAGTRVWVNLWKLQRDPHAWPNPSEFRPERFLAVENDCKQQGTCDGEAANMDFRGQHFEYMPFGSGRRMCPGINFAIQIIHMTLARLLHSFELRVPEEEVIDMAEDSGLTISKVTPLELLLTPRLPLPLYI

SEQ ID NO: 47 - protein, 561 aa, Chelidonium majus
MDLFIFFSRFQYIVGLLAFLTFFYYLWRVSITGTRIKTNQNIMNGTNMMAPEAAGAWPIVGHLPQLVGPQPLFKILGDMADKYGSIFMVRFGMHPTLVVSSWEMAKECFTTNDKFLASRPTSAGGKYLTYDFAMFGFSFYGPYWREIRKISTLELLSHRRVELLKHVPYTEIGGSIKQLYKLWMETQNQNKQRDDHQVKVDMSQVFGYLTLNTVLKLVVGKGLFNNNDMNHEQEEGRKLHETVLEFFKLAGVSVASDALPFLGWLDVDGQKRSMKRIAKEMDLIAERWLQEHRQKRLTSNNKASSGHDDFMSVLLSILDDDSNFFNYNRDTVIKATSLNLILAASDTTSVSLTWVLSLLVTNPGALKKVQDELDTKVGRNRHVEERDIEKLVYLQATVKETLRMYPAGPLSVPHEATQDCTVGGYQVTAGTRLVVNVWKLQRDPRVWPNPSEFKPERFLPDGCEVGCGEAANMDFRGQHFEYIPFGSGRRMCPGIDFAIQIIHMTLACLLHAFEFQVPSSLDKHLVPAVIDMSEGSGLTMPKVTPLEVLLNPRLPLPLYEL

SEQ ID NO: 48 - protein, 561 aa, Eschscholzia californica
MEKPILLQLQPGILGLLALMCFLYYVIKVSLSTRNCNQLVRHPPEAAGSWPIVGHLPQLVGSGKPLFRVLGDMADKFGPIFMVRFGVHPTLVVSSWEMAKECFTSNDKFLASRPPSAASIYMAYDHAMLGFSSYGPYWREIRKISTLHLLSHRRLELLKHVPHLEIHNFIKGLYGIWKDHQKQQQQPTARDDQDSVMLEMSQLFGYLTLNIVLSLVVGKRVCNYHADGHLDDGEEAGQGQKLHQTITDFFKLSGVSVASDALPFLGLFDLDGQKKIMKRVAKEMDFVAERWLQDKKSSLLLSSKSNNKQNEAGEGDVDDFMDVLMSTLPDDDDSFFTKYSRDTVIKANSLSMVVAGSDTTSVSLTWALSLLLNNIQVLRKAQDELDTKVGRDRHVEEKDIDNLVYLQAIVKETLRMYPAGPLSVPHEAIEDCNVGGYHIKTGTRLLVNIWKLQRDPRVWSNPSEFRPERFLDNQSNGTLLDFRGQHFEYIPFGSGRRMCPGVNLATPILHMTLARLLQSFDLTTPSSSPVDMTEGSGLTMPKVTPLKVLLTPRLPLPLYDY

SEQ ID NO: 49 - protein, 567 aa, Papaver bracteatum
MDVAIIVDHHYLQPFVSIAGLLALLSFFYCIWVFIIRPRIIKSNLDERKLSPSSPPEVAGAWPIVGHLPQLIGSTPLFKILADMSNKYGPIFMVRFGMYPTLVVSSWEMSKECFTTNDRLFATRPPSAAGKYLTKALFAFSVYGPYWREIRKISTIHLLSLRRLELLKHGRYLEIDKCMKRLFEYWMEHHKNIISTTSSVKVNMSQVFAELSLNVVLKIIVGKTLFIKNGNEDYTKEEEEGQKLHKTILKFMELAGVSVASDVLPFLGWLDVDGQKKQMKRVYKEMNLIASKWLGEHRERKRLQIIQKRGAARGSNYDDGNDFMDVLMSILDEENDDLFFGYSRDTVIKSTCLQLIVAASDTTSLAMTWALSLLLTNPNVLQKAQDELDTKVGRDRIIEEHDIECLVYLQAIVKETLRLYPPAPLSLPHEAMEDCTVGGYQVKAGTRLVVNLWKLQRDPRVWSNPLEFKPERFLPQSDGGFGGEEARMDFRGQHFEYTPFGSGRRICPGIDFFLQTVHMALARLLQAFDFNTAGGLVIDMVEGPGLTMPKVTPLEVHLNPRLPVTLY

SEQ ID NO: 50 - protein, 558 aa, Papaver bracteatum
MQVDWPNILQKYYPIITCSLLTLLSFYYIWVSITKPSRNSKTKLPPPEVAGSWPIVGHLPQLVGSTPLFKILANMSDKYGPIFMVRFGMHPTLVVSSWEMSKECFTTNDKFLASRPPSASAKYLGYDNAMFVFSDYGPYWREIRKISTLQLLTHKRLDSLKNIPYLEINSCVKTLYTRWAKTQSQIKQNVGGAADDFVKVDMTEMFGHLNLNVVLRLVVGKPIFIQKDNADEDYTKDGHNKEELGQKLHKTIIEFFELAGASVASDVLPYLGWLDVDGQKKRMKKIAMEMDLFAQKWLEEHRQKGINHDNENDFMAVLISVLGEGKDDHIFGYSRDTVIKATCLTLIVAATDTTLVSLTWALSLLLTNPRVLSKAQDELDTVVGKERNVEDRDVNHLVYLQAVIKETLRLYPPSPLAVPHEAIENCNVGGYEVKARTRLLVNLWKIHRDPRVWSNPLEFKPERFLPKLDGGTGEASKLDFKGQDFVYTPFGSGRRMCPGINFASQTLHMTLARLLHAFDFDIESNGLVIDMTEGSGLTMPKVTPLQVHLRPRLPATLY

SEQ ID NO: 51 - protein, 534 aa, Menispermum canadense; identical to SEQ ID NO: 40

SEQ ID NO: 52 - protein, 527 aa, Nigella sativa; identical to SEQ ID NO: 41

SEQ ID NO: 53 - protein, 525 aa, Hydrastis canadensis; identical to SEQ ID NO: 42

SEQ ID NO: 54 - protein, 550 aa, Eschscholzia californica; identical to SEQ ID NO: 43

SEQ ID NO: 55 - protein, 537 aa, Glaucium flavum; identical to SEQ ID NO: 44

SEQ ID NO: 56 - protein, 568 aa, Stylophorum diphyllum; identical to SEQ ID NO: 45

SEQ ID NO: 57 - protein, 561 aa, Stylophorum diphyllum; identical to SEQ ID NO: 46

SEQ ID NO: 58 - protein, 561 aa, Chelidonium majus; identical to SEQ ID NO: 47

SEQ ID NO: 59 - protein, 561 aa, Eschscholzia californica; identical to SEQ ID NO: 48

SEQ ID NO: 60 - protein, 567 aa, Papaver bracteatum; identical to SEQ ID NO: 49

SEQ ID NO: 61 - protein, 558 aa, Papaver bracteatum; identical to SEQ ID NO: 50

SEQ ID NO: 62 - protein, 571 aa, Papaver bracteatum
MMDLAMFIDQYFSLAKIAGLLALLSFFYYLWISTLWSPRNPKLSSVSPPEVAGAWPILGHLPQLLGSRPLFKILADMSDNYGPIFMVRFGMHPTLVVSSWEMAKECFTTNDRFLAGRPSGAANKYLTFALFGFSTYGPYWREIRKIATLHLLSHRRLELLKHVPDLEVTNCMKHLHRRWIDSQNQIKQNDAAAGSVKVDMGRVFGELTLNVVLKLVAGKSIFFKNDNTRQYDSKDGHNKEEEEGKKLHKTIIDFYSLAGASVASDVLPFLGWLDVDGQKKRMKRVAKDMDFIAAKWLEEHRHQKRQTVLSSSATLGSSNHDDAKDFMDVLMSILDGENDDLFFGYSRDTVIKTTCLQLIAAAADTTSVTMTWALALLITNPTILRKAQDELDTKVGKDRNIEERDINDLVYLQAIVKETLRMYPAGPLNVPHEAIADCNIGGYEVRAGTRLLVNLWKMHRDPRVWSNPSEFKPERFLPQLDGGSGGEAANLDFRGQDFEYLPFSAGRRMCPGIDFSLQTLHMTLARLLHGFDFNNDSAGIIIDMEEGSGLTMPKLTPLEIYLCPRLPAKLY

SEQ ID NO: 63 - protein, 361 aa, Thalictrum flavum
MAVEGKQVAPKKAIIVELLKKLELGLVPDDEIKKLIRIQLGRRLQWGCKSTYEEQIAQLVNLTHSLRQMKIATEVETLDDQMYEVPIDFLKIMNGSNLKGSCCYFKNDSTTLDEAEIAMLELYCERAQIKDGHSVLDLGCGQGALTLYVAQKYKNSRVTAVTNSVSQKEFIEEESRKRNLSNVEVLLADITTHKMPDTYDRILVVELFEHMKNYELLLRKIKEWMAKDGLLFVEHICHKTFAYHYEPIDEDDWFTEYVFPAGTMIIPSASFFLYFQDDVSVVNHWTLSGKHFSRTNEEWLKRLDANVELIKPMFVTITGQCRQEAMKLINYWRGFCLSGMEMFGYNNGEEWMASHVLFKKK

SEQ ID NO: 64 - protein, 358 aa, Coptis japonica
MAVEAKQTKKAAIVELLKQLELGLVPYDDIKQLIRRELARRLQWGYKPTYEEQIAEIQNLTHSLRQMKIATEVETLDSQLYEIPIEFLKIMNGSNLKGSCCYFKEDSTTLDEAEIAMLDLYCERAQIQDGQSVLDLGCGQGALTLHVAQKYKNCRVTAVTNSVSQKEYIEEESRRRNLLNVEVKLADITTHEMAETYDRILVIELFEHMKNYELLLRKISEWISKDGLLFLEHICHKTFAYHYEPLDDDDWFTEYVFPAGTMIIPSASFFLYFQDDVSVVNHWTLSGKHFSRTNEEWLKRLDANLDVIKPMFETLMGNEEEAVKLINYWRGFCLSGMEMFGYNNGEEWMASHVLFKKK

SEQ ID NO: 65 - protein, 351 aa, Papaver somniferum
MQLKAKEELLRNMELGLIPDQEIRQLIRVELEKRLQWGYKETHEEQLSQLLDLVHSLKGMKMATEMENLDLKLYEAPMEFLKIQHGSNMKQSAGYYTDESTTLDEAEIAMLDLYMERAQIKDGQSVLDLGCGLGAVALFGANKFKKCQFTGVTSSVEQKDYIEGKCKELKLTNVKVLLADITTYETEERFDRIFAVELIEHMKNYQLLLKKISEWMKDDGLLFVEHVCHKTLAYHYEPVDAEDWYTNYIFPAGTLTLSSASMLLYFQDDVSVVNQWTLSGKHYSRSHEEWLKNMDKNIVEFKEIMRSITKTEKEAIKLLNFWRIFCMCGAELFGYKNGEEWMLTHLLFKKK

SEQ ID NO: 66 - protein, 358 aa, Papaver somniferum
MGSIDEVKKESAGETLGRLLKGEIKDEELKKLIKFQFEKRLQWGYKSSHQEQLSFNLDFIKSLKKMEMSGEIETMNKETYELPSEFLEAVFGKTVKQSMCYFTHESATIDEAEEAAHELYCERAQIKDGQTVLDIGCGQGGLVLYIAQKYKNCHVTGLTNSKAQVNYLLKQAEKLGLTNVDAILADVTQYESDKTYDRLLMIEAIEHMKNLQLFMKKLSTWMTKESLLFVDHVCHKTFAHFFEAVDEDDWYSGFIFPPGCATILAANSLLYFQDDVSVVDHWVVNGMHMARSVDIWRKALDKNMEAAKEILLPGLGGSHETVNGVVTHIRTFCMGGYEQFSMNNGDEWMVAQLLFKKK

SEQ ID NO: 67 - protein, 350 aa, Eschscholzia californica
MGSSAGEIMGRLMKGEIEDEELKKLIRHQWDRRIEWGYKPTHEKQLAFNLDFIKGLKEMVMSGEIDTMNKETYELPTAFLEAVFGKTVKQSCCYFKDENSTIDEAEEAAHELYCERAQIKDGQTVLDIGCGQGGLVLYIAEKYKNCHVTGLTNSKAQANYIEQQAEKLELTNVDVIFADVTKFDTDKTYDRILVVETIEHMKNIQLFMKKLSTWMTEDSLLFVDHISHKTFNHNFEALDEDDWYSGFIFPKGCVTILSSSTLLYFQDDVSALDHWVVNGMHMARSVEAWRKKLDETIEAAREILEPGLGSKEAVNQVITHIRTFCIGGYEQFSYNNGEEWMITQILFKKK

SEQ ID NO: 68 - protein, 363 aa, Papaver somniferum
MSTTMETTKISQQDDLWKNMELGQISDEEVRRLMKIGIEKRIKWGTKPTQQEQLAQLLDFNKSLRGMKMATEIDTLENHKIYETPESFNQIIGGKESAGLFTDETTTTMEEANTKMMDLYCERAGLKDGHTILDLGCGAGLLVLHLAKKYKKSKITGITNTSSHKEYILKQCKNLNLSNVEIILADVTKVDIESTFDRVFVIGLIEHMKNFELFLRKISKWMKDDGLLLLEHLCHKSFSDHWEPLSEDDWYAKNFFPSGTLVIPSATCLLYFQEDVTVIDHWILSGNNFARSNEVILKRIDGKIEEVKDIFMSFYGIGREEAVKLINWWRLLCITANELFKYNNGEEWLISQLLFKKKLMTCI

SEQ ID NO: 69 - protein, 356 aa, Thalictrum flavum
METKQTKKEAVANLIKRIEHGEVSDEEIRGMMKIQVQKRLKWGYKPTHEQQLAQLVTFAQSLKGMEMAEEVDTLDAELYEIPLPFLHIMCGKTLKFSPGYFKDESTTLDESEVYMMDLYCERAQIKDGQSILDLGCGHGSLTLHVAQKYRGCKVTGITNSVSQKEFIMDQCKKLDLSNVEIILEDVTKFETEITYDRIFAVALIEHMKNYELFLKKVSTWIAQYGLLFVEHHCHKVFAYQYEPLDEDDWYTEYIFPSGTLVMSSSSILLYFQEDVSVVNHWTLSGKHPSLGFKQWLKRLDDNIDEVKEIFESFYGSKEKAMKFITYWRVFCIAHSQMYSTNNGEEWMLSQVLFKKK

SEQ ID NO: 70 - protein, 358 aa, Papaver bracteatum
MGSIDEVKKESAGETLGRLLKGEIKDEELKKLIKFQFEKRLQWGYKSSHQEQLSFNLDFIKSLKKMEMSGEIETMNKETYELPSEFLEAVFGKTVKQSMCYFKHESATIDEAEEAAHELYCERAQIKDGQTVLDIGCGQGGLVLYIARKYKKCHVTGLTNSKAQVNYLLKQAEKLGLTNVDAILADVTQYESDKTYDRLLMIEAIEHMKNLQLFMKKLSTWMTEESLLFVDHVCHKTFAHFFEAVDEDDWYSGFIFPPGCATILAANSLLYFQDDVSVVDHWVVNGMHMARSVDIWRKALDKNMEAAKEILLPGLGGSHEAVNGVVTHIRTFCMGGYEQFSMNDGDEWMVAQLLFKKK

SEQ ID NO: 71 - protein, 358 aa, Papaver bracteatum
MGSIEEVKKESAEETLGRLLRGEINDEELKKLIKYQLEKRLQWGYKSSHQEQLSFNLDFINSLKKMGMSGQVEAFTNEVYELPTECFEAAYGKSMKLSGCYFKHESSTIDEAEEASHELYCERAQIKDGQTVLDIGCGQGGLVLYVAQKYKNCHVTGLTNSKEQVNYILKQAEKLGLRNVDVILADVTQYESDKTYDRILVIGVVEHMKNMQLFIKKLSTWMAEDSLLFVDHSCHKTFNHFFEALDEDDWYSGYIFPPGCATFLSADSLLYFQDDVSVVDHWVVNGMHFARTVDAWRKKLDKNMEAVKEILLPGLGGNHEAVNGVITHIRTCCVGGYVQFSLNDGDEWMNAQLLFKKK

SEQ ID NO: 72 - protein, 368 aa, Argemone mexicana
MCLFFAEKMGLMAEANNQQQLKKEDLLKNMELGLIPDEEIRKLIRVQLEKRLNWGYKSTHEQQLSQLLHLVHSLKKMKIATEMENLDLKLYEAPFSFVQIQHGSTIKESSGLFKDESTTLDEAEIAMLDLYTKRAKIEDGQSVLDLGCGLGAVTLYVAQKFKNCYVTGITSSVEQKDFIEGRCKELKLSNVKVILADITTYETEEKYNRIFAVELIEHMKNYELLLRKISEWMKQDGLLFIEHVCHKTLAYHYEPLDEEDWYTNYIFPAGTLTLSSATLLLYFQDDVAVVDQWTLSGKHYSRSHEEWLKRIDGNIEEVKEIMKSITKSEEEAKKLLNFWRIFCMCGAELFGYKNGEEWMMTHILFKKK

SEQ ID NO: 73 - protein, 364 aa, Glaucium flavum
MDLMATSKQVKKKEELLKNMELGLVPDEEIRRLIRIELEKRLKWGYKPTHQQQLAQLLDLVHSLKKMKIATEMESLDLKLYEAPFSFVQIKHGSTIKESSSYFKDESMTLDEAEIAMLDLYVERAQIEDGQSVLDLGCGLGAVTLHVAKKYKNCHVTGLTNSVEQKDFIEGKCKELNLSNVKVILADVTSHEMEDKFDRIFAVELIEHMKNYELLLRRISKWMKDDGLLFIEHVCHKTFAYHYEPIDEDDWYTEYIFPAGTLTLSSASLLLYFQDDVSVVNHWTLSGKHYSRSHEEWLKRIDGNMDAVKEIMKSITKTEEEAVKLINFWRIFCMCGAELFGYKDGEEWMMSHVLFKKKQLLQQC

SEQ ID NO: 74 - protein, 353 aa, Eschscholzia californica
MVDLKVEKEELLKSMELGLVPDEDIRKHIRSQLEKRLKWGYKPNHEQQLAQLLDVIHSLKKMKISKEYESFDLRLYEAPFDFHKIQLGTHLKESCSYYKDESTTLDEAEGAMLDLYTQKAKIEDGQSILDLGCGVGAVTLFVANKYKNCKVTGITSCQWQKDFIENKCKELNLTNVRVIIGDVTAYEMEETFDRIFAIELIEHMKNYELLLRKISKWMKDDGLLFIEHVCHKILAYPYEPIDEEDWFTEYIFPGGTLTLSSASLLLYFQDDVSVVEHSSLNGKHYSRSHGEWLKNIDANIDEVKGIMRSITKTEEEAVRLVNFWRIFCMCGIELFGYNNGEEWMVSHILLKKK

SEQ ID NO: 75 - protein, 359 aa, Eschscholzia californica
MAADLVVKKWNNKKELIDEMELGLVGDEEIRELIRNDLEKRLKWGYKSNHEQQLAQLLHFVHSLRGMKIAADEVESFNIKVYEAPFSFNKIQLGSSLKESSCYYKHDETTLDEGEIAMMELYTEKAQIKDGQSVLDLGCGLGSLTLYVANKYPNCKVTGTTASLWHKDFIESKCKEQELTNVKIVLGDATTHEMEERFDRILAIGLIEHLKNYGLLLGRISKWLKDDGFLFIQHVCHKTLAYPLVPVDEEDWIGEYIFPGGTLTMPSASLLLYFQDELSVVDHSTLNGKHFSRTHEEWLKNIDAKIDEVKEILKSVTKTEEEVVRLTNFWRIFCMFGVEMFGYNEGEEWMLSQILFKKK

SEQ ID NO: 76 - protein, 353 aa, Chelidonium majus
MASGKVVDLLKRLDSGLVSDEELRRVIRFELERRLKWGYKPTHEQQLAELLNLAHATKQMEIATKIDTLNSTMYEVPNSFLEIQLGSTLKESCLYFKDESTTVDEAEIAMMDLYLERAQIKDGQIILDLGCGLGALAFHIAQKYTNCNVTSVTNSVKQKEFIEEKCKILNVSNVKVILTDICTLEMEATFDRIFAIGLIEHMKNYELLLRKFSAWMKQDGLLFIEHLCHKTLGYHNEPIDEDDWYTAYFFPAGTLTFIPSSFLLYFQDDVSVVNHWTLSGKHFSRSNEEWLKRMDNKIDEVKEIYKAAASETKDDDIMKLIRLWRFLSISAAEMFGYKDGEEWMISQVLFKKK

SEQ ID NO: 77 - protein, 366 aa, Eschscholzia californica
MASLVEEGSFVNNKESVKERVSELVKRLKNGLVSDEELRKLMRVELEKRLEWGYKSTHEQQLSQLIDLAHSMKKMEIAMEIDALNSTVYEVPLSFLQIIHGTTIKESCLYFKDESTTVDEAEIAMMDLYLERAQIKDGQSILDLGCGLGGFSFHIASKFTGCNITAVTNSVKQKEFIEEKCKTLNVPNIKVILADICTTEIENVFDRIIAIGLIEHMKNYELLLKKFSKWMTQDGLLFIEHLCHKTFGYHNEPLDEDDWYTTYFFPAGTLTFIPSSFLLYFQDDVSVVDHWTLNGKHFARSNEEWLKRMDEKMDEVKQIFRSNLKSENEVTKTIGEWRFLSMSAAEMFGYNNGEEWMVSQLLFKKK

SEQ ID NO: 78 - protein, 362 aa, Glaucium flavum
MGSNETNGELKTKEMVPDLLKRLESGLVADEELRKLIRFELERRLKWGYKPTHEQQLAELLKLAHSTKQMKIATETDSLNSTMYEVPIPFLQLQFGSAIKESCCYFKDESTTLDEAEVAMMDLYLERTQIKDGQSILDLGCGLGALAFHIVQKYPNCNVLAITNSVEQKEFIEEKCKIRKVENVKVSLADICTLEMKTTFDRIFAIGLLEHMKNYQLLLKKFSNWMKQDGLLFIEHLCHKTLAYHYEPLDEDDWYTEYFFPAGTLTIISSSFLLYFQDDVSIVNHWSLSGKHFSRSNEEWLKRMDMKIDEVKEILEAAFENKDHDITKLINHWRFLAINATEMFGYNNGEEWMVSQVLFKKK

SEQ ID NO: 79 - protein, 365 aa, Sanguinaria canadensis
MASDHEVSNKELKKKKEVITELLKRLESGLVSDEELRGLIRFELERRLRWGYKPTHEQQLAQLLNLAHSMKQMKIATEIDALNSTMYEVPIPFLQIQLGSTLKESCCYFKDESTTVDEAEIAMMDLYLERAQIKDGQSILDLGCGLGALAFHIAQKYTNCNITAITNSVRQKEFIEEKCKILNVSNVKVSLADICTLEMEATFDRIFAIGLIEHMKNYELLLKKFSEWMKQDGLIFIEHLCHKTLAYHYEPLDEDDWYTEYFFPAGTLTLISSSFLLYFQDDVSVVDHWTLSGKHFSRSNEEWLKRMDEKIDEVKEIFESVSDSKDDDVTKLINHWRFFCISSAEMFGYNNGEEWMISQVLFKKK

SEQ ID NO: 80 - protein, 371 aa, Corydalis cheilanthifolia
MIKKSKIMAFSDHHHEVVKNHSKKEMIADLLKRLEAGLVPDEEMRNLFRFELERRLQWGYKSIHQEQLSQLLKLAHSTKEMTIVAEMDALNSSMYELPISFLQIQLGSNLKQSSLYFKDELTTVDEAEVAIMDLYLERAQIEDGQSILDLGCGLGAFSFHVARKYTNCNITAVTNSLTQKEFIEKKSKILNIQNVKVIFADVTTVEMETTFDRVFAIGLIEHMQNYELFLKKLSKWMKQDGLLFIEHFCHKTLAYHYKPIDEDDWFTNLLYPNGTVISSSLLLYFQDDVSVVDHWSLSGKHFSRASEESLKRMDAKMDEMKEIFESITDSKEEAMKLINQWRIFCISCAEMFGYNNGEEWMTSHFLFKKKL

SEQ ID NO: 81 - protein, 373 aa, Corydalis cheilanthifolia
MGSSTASDHEMVIMENDSKNKQVVIADLLKRLVGGLVPDEEMRNMFRFELEKRLKWGYKSTHQQQLSQLLNLVELNKGIAKIAPEMDALNSAMYEVPIPYLKLMLGSTLKQSCLYFKDESTTLDEAEIEMMDLYLERADIQDGQSILDLGCGLGGLGFHIAQKYISCNITALTNSLTQKEFIEEKCKTLNIPNVKVILADVTTVEIETTFDRLFAIGLVEHMENYELFLRKLSKWMKQDGLLFIEHLCHKTLAYHYKPIDEDDWYSNLLYPTGTLTSASFLLYFQDDLSVVDHWSLSGKHFSRATEEWLKMIDANMDKIREIYESVTESKEEATRSINQWRIFCISCAEMFGYNDGEEWMISHFLFKNKKQIE

SEQ ID NO: 82 - protein, 366 aa, Corydalis cheilanthifolia
MATSDQEVKTSKMEMIADLLKRLEAGLVPDDEIRSLIRVELERRLKWGYKSTHQEQLDQLLNLAHSIKKMKIASTEMDGLTSTMYEVPISLVQIQLGSHLKESCLYFKDETTTVDEAEIAMMDLYLERAQIKDGQSILDLGCGLGAVSFHIAQKYTSCNITAVTNSVRQKEFIEEKSKTLNVPNVKVLLADITTLEMEHTFDRLFAISLIEHMENYELLLRKLSEWMKQDGLLFIEHLCHKTLSYHFEPMDEDDWYTNLLFPAGTLTLVSASFLLYFQDDLSVVNQWVMSGKHFSRANEEWLKNMDAKMDEMREIFESITDSEEEVVKLINHWRIFCISSAEMFAYNDGEEWMNSHVLFKKKKQIQ

SEQ ID NO: 83 - protein, 359 aa, Corydalis cheilanthifolia
MAGSGANKEMIADLLKRLEVGLVPDEEIRSLIRFQLKRRLKWGYKTTHQEQLEQLLSLAHSIRKMKIATEMDALNSTMYEVPISFMQIVFGSTLKESCLYFKDEATTVNEAEIAMMDLYLERAQIKDGQSILDLGCGMGSLCFHIARKYTNCNITAVTNSVSQKEFIEEKSKTLNLPNVKVILADITTLEMDDTYDCLFAIGLIEHMKNYELLLRKLSNWMKQDSLLFIDHVCHKTLAYHYEPIDEDDWYTNLLFPAGTLTLVSASFLLYFQDDLSLVDHWSMSGKHFSRTNKEWLKNIDGKMDKIREIVKSITDSEEEVVKLINHWRMLCINSSEMFGFNDGEEWMNSHVLFKKKKQI

SEQ ID NO: 84 - protein, 353 aa, Sanguinaria canadensis
MEMIADLLKRLEAGLVPDDEIRSLIRVELERRLKWGYKSTHQEQLDQLLNLAHSIKKMKIASTEMDGLTSTMYEVPISLVQIQLGSHLKESCLYFKDETTTVDEAEIAMMDLYLERAQIKDGQSILDLGCGLGSVCFHIARKYTSCNITAVTNSVSQKEFIEEKSKTLNVPNVKVLLADITTLEMDDTFDCLFAIGLIEHMENYELLLRKLSDWMKQDGLLFIDHVCHKTLSYHFEPMDEDDWYTNLLFPAGTLTLVSASFLLYFQDDLSLVDHWSMSGKHFSRTNKEWLKNIDGKMDKIREIVKSITDSEEEVVKLINHWRMLCINSSEMFGFNDGEEWMNSHVLFKKKKQI

SEQ ID NO: 85 - protein, 363 aa, Papaver bracteatum
MCTTMDTTKISQQDDLWKNMELGLISDEEVRRLMKIETEKRIKWGTKPTQQEQLAQLLDFNKSLRGMKMATEVHALENHKIYEIPDSFNQIIGGKESAGLFTDEATTTIEEANTKMMDLYCERAGLKDGQTILDIGCGAGLLVLHLAKKYKNCKITGVTNTSWHKEHILEQCKNLNLSNVEVILADVTTVDIERTFDRVFVIGLIEHMKNFELFLRKISKWMKDDGLLFLEHLCHKSFSDHWEPLSEDDWYAKNFFPSGTLVIPSATCLLYFQEDVTVKDHWLLSGNNFARSNEAILKRIDSKIEEVKDIFMSFYGIGEEEAVKLINWWRLLCITANELFKYNNGEEWLISQLLFKKKLMTCI

SEQ ID NO: 86 - protein, 366 aa, Papaver bracteatum
MVKGDQFQTTTMEETKISQENDLWTNMELGLIPDEEVRRLMKIEIEKRIEWGMKPTQHQQLAQLLDFTKSLRGMKMATELDKLDSKLYETPHSFNQIVNGSTLKESSGLYTDVTTTMDEASIKMMDLYCERANIKDGQTILDLGCGPGPLVLHIAKKYSNCKITGVTNAFSQREYILEECKKLSLSNVEIILADVTSLDLETTFDRVFVIGFIEHMKNFELFLRKISKWMKDDAVLFLEHFCHKSFSYHGEPLSEDDWYAKNFFAPGTLVIPSATCLLYFQEDLAVIDHWFLSGNHFARTNEEMLKGIDGKIEEIKDIFMSFYGINEAEAVKLINWWRLFCITGAEMFSYNNGEEWFISQLLFKKK

SEQ ID NO: 87 - protein, 364 aa, Eschscholzia californica
MALEQEDSMSVPERNEGVADLIKRMELGLVNDEEIRRLMRIQIENRLKWGYKPTHDQQLAQHLHFINSLKEMKMATEMDSLDSQVYESPNSFQQIMCGRSMKESAGLFMDDVTTVEEAHIRMMDLYCDKATFEDGQKILDLGCGHGSVVLHVAQKYKGCQVTGVTNSSAQKQYILEQCKKLDLSNVEIILADVTTLEMEEKFDRVIIIGLIEHMKNFKLFFQKVSKWMKEGGLLFLENYFHKDFAYHCEKIDEDDWYDGYIFPPGSLLMPSASTLLYFQEDLTVADHWVLPGTHFAKTFEEFLKKIDLRIEEVREIFEAFYGISKEEAMKLSNYWRNFCISAMEIFNYNNGQEWMISHLLYTKK

SEQ ID NO: 88 - protein, 368 aa, Chelidonium majus
METGKNNQNMKTTIDDLWNQMMLGIVPDKEIRRLMKIELKKRLDWGYRPTHQQQLSQLLDFAKGLCNYCWTALRCMKMSAEFDTLDSKVYETPKSFQQIMCGTTIKESSGLFMNESTTLDQAQISMLDLYFDKAKIKDGQSILDLGCGHGALILYLAQKYQNCNITGVTNSLSQKEFIVEKCKKLGLSNVEILLADVTKLEMEDMFDRVFVIGLIEHMKNFELFLRKISEWMKPDGLLFLEHYCHKSFAHQWEPIDEEDWFSKYIFPPGTVIIPSASFLLYFQEDVKVIDHWTLSGNHFARTQEEWLKGIDGHIDEVEKTFESFYGISKEEAVKLINFWRVFCLSGVEMFGYNNGEEWMISHLLFKKK

SEQ ID NO: 89 - protein, 361 aa, Glaucium flavum
MTMEANNAKKEAIENLWEQMMMGLVPDHEITRLMKSELQKRLNWGYKPTHQQQISQLLDFAKSLRRMEMSLDFDNLELDTKMYETPESFQLIMSGTTLKESSGLFTDETATLDQTQIRMMDLYLEKAKIKDGQSILDLGCGHGALILHVAQKYRNCNVTGVTNSIAQKEFIFKQCKKLGLSNVEMVLADVTKCEMKATFDHIFVIGLIEHMKNFELFLRKVSEWMKSDGLLFMEHYCHKSFAYQWEPMDDDDLFSKYVFPPGSAIIPSASFLLYFQDDLTVVDHWTLSGNHFARTHQEWLKRIDSQSDEIKGIFESFYGISKEEAVKLINYWRVFCLFGVEMFGYNNGEEWMISHLLFKKK

SEQ ID NO: 90 - protein, 363 aa, Corydalis cheilanthifolia
MEVVATSSARNPKKEIVDLWKRMELGLIPDEEIRDLMKIGLEKRLKWGYKPTHEQQLSQLLHFAKSLRSMKMASEMETLDDQMYETPTAFQQLMCGSTIKESAGFFKDESTTLDEAEIKMLDLYCEKARIEDGQKILDLGCGHGAVMLHIAQKYKNCNVTGVTNSISQQQFIVQRSKELNLSNVNMILADVTMLEMDATYDRIFIIGLIEHMKNFELFLRKISKWITKEGLLFLEHYCHKTFAYQCEPVDEDDWYNMFIFPPGTLILPSASFLLYFQDDLIVVDRWTLNGNHYARTQEEWLKRIDANVDGVKQMFESVCDGNKEEAVKLMNFWRIFCISGAEMLAYNNGEEWMISHYLFKKRN

SEQ ID NO: 91 - protein, 357 aa, Nigella sativa
MEATQITKKQGVAELIKRIENGQVPDEEITRMMKIQIQKRLKLGYKSTHEQQLAQLLHFVHSLQKMEMAEEVDTLDSELYEIPLPFLHIMCGKALKFSPGYFKDESTTLDESEVNMLDLYCERAQIEDGQTILDLGCGHGSLTLHVAKKYRGCKVTGITNSVSQKDFIMEECKKLNLSNVEIILEDVTKFETGTTYDRIFAVALIEHMKNYELFLKKVSAWMAQDGLLFVEHHCHKVFAYKYEPIDDDDWYTEYIFPTGTLVMSSSSILLYFQEDVSVVNHWTLSGKHPSLGFKQWLKRIDDNIDEIKEIFESFYGSKEKATKFITYWRVFCIAHSEMYATNGGEEWMLSQVLFKRK

SEQ ID NO: 92 - protein, 349 aa, Sanguinaria canadensis
MGGVADLLKKMELGLVPEEEIRRLMRIIIEKRLEWGYKPTHAEQLDHLTNFIQCLRGMKMADEIDALDAKMYEIPLPFMQTICGSTLKFSPGYFKDESTTLDESEIHMMDLYCERAEVKDGHSILDLGCGHGGFVLHVAQKYKNSIVTGVTNSVAEKEFIMTQCKKLCLSNVEIILADVTKFEPETTYDRVFAIALIEHMKNYELVLEKLSKWVAQDGFLFVEHHCHKVFPYKYEPLDEDDWYTEYIFPGGTIVLPSASILLYFQKDVSVVNHWSLNGKHPARGFKEWLKRLDENMDAVKAIFEPFYGSKEEAMKWITYWRVFCITHSEMYAYNNGEEWMLSQVLFKRK

SEQ ID NO: 93 - protein, 350 aa, Jeffersonia diphylla
MSKGVAKLVERMELGLVSDDEVRRLMRILIEKRLKWGYKPTHEEQLTYLTNFIQGLKGMKIAEEIDALDAKMYEIPIAFMQILCGYSLKFSPGFFEDESTTLDESETIMMDLYCERAQVQDGQSILDLGCGHGGFVLHVAQKYKNCKVTGVTNSVSETEYIMEQCKKLGLSNVEIIIADVTKFEPEVTYDRVFAIALIEHMKNYELVLQKLSKWVAQDGFLFVDHHCHKVFPYKYEPIDEDDWYTQYIFPGGTLVLPSASILLYFQEDVSIVNHWTLSGNHPARGFKEWLKRLDDNMDEIKAIFEPFYGSKEEAMKWITYWRVFCITHSEMYAYNGGEEWMISQVLFKRK

SEQ ID NO: 94 - protein, 357 aa, Berberis thunbergii
MEVKQAGKEGVTELLVKRMELGLVPEEEIRRLMRIQIQKRLDWGYKPTHEEQLAHLTKFIQNIRGMKMADEIDALDAKMYEIPLPFLQTICGKTLKFSPGYFKDESTTLDESETLMMDLYCERAQVKDGQSILDLGCGHGGFVLHLAQKYRNSVVTGVTNSVSETEYIKEQCKKLGLSNVEIIIADVTKFEPEVTYDRVFAIALIEHMKNYALVLNKISKWVAQDGYLFVEHHCHKVFPYKYEPLDEDDWYTNYIFPGGTLILPSASILLYFQEDVTVLNHWSLSGKHPSRGFIEWLKRLDENIDVIMGIFEPFYGSKEEATKWINYWRVFCMTHSEMYAYGNGEEWMLSQVLLKRK

SEQ ID NO: 95 - protein, 339 aa, Berberis aquifolium
MELGLVPEKEIRRLMRIQIQKRLEWGYKPTHEEQLAHLTKFIQNIRGMKMADEIDALDAKMYEIPLPFLQTICGKTLKFSPGYFKDESTTLDESETLMMDLYCERAQVKDGQSILDLGCGHGGFVLHLAQKYRNSIVTGVTNSVSETEYIKEQCKKLGLSNVEIIIADVTKFEPEVTYDRVFAIALIEHMKNYALVLNKISKWVAQDGYLFVEHHCHKVFPYKYEPLDEDDWYTNYIFPGGTLILPSASILLYFQEDVTVLNHWSLSGKHPSRGFIEWLKRLDENIDVIMGIFEPFYGSKEEATKWINYWRVFCITHSEMYAYGNGEEWMLSQVLLKRK

SEQ ID NO: 96 - protein, 355 aa, Menispermum canadense
  SITE 86: note = variant - Any amino acid
  SITE 96: note = variant - Any amino acid
  SITE 127: note = variant - Any amino acid
  SITE 138: note = variant - Any amino acid
  SITE 153: note = variant - Any amino acid
  SITE 292: note = variant - Any amino acid
MDKANERELKRAELFKKLEDDLVTYDEIKQVMRTELAKRLEWGYKPTHQQQLAHLLDFAHALEGMKIANEVETLASEVYETPLPFXEIVLGPAKKXSSCLFEDESTTLEQAEIAMLDLYFERAQIRXGMSVLDLGCGXGSVGLHIARKYKNCXVTCITNSISQKQYIENQCKLYNLSNVKIILADIVAHDTDDTFDVVLVIGVIEHMKNYALLLNKISKWMAKDGLLFVEHLCHKTFPYHFEPLDEDDWYSNFVFPTGTLTMPSVSFLLYFQADVSILNHWILSGKNFSRTXEEFLKRIDANVDAIKDGLKPSLGSEGVAKLISYWRGFCLTGMEMFGYNNGEEWMVSQVLFKNK

SEQ ID NO: 97 - protein, 366 aa, Tinospora cordifolia
MEDNNNLLQEEMNVVELLQRPELGLVPDEKIRKLTRLQLQKRLKWGYKPTHEAQLSHLFQFIHSLPSLNMESEDENPKSWLYETPTSFLQLLYGDCIKESDTYYKEDTATLEEAVINMLELYCERARITEGLSVLDLGCGYGALTLHVAQKYKSCKVTGVTSSISQKQYIMEKCKKLNLTNVEIILADVATIEIEAASYDRIFALGIFEHVNDYKLFLGKLSKWMKQDGLLFVEYLCHKTFPYQNKPLDKGDKWYNEYVFPSGGLIIPSASFILYFQNDVSVVRQWTQGGQHSARTFEELLKRIDGNIDKIKEIFIESYGSKEDAVRFINYWRVFLITGVEMFSYNDGEEWMGAHFLFKKKFIMQE

SEQ ID NO: 98 - protein, 359 aa, Cissampelos mucronata
MEVKQSKGDELRSRVAELLERPELGLVPDEEIRRLAKARLEKRLKWGYKATHGEQLSSLLQFVESLPSLNMASEDDSPKAWLYETPTSFLQLIYGDIIKESGSYYKDESTTLEEAMIHNMNLCCERANIKEGQSVVDLGCGYGAFILHVAQKYKTCRVTGITSSISQKHYIMEQCKKLNLSNVEVILADVATIKLDATFDRVFAAGMFEHVNDYKSFLRKITNWMKPDGRLFVEHLCNKTFPYQNKPLDDGDNWGEYVFPSGGLIIPSASLLLYFQEDVSIVNHWTFSGKHAANKFEELLKRIDAKIDAIKRIFNECYGSKDSIRFINYWRVFLITAAEMFGYNNGEEWMGVHLLFKKK

SEQ ID NO: 99 - protein, 352 aa, Cocculus trilobus
GLKSSVAELLERPELGLVPDGEIRKLTKTRLAKRLEWGYKATHEDQLSHLLRFIHSLPSLNMASEDDSPKAWLYETPTSFLQLIYGDIIKESGTYYKDESSTLEEAIIHNMDLCCERARIKEGQSVLDLGCGYGAFTLHVAQKYKSCSVTGITSSISQKDYIMEQCKKLNLSNVEVILADVATIKMNTTFDRVFALGMFEHINDYKLFLRRISNWMKHDGLLFVEHLCNKTFAYQNKPLDDGDDWFNEYVFPSAGLIIPSASLLLYFQEDVSIVHHWTFSGKHAAYKFEELLERIDAKIEAIKEIFIECYGSKEDAIRFINYWRVFLITAAEMFAYRDGEEWMGSHVLFKKK

SEQ ID NO: 100 - protein, 364 aa, Cissampelos mucronata
MEAKQHESNNNIDEELKNRVNIGEQEERPGFEDEEIRRLAKAQLAKRLKWGYKPTHEQQLSHLLQFLQSLPSLNMASEDESSKAWLYETPTSFLQLLFGNVIKFSGYYYKHESSTFEESMIHNMDLCCERANIKEGQNVIDLGCGYGAFVLHVAQKYKSCSVTGITCSITQKHHIMEECKKLNLCNVKVILADVATIELGTAFDRVFAFGMFEEINDYKLILRKISNWMKPDGLFFVEHLCHKTLAYQNKLIDDQDWYEEYIFPSGGLIVPSASLLLYFQDDLSVVYHWTYNGKHGARSFEKMLERTDANIDTIKDMFTEFYGSKEKAIKFINYWRVFFITAAEMFAYNDGEEWMCSQLLFKKK

SEQ ID NO: 101 - protein, 364 aa, Cissampelos mucronata
MEHKIEDIRKLKSRVEEQLERPELGLVKDEDIKTLAKAKLEKRLKWGYKPTYAEQLSNLLQFAQSLPSLKMENVDDQGSSKQWLYGVPSEFLQIIYGGIIKMSGSYYEDESTTLEESMIKDMDSCCEKANVKEGHSVLDIGCGYGSLIIHIAKKYRTCNVTGITNFVEQKQYIMEECKKLNLSNVEVIVGDGTTINLNTTTFDRVFVTGMLEEINDYKLFLKSVSDWMKPDGLLLVTHFCHKTFAYQNNKALDDEDWHNEYIFPSGNLIVPSASLLLYFQEDLSVVSHWATNGTHTGRTCKKLVERIDANIEKIKEIFSEFYGSKEDAIRMINYWRVLCITGAEMYTCKDGEEWMDVYYLFKKK

SEQ ID NO: 102 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETATLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 103 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIIAFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETATLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 104 - protein, 1049 aa, synthetic construct; identical to SEQ ID NO: 37
  REGION 1..1049: note = Description of Artificial Sequence: Synthetic polypeptide

SEQ ID NO: 105 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 106 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIIAFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 107 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctttgaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctttggatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccgctaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 108 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctttgaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcgctttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccgctaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 109 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 110 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctgctgttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 111 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctgctgttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcgctttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 112 - DNA, 5372 bp, synthetic construct
  misc_feature 1..5372: note = Description of Artificial Sequence: Synthetic polynucleotide
  source 1..5372: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 113 - DNA, 4977 bp, synthetic construct
  misc_feature 1..4977: note = Description of Artificial Sequence: Synthetic polynucleotide
  source 1..4977: mol_type = other DNA
aacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgagagcgttggttggtggatcaagcccacgcgtaggcaatcctcgagcagatccgccaggcgtgtatatatagcgtggatggccaggcaactttagtgctgacacatacaggcatatatatatgtgtgcgacaacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcg

SEQ ID NO: 114 - DNA, 5079 bp, synthetic construct
  misc_feature 1..5079: note = Description of Artificial Sequence: Synthetic polynucleotide
  source 1..5079: mol_type = other DNA
acgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgactcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcga

SEQ ID NO: 115 - protein, 120 aa, synthetic construct
  REGION 1..120: note = Description of Artificial Sequence: Synthetic polypeptide
MEESLWVVTATVVVVFALAKLLKKSSSISTMEWPKGPKKLPIIGNLHQLGGEAFHVVLANLAKIHGTVMTIWVGAWRPMIVISDIDKAWEVLVNKSSDYAGRDFPEITKIISANWKNISC

SEQ ID NO: 116 - protein, 120 aa, synthetic construct
  REGION 1..120: note = Description of Artificial Sequence: Synthetic polypeptide
MAPINIEGNDFWMLACTVILVFALVKFMFSKISFYQSANTTEWPAGPKTLPIIGNLHQLGGGVPLQVALANLAKVYGGAFTIWIGSWVPMIVISDIDNAREVLVNKSADYSARDVPDILK

SEQ ID NO: 117 - protein, 10 aa, synthetic construct
  REGION 1..10: note = Description of Artificial Sequence: Synthetic peptide
MEESLWVVTA